(19) United States
(12) Patent Application Publication
(10) Pub. No.: US 2007/0032475 A1
(43) Pub. Date: Feb. 8, 2007

(54) Novel compounds useful for bradykinin B1 receptor antagonism

(76) Inventors: Xiaocong Michael Ye, Palo Alto, CA (US); Albert W. Garofalo, South San Francisco, CA (US); Rose D. Lawler, San Francisco, CA (US); Juri Y. Fukuda, Oakland, CA (US); Andrei W. Konradi, Burlingame, CA (US); Ryan Holcomb, Bellevue, WA (US); Kassandra I. Rossiter, San Jose, CA (US); David W.G. Wone, Newark, CA (US); Jing Wu, Redwood City, CA (US)

Correspondence Address: ELAN PHARMACEUTICALS/FINNEGAN, HENDERSON;FARABOW, GARRETT & DUNNER, LLP, 901 NEW YORK AVENUE, N.W., WASHINGTON, DC, 20001-4413, US

(21) Appl. No.: 11/398,711

(22) Filed: Apr. 6, 2006

Related U.S. Application Data
(60) Provisional application No. 60/671,537, filed on Apr. 15, 2005.

(51) Int. Cl. A61K 31/5513; A61K 31/55; A61K 31/4709; A61K 31/4545; A61K 31/454; C07D 217/22; C07D 211/68

(52) U.S. Cl. 514/217.12; 514/221; 514/316; 514/318; 540/504; 540/605; 546/193; 546/142; 514/309

(57) ABSTRACT

Disclosed are compounds that are bradykinin B₁ receptor antagonists and are useful for treating diseases, or relieving adverse symptoms associated with disease conditions, in mammals mediated by bradykinin B₁ receptor.

### CROSS-REFERENCE TO RELATED APPLICATION

This application claims the benefit of priority under 35 U.S.C. § 119(e) to U.S. Provisional Application 60/671,537, filed Apr. 15, 2005.

### FIELD OF THE PRESENT INVENTION

The present invention is directed to compounds and methods useful as bradykinin B₁ receptor antagonists which may relieve adverse symptoms in mammals mediated, at least in part, by a bradykinin B₁ receptor including pain, inflammation, septic shock, scarring processes, and the like.

### BACKGROUND OF THE PRESENT INVENTION

Bradykinin (“BK”) or kinin-9 is a kinin that plays an important role in the patho-physiological processes accompanying acute and chronic pain and inflammation. BKs, like other related kinins, are autocoid peptides produced by the catalytic action of kallikrein enzymes on plasma and tissue precursors termed kininogens.

BK is a vasoactive nine-amino acid peptide (Arg-Pro-Pro-Gly-Phe-Ser-Pro-Phe-Arg) that is formed locally in body fluids and tissues from the plasma precursor kininogen during inflammatory processes. It is a potent but short-lived agent of arteriolar dilation and increased capillary permeability. BK is also known to be one of the most potent naturally occurring stimulators of C-fiber afferents mediating pain, and a physiologically active component of the kallikrein-kinin system.

BK, the nonapeptide sequence pH-Arg¹-Pro²-Pro³-Gly⁴-Phe⁵-Ser⁶-Pro⁷-Phe⁸-Arg⁹-OH (“SEQ. ID. NO. 1”) is formed by the action of plasma kallikrein, which hydrolyses the sequence out of the plasma globulin kininogen. Plasma kallikrein circulates as an inactive zymogen, from which active kallikrein is released by Hageman factor. Glandular kallikrein cleaves kininogen one residue earlier to give the decapeptide Lys-bradykinin (kallidin, Lys-BK) (“SEQ. ID. NO. 2”). Met-Lys-bradykinin (“SEQ. ID. NO. 3”) is also formed, perhaps by the action of leukocyte kallikrein. Pharmacologically important analogues include des-Arg⁹ (amino acid 1-8 of SEQ. ID. NO. 1) or BK₁₋₈ and Ile-Ser-bradykinin (or T-kinin) (“SEQ. ID. NO. 4”), [Hyp³]bradykinin (“SEQ. ID. NO. 5”), and [Hyp⁴]bradykinin (“SEQ. ID. NO. 6”). See e.g., Oxford Dictionary of Biochemistry and Molecular Biology, Oxford University Press (2001). BK is also released from mast cells during asthma attacks, from gut walls as a gastrointestinal vasodilator, from damaged tissues as a pain signal, and may be a neurotransmitter.

BK is also a powerful blood-vessel dilator, increasing vascular permeability and causing a fall in blood pressure, an edema-producing agent, and a stimulator of various vascular and non-vascular smooth muscles in tissues such as uterus, gut and bronchiole. BK is formed in a variety of inflammatory conditions and in experimental anaphylactic shock. The kinin/kininogen activation pathway has also been described as playing a pivotal role in a variety of physiologic and pathophysiologic processes, being one of the first systems to be activated in the inflammatory response and one of the most potent simulators of: (i) phospholipase A₂ and, hence, the generation of prostaglandins and leukotrienes; and (ii) phospholipase C, and thus, the release of inositol phosphates and diacylgylcerol. These effects are mediated predominantly via activation of BK receptors of the BK₂ type.

A BK receptor is any membrane protein that binds BK and mediates its intracellular effects. Two recognized types of receptors are B₁ and B₂. On B₁ the order of potency is,

des-Arg⁹-bradykinin (BK₁₋₈ or amino acid 1-8 of SEQ. ID. NO. 1)=kallidin (SEQ. ID. NO. 2)>BK (SEQ. ID. NO. 1).

On B₂ the order of potency is,

kallidin (SEQ. ID. NO. 2)>BK (SEQ. ID. NO. 1)>>BK₁₋₈.

Hence, BK₁₋₈ is a powerful discriminator. See e.g., Oxford Dictionary of Biochemistry and Molecular Biology, Oxford University Press (2001).

B₁ receptors are considerably less common than B₂ receptors, which are present in most tissues. The rat B₂ receptor is a seven-transmembrane-domain protein that has been shown on activation to stimulate phosphoinositide turnover. Inflammatory processes induce the B1 subtype. See, e.g., Marceau, Kinin B₁ Receptors: A Review, Immunopharmacology, 30:1-26 (1995) (incorporated herein by reference in full). The distribution of receptor B₁ is very limited since this receptor is only expressed during states of inflammation.

BK receptors have been cloned for different species, notably the human B₁ receptor (See e.g., J. G. Menke, et al., J. Biol. Chem., 269(34):21583-21586 (1994) (incorporated herein by reference in full) and J. F. Hess, Biochem. Human B₂ Receptor, Biophys. Res. Commun., 184:260-268 (1992) (incorporated herein by reference in full)). Examples of such receptors include B₁, database code BRB1_HUMAN, 353 amino acids (40.00 kDa); and B₂, database code BRB2_HUMAN, 364 amino acids (41.44 kDa). See, e.g., Oxford Dictionary of Biochemistry and Molecular Biology, Oxford University Press (2001).

Two major kinin precursor proteins, high molecular weight and low molecular weight kininogen, are synthesized in the liver, circulate in plasma, and are found in secretions such as urine and nasal fluid. High molecular weight kininogen is cleaved by plasma kallikrein, yielding BK, or by tissue kallikrein, yielding kallidin. Low molecular weight kininogen, however, is a substrate only for tissue kallikrein. In addition, some conversion of kallidin to BK may occur inasmuch as the amino terminal lysine residue of kallidin is removed by plasma aminopeptidases. Plasma half-lives for kinins are approximately 15 seconds, with a single passage through the pulmonary vascular bed resulting in 80-90% destruction. The principle catabolic enzyme in vascular beds is the dipeptidyl carboxypeptidase kininase II or angiotensin-converting enzyme (ACE). A slower acting enzyme, kininase I, or carboxypeptidase N, which removes the carboxyl terminal Arg, circulates in plasma in great abundance. This suggests that it may be the more important catabolic enzyme physiologically. Des-Arg⁹-bradykinin (amino acid 1-8 of SEQ. ID. NO. 1) as well as des-Arg¹⁰-kallidin (amino acid 1-9 of SEQ. ID. NO. 2) formed by kininase I acting on BK or kallidin, respectively, are acting BK₁ receptor agonists, but are relatively inactive at the more abundant BK₂ receptor at which both BK and kallidin are potent agonists.

Direct application of BK to denuded skin or intra-arterial or visceral injection results in the sensation of pain in mammals, including humans. Kinin-like materials have been isolated from inflammatory sites produced by a variety of stimuli. In addition, BK receptors have been localized to nociceptive peripheral nerve pathways and BK has been demonstrated to stimulate central fibers mediating pain sensation. BK has also been shown to be capable of causing hyperalgesia in animal models of pain. (See, e.g., R. M. Burch, et al., Bradykinin Receptor Antagonists, Med. Res. Rev., 10(2):237-269 (1990) (incorporated herein by reference in full); Clark, W. G. Kinins and the Peripheral Central Nervous Systems, Handbook of Experimental Pharmacology, Vol. XXV: Bradykinin, Kallidin, and Kallikrein. Erdo, E. G. (Ed.), 311-322 (1979) (incorporated herein by reference in full)).

Several lines of evidence suggest that the kallikrein/kinin pathway may be involved in the initiation or amplification of vascular reactivity and sterile inflammation in migraine. (See, e.g., Back, et al., Determination of Components of the Kallikrein-Kinin System in the Cerebrospinal Fluid of Patients with Various Diseases, Res. Clin. Stud. Headaches, 3:219-226 (1972) (incorporated herein by reference in full). Because of the limited success of both prophylactic and non-narcotic therapeutic regimens for migraine, as well as the potential for narcotic dependence in these patients, the use of BK antagonists offers a highly desirable alternative approach to the therapy of migraine.

BK is produced during tissue injury and can be found in coronary sinus blood after experimental occlusion of the coronary arteries. In addition, when directly injected into the peritoneal cavity, BK produces a visceral type of pain. (See, e.g., Ness, et al., Visceral pain: a Review of Experimental Studies, Pain, 41:167-234 (1990) (incorporated herein by reference in full). While multiple other mediators are also clearly involved in the production of pain and hyperalgesia in settings other than those described above, it is also believed that antagonists of BK have a place in the alleviation of such forms of pain as well.

Shock related to bacterial infections is a major health problem. It is estimated that 400,000 cases of bacterial sepsis occur in the United States yearly; of those, 200,000 progress to shock and 50% of these patients die. Current therapy is supportive, with some suggestion in recent studies that monoclonal antibodies to Gram-negative endotoxin may have a positive effect on disease outcome. Mortality is still high, even in the face of this specific therapy, and a significant percentage of patients with sepsis are infected with Gram-positive organisms that would not be amenable to anti-endotoxin therapy.

Multiple studies have suggested a role for the kallikrein/kinin system in the production of shock associated with endotoxin. See, e.g., Aasen, et al., Plasma kallikrein Activity and Prekallikrein Levels during Endotoxin Shock in Dogs, Eur. Surg., 10:5062(1977) (incorporated herein by reference in full); Aasen, et al., Plasma Kallikrein-Kinin System in Septicemia, Arch. Surg., 118:343-346 (1983) (incorporated herein by reference in full); Katori, et al., Evidence for the Involvement of a Plasma Kallikrein/Kinin System in the Immediate Hypotension Produced by Endotoxin in Anaesthetized Rats, Br. J. Pharmacol., 98:1383-1391 (1989) (incorporated herein by reference in full); Marceau, et al., Pharmacology of Kinins: Their Relevance to Tissue Injury and Inflammation, Gen. Pharmacol., 14:209-229 (1982) (incorporated herein by reference in full). Recent studies using newly available BK antagonists have demonstrated in animal models that these compounds can profoundly affect the progress of endotoxic shock. See, e.g., Weipert, et al., Brit J. Pharm., 94:282-284 (1988) (incorporated herein by reference in full). Less data is available regarding the role of BK and other mediators in the production of septic shock due to Gram-positive organisms. However, it appears likely that similar mechanisms are involved. Shock secondary to trauma, while frequently due to blood loss, is also accompanied by activation of the kallikrein/kinin system. See, e.g., Haberland, The Role of Kininogenases, Kinin Formation and Kininogenase Inhibitor in Post Traumatic Shock and Related Conditions, Klinische Woochen-Schrift, 56:325-331 (1978) (incorporated herein by reference in full).

Numerous studies have also demonstrated significant levels of activity of the kallikrein/kinin system in the brain. Both kallikrein and BK dilate cerebral vessels in animal models of CNS injury. See, e.g., Ellis, et al., Inhibition of Bradykinin-and Kallikrein-Induced Cerebral Arteriolar Dilation by Specific Bradykinin Antagonist, Stroke, 18:792-795 (1987) (incorporated herein by reference in full); and Kamitani, et al., Evidence for a Possible Role of the Brain Kallikrein-Kinin System in the Modulation of the Cerebral Circulation, Circ. Res., 57:545-552 (1985) (incorporated herein by reference in full). BK antagonists have also been shown to reduce cerebral edema in animals after brain trauma. Based on the above, it is believed that BK antagonists should be useful in the management of stroke and head trauma.

Other studies have demonstrated that BK receptors are present in the lung, that BK can cause bronchoconstriction in both animals and man, and that a heightened sensitivity to the bronchoconstrictive effect of BK is present in asthmatics. Some studies have been able to demonstrate inhibition of both BK and allergen-induced bronchoconstriction in animal models using BK antagonists. These studies indicate a potential role for the use of BK antagonists as clinical agents in the treatment of asthma. See, e.g., Barnes, Inflammatory Mediator Receptors and Asthma, Am. Rev. Respir. Dis., 135:S26-S31 (1987) (incorporated herein by reference in full); R. M. Burch, et al., Bradykinin Receptor Antagonists, Med. Res. Rev., 10(2):237-269 (1990) (incorporated herein by reference in full); Fuller, et al., Bradykinin-induced Bronchoconstriction in Humans, Am. Rev. Respir. Dis., 135:176-180 (1987) (incorporated herein by reference in full); Jin, et al., Inhibition of Bradykinin-Induced Bronchoconstriction in the Guinea-Pig by a Synthetic B₂ Receptor Antagonist, Br. J. Pharmacol., 97:598-602 (1989) (incorporated herein by reference in full), and Polosa, et al., Contribution of Histamine and Prostanoids to Bronchoconstriction Provoked by Inhaled Bradykinin in Atopic Asthma, Allergy, 45:174-182 (1990) (incorporated herein by reference in full). BK has also been implicated in the production of histamine and prostanoids to bronchoconstriction provoked by inhaled BK in atopic asthma. See, e.g., Polosa, et al., Contribution of Histamine and Prostanoids to Bronchoconstriction Provoked by Inhaled Bradykinin in Atopic Asthma, Allergy, 45:174-182 (1990) (incorporated herein by reference in full). BK has also been implicated in the production of symptoms in both allergic and viral rhinitis. These studies include the demonstration of both kallikrein and BK in nasal lavage fluids and show that levels of these substances correlate well with symptoms of rhinitis. See, e.g., Baumgarten, et al., Concentrations of Glandular Kallikrein in Human Nasal Secretions Increase During Experimentally Induced Allergic Rhinitis, J. Immunology, 137:1323-1328 (1986) (incorporated herein by reference in full); Jin, et al., Inhibition of Bradykinin-Induced Bronchoconstriction in the Guinea-Pig by a Synthetic B₂ Receptor Antagonist, Br. J. Pharmacol., 97:598-602 (1989), and Proud, et al., Nasal Provocation with Bradykinin Induces Symptoms of Rhinitis and a Sore Throat, Am. Rev. Respir Dis., 137:613-616 (1988) (incorporated herein by reference in full).

In addition, studies have demonstrated that BK itself can cause symptoms of rhinitis. Steward et. al, discusses peptide BK antagonists and their possible use against effects of BK. See, e.g., Steward and Vavrek in Chemistry of Peptide Bradykinin Antagonists Basic and Chemical Research, R. M. Burch (Ed.), pages 51-96 (1991) (incorporated herein by reference in full).

These observations have led to considerable attention being focused on the use of BK antagonists as analgesics. A number of studies have demonstrated that BK antagonists are capable of blocking or ameliorating both pain as well as hyperalgesia in mammals including humans. See, e.g., Ammons, W. S., et al., Effects of Intracardiac Bradykinin on T₂-T₅ Medial Spinothalamic Cells, American Journal of Physiology, 249, R145-152 (1985) (incorporated herein by reference in full); Clark, W. G. Kinins and the Peripheral Central Nervous Systems, Handbook of Experimental Pharmacology, Vol. XXV: Bradykinin, Kallidin, and Kallikrein. Erdo, E. G. (Ed.), 311-322 (1979) (incorporated herein by reference in full)); Costello, A. H. et al., Suppression of Carageenan-Induced Hyperalgesia, Hyperthermia and Edema by a Bradykinin Antagonist, European Journal of Pharmacology, 171:259-263 (1989) (incorporated herein by reference in full); Laneuville, et al., Bradykinin Analogue Blocks Bradykinin-induced Inhibition of a Spinal Nociceptive Reflex in the Rat, European Journal of Pharmacology, 137:281-285 (1987) (incorporated herein by reference in full); Steranka, et al., Antinociceptive Effects of Bradykinin Antagonists, European Journal of Pharmacology, 136:261-262 (1987) (incorporated herein by reference in full); and Steranka, et al., Bradykinin as a Pain Mediator: Receptors are Localized to Sensory Neurons, and Antagonists have Analgesic Actions, Neurobiology, 85:3245-3249 (1987) (incorporated herein by reference in full).

Currently accepted therapeutic approaches to analgesia have significant limitations. While mild to moderate pain can be alleviated with the use of non-steroidal anti-inflammatory drugs and other mild analgesics, severe pain, such as that accompanying surgical procedure, burns and severe trauma require the use of narcotic analgesics. These drugs carry the limitations of potential abuse, physical and psychological dependence, altered mental status and respiratory depression, which significantly limit their usefulness.

Prior efforts in the field of BK antagonists indicate that such antagonists can be useful in a variety of roles. These include use in the treatment of burns, perioperative pain, migraine and other forms of pain, shock, central nervous system injury, asthma, rhinitis, premature labor, inflammatory arthritis, inflammatory bowel disease, neuropathic pain, etc. For example, Whalley, et al., has demonstrated that BK antagonists are capable of blocking BK-induced pain in a human blister base model. See Whalley, et al., in Naunyn Schmiederberg's Arch. Pharmacol., 336:652-655 (1987) (incorporated herein by reference in full). This suggests that topical application of such antagonists would be capable of inhibiting pain in burned skin, e.g., in severely burned patients that require large doses of narcotics over long periods of time and for the local treatment of relatively minor burns or other forms of local skin injury.

The management of perioperative pain requires the use of adequate doses of narcotic analgesics to alleviate pain while not inducing excessive respiratory depression. Post-operative narcotic-induced hypoventilation predisposes patients to collapse of segments of the lungs (a common cause of post-operative fever), and frequently delays discontinuation of mechanical ventilation. The availability of a potent non-narcotic parenteral analgesic could be a significant addition to the treatment of perioperative pain. While no currently available BK antagonist has the appropriate pharmacodynamic profile to be used for the management of chronic pain, anesthesiologists and surgeons in the management of perioperative pain already commonly use frequent dosing and continuous infusions.

A great deal of research effort has been expended towards developing such antagonists with improved properties. However, notwithstanding extensive efforts to find such improved BK antagonists, there remains a need for additional and more effective BK antagonists.

Two generations of peptidic antagonists of the B2 receptor have been developed. The second generation has compounds two orders of magnitude more potent as analgesics than first generation compounds. The most important derivative was icatibant. The first non-peptidic antagonist of the B2 receptor, described in 1993, has two phosphonium cations separated by a modified amino acid. Many derivatives of this di-cationic compound have been prepared. Another non-peptidic compound antagonist of B2 is the natural product Martinelline. See, e.g., Elguero, et al., Nonconventional Analgesics: Bradykinin Antagonists, An. R. Acad. Farm., 63(1):173-90 (Spa) (1997) (incorporated herein by reference in full); and Seabrook, et al., Expression of B1 and B2 Bradykinin Receptor mRNA and Their Functional Roles in Sympathetic Ganglia and Sensory Dorsal Root Ganglia Neurons from Wild-type and B2 Receptor Knockout Mice, Neuropharmacology, 36(7):1009-17 (1997) (incorporated herein by reference in full).

U.S. Pat. No. 3,654,275 teaches that certain 1,2,3,4-tetrahydro-1-acyl-3-oxo-2-quinoxalinecarboxamides have anti-inflammatory activity. See, e.g., McManus, U.S. Pat. No. 3,654,275, Quinoxalinecarboxamide Antiinflammatory Agents, issued Apr. 4, 1972 (incorporated herein by reference in full). International Patent Application WO 03/007958 filed on Jul. 2, 2002 and published on Jan. 30, 2003 discloses tetrahydroquinoxalines acting as BK antagonists. See, e.g., Beyreuther, B.; et al., International Patent Application WO 03/007958 A1 published on Jan. 30, 2003 (incorporated herein by reference in full). U.S. Pat. No. 5,916,908 teaches the use of 3,5-disubstituted pyrazoles or 3,4,5-trisubstituted pyrazoles as kinase inhibitors. See, e.g., Giese, et al., U.S. Pat. No. 5,916,908, issued Jun. 29, 1999 (incorporated herein by reference in full). Japanese Patent Application Serial No. 49100080 teaches 2-aminopyrazoles as anti-inflammatory agents. See, e.g., Yoshida, et al., Japanese Patent Application Serial No. 49100080 (incorporated herein by reference in full).

It has also been demonstrated that B1 receptors are upregulated by T lymphocytes in patients with secondary progressive multiple sclerosis and relapsing-remitting patients in active relapse. See, e.g., Prat, A.; Weinrib, L.; Becher, B.; Poirier, J.; Duquette, P.; Couture, R.; Antel, J. P. Bradykinin B1 receptor expression and function on T lymphocytes in active multiple sclerosis. Neurology, 53(9), 2087-2092 (1999).

Currently there are no marketed therapeutic agents for the inhibition of bradykinin B₁ receptor. In view of the above, compounds which are bradykinin B₁ receptor antagonists would be particularly advantageous in treating those diseases mediated by the bradykinin B₁ receptor.

Accordingly, it is an object of the present invention to provide compounds and methods of treatment useful as therapeutic agents for the inhibition of the bradykinin B₁ receptor. It is also an object of the present invention to provide compounds and methods of treatment useful in treating diseases, disorders, and conditions, which benefit from inhibition of the bradykinin B₁ receptor.

The present invention accomplishes one or more of these objectives and provides further related advantages.

### BRIEF SUMMARY OF THE PRESENT INVENTION

The present invention is directed to methods and compounds useful in treating diseases, disorders, and conditions, which benefit from inhibition of the bradykinin B₁ receptor.

### SUMMARY OF THE INVENTION

This invention is directed, in part, to compounds that are bradykinin B₁ receptor antagonists. It is also directed to compounds that are useful for treating diseases or relieving adverse symptoms associated with disease conditions in mammals, where the disease is mediated at least in part by bradykinin B₁ receptor. For example, inhibition of the bradykinin B₁ receptor is useful for the moderation of pain, inflammation, septic shock, the scarring process, etc. These compounds are preferably selective for antagonism of the B₁ receptor over the B₂ receptor. This selectivity may be therapeutically beneficial due to the up-regulation of the B₁ receptor following tissue damage or inflammation. Certain of the compounds exhibit increased potency and are expected to also exhibit an increased duration of action.

In an embodiment, the present invention provides a method of preventing or treating at least one condition which benefits from inhibition of the bradykinin B1 receptor, comprising:

administering to a host in need thereof a composition comprising a therapeutically effective amount of at least one compound of formula (I), 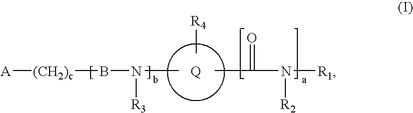
or a pharmaceutically acceptable salt thereof, wherein
a is 0 or 1;
b is 0 or 1;
c is 0, 1 or 2;
Q is an aryl, heteroaryl, cycloalkyl, or heterocycloalkyl ring;
R₁ is selected from
   —NR^{a}R^{b}
   -alkyl,
   -cycloalkyl,
   -heterocycloalkyl,
   -alkoxy,
   -aryl, and
   -heteroaryl;
   wherein the alkyl, cycloalkyl, heterocycloalkyl, alkoxy, aryl, and heteroaryl within R₁ are each optionally substituted with at least one group independently selected from R₂₀₀;
   wherein when R1 is an N-linked compound, then a is 0;
R^{a} and R^{b} are independently selected from
   -hydrogen, wherein R^{a} and R^{b} are not simultaneously hydrogen,
   -alkyl,
   -alkoxy,
   -cycloalkyl,
   -aryl,
   -heteroaryl, and
   -heterocycloalkyl;
   wherein the alkyl, alkoxy, cycloalkyl, aryl, heteroaryl and heterocycloalkyl within R^{a} and R^{b} are each optionally substituted with at least one group independently selected from R₂₀₀;
or R^{a} and R^{b} together with the nitrogen atom to which they are attached form a heteroaryl (optionally substituted with at least one group independently selected from R₂₀₀) or heterocycloalkyl (optionally substituted with at least one group independently selected from R₂₀₀);
R₂ is selected from hydrogen, alkyl, cycloalkyl, heterocycloalkyl, aryl, and heteroaryl;
   wherein the alkyl, cycloalkyl, heterocycloalkyl, aryl, and heteroaryl within R₂ are each optionally substituted with at least one group independently selected from R₂₀₀;
or R₁ and R₂ together with the nitrogen to which they are attached form a heterocycloalkyl (optionally substituted with at least one group independently selected from R₂₀₀) or a heteroaryl (optionally substituted with at least one group independently selected from R₂₀₀);
R₃ is selected from hydrogen and alkyl;
R₄ is selected from hydrogen, OH, alkyl, aryl, halogen, alkoxy, nitro, CN, cycloalkyl, amino, monoalkylamino, dialkylamino, amino carbonyl, monoalkylamino carbonyl, and dialkylaminocarbonyl;
B is selected from —C(O)— and —S(O)₂—;
A is selected from aryl substituted with formula A(a), wherein the aryl is optionally substituted with at least one group selected from R₅₀, heteroaryl (optionally substituted with at least one R₅₀ group), and formula A(a), 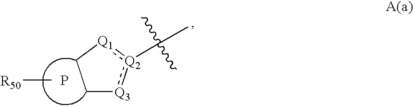
Q₁ and Q₃ are each independently selected from —C(R₆₀)₁₋₂—, —C(O)—, —O—, —N(R₆₀)₀₋₁—, and —S—;
Q₂ is selected from —CH—, —C— and —N—;
P is an aromatic or heteroaromatic ring;
wherein a dashed line in A(a) is optionally a double bond;
R₅₀ is selected from hydrogen, halogen, cyano, alkyl, alkylcycloalkyl, cycloalkyl, cycloalkoxy, alkoxy, alkylthio, hydroxy, amino, monoalkylamino, dialkylamino, heterocycloalkyl, nitro, haloalkyl, —CF₃, haloalkoxy, aryl, —COOR₅₁, and —C(O)R₅₂;
R₅₁ is selected from hydrogen and alkyl;
R₅₂ is selected from alkyl, amino, monoalkylamino, dialkylamino, and heterocycloalkyl; and
R₆₀ at each occurrence is independently selected from hydrogen, halogen, hydroxy, C₁-C₅ alkyl, cycloalkyl, heterocycloalkyl, heteroaryl, cycloalkoxy, and haloalkyl, or two R₆₀ groups together with the atom to which they are attached form a cycloalkyl or heterocycloalkyl ring;
R₂₀₀ at each occurrence is independently selected from
   -alkyl optionally substituted with at least one group independently selected from R₂₀₅,
   —OH,
   —NH₂,
   -halogen,
   —CN,
   —(C₁-C₄ alkyl)₀₋₁-C(O)—NR₂₁₀R₂₁₅,
   —(C₁-C₄ alkyl)₀₋₁-(C(O))₀₋₁—R₂₀₅,
   —(C₁-C₄ alkyl)₀₋₁-(C(O))₀₋₁-cycloalkyl,
   —(C₁-C₄ alkyl)₀₋₁-(C(O))₀₋₁-heterocycloalkyl,
   —(C₁-C₄ alkyl)₀₋₁-(C(O))₀₋₁-heterocycloalkyl-heteroaryl,
   —(C₁-C₄ alkyl)₀₋₁-(C(O))₀₋₁-aryl,
   —(C₁-C₄ alkyl)₀₋₁-(C(O))₀₋₁-heteroaryl,
   —(C₁-C₄ alkyl)₀₋₁-N(H or R₂₀₅)—C(O)—R₂₁₀,
   —(C₁-C₄ alkyl)₀₋₁-NR₂₁₀R₂₁₅,
   —(C₁-C₄ alkyl)₀₋₁-O—(R₂₀₅),
   —(C₁-C₄ alkyl)₀₋₁-S—(R₂₀₅), and
   —(C₁-C₄ alkyl)₀₋₁-O-(alkyl optionally substituted with at least one halogen);
   wherein each aryl or heteroaryl group included within R₂₀₀ is optionally substituted with at least one group independently selected from R₂₀₅ and alkyl (optionally substituted with at least one group independently selected from R₂₀₅);
   wherein each cycloalkyl or heterocycloalkyl group included within R₂₀₀ is optionally substituted with at least one group independently selected from R₂₀₅ and alkyl (optionally substituted with at least one group independently selected from R₂₀₅);
R₂₀₅ at each occurrence is independently selected from
   -alkyl,
   -heteroaryl,
   -heterocycloalkyl,
   -aryl,
   —(CH₂)₀₋₃-cycloalkyl,
   -halogen,
   —(C₁-C₆ alkyl)₀₋₁-CN,
   —OH,
   —O-alkyl, and
   —NR₂₁₀R₂₁₅,
R₂₁₀ and R₂₁₅ at each occurrence are independently selected from
   —H,
   -alkyl,
   -aminoalkyl,
   —(C₁-C₄ alkyl)₀₋₁-C(O)—NH₂,
   —(C₁-C₄ alkyl)₀₋₁-C(O)—NH(alkyl) (wherein alkyl is optionally substituted with at least one group independently selected from R₂₀₅),
   —(C₁-C₄ alkyl)₀₋₁-C(O)—N(alkyl)(alkyl),
   —(CH₂)₀₋₂-cycloalkyl,
   -alkyl-O-alkyl,
   —O-alkyl,
   -aryl,
   -heteroaryl, and
   -heterocycloalkyl; or
R₂₁₀ and R₂₁₅ and the nitrogen to which they are attached form a heterocycloalkyl optionally substituted with at least one R₂₀₅ group;
   wherein the aryl, heteroaryl and heterocycloalkyl groups included within R₂₁₀ and R₂₁₅ are each optionally substituted with at least one group independently selected from R₂₀₅.

Another embodiment of the present invention is a compound of formula (I), 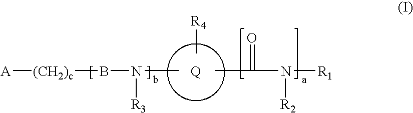
or a pharmaceutically acceptable salt thereof, wherein
a is 1;
b is 1;
c is 0, 1 or 2;
Q is selected from structures Q(a), Q(b), and Q(c), 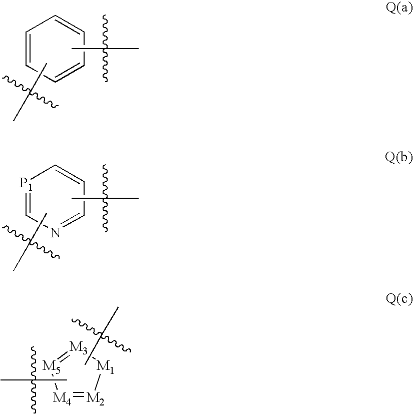
   wherein the two 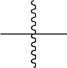
in structures Q(a), Q(b), and Q(c) are not attached to adjacent atoms;
wherein structures Q(a), Q(b), and Q(c) are optionally substituted with at least one group independently selected from alkyl, halogen, —CF₃, and —OH;
M₁ is selected from —NH—, —O—, and —S—;
M₂, M₃, M₄, and M₅ are each independently selected from —C—, —CH—, and —N—;
P₁ is selected from —CH— and —N—;
R₁ is selected from
   —NR^{a}R^{b},
   -alkyl,
   -cycloalkyl,
   -heterocycloalkyl,
   -alkoxy,
   -aryl, and
   -heteroaryl;
   wherein the alkyl, cycloalkyl, heterocycloalkyl, alkoxy, aryl, and heteroaryl within R₁ are each optionally substituted with at least one group independently selected from R₂₀₀;
R^{a} and R^{b} are independently selected from
   -hydrogen (wherein R^{a} and R^{b} are not simultaneously hydrogen),
   -alkyl,
   -alkoxy,
   -cycloalkyl, and
   -heterocycloalkyl;
   wherein the alkyl, alkoxy, cycloalkyl, and heterocycloalkyl within R^{a} and R^{b} are each optionally substituted with at least one group independently selected from R₂₀₀;
or R^{a} and R^{b} together with the nitrogen atom to which they are attached form a heteroaryl (optionally substituted with at least one group independently selected from R₂₀₀) or heterocycloalkyl (optionally substituted with at least one group independently selected from R₂₀₀);
R₂ is selected from
   —H,
   -alkyl,
   -cycloalkyl,
   -heterocycloalkyl,
   -aryl, and
   -heteroaryl;
   wherein the alkyl, cycloalkyl, heterocycloalkyl, aryl, and heteroaryl within R₂ are each optionally substituted with at least one group independently selected from R₂₀₀;
or R₁ and R₂ together with the nitrogen to which they are attached form a heterocycloalkyl (optionally substituted with R₂₀₀) or a heteroaryl (optionally substituted with R₂₀₀);
or R₁ and R₂ together with the nitrogen to which they are attached form 9-pyridin-4-yl-3,9-diaza-spiro[5.5]undecan-3-yl, optionally substituted with at least one group independently selected from R₂₀₀;
R₃ is selected from hydrogen and alkyl;
R₄ is selected from hydrogen, OH, alkyl, aryl, halogen, alkoxy, nitro, CN, cycloalkyl, amino, monoalkylamino, dialkylamino, amino carbonyl, monoalkylamino carbonyl, and dialkylaminocarbonyl;
B is selected from —C(O)— and —S(O)₂—; and
A is selected from structure A(a), 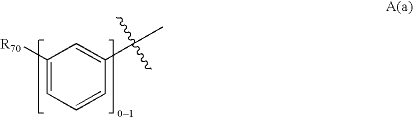
R₇₀ is 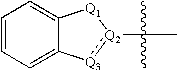
Q₁ is selected from —C(R₆₀)₂—, —O—, —S—, —N(R₆₀)—, and —C(O)—;
Q₂ is selected from —C—, —CH— and —N—; and
Q₃ is selected from —C(R₆₀)₁₋₂— and —N(R₆₀)₀₋₁—;
   wherein structure A(a) is optionally substituted with at least one group independently selected from halogen and alkyl;
   wherein the dashed line in R₇₀ is optionally a double bond;
   R₆₀ at each occurrence is independently selected from hydrogen, halogen, hydroxy, C₁-C₅ alkyl, cycloalkyl, heterocycloalkyl, heteroaryl, cycloalkoxy, and haloalkyl, or two R₆₀ groups together with the atom to which they are attached form a cycloalkyl or heterocycloalkyl ring;
R₂₀₀ at each occurrence is independently selected from
   -alkyl optionally substituted with at least one group independently selected from R₂₀₅,
   —OH,
   —NH₂,
   -halogen,
   —CN,
   —(C₁-C₄ alkyl)₀₋₁-C(O)—NR₂₁₀R₂₁₅,
   —(C₁-C₄ alkyl)₀₋₁-(C(O))₀₋₁—R₂₀₅,
   —(C₁-C₄ alkyl)₀₋₁-(C(O))₀₋₁-cycloalkyl,
   —(C₁-C₄ alkyl)₀₋₁-(C(O))₀₋₁-heterocycloalkyl,
   —(C₁-C₄ alkyl)₀₋₁-(C(O))₀₋₁-heterocycloalkyl-heteroaryl,
   —(C₁-C₄ alkyl)₀₋₁-(C(O))₀₋₁-aryl,
   —(C₁-C₄ alkyl)₀₋₁-(C(O))₀₋₁-heteroaryl,
   —(C₁-C₄ alkyl)₀₋₁-N(H or R₂₀₅)—C(O)—R₂₁₀,
   —(C₁-C₄ alkyl)₀₋₁-NR₂₁₀R₂₁₅,
   —(C₁-C₄ alkyl)₀₋₁-O—(R₂₀₅),
   —(C₁-C₄ alkyl)₀₋₁-S—(R₂₀₅), and
   —(C₁-C₄ alkyl)₀₋₁-O-(alkyl optionally substituted with at least one halogen);
   wherein each aryl or heteroaryl group included within R₂₀₀ is optionally substituted with at least one group independently selected from R₂₀₅ and alkyl (optionally substituted with at least one group independently selected from R₂₀₅);
   wherein each cycloalkyl or heterocycloalkyl group included within R₂₀₀ is optionally substituted with at least one group independently selected from R₂₀₅ and alkyl (optionally substituted with at least one group independently selected from R₂₀₅);
R₂₀₅ at each occurrence is independently selected from
   -alkyl,
   -heteroaryl,
   -heterocycloalkyl,
   -aryl,
   —(CH₂)₀₋₃-cycloalkyl,
   -halogen,
   —(C₁-C₆ alkyl)₀₋₁-CN,
   —OH,
   —O-alkyl, and
   —NR₂₁₀R₂₁₅,
R₂₁₀ and R₂₁₅ at each occurrence are independently selected from
   —H,
   -alkyl,
   -aminoalkyl,
   —(C₁-C₄ alkyl)₀₋₁-C(O)—NH₂,
   —(C₁-C₄ alkyl)₀₋₁-C(O)—NH(alkyl) (wherein alkyl is optionally substituted with at least one group independently selected from R₂₀₅),
   —(C₁-C₄ alkyl)₀₋₁-C(O)—N(alkyl)(alkyl),
   —(CH₂)₀₋₂-cycloalkyl,
   -alkyl-O-alkyl,
   —O-alkyl,
   -aryl,
   -heteroaryl, and
   -heterocycloalkyl; or
R₂₁₀ and R₂₁₅ and the nitrogen to which they are attached form a heterocycloalkyl optionally substituted with at least one R₂₀₅ group;
   wherein the aryl, heteroaryl and heterocycloalkyl groups included within R₂₁₀ and R₂₁₅ are each optionally substituted with at least one group independently selected from R₂₀₅.

An embodiment of the present invention is a compound of formula (I), 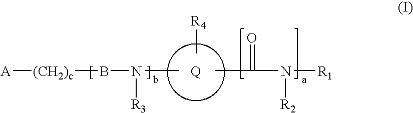
or a pharmaceutically acceptable salt thereof, wherein
a is 1;
b is 0;
c is 0, 1 or 2;
Q is selected from structures Q(a), Q(b), and Q(c), 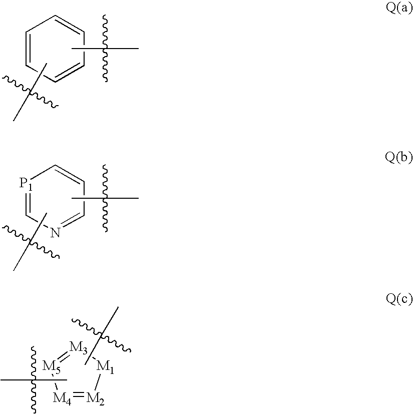
   wherein the two 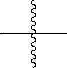
   in structures Q(a), Q(b), and Q(c) are not attached to adjacent atoms;
   wherein structures Q(a), Q(b), and Q(c) are optionally substituted with at least one group independently selected from alkyl, halogen, —CF₃, and —OH;
M₁ is selected from —NH—, —O—, and —S—; and
M₂, M₃, M₄, and M₅ are each independently selected from —C—, —CH—, and —N—;
P₁ is selected from —CH— and —N—;
R₁ is selected from
   —NR^{a}R^{b}
   -alkyl,
   -cycloalkyl,
   -heterocycloalkyl,
   -alkoxy,
   -aryl, and
   -heteroaryl;
   wherein the alkyl, cycloalkyl, heterocycloalkyl, alkoxy, aryl, and heteroaryl within R₁ are each optionally substituted with at least one group independently selected from R₂₀₀;
R^{a} and R^{b} are independently selected from
   -hydrogen (wherein R^{a} and R^{b} are not simultaneously hydrogen),
   -alkyl,
   -alkoxy,
   -cycloalkyl, and
   -heterocycloalkyl,
   wherein the alkyl, alkoxy, cycloalkyl, and heterocycloalkyl within R^{a} and R^{b} are each optionally substituted with at least one group independently selected from R₂₀₀;
or R^{a} and R^{b} together with the nitrogen atom to which they are attached form a heteroaryl (optionally substituted with at least one group independently selected from R₂₀₀) or heterocycloalkyl (optionally substituted with at least one group independently selected from R₂₀₀);
R₂ is selected from
   —H,
   -alkyl,
   -cycloalkyl,
   -heterocycloalkyl,
   -aryl, and
   -heteroaryl;
   wherein the alkyl, cycloalkyl, heterocycloalkyl, aryl, and heteroaryl within R₂ are each optionally substituted with at least one group independently selected from R₂₀₀;
or R₁ and R₂ together with the nitrogen to which they are attached form a heterocycloalkyl optionally substituted with at least one group independently selected from R₂₀₀ or a heteroaryl optionally substituted with at least one group independently selected from R₂₀₀;
R₄ is selected from hydrogen, OH, alkyl, aryl, halogen, alkoxy, nitro, CN, cycloalkyl, amino, monoalkylamino, dialkylamino, amino carbonyl, monoalkylamino carbonyl, and dialkylaminocarbonyl;
B is selected from —C(O)— and —S(O)₂—; and
A is selected from structure A(a), 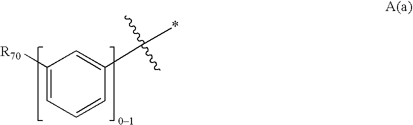
R₇₀ is 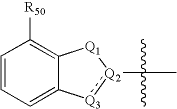
wherein structure A(a) is substituted with at least one R₅₀ group;
   wherein the dashed line in R₇₀ is optionally a double bond;
   R₅₀ is selected from hydrogen, halogen, and alkyl; and
Q₁ is selected from —CH₂—, —O—, —S—, and —NH—;
Q₂ is selected from —C—, —CH— and —N—; and
Q₃ is selected from —CH—, —CH₂—, —N—, and —NH—; or
Q₁ is selected from —C(O)—;
Q₂ is selected from —C—, —CH— and —N—; and
   Q₃ is selected from —C(R₆₀)₁₋₂— and —N(R₆₀)₀₋₁—;
R₆₀ at each occurrence is independently selected from hydrogen, halogen, hydroxy, C₁-C₅ alkyl, cycloalkyl, heterocycloalkyl, heteroaryl, cycloalkoxy, and haloalkyl, or two R₆₀ groups together with the atom to which they are attached form a cycloalkyl or heterocycloalkyl ring;
wherein when A(a) is 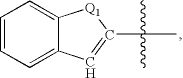
   Q₁ is not —NH— or —N(R₅₀)—;
   wherein when Q is Q(a), A is A(a), Q₁ is —C(O)—, Q₂ is —C(H)—, and Q₃ is —CH₂—, then R₅₀ is selected from halogen and alkyl, or Q₃ is substituted with alkyl, cycloalkyl, heterocycloalkyl, or heteroaryl, each optionally substituted with at least one group independently selected from R₂₀₀;
R₂₀₀ at each occurrence is independently selected from
   -alkyl optionally substituted with at least one group independently selected from R₂₀₅,
   —OH,
   —NH₂,
   -halogen,
   —CN,
   —(C₁-C₄ alkyl)₀₋₁-C(O)—NR₂₁₀R₂₁₅,
   —(C₁-C₄ alkyl)₀₋₁-(C(O))₀₋₁—R₂₀₅,
   —(C₁-C₄ alkyl)₀₋₁-(C(O))₀₋₁-cycloalkyl,
   —(C₁-C₄ alkyl)₀₋₁-(C(O))₀₋₁-heterocycloalkyl,
   —(C₁-C₄ alkyl)₀₋₁-(C(O))₀₋₁-heterocycloalkyl-heteroaryl,
   —(C₁-C₄ alkyl)₀₋₁-(C(O))₀₋₁-aryl,
   —(C₁-C₄ alkyl)₀₋₁-(C(O))₀₋₁-heteroaryl,
   —(C₁-C₄ alkyl)₀₋₁-N(H or R₂₀₅)—C(O)—R₂₁₀,
   —(C₁-C₄ alkyl)₀₋₁-NR₂₁₀R₂₁₅,
   —(C₁-C₄ alkyl)₀₋₁-O—(R₂₀₅),
   —(C₁-C₄ alkyl)₀₋₁-S—(R₂₀₅), and
   —(C₁-C₄ alkyl)₀₋₁-O-(alkyl optionally substituted with at least one halogen);
   wherein each aryl or heteroaryl group included within R₂₀₀ is optionally substituted with at least one group independently selected from R₂₀₅ and alkyl (optionally substituted with at least one group independently selected from R₂₀₅);
   wherein each cycloalkyl or heterocycloalkyl group included within R₂₀₀ is optionally substituted with at least one group independently selected from R₂₀₅ and alkyl (optionally substituted with at least one group independently selected from R₂₀₅);
R₂₀₅ at each occurrence is independently selected from
   -alkyl,
   -heteroaryl,
   -heterocycloalkyl,
   -aryl,
   —(CH₂)₀₋₃-cycloalkyl,
   -halogen,
   —(C₁-C₆ alkyl)₀₋₁-CN,
   —OH,
   —O-alkyl, and
   —NR₂₁₀R₂₁₅,
R₂₁₀ and R₂₁₅ at each occurrence are independently selected from
   —H,
   -alkyl,
   -aminoalkyl,
   —(C₁-C₄ alkyl)₀₋₁-C(O)—NH₂,
   —(C₁-C₄ alkyl)₀₋₁-C(O)—NH(alkyl) (wherein alkyl is optionally substituted with at least one group independently selected from R₂₀₅),
   —(C₁-C₄ alkyl)₀₋₁-C(O)—N(alkyl)(alkyl),
   —(CH₂)₀₋₂-cycloalkyl,
   -alkyl-O-alkyl,
   —O-alkyl,
   -aryl,
   -heteroaryl, and
   -heterocycloalkyl; or
R₂₁₀ and R₂₁₅ and the nitrogen to which they are attached form a heterocycloalkyl optionally substituted with at least one R₂₀₅ group;
   wherein the aryl, heteroaryl and heterocycloalkyl groups included within R₂₁₀ and R₂₁₅ are each optionally substituted with at least one group independently selected from R₂₀₅.

Another embodiment of the present invention is a compound of formula (I), 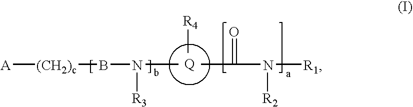
or a pharmaceutically acceptable salt thereof, wherein
a is 0;
b is 0;
c is 0, 1 or 2;
Q is selected from structures Q(b) and Q(c), 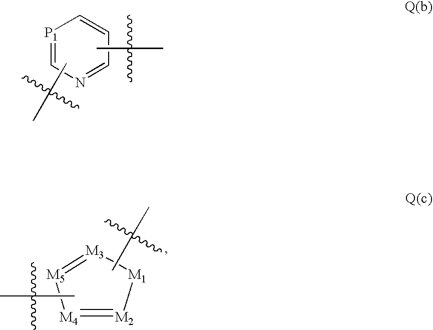
   wherein structures Q(b) and Q(c) are optionally substituted with at least one group independently selected from alkyl, halogen, —CF₃, and —OH;
M₁ is selected from —NH—, —O—, and —S—; and
M₂, M₃, M₄, and M₅ are each independently selected from —C—, —CH—, and —N—;
P₁ is selected from —CH— and —N—;
R₁ is selected from
   —NR^{a}R^{b}
   -alkyl,
   -cycloalkyl,
   -heterocycloalkyl,
   -alkoxy,
   -aryl, and
   -heteroaryl;
   wherein the alkyl, cycloalkyl, heterocycloalkyl, alkoxy, aryl, and heteroaryl within R₁ are each optionally substituted with at least one group independently selected from R₂₀₀;
R^{a} and R^{b} are independently selected from
   -hydrogen (wherein R^{a} and R^{b} are not simultaneously hydrogen),
   -alkyl,
   -alkoxy,
   -cycloalkyl, and
   -heterocycloalkyl;
   wherein the alkyl, alkoxy, cycloalkyl, and heterocycloalkyl within R^{a} and R^{b} are each optionally substituted with at least one group independently selected from R₂₀₀;
or R^{a} and R^{b} together with the nitrogen atom to which they are attached form a heteroaryl (optionally substituted with at least one group independently selected from R₂₀₀) or heterocycloalkyl (optionally substituted with at least one group independently selected from R₂₀₀);
R₃ is selected from hydrogen and alkyl;
R₄ is selected from hydrogen, OH, alkyl, aryl, halogen, alkoxy, nitro, CN, cycloalkyl, amino, monoalkylamino, dialkylamino, amino carbonyl, monoalkylamino carbonyl, and dialkylaminocarbonyl;
B is selected from —C(O)— and —S(O)₂—; and
A is selected from structure A(b), 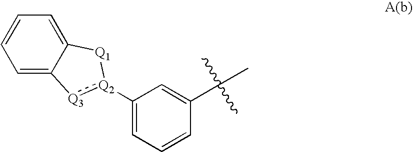
Q₁ is selected from —CH₂—, —O—, —S—, and —NH—;
Q₂ is selected from —C—, —CH— and —N—; and
Q₃ is selected from —CH—, —CH₂—, —N—, and —NH—; or
Q₁ is selected from —C(O)—;
Q₂ is selected from —C—, —CH— and —N—; and
Q₃ is selected from —C(R₆₀)₁₋₂— and —N(R₆₀)₀₋₁—;
R₆₀ at each occurrence is independently selected from hydrogen, halogen, hydroxy, C₁-C₅ alkyl, cycloalkyl, heterocycloalkyl, heteroaryl, cycloalkoxy, and haloalkyl, or two R₆₀ groups together with the atom to which they are attached form a cycloalkyl or heterocycloalkyl ring;
structure A(b) is optionally substituted with at least one group independently selected from halogen and alkyl;
R₂₀₀ at each occurrence is independently selected from
   -alkyl optionally substituted with at least one group independently selected from R₂₀₅,
   —OH,
   —NH₂,
   -halogen,
   —CN,
   —(C₁-C₄ alkyl)₀₋₁-C(O)—NR₂₁₀R₂₁₅,
   —(C₁-C₄ alkyl)₀₋₁-(C(O))₀₋₁—R₂₀₅,
   —(C₁-C₄ alkyl)₀₋₁-(C(O))₀₋₁-cycloalkyl,
   —(C₁-C₄ alkyl)₀₋₁-(C(O))₀₋₁-heterocycloalkyl,
   —(C₁-C₄ alkyl)₀₋₁-(C(O))₀₋₁-heterocycloalkyl-heteroaryl,
   —(C₁-C₄ alkyl)₀₋₁-(C(O))₀₋₁-aryl,
   —(C₁-C₄ alkyl)₀₋₁-(C(O))₀₋₁-heteroaryl,
   —(C₁-C₄ alkyl)₀₋₁-N(H or R₂₀₅)—C(O)—R₂₁₀,
   —(C₁-C₄ alkyl)₀₋₁-NR₂₁₀R₂₁₅,
   —(C₁-C₄ alkyl)₀₋₁-O—(R₂₀₅),
   —(C₁-C₄ alkyl)₀₋₁-S—(R₂₀₅), and
   —(C₁-C₄ alkyl)₀₋₁-O-(alkyl optionally substituted with at least one halogen);
   wherein each aryl or heteroaryl group included within R₂₀₀ is optionally substituted with at least one group independently selected from R₂₀₅ and alkyl (optionally substituted with at least one group independently selected from R₂₀₅);
   wherein each cycloalkyl or heterocycloalkyl group included within R₂₀₀ is optionally substituted with at least one group independently selected from R₂₀₅ and alkyl (optionally substituted with at least one group independently selected from R₂₀₅);
R₂₀₅ at each occurrence is independently selected from
   -alkyl,
   -heteroaryl,
   -heterocycloalkyl,
   -aryl,
   —(CH₂)₀₋₃-cycloalkyl,
   -halogen,
   —(C₁-C₆ alkyl)₀₋₁-CN,
   —OH,
   —O-alkyl, and
   —NR₂₁₀R₂₁₅,
R₂₁₀ and R₂₁₅ at each occurrence are independently selected from
   —H,
   -alkyl,
   -aminoalkyl,
   —(C₁-C₄ alkyl)₀₋₁-C(O)—NH₂,
   —(C₁-C₄ alkyl)₀₋₁-C(O)—NH(alkyl) (wherein alkyl is optionally substituted with at least one group independently selected from R₂₀₅),
   —(C₁-C₄ alkyl)₀₋₁-C(O)—N(alkyl)(alkyl),
   —(CH₂)₀₋₂-cycloalkyl,
   -alkyl-O-alkyl,
   —O-alkyl,
   -aryl,
   -heteroaryl, and
   -heterocycloalkyl; or
R₂₁₀ and R₂₁₅ and the nitrogen to which they are attached form a heterocycloalkyl optionally substituted with at least one R₂₀₅ group;
   wherein the aryl, heteroaryl and heterocycloalkyl groups included within R₂₁₀ and R₂₁₅ are each optionally substituted with at least one group independently selected from R₂₀₅.

Another embodiment of the present invention is a compound of formula (I), 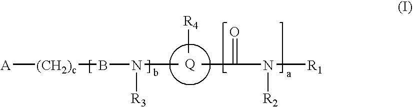
or a pharmaceutically acceptable salt thereof, wherein
a is 0;
b is 1;
c is 0, 1 or 2;
Q is selected from structures Q(b) and Q(c), 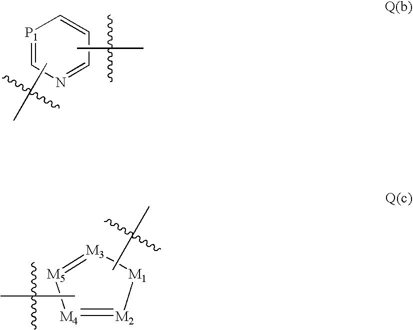
wherein the two 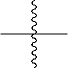
in structures Q(b) and Q(c) are not attached to adjacent atoms;
   wherein structures Q(b) and Q(c) are optionally substituted with at least one group independently selected from alkyl, halogen, —CF₃, and —OH;
M₁ is selected from —NH—, —O—, and —S—;
M₂, M₃, M₄, and M₅ are each independently selected from —C—, —CH—, and —N—;
P₁ is selected from —CH— and —N—;
R₁ is selected from
   —NR^{a}R^{b}
   -alkyl,
   -cycloalkyl,
   -heterocycloalkyl,
   -alkoxy,
   -aryl, and
   -heteroaryl;
   wherein the alkyl, cycloalkyl, heterocycloalkyl, alkoxy, aryl, and heteroaryl within R₁ are each optionally substituted with at least one group independently selected from R₂₀₀;
R^{a} and R^{b} are independently selected from
   -hydrogen (wherein R^{a} and R^{b} are not simultaneously hydrogen),
   -alkyl,
   -alkoxy,
   -cycloalkyl, and
   -heterocycloalkyl;
   wherein the alkyl, alkoxy, cycloalkyl, and heterocycloalkyl within R^{a} and R^{b} are each optionally substituted with at least one group independently selected from R₂₀₀;
or R^{a} and R^{b} together with the nitrogen atom to which they are attached form a heteroaryl (optionally substituted with at least one group independently selected from R₂₀₀) or heterocycloalkyl (optionally substituted with at least one group independently selected from R₂₀₀);
R₃ is selected from hydrogen and alkyl;
R₄ is selected from hydrogen, OH, alkyl, aryl, halogen, alkoxy, nitro, CN, cycloalkyl, amino, monoalkylamino, dialkylamino, amino carbonyl, monoalkylamino carbonyl, and dialkylaminocarbonyl;
B is selected from —C(O)— and —S(O)₂—; and
A is selected from structure A(b), 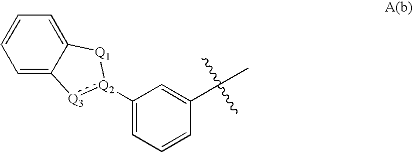
   wherein structure A(b) is optionally substituted with at least one group independently selected from halogen and alkyl,
Q₁ is selected from —C(R₆₀)₂—, —O—, —S—, —N(R₆₀)—, and —C(O)—;
Q₂ is selected from —C—, —CH—, and —N—; and
Q₃ is selected from —C(R₆₀)₁₋₂— and —N(R₆₀)₀₋₁₋₂—;
R₆₀ at each occurrence is independently selected from hydrogen, halogen, hydroxy, C₁-C₅ alkyl, cycloalkyl, heterocycloalkyl, heteroaryl, cycloalkoxy, and haloalkyl, or two R₆₀ groups together with the atom to which they are attached form a cycloalkyl or heterocycloalkyl ring;
R₂₀₀ at each occurrence is independently selected from
   -alkyl optionally substituted with at least one group independently selected from R₂₀₅,
   —OH,
   —NH₂,
   -halogen,
   —CN,
   —(C₁-C₄ alkyl)₀₋₁-C(O)—NR₂₁₀R₂₁₅,
   —(C₁-C₄ alkyl)₀₋₁-(C(O))₀₋₁—R₂₀₅,
   —(C₁-C₄ alkyl)₀₋₁-(C(O))₀₋₁-cycloalkyl,
   —(C₁-C₄ alkyl)₀₋₁-(C(O))₀₋₁-heterocycloalkyl,
   —(C₁-C₄ alkyl)₀₋₁-(C(O))₀₋₁-heterocycloalkyl-heteroaryl,
   —(C₁-C₄ alkyl)₀₋₁-(C(O))₀₋₁-aryl,
   —(C₁-C₄ alkyl)₀₋₁-(C(O))₀₋₁-heteroaryl,
   —(C₁-C₄ alkyl)₀₋₁-N(H or R₂₀₅)—C(O)—R₂₁₀,
   —(C₁-C₄ alkyl)₀₋₁-NR₂₁₀R₂₁₅,
   —(C₁-C₄ alkyl)₀₋₁-O—(R₂₀₅),
   —(C₁-C₄ alkyl)₀₋₁-S—(R₂₀₅), and
   —(C₁-C₄ alkyl)₀₋₁-O-(alkyl optionally substituted with at least one halogen);
   wherein each aryl or heteroaryl group included within R₂₀₀ is optionally substituted with at least one group independently selected from R₂₀₅ and alkyl (optionally substituted with at least one group independently selected from R₂₀₅);
   wherein each cycloalkyl or heterocycloalkyl group included within R₂₀₀ is optionally substituted with at least one group independently selected from R₂₀₅ and alkyl (optionally substituted with at least one group independently selected from R₂₀₅);
R₂₀₅ at each occurrence is independently selected from
   -alkyl,
   -heteroaryl,
   -heterocycloalkyl,
   -aryl,
   —(CH₂)₀₋₃-cycloalkyl,
   -halogen,
   —(C₁-C₆ alkyl)₀₋₁-CN,
   —OH,
   —O-alkyl, and
   —NR₂₁₀R₂₁₅,
R₂₁₀ and R₂₁₅ at each occurrence are independently selected from
   —H,
   -alkyl,
   -aminoalkyl,
   —(C₁-C₄ alkyl)₀₋₁-C(O)—NH₂,
   —(C₁-C₄ alkyl)₀₋₁-C(O)—NH(alkyl) (wherein alkyl is optionally substituted with at least one group independently selected from R₂₀₅),
   —(C₁-C₄ alkyl)₀₋₁-C(O)—N(alkyl)(alkyl),
   —(CH₂)₀₋₂-cycloalkyl,
   -alkyl-O-alkyl,
   —O-alkyl,
   -aryl,
   -heteroaryl, and
   -heterocycloalkyl; or
R₂₁₀ and R₂₁₅ and the nitrogen to which they are attached form a heterocycloalkyl optionally substituted with at least one R₂₀₅ group;
   wherein the aryl, heteroaryl and heterocycloalkyl groups included within R₂₁₀ and R₂₁₅ are each optionally substituted with at least one group independently selected from R₂₀₅.

Another embodiment of the present invention is a compound of formula (I), 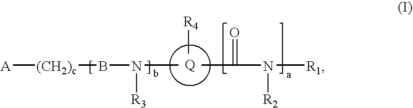
or a pharmaceutically acceptable salt thereof, wherein
a is 0;
b is 0;
c is 0, 1, or 2;
Q is structure Q(a), 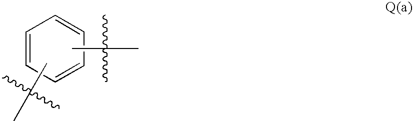
wherein the two 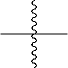
in structure Q(a) are not attached to adjacent atoms;
R₁ is selected from
   —NR^{a}R^{b}
   -alkyl,
   -cycloalkyl,
   -heterocycloalkyl,
   -alkoxy,
   -aryl, and
   -heteroaryl;
   wherein the alkyl, cycloalkyl, heterocycloalkyl, alkoxy, aryl, and heteroaryl within R₁ are each optionally substituted with at least one group independently selected from R₂₀₀;
R^{a} and R^{b} are independently selected from
   -hydrogen (wherein R^{a} and R^{b} are not simultaneously hydrogen),
   -alkyl,
   -alkoxy,
   -cycloalkyl, and
   -heterocycloalkyl;
   wherein the alkyl, alkoxy, cycloalkyl, and heterocycloalkyl within R^{a} and R^{b} are each optionally substituted with at least one group independently selected from R₂₀₀;
or R^{a} and R^{b} together with the nitrogen atom to which they are attached form a heteroaryl (optionally substituted with at least one group independently selected from R₂₀₀) or heterocycloalkyl (optionally substituted with at least one group independently selected from R₂₀₀);
R₃ is selected from hydrogen and alkyl;
wherein when A is A(b), R₄ is selected from hydrogen, OH, alkyl, aryl, halogen, alkoxy, nitro, CN, cycloalkyl, amino, monoalkylamino, dialkylamino, amino carbonyl, monoalkylamino carbonyl, and dialkylaminocarbonyl;
wherein when A is A(d), R₄ is selected from hydrogen, OH, alkyl, aryl, alkoxy, nitro, CN, cycloalkyl, amino carbonyl, monoalkylamino carbonyl, and dialkylaminocarbonyl;
B is selected from —C(O)— and —S(O)₂—; and
A is selected from structures A(b) and A(d), 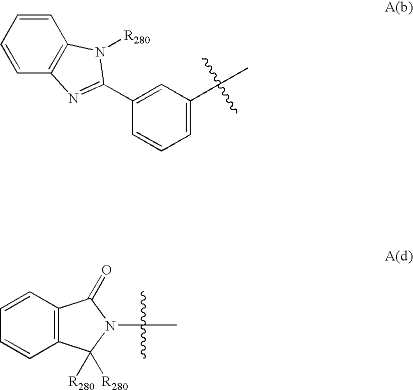
   wherein structures A(b) and A(d) are optionally substituted with at least one group independently selected from halogen and alkyl;
R₂₈₀ at each occurrence is independently selected from hydrogen, halogen, hydroxy, C₁-C₅ alkyl, cycloalkyl, heterocycloalkyl, heteroaryl, cycloalkoxy, and haloalkyl, or two R₂₈₀ groups together with the atom to which they are attached form a cycloalkyl or heterocycloalkyl ring;
R₂₀₀ at each occurrence is independently selected from
   -alkyl optionally substituted with at least one group independently selected from R₂₀₅,
   —OH,
   —NH₂,
   -halogen,
   —CN,
   —(C₁-C₄ alkyl)₀₋₁-C(O)—NR₂₁₀R₂₁₅,
   —(C₁-C₄ alkyl)₀₋₁-(C(O))₀₋₁—R₂₀₅,
   —(C₁-C₄ alkyl)₀₋₁-(C(O))₀₋₁-cycloalkyl,
   —(C₁-C₄ alkyl)₀₋₁-(C(O))₀₋₁-heterocycloalkyl,
   —(C₁-C₄ alkyl)₀₋₁-(C(O))₀₋₁-heterocycloalkyl-heteroaryl,
   —(C₁-C₄ alkyl)₀₋₁-(C(O))₀₋₁-aryl,
   —(C₁-C₄ alkyl)₀₋₁-(C(O))₀₋₁-heteroaryl,
   —(C₁-C₄ alkyl)₀₋₁-N(H or R₂₀₅)—C(O)—R₂₁₀,
   —(C₁-C₄ alkyl)₀₋₁-NR₂₁₀R₂₁₅,
   —(C₁-C₄ alkyl)₀₋₁-O—(R₂₀₅),
   —(C₁-C₄ alkyl)₀₋₁-S—(R₂₀₅), and
   —(C₁-C₄ alkyl)₀₋₁-O-(alkyl optionally substituted with at least one halogen);
   wherein each aryl or heteroaryl group included within R₂₀₀ is optionally substituted with at least one group independently selected from R₂₀₅ and alkyl (optionally substituted with at least one group independently selected from R₂₀₅);
   wherein each cycloalkyl or heterocycloalkyl group included within R₂₀₀ is optionally substituted with at least one group independently selected from R₂₀₅ and alkyl (optionally substituted with at least one group independently selected from R₂₀₅);
R₂₀₅ at each occurrence is independently selected from
   -alkyl,
   -heteroaryl,
   -heterocycloalkyl,
   -aryl,
   —(CH₂)₀₋₃-cycloalkyl,
   -halogen,
   —(C₁-C₆ alkyl)₀₋₁-CN,
   —OH,
   —O-alkyl, and
   —NR₂₁₀R₂₁₅,
R₂₁₀ and R₂₁₅ at each occurrence are independently selected from
   —H,
   -alkyl,
   -aminoalkyl,
   —(C₁-C₄ alkyl)₀₋₁-C(O)—NH₂,
   —(C₁-C₄ alkyl)₀₋₁-C(O)—NH(alkyl) (wherein alkyl is optionally substituted with at least one group independently selected from R₂₀₅),
   —(C₁-C₄ alkyl)₀₋₁-C(O)—N(alkyl)(alkyl),
   —(CH₂)₀₋₂-cycloalkyl,
   -alkyl-O-alkyl,
   —O-alkyl,
   -aryl,
   -heteroaryl, and
   -heterocycloalkyl; or
R₂₁₀ and R₂₁₅ and the nitrogen to which they are attached form a heterocycloalkyl optionally substituted with at least one R₂₀₅ group;
   wherein the aryl, heteroaryl and heterocycloalkyl groups included within R₂₁₀ and R₂₁₅ are each optionally substituted with at least one group independently selected from R₂₀₅.

Another embodiment of the present invention are compounds of formula (I), 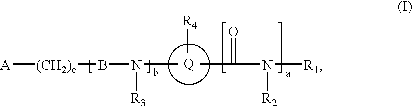
or a pharmaceutically acceptable salt thereof, wherein
a is 0;
b is 1;
c is 0, 1, or 2;
Q is structure Q(a), 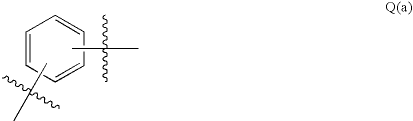
wherein the two 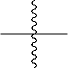
in structure Q(a) are not attached to adjacent atoms;
R₁ is selected from
   —NR^{a}R^{b}
   -alkyl,
   -cycloalkyl,
   -heterocycloalkyl,
   -alkoxy,
   -aryl, and
   -heteroaryl;
   wherein the alkyl, cycloalkyl, heterocycloalkyl, alkoxy, aryl, and heteroaryl within R₁ are each optionally substituted with at least one group independently selected from R₂₀₀;
R^{a} and R^{b} are independently selected from
   -hydrogen (wherein R^{a} and R^{b} are not simultaneously hydrogen),
   -alkyl,
   -alkoxy,
   -cycloalkyl, and
   -heterocycloalkyl;
   wherein the alkyl, alkoxy, cycloalkyl, and heterocycloalkyl within R^{a} and R^{b} are each optionally substituted with at least one group independently selected from R₂₀₀;
or R^{a} and R^{b} together with the nitrogen atom to which they are attached form a heteroaryl (optionally substituted with at least one group independently selected from R₂₀₀) or heterocycloalkyl (optionally substituted with at least one group independently selected from R₂₀₀);
R₃ is selected from hydrogen and alkyl;
wherein when A is A(b), R₄ is selected from hydrogen, OH, alkyl, aryl, halogen, alkoxy, nitro, CN, cycloalkyl, amino, monoalkylamino, dialkylamino, amino carbonyl, monoalkylamino carbonyl, and dialkylaminocarbonyl;
wherein when A is A(d), R₄ is selected from hydrogen, OH, alkyl, aryl, alkoxy, nitro, CN, cycloalkyl, amino carbonyl, monoalkylamino carbonyl, and dialkylaminocarbonyl;
B is selected from —C(O)— and —S(O)₂—; and
A is selected from structures A(b) and A(d), 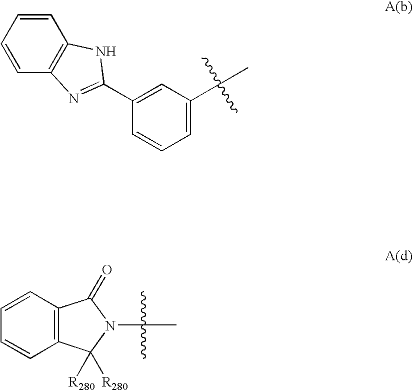
   wherein structures A(b) and A(d) are optionally substituted with at least one group independently selected from halogen and alkyl;
R₂₈₀ at each occurrence is independently selected from hydrogen, halogen, hydroxy, C₁-C₅ alkyl, cycloalkyl, heterocycloalkyl, heteroaryl, cycloalkoxy, and haloalkyl, or two R₂₈₀ groups together with the atom to which they are attached form a cycloalkyl or heterocycloalkyl ring;
R₂₀₀ at each occurrence is independently selected from
   -alkyl optionally substituted with at least one group independently selected from R₂₀₅,
   —OH,
   —NH₂,
   -halogen,
   —CN,
   —(C₁-C₄ alkyl)₀₋₁-C(O)—NR₂₁₀R₂₁₅,
   —(C₁-C₄ alkyl)₀₋₁-(C(O))₀₋₁—R₂₀₅,
   —(C₁-C₄ alkyl)₀₋₁-(C(O))₀₋₁-cycloalkyl,
   —(C₁-C₄ alkyl)₀₋₁-(C(O))₀₋₁-heterocycloalkyl,
   —(C₁-C₄ alkyl)₀₋₁-(C(O))₀₋₁-heterocycloalkyl-heteroaryl,
   —(C₁-C₄ alkyl)₀₋₁-(C(O))₀₋₁-aryl,
   —(C₁-C₄ alkyl)₀₋₁-(C(O))₀₋₁-heteroaryl,
   —(C₁-C₄ alkyl)₀₋₁-N(H or R₂₀₅)—C(O)—R₂₁₀,
   —(C₁-C₄ alkyl)₀₋₁-NR₂₁₀R₂₁₅,
   —(C₁-C₄ alkyl)₀₋₁-O—(R₂₀₅),
   —(C₁-C₄ alkyl)₀₋₁-S—(R₂₀₅), and
   —(C₁-C₄ alkyl)₀₋₁-O-(alkyl optionally substituted with at least one halogen);
   wherein each aryl or heteroaryl group included within R₂₀₀ is optionally substituted with at least one group independently selected from R₂₀₅ and alkyl (optionally substituted with at least one group independently selected from R₂₀₅);
   wherein each cycloalkyl or heterocycloalkyl group included within R₂₀₀ is optionally substituted with at least one group independently selected from R₂₀₅ and alkyl (optionally substituted with at least one group independently selected from R₂₀₅);
R₂₀₅ at each occurrence is independently selected from
   -alkyl,
   -heteroaryl,
   -heterocycloalkyl,
   -aryl,
   —(CH₂)₀₋₃-cycloalkyl,
   -halogen,
   —(C₁-C₆ alkyl)₀₋₁-CN,
   —OH,
   —O-alkyl, and
   —NR₂₁₀R₂₁₅,
R₂₁₀ and R₂₁₅ at each occurrence are independently selected from
   —H,
   -alkyl,
   -aminoalkyl,
   —(C₁-C₄ alkyl)₀₋₁-C(O)—NH₂,
   —(C₁-C₄ alkyl)₀₋₁-C(O)—NH(alkyl) (wherein alkyl is optionally substituted with at least one group independently selected from R₂₀₅),
   —(C₁-C₄ alkyl)₀₋₁-C(O)—N(alkyl)(alkyl),
   —(CH₂)₀₋₂-cycloalkyl,
   -alkyl-O-alkyl,
   —O-alkyl,
   -aryl,
   -heteroaryl, and
   -heterocycloalkyl; or
R₂₁₀ and R₂₁₅ and the nitrogen to which they are attached form a heterocycloalkyl optionally substituted with at least one R₂₀₅ group;
   wherein the aryl, heteroaryl and heterocycloalkyl groups included within R₂₁₀ and R₂₁₅ are each optionally substituted with at least one group independently selected from R₂₀₅.

In another embodiment, Q₁ and Q₃ are each independently selected from —C(R₆₀)₂—, wherein each R₆₀ is methyl.

In an embodiment, R₇₀ is selected from structures R₇₀(a), R₇₀(b), R₇₀(c), and R₇₀(d),

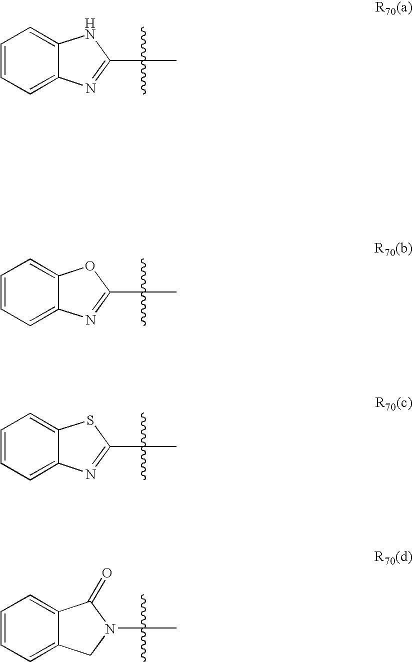

In an embodiment, R₁ and R₂ together with the nitrogen to which they are attached form a spiro-piperidine optionally substituted with a group selected from heterocycloalkyl, aryl, heteroaryl and alkyl.

In another embodiment, R₁ and R₂ together with the nitrogen to which they are attached form (9-pyridin-4-yl-)3,9-diaza-spiro[5.5]undecan-3-yl.

In another embodiment, R₁ and R₂ together with the nitrogen to which they are attached form a ring structure selected from 9-Pyridin-4-yl-3,9-diaza-spiro[5.5]undec-3-yl, 9-Methyl-3,9-diaza-spiro[5.5]undec-3-yl, 9-Isopropyl-3,9-diaza-spiro[5.5]undec-3-yl, 9-tert -Butoxycarbonyl-3,9-diaza-spiro[5.5]undec-3-yl, 4-Pyridin-4-yl-piperazin-1-yl, (3′,4′,5′,6′,3″,4″,5″,6″-Octahydro-2′H,2″H-[4,1′;4′,4″]terpyridinyl), (3′,4′,5′,6′,3″,4″,5″,6″-Octahydro-2′H,2″H-[2,1′;4′,4″]terpyridinyl), 1′-Isopropyl-[4,4′]bipiperidinyl, 1′-Methyl-[4,4′]bipiperidinyl, [4,4′]Bipiperidinyl, 4-Amino-[1,4′]bipiperidinyl, 4-(2-Imidazol-1-yl-ethyl)-piperaz-1-yl, 4-(1-Methyl-piperidin-4-ylmethyl)-piperaz-1-yl, 4-(3-Pyrrolidin-1-yl-propyl)-piperaz -1-yl, 4-phenethyl-piperaz-1-yl, 4-Cyclohexylmethyl-piperaz-1-yl, 4-Cyclohexyl-piperaz -1-yl, 4-(2-Dimethylamino-ethyl)-piperaz-1-yl, 4-(pyridin-2-ylcarbamoylmethyl)-piperaz -1-yl, 4-Benzyl-piperaz-1-yl, 4-(pyrrolidine-1-carbonyl)-piperaz-1-yl, 4-pyridin-2-yl-piperaz -1-yl, 4-Isopropyl-piperaz-1-yl, 4-phenyl-piperaz-1-yl, 4-pyrimidin-2-yl-piperaz-1-yl, 4-(2-pyrrol-1-yl-ethyl)-piperaz-1-yl, 4-(pyridin-4-yloxy)-piperidyl, 4-(4-isopropylpiperazin-1-yl)piperidyl, and 4-(1,2,3,4-tetrahydroisoquinolin-5-yloxy)piperidyl.

In an embodiment, R₁ is selected from 4-Pyridin-4-yl-piperazin-1-ylmethyl, 2-(3,4,5,6-Tetrahydro-2H-[1,4′]bipyridinyl-4-yl)-ethyl, 1-(4-Pyridin-4-yl-piperazin-1-yl)-ethyl, 3,4,5,6-Tetrahydro-2H-[1,4′]bipyridinyl-4-ylmethyl, 3,4,5,6-Tetrahydro-2H-[1,4′]bipyridinyl-4-ylamino, Piperidin-4-ylidenemethyl, 4-Pyridin-4-yl-piperazin-1-yl, 3,4,5,6-Tetrahydro-2H-[1,4′]bipyridinyl-4-yl, 2-(4-Pyridin-4-yl-piperazin-1-yl)-ethyl 4-(4-Pyridin-4-yl -piperazin-1-yl)-phenyl, 2-piperidin-4-ylvinyl, 2-piperidin-4-yl-ethyl, piperidin-4-ylmethoxy, 1-(tert-butoxycarbonyl)piperidin -4-yloxy, piperidin-4-yloxy, 3,4,5,6-tetrahydro-2H-[1,4′]bipyridinyl -4-ylmethoxy, 3,4,5,6-tetrahydro-2H-[1,4′]bipyridinyl-4-yloxy, 2,3,5,6-tetrahydro-[1,4′]bipyridinyl-4-ylidenemethyl, 2-piperidin-4-ylethoxy, 2-(3,4,5,6-tetrahydro-2H-[1,4′]bipyridinyl-4-yl)ethoxy, and (2S,6R)-dimethyl-4-pyridin-4-ylpiperazin-1-ylmethyl.

In an embodiment, R₁ is selected from 4-Pyridin-4-yl-piperazin-1-ylmethyl, 2-(3,4,5,6-Tetrahydro-2H-[1,4′]bipyridinyl-4-yl)-ethyl, 1-(4-Pyridin-4-yl-piperazin-1-yl)-ethyl, 3,4,5,6-Tetrahydro-2H-[1,4′]bipyridinyl-4-ylmethyl, 3,4,5,6-Tetrahydro-2H-[1,4′]bipyridinyl -4-ylamino, 2-Piperidin-4-ylidenemethyl, 4-Pyridin-4-yl-piperazin-1-yl, 3,4,5,6-Tetrahydro-2H-[1,4′]bipyridinyl-4-yl, 4-(4-Pyridin-4-yl-piperazin-1-yl)-phenyl, 2-[1-(1H 4-yl]-ethyl, 2-(4-Pyridin-4-yl-piperazin-1-yl)-ethyl, 2-[1-(1H-Benzoimidazol-2-yl)-4 -yl]-ethyl, 3-(3,4,5,6-Tetrahydro-2H-[1,4′]bipyridinyl-4-yl)-propyl, 2-(3′-Methyl-3 3,4,5,6-tetrahydro-2H-[1,2′]bipyridinyl-4-yl)-ethyl, 2-[4-(4-Methyl-piperazin-1-yl)-pheny -ethyl, 2-(4-Pyridin-4-yl-phenyl)-ethyl, 4-(3-Amino-propyl)-phenyl, 2-(1-Methyl-piperidin-4-yl)-ethyl, 2-(4-Acetylamino-phenyl)-ethyl, Azepan-3-yl, 2-(4-Amino-phenyl)-ethyl, 2-(2′-Cyano-3,4,5,6-tetrahydro-2H-[1,4′]bipyridinyl-4-yl)-ethyl, 3-(5,6,7,8-Tetrahydro-[1,8]naphthyridin-2-yl)-propyl, and 2-Oxo-5-phenyl-2,3-dihydro-1H-benzo[e][1,4]diazepin-3-yl.

In another embodiment, R₁ is selected from (1-(benzyloxyacetyl)-azepan-3-yl)amino; (1,5-dimethyl-2,3,4,5-tetrahydro-1H-benzo[b][1,4]diazepin-3-yl)amino; (1,5-dimethyl-2-oxo-2,3,4,5-tetrahydro-1H-benzo[b][1,4]diazepin-3-yl)amino; (1-cyclopropylmethyl-2-oxo-azepan-3-yl)amino; (1-cyclopropylmethyl-5-methyl-2-oxo-2,3,4,5-tetrahydro-1H-benzo[b][1,4]diazepin-3-yl)amino; (1-cyclopropylmethyl-azepan-3-yl)amino; (1-ethyl-2-oxo-5-phenyl-2,3-dihydro-1H-benzo[e][1,4]diazepin-3-yl)amino; (1′-methyl-[1,4′]bipiperidinyl-4-yl)methylamino; (1-methyl-piperidin-4-ylmethyl)amino; (1-pyridin-4-ylmethyl-piperidin-4-yl)amino; (1-pyridin-4-ylmethyl-piperidin-4-ylmethyl)amino; (2-oxo-1-propyl-azepan-3-yl)amino; (2-oxo-5-phenethyl-1-propyl-2,3-dihydro-1H-benzo[e][1,4]diazepin-3-yl)amino; (3,4,5,6-tetrahydro-2H-[1,4′]bipyridinyl-4-yl)methylamino; (5-methyl-2-oxo-2,3,4,5-tetrahydro-1H-benzo[b][1,4]diazepin-3-yl)amino; (5-methyl-6-oxo -4-yl)amino; (N-(pyridin-4-ylcarbonyl)piperidin-4-yl)methylamino; [1-(2-dimethylamino-ethyl)-2-oxo-azepan-3-yl]amino; [1-(2-pyridin-4-yl-ethyl)-piperidin-4-yl]amino; [1-(2-pyridin-4-yl-ethyl)-piperidin-4-ylmethyl]amino; [1-(pyridin-4-ylcarbonyl)-piperidin-4-yl]amino; [2-(1′-methyl-[1,4′]bipiperidinyl-4-yl)-ethyl]amino; [2-(1-pyridin -4-ylmethyl-piperidin-4-yl)-ethyl]amino; [2-(4-pyridin-4-yl-piperazin-1-yl)ethyl]amino; [2-(pyridine-4-yl)ethyl]amino; [5-(3-aza-bicyclo[3.2.2]non-3-yl)-1-methyl-2-oxo-2,3-dihydro-1H-benzo[e][1,4]diazepin-3-yl]amino; [5-(benzyloxycarbonyl)-2-oxo-2,3,4,5-tetrahydro-1H-benzo[b][1,4]diazepin-3-yl]amino; {2-[1-(2-pyridin-4-yl-ethyl)-piperidin-4-yl]-ethyl}amino; {2-[1-(N,N-dimethylaminocarbonyl)-piperidin-4-yl]ethyl}amino; {2-[1-(pyridin-4-ylcarbonyl)-piperidin -4-yl]ethyl}amino; 2-(3-methoxy-4-hydroxy-phenyl)ethylamino; 2-(N-(4-1H-benzimidazol -2-yl)piperin-4-yl)ethylamino; 2-(N-(4-benzimidazol-2-yl)piperin-4-yl)ethylamino; 2-(N-methyl-N-pyridin-4-yl)ethylamino; 2-[1,4′]bipiperidinyl-2-cyano-ethylamino; 2-[1,4′]bipiperidinylethylamino; 2-[2-phenyl-1H-benzo[d]imidazole]ethylamino; 2-[4-(pyridin-4-yl)piperidin-1-yl]ethylamino; 2-[N-((pyridin-4-yl)acetyl)piperidin-4-yl]ethylamino; 2-[N-(2,2,2-trichloroethoxyacetyl)piperidin-4-yl]ethylamino; 5-(t-butoxycarbonyl)aminopentylamino; 5-aminopentylamino; N-((pyridin-4-yl)acetyl)piperidin-4-ylamino; and piperidin-4-ylamino.

In another embodiment, R₁ is selected from 1-(2-Aminoethyl)piperidine; 1-(2-Pyridinyl)-4-piperidinamine; 1-(2-Pyridinyl)-4-piperidinethanamine; 1-(4-Chlorophenyl)ethylamine; 1-(4-Fluorophenyl)ethylamine; 1-(4-Methoxyphenyl)ethylamine; 1-(4-Methyl)-4-piperidinepropan-2-amine; 1-(4-Pyridinyl)-4-piperidinamine; 1-(4-pyridyl)-4-piperidineethanamine; 1,5-Dimethyl-1H-pyrazole-3-methanamine; 1-Amino-2-indanol; 1-Aminopiperidine; 1-Benzyl-3-aminopyrrolidine; 1-Dimethylamino-2-propylamine; 1-Methyl-1H-pyrrole-2-methanamine; 1-Methyl-3-piperidinamine; 1-Methyl-4-piperidineethanamine; 1-Methylpiperazine; 1-phenyl-4-(2-aminoethyl)piperidine; 1-Phenylpiperazine; alpha-methyl-1-Piperidineethanamine; 2-(2-aminoethyl)-1-methylpyrrolidine; 2-(4-Benzylpiperazin-1-yl)ethylamine; 2-(4-Methylpiperazin-1-yl)ethylamine; 2-(Aminomethyl)-1-ethylpyrrolidine; 2-(Aminomethyl)-5-methylpyrazine; 2-Amino-4-phenyl-1-piperidin-1-ylbutane; 2-Benzyloxycyclopentylamine; 2-Methylcyclohexylamine; 2-phenylglycinol; 2-Picolylamine; 3-(1H-Pyrrol-1-yl)-benzenemethanamine; 3-amino-1,3,4,5-tetrahydro-2H-1-benzazepin-2-one; 3-Amino-1,3-dihydro-1-methyl-5-phenyl-2H-1,4-benzodiazepin-2-one; 3-Amino-1,3-dihydro-5-phenyl-2H-1,4-benzodiazepin-2-one; 3-Amino-1,3-dihydro-5-cyclohexyl-2H-1,4-benzodiazepin-2-one; 3-Amino-1-ethylhexahydro-2H-azepin-2-one; 3-Amino-1-methyl-2-piperidinone; 3-Amino-2-oxo-1,2,3,4-tetrahydroquinoline; 3-Amino-3-methyl-2-piperidone; 3-Amino-7-chloro-1,3-dihydro-5-phenyl-2H-1,4-benzodiazepin-2-one; 3-Amino-7-chloro-5-(2-chlorophenyl)-1,3-dihydro-2H-1,4-benzodiazepin-2-one; 3-Aminohexahydro-1-(phenylmethyl)-2H-azepin-2-one; 3-Aminomethylbenzothiophene; 3-aminoquinuclidine; 3-Dimethylamino-1-propylamine; 3-Morpholinopropylamine; 3-Picolylamine; 4-(1-Aminoethyl)phenol; 4-(2-Aminoethyl)morpholine; 4-(2-Aminoethyl)pyridine; 4-Amino-1-benzylpiperidine; 4-Amino-2-butanol; 4-Picolylamine; 1-methyl-4-Piperidinamine; 5-Methyl-3-Isoxazolemethanamine; Alaminol; alpha-N,N-Dimethylbenzylamine; alpha-Amine-epsilon-N-methyl-caprolactam; alpha-Aminodiphenylmethane; alpha-Amino-epsilon-caprolactam; alpha-methyl-4-Morpholineethanamine; alpha-Methylbenzylamine; Azepan-3-ylamine; benzylamine; beta-methyl-1-pyrrolidineethanamine; Cumylamine; cyclohexylamine; endo-8-Methyl-8-azabicyclo[3.2.1]octan-3-amine; Ethanolamine; Hexahydro-1-methyl-1H-azepin-3-amine; histamine; Isopropylamine; methylamine; morpholine; N-(2-aminoethyl)-2-Benzyl-N-methylaniline; N-(2-Aminoethyl)acetamide; N-(2-Aminoethyl)pyrrolidine; N,N,N′-Trimethylethylenediamine; N,N-Dimethylethylenediamine; N,O-Dimethylhydroxylamine; N-alpha-dimethylbenzylamine; phenethylamine; trans-2-Aminocyclohexanol; trans-4-Aminocyclohexanol; Tryptamine; Tyramine; Valinol; N,N-diethyl-1,2-propanediamine; N-ethyl-N-methyl-1,2-propanediamine; 1-phenylsulfonyl-4-piperidineamine; alpha-phenyl-1-piperidineethanamine; N,N-dimethyl-1,2-butanediamine; 3,4-dihydro-1-(2H)-quinolineethanamine; 1-Amino-2-propanol; beta-alaninamide; beta-alanine t-butyl ester; alpha-methyl-4-(methylsulfonyl)-benzenemethanamine; 1-[2-pyrrolidinylmethyl]-pyrrolidine; alpha-methylbenzylamine; alpha methyl-1-pyrrolidineethanamine; N,N-dimethyl-4-phenyl-1,2-butanediamine; N-acetyl-N-methyl-1,2-propanediamine; N-methyl-N-phenyl-1,2-ethanediamine; N-cyclopropyl-N-methyl-1,2-propanediamine; (4-Phenyl-morpholin-2-yl)-methylamine; 1-(1-Naphthyl)ethylamine; 1,2,3,4-Tetrahydro-1-naphthylamine; 1-Aminoethylphosphonic acid; 1-Cyclohexylethylamine; 1-Ethynylcyclohexylamine; 1-Methoxy-3-phenyl-2-propylamine; 2-(Aminomethyl)benzimidazole; 2-(Diisobutylamino)ethylamine; 2-(Diisopropylamino)ethylamine; 2,2,2-Trifluoroethylamine; 2,2-Diphenylethylamine; 2,6-Bis(dimethylamino)benzylamine; 2-[2-(Aminomethyl)phenylthio]benzyl alcohol; 2-amino-1,2-diphenylethanol; 2-Amino-4′-bromoacetophenone; 2-Aminoacetophenone; 2-(Aminoethyl)-2-thiopseudourea; 2-Aziridinoethylamine; 2-Methoxyisopropylamine; 2-Methylallylamine; 3,3-Diphenylpropylamine; 3,4-Methylenedioxyamphetamine; 3-Aminocyclohexanecarboxylic acid; 3-Aminopyrrolidine; 3-Nitrophenacylamine; 4-(2-aminoethyl)-1-methylpiperidine; 4-(2-Aminoethyl)benzenesulfonamide; 4-Amino-1-diethylaminopentane; 7-Amino-5-methyl-5H,7H-dibenzo[b,d]azepin-6-one; Agmatine; alpha-1-Amino-2-propanol; alpha-Ethylbenzylamine; Aminoacetamidine; Aminoacetonitrile; beta-Methylphenethylamine; Cathinone; Cyclobutylamine; Cyclohexanemethylamine; Cyclopropylamine; Cycloserine; Homocysteine thiolactone; Menthylamine; Methioninol; Muscimol; N-(3′-Aminopropyl)-2-pyrrolidinone; N-(3-Aminopropyl)diethanolamine; N,N-Dimethyl-1,4-diaminobutane; N-Benzylethylenediamine; N-Ethyl-N-Butylethylenediamine; Norephedrine; O-Benzylhydroxylamine; Phenylisopropylamine; p-Methoxyamphetamine; and Tetrahydrofurfurylamine.

Among the compounds of formula (I), examples include 2-[3-([4,4′]Bipiperidinyl-1-carbonyl)-phenyl]-7-chloro-2,3-dihydro-isoindol-1-one, 7-Chloro-2-[3-(9-methyl-3,9-diaza-spiro[5.5]undecane-3-carbonyl)-phenyl]-2,3-dihydro-isoindol-1-one, 7-Chloro-2-{3-[4-(pyridin-4-yloxy)-piperidine-1-carbonyl]-phenyl}-2,3-dihydro-isoindol-1-one, 7-Chloro-2-[3-(1′-methyl-[4,4′]bipiperidinyl-1-carbonyl)-phenyl]-2,3-dihydro-isoindol-1-one, 2-[3-(4-Amino-[1,4′]bipiperidinyl-1′-carbonyl)-phenyl]-7-chloro-2,3-dihydro-isoindol-1-one, 3-(7-Chloro-1H-benzoimidazol-2-yl)-N-[2-(3,4,5,6-tetrahydro-2H-[1,4′]bipyridinyl-4-yl)-ethyl]-benzamide, 3-(7-Chloro-1-oxo-1,3-dihydro-isoindol-2-yl)-N-[3-(5,6,7,8-tetrahydro-[1,8]naphthyridin-2-yl)-propyl]-benzamide, 7-Chloro-2-{3-[4-(pyridin-4-ylmethoxy)-piperidine-1-carbonyl]-phenyl}-2,3-dihydro-isoindol-1-one, 5-(7-Chloro-1-oxo-1,3-dihydro-isoindol-2-yl)-1H-pyrazole-3-carboxylic acid [2-(3,4,5,6-tetrahydro-2H-[1,4′]bipyridinyl-4-yl)-ethyl]-amide, 3-(4-Chloro-1H-benzoimidazol-2-yl)-piperidine-1-carboxylic acid [2-(3,4,5,6-tetrahydro-2H-[1,4′]bipyridinyl-4-yl)-ethyl]-amide, 4-Chloro-2-{3-[5-(4-pyridin-4-yl-piperazin-1-ylmethyl)-furan-2-yl]-phenyl}-1H-benzoimidazole, 4-Chloro-2-{3-[5-(4-pyridin-4-yl-piperazin-1-ylmethyl)-1H-imidazol-2-yl]-phenyl}-1H-benzoimidazole, 4-{5-[3-(4-Chloro-1H-benzoimidazol-2-yl)-phenyl]-4H-[1,2,4]triazol-3-ylmethyl}-3,4,5,6-tetrahydro-2H-[1,4′]bipyridinyl, 4-{5-[3-(4-Chloro-1H-benzoimidazol-2-yl)-phenyl]-tetrazol-2-ylmethyl}-3,4,5,6-tetrahydro-2H-[1,4′]bipyrdinyl, 3-(7-Chloro-1-oxo-1,3-dihydro-isoindol-2-yl)-N-[2-(3,4,5,6-tetrahydro-2H-[1,4′]bipyridinyl-4-yl)ethyl]-benzamide, 3-(4-Chloro-benzothiazol-2-yl)-N-[2-(3,4,5,6-tetrahydro-2H-[1,4′]bipyridinyl-4-yl)-ethyl]-benzamide, 7-Chloro-2-[3-(1′-isopropyl-[4,4′]bipiperidinyl-1-carbonyl)-phenyl]-2,3-dihydro-isoindol-1-one, 4-Chloro-2-{3-[5-(4-pyridin-4-yl-piperazin-1-ylmethyl)-pyridin-3-yl]-phenyl}-1H-benzoimidazole, 4-Chloro-2-{3-[4-(4-pyridin-4-yl-piperazin-1-ylmethyl)-pyridin-2-yl]-phenyl}-1H-benzoimidazole, 4-Chloro-2-{3-[6-(4-pyridin-4-yl-piperazin-1-ylmethyl)-pyridin-2-yl]-phenyl}-1H-benzoimidazole, 5-(7-Chloro-1-oxo-1,3-dihydro-isoindol-2-yl)-thiophene-2-carboxylic acid [2-(3,4,5,6-tetrahydro-2H-[1,4′]bipyridinyl-4-yl)-ethyl]-amide, 3-(7-Chloro-1-oxo-1,3-dihydro-isoindol-2-yl)-N-[2-(4-piperidin-1-ylmethyl-phenyl)-ethyl]-benzamide, 5-(7-Chloro-1-oxo-1,3-dihydro-isoindol-2-yl)-2-hydroxy-N-[2-(3,4,5,6-tetrahydro-2H-[1,4′]bipyridinyl-4-yl)-ethyl]-benzamide, 4-Chloro-3-(4-chloro-1H-benzoimidazol-2-yl)-N-[2-(3,4,5,6-tetrahydro-2H-[1,4′]bipyridinyl-4-yl)-ethyl]-benzamide, {2-[3-(4-Chloro-1H-benzoimidazol-2-yl)-phenyl]-pyridin-4-yl}-(4-pyridin-4-yl-piperazin-1-yl)-methanone, 4-Chloro-2-(3-{5-[1-(4-pyridin-4-yl-piperazin-1-yl)-ethyl]-pyridin-3-yl}-phenyl)-1H-benzoimidazole, 7-Chloro-2-{3-[5-(4-pyridin-4-yl-piperazin-1-ylmethyl)-pyridin-3-yl]-phenyl}-2,3-dihydro-isoindol-1-one, 7-Chloro-2-{3-[4-(1-isopropyl-piperidin-4-yloxy)-piperidine-1-carbonyl]-phenyl}-2,3-dihydro-isoindol-1-one, 4-Chloro-2-{3-[5-(4-pyridin-4-yl-piperazin-1-ylmethyl)-pyridin-3-yl]-phenyl}-benzothiazole, 3-(7-Chloro-1-oxo-1,3-dihydro-isoindol-2-yl)-N-[2-(2′-cyano-3,4,5,6-tetrahydro-2H-[1,4′]bipyridinyl-4-yl)-ethyl]-benzamide, 7-Chloro-2-[3-(9-pyridin-4-yl-3,9-diaza-spiro[5.5]undecane-3-carbonyl)-phenyl]-2,3-dihydro-isoindol-1-one, [3-(4-Chloro-benzothiazol-2-yl)-phenyl]-(9-pyridin-4-yl-3,9-diaza-spiro[5.5]undec-3-yl)-methanone, 7-Chloro-2-[5-(9-pyridin-4-yl-3,9-diaza-spiro[5.5]undecane-3-carbonyl)-thiophen-2-yl]-2,3-dihydro-isoindol-1-one, 7-Chloro-2-[3-(3′,4′,5′,6′,3″,4″,5″,6″-octahydro-2′H,2″H-[4,1′;4′,4″]terpyridine-1″-carbonyl)-phenyl]-2,3-dihydro-isoindol-1-one, 3-(1H-Indol-2-yl)-N-[2-(3,4,5,6-tetrahydro-2H-[1,4′]bipyridinyl-4-yl)-ethyl]-benzamide, 3-Benzothiazol-2-yl-N-[2-(3,4,5,6-tetrahydro-2H-[1,4′]bipyridinyl-4-yl)-ethyl]-benzamide, 2-{3-[4-(4-Amino-phenyl)-piperidine-1-carbonyl]-phenyl}-7-chloro-2,3-dihydro-isoindol-1-one, 3-(7-Chloro-1-oxo-1,3-dihydro-isoindol-2-yl)-N-[2-(5,6,7,8-tetrahydro-[1,8]naphthyridin-2-yl)-ethyl]-benzamide, 7-Chloro-2-{3-[4-(2-imidazol-1-yl-ethyl)-piperazine-1-carbonyl]-phenyl}-2,3-dihydro-isoindol-1-one, 7-Chloro-2-{3-[4-(1-methyl-piperidin-4-yl)-piperazine-1-carbonyl]-phenyl}-2,3-dihydro-isoindol-1-one, 7-Chloro-2-[3-(9-isopropyl-3,9-diaza-spiro[5.5]undecane-3-carbonyl)-phenyl]-2,3-dihydro-isoindol-1-one, 7-Chloro-2-{3-[4-(3-pyrrolidin-1-yl-propyl)-piperazine-1-carbonyl]-phenyl}-2,3-dihydro-isoindol-1-one, 7-Chloro-2-(3-[4-(1-methyl-piperidin-4-ylmethyl)-piperazine-1-carbonyl]-phenyl)-2,3-dihydro-isoindol-1-one, 7-Chloro-2-{3-[4-(piperidin-4-yloxymethyl)-piperidine-1-carbonyl]-phenyl}-2,3-dihydro-isoindol-1-one, 7-Chloro-2-[3-(5-pyridin-4-yl-3,4-dihydro-1H-isoquinoline-2-carbonyl)-phenyl]-2,3-dihydro-isoindol-1-one, 4-Chloro-2-[3′-(4-pyridin-4-yl-piperazin-1-ylmethyl)-biphenyl-3-yl]-1H-benzoimidazole, 7-Chloro-2-[3-(4-dimethylaminomethyl-[1,4′]bipiperidinyl-1′-carbonyl)-phenyl]-2,3-dihydro-isoindol-1-one, 7-Chloro-2-[3-(3′,4′,5′,6′,3″,4″,5″,6″-octahydro-2′H,2″H-[2,1′;4′,4″]terpyridine-1″-carbonyl)-phenyl]-2,3-dihydro-isoindol-1-one, 9-[3-(7-Chloro-1-oxo-1,3-dihydro-isoindol-2-yl)-benzoyl]-3,9-diaza-spiro[5.5]undecane-3-carboxylic acid tert-butyl ester, 7-Chloro-2-(3-pyridin-3-yl-phenyl)-2,3-dihydro-isoindol-1-one, 7-Chloro-2-[3-(4-isopropyl-piperazine-1-carbonyl)-phenyl]-2,3-dihydro-isoindol-1-one, 4-Chloro-2-[3-(5-piperidin-4-ylidenemethyl-pyridin-3-yl)-phenyl]-1H-benzoimidazole, 7-Chloro-2-[3-(3,9-diaza-spiro[5.5]undecane-3-carbonyl)-phenyl]-2,3-dihydro-isoindol-1-one, 3-Benzofuran-2-yl-N-[2-(3,4,5,6-tetrahydro-2H-[1,4′]bipyridinyl-4-yl)-ethyl]-benzamide, 3-(1H-Benzoimidazol-2-yl)-N-[2-(3,4,5,6-tetrahydro-2H-[1,4′]bipyridinyl-4-yl)-ethyl]benzamide, 3-Benzooxazol-2-yl-N-[2-(3,4,5,6-tetrahydro-2H-[1,4′]bipyridinyl-4-yl)-ethyl]-benzamide, 7-Chloro-2-{3-[4-(2-dimethylamino-ethyl)-piperazine-1-carbonyl]-phenyl}-2,3-dihydro-isoindol-1-one, 7-Chloro-2-[3-(4-pyridin-4-yl-piperazine-1-carbonyl)-phenyl]-2,3-dihydro-isoindol-1-one, 7-Chloro-2-[3-(4-pyridin-2-yl-piperazine-1-carbonyl)-phenyl]-2,3-dihydro-isoindol-1-one, 7-Chloro-2-[3-(4-pyrimidin-2-yl-piperazine-1-carbonyl)-phenyl]-2,3-dihydro-isoindol-1-one, 7-Chloro-2-[3-(4-cyclohexyl-piperazine-1-carbonyl)-phenyl]-2,3-dihydro-isoindol-1-one, 2-[3-([1,4′]Bipiperidinyl-1′-carbonyl)-phenyl]-7-chloro-2,3-dihydro-isoindol-1-one, 3-(1-Methyl-1H-benzoimidazol-2-yl)-N-[2-(3,4,5,6-tetrahydro-2H-[1,4′]bipyridinyl-4-yl)-ethyl]benzamide, 3-Benzo[b]thiophen-2-yl-N-[2-(3,4,5,6-tetrahydro-2H-[1,4′]bipyridinyl-4-yl)-ethyl]-benzamide, 2-[3-(4-Benzyl-piperazine-1-carbonyl)-phenyl]-7-chloro-2,3-dihydro-isoindol-1-one, 7-Chloro-2-[3-(4-phenoxy-piperidine-1-carbonyl)-phenyl]-2,3-dihydro-isoindol-1-one, 7-Chloro-2-{3-[4-(2-pyrrol-1-yl-ethyl)-piperazine-1-carbonyl]-phenyl}-2,3-dihydro-isoindol-1-one, 7-Chloro-2-[3-(4-cyclohexylmethyl-piperazine-1-carbonyl)-phenyl]-2,3-dihydro-isoindol-1-one, 7-Chloro-2-{3-[4-(pyrrolidine-1-carbonyl)-piperazine-1-carbonyl]-phenyl}-2,3-dihydro-isoindol-1-one, 7-Chloro-2-{3-[4-(piperidin-4-yloxy)-piperidine-1-carbonyl]-phenyl}-2,3-dihydro-isoindol-1-one, 7-Chloro-2-[3-(4-phenethyl-piperazine-1-carbonyl)-phenyl]-2,3-dihydro-isoindol-1-one, 1-[3-(4-Chloro-1H-benzoimidazol-2-yl)-piperidin-1-yl]-4-(3,4,5,6-tetrahydro-2H-[1,4′]bipyridinyl-4-yl)-butan-1-one, 4-{3-[3-(4-Chloro-1H-benzoimidazol-2-yl)-phenyl]-[1,2,4]oxadiazol-5-ylmethyl}-3,4,5,6-tetrahydro-2H-[1,4′]bipyridinyl, 3-(7-Chloro-benzothiazol-2-yl)-N-[2-(3,4,5,6-tetrahydro-2H-[1,4′]bipyridinyl-4-yl)-ethyl]-benzamide, 3-Benzothiazol-2-yl-4-chloro-N-[2-(3,4,5,6-tetrahydro-2H-[1,4′]bipyridinyl-4-yl)-ethyl]-benzamide, {6-[3-(4-Chloro-1H-benzoimidazol-2-yl)-phenyl]-pyridin-2-yl}-(3,4,5,6-tetrahydro-2H-[1,4′]bipyridinyl-4-yl)-amine, 2-[3-(4-Chloro-1H-benzoimidazol-2-yl)-piperidin-1-yl]-N-[2-(3,4,5,6-tetrahydro-2H-[1,4′]bipyridinyl-4-yl)-ethyl]-acetamide, 4-Chloro-2-{3-[5-(4-pyridin-4-yl-piperazin-1-ylmethyl)-thiophen-2-yl]-phenyl}-1H-benzoimidazole, 2-{4-[3-(7-Chloro-1-oxo-1,3-dihydro-isoindol-2-yl)-benzoyl]-piperazin-1-yl}-N-pyridin-2-yl-acetamide, {5-[3-(4-Chloro-1H-benzoimidazol-2-yl)-phenyl]-pyridin-3-yl}-(4-piperidin-1-ylmethyl-phenyl)-amine, 5-(7-Chloro-1-oxo-1,3-dihydro-isoindol-2-yl)-1H-pyrazole-3-carboxylic acid (2-oxo-5-phenyl-2,3-dihydro-1H-benzo[e][1,4]diazepin-3-yl)-amide, [6-Chloro-3′-(4-chloro-1H-benzoimidazol-2-yl)-biphenyl-3-yl]-(4-pyridin-4-yl-piperazin-1-yl)-methanone, 4-Bromo-5-(7-chloro-1-oxo-1,3-dihydro-isoindol-2-yl)-1H-pyrazole-3-carboxylic acid (2-oxo-5-phenyl-2,3-dihydro-1H-benzo[e][1,4]diazepin-3-yl)-amide, 5-(7-Chloro-1-oxo-1,3-dihydro-isoindol-2-yl)-1H-pyrazole-3-carboxylic acid [2-(3,4,5,6-tetrahydro-2H-[1,4′]bipyridinyl-4-yl)-ethyl]-amide, 5-(7-Chloro-1-oxo-1,3-dihydro-isoindol-2-yl)-1H-pyrazole-3-carboxylic acid (2-oxo-5-phenyl-2,3-dihydro-1H-benzo[e][1,4]diazepin-3-yl)-amide, 4-Bromo-5-(7-chloro-1-oxo-1,3-dihydro-isoindol-2-yl)-1H-pyrazole-3-carboxylic acid (2-oxo-5-phenyl-2,3-dihydro-1H-benzo[e][1,4]diazepin-3-yl)-amide, 7-Chloro-2-{5-[4-(pyridin-4-yloxy)piperidine-1-carbonyl]thiophen-2-yl)-2,3-dihydro-isoindol-1-one, 7-Chloro-2-{3-[5-(2-piperidin-4-ylvinyl)pyridin-3-yl]phenyl}-2,3-dihydroisoindol-1-one, 7-Chloro-2-[5-(1′-isopropyl-[4,4′]bipiperidinyl-1-carbonyl)thiophen-2-yl]-2,3-dihydroisoindol-1-one, 7-Chloro-2-{3-[5-(2-piperidin-4-yl-ethyl)pyridin-3-yl]phenyl}-2,3-dihydroisoindol-1-one, 7-Chloro-2-(3-[4-(4-isopropylpiperazin-1-yl)piperidine-1-carbonyl]phenyl}-2,3-dihydroisoindol-1-one, 5-{1-[3-(7-Chloro-1-oxo-1,3-dihydro-isoindol-2-yl)benzoyl]piperidin-4-yloxy}-3,4-dihydro-1H-isoquinoline-2-carboxylic acid tert-butyl ester, 2-Chloro-N-[5-(9-pyridin-4-yl-3,9-diaza-spiro[5.5]undecane-3-carbonyl)thiazol-2-yl]benzamide, 2-(2-Chlorobenzoylamino)thiazole-5-carboxylic acid [2-(3,4,5,6-tetrahydro-2H-[1,4′]bipyridinyl-4-yl)ethyl]amide, 7-Chloro-2-{3-[4-(1,2,3,4-tetrahydroisoquinolin-5-yloxy)piperidine-1-carbonyl]phenyl}-2,3-dihydroisoindol-1-one, 7-Chloro-2-{3-[5-(piperidin-4-ylmethoxy)pyridin-3-yl]phenyl)-2,3-dihydroisoindol-1-one, 4-{5-[3-(7-Chloro-1-oxo-1,3-dihydroisoindol-2-yl)phenyl]pyridin-3-yloxy}piperidine-1-carboxylic acid tert-butyl ester, 2-Chloro-N-[4-methyl-5-(9-pyridin-4-yl-3,9-diaza-spiro[5.5]undecane-3-carbonyl)thiazol-2-yl]benzamide, 7-Chloro-2-{3-[5-(piperidin-4-yloxy)pyridin-3-yl]phenyl}-2,3-dihydroisoindol-1-one, 7-Chloro-2-[3-(5-piperidin-4-ylidenemethylpyridin-3-yl)phenyl]-2,3-dihydroisoindol-1-one, 7-Chloro-2-[5-(9-pyridin-4-yl-3,9-diaza-spiro[5.5]undecane-3-carbonyl)furan-2-yl]-2,3-dihydroisoindol-1-one, 7-Chloro-2-{3-[5-(3,4,5,6-tetrahydro-2H-[1,4′]bipyridinyl-4-ylmethoxy)pyridin-3-yl]phenyl}-2,3-dihydroisoindol-1-one, 7-Chloro-2-{3-[5-(4-pyridin-4-yl-piperazin-1-ylmethyl)thiazol-2-yl]phenyl}-2,3-dihydroisoindol-1-one, 7-Chloro-2-{3-[2-(4-pyridin-4-yl-piperazin-1-ylmethyl)thiazol-4-yl]phenyl}-2,3-dihydroisoindol-1-one, 7-Chloro-2-[3-[5-(4-pyridin-4-yl-piperazin-1-ylmethyl)furan-2-yl]phenyl)-2,3-dihydroisoindol-1-one, 7-Chloro-2-(3-[4-(piperidin-4-ylmethoxy)pyridin-3-yl]phenyl}-2,3-dihydroisoindol-1-one, 7-Chloro-2-{3-[4-(3,4,5,6-tetrahydro-2H-[1,4′]bipyridinyl-4-ylmethoxy)pyridin-3-yl]phenyl)-2,3-dihydroisoindol-1-one, 7-Chloro-2-{3-[4-(piperidin-4-yloxy)pyridin-3-yl]phenyl}-2,3-dihydroisoindol-1-one, 7-Chloro-2-{3-[4-(3,4,5,6-tetrahydro-2H-[1,4′]bipyridinyl-4-yloxy)pyridin-3-yl]phenyl}-2,3-dihydroisoindol-1-one, 7-Chloro-2-{3-[5-(3,4,5,6-tetrahydro-2H-[1,4′]bipyridinyl-4-yloxy)pyridin-3-yl]phenyl}-2,3-dihydroisoindol-1-one, 7-Chloro-2-[3-(9-pyridin-4-yl-3,9-diaza-spiro[5.5]undec-3-ylmethyl)phenyl]-2,3-dihydroisoindol-1-one, 7-Chloro-2-{3-[4-(4-pyridin-4-yl-piperazin-1-ylmethyl)pyridin-2-yl]phenyl}-2,3-dihydroisoindol-1-one, 7-Chloro-2-[3-(4-hydroxymethylpyridin-2-yl)phenyl]-2,3-dihydroisoindol-1-one, 2-[3-(9-Pyridin-4-yl-3,9-diaza-spiro[5.5]undecane-3-carbonyl)phenyl]-2,3-dihydroisoindol-1-one, 4-(2-{5-[3-(7-Chloro-1-oxo-1,3-dihydroisoindol-2-yl)phenyl]pyridin-3-yloxyethyl)piperidine-1-carboxylic acid tert-butyl ester, 7-Chloro-2-{3-[5-(2,3,5,6-tetrahydro-[1,4′]bipyridinyl-4-ylidenemethyl)pyridin-3-yl]phenyl}-2,3-dihydroisoindol-1-one, 7-Chloro-2-{3-[4-(2-piperidin-4-ylethoxy)pyridin-3-yl]phenyl}-2,3-dihydroisoindol-1-one, 7-Chloro-2-{3-[5-(2-piperidin-4-ylethoxy)pyridin-3-yl]phenyl}-2,3-dihydroisoindol-1-one, 7-Chloro-2-(3-{4-[2-(3,4,5,6-tetrahydro-2H-[1,4′]bipyridinyl-4-yl)ethoxy]pyridin-3-yl}phenyl)-2,3-dihydroisoindol-1-one, 7-Chloro-2-(3-{5-[2-(3,4,5,6-tetrahydro-2H-[1,4′]bipyridinyl-4-yl)ethoxy]pyridin-3-yl}phenyl)-2,3-dihydro-isoindol-1-one, 7-Chloro-2-[3-(2-pyridin-4-yl-ethoxy)phenyl]-2,3-dihydroisoindol-1-one, 7-Chloro-2-(3-[2-(4-methylpiperazin-1-yl)ethoxy]phenyl}-2,3-dihydroisoindol-1-one, 4-[6-[3-(7-Chloro-1-oxo-1,3-dihydroisoindol-2-yl)phenyl]pyrimidin-4-ylamino]piperidine-1-carboxylic acid tert-butyl ester, 7-Chloro-2-{3-[6-(piperidin-4-ylamino)pyrimidin-4-yl]phenyl}-2,3-dihydroisoindol-1-one, 7-Chloro-2-(3-[5-[(2S,6R)-dimethyl-4-pyridin-4-ylpiperazin-1-ylmethyl]pyridin-3-yl]phenyl}-2,3-dihydro-isoindol-1-one, and 7-Chloro-2-{3-[5-(3,4,5,6-tetrahydro-2H-[1,4′]bipyridinyl-4-ylmethyl)pyridin-3-yl]phenyl}-2,3-dihydroisoindol-1-one.

In an embodiment, compounds of formula (I) include 2-[3-([4,4′]Bipiperidinyl-1-carbonyl)-phenyl]-7-chloro-2,3-dihydro-isoindol-1-one, 7-Chloro-2-[3-(9-methyl-3,9-diaza-spiro[5.5]undecane-3-carbonyl)-phenyl]-2,3-dihydro-isoindol-1-one, 7-Chloro-2-{3-[4-(pyridin-4-yloxy)-piperidine-1-carbonyl]-phenyl}-2,3-dihydro-isoindol-1-one, 7-Chloro-2-[3-(1′-methyl-[4,4′]bipiperidinyl-1-carbonyl)-phenyl]-2,3-dihydro-isoindol-1-one, 2-[3-(4-Amino-[1,4′]bipiperidinyl-1′-carbonyl)-phenyl]-7-chloro-2,3-dihydro-isoindol-1-one, 3-(7-Chloro-1H-benzoimidazol-2-yl)-N-[2-(3,4,5,6-tetrahydro-2H-[1,4′]bipyridinyl-4-yl)-ethyl]-benzamide, 3-(7-Chloro-1-oxo-1,3-dihydro-isoindol-2-yl)-N-[3-(5,6,7,8-tetrahydro-[1,8]naphthyridin-2-yl)-propyl]-benzamide, 7-Chloro-2-{3-[4-(pyridin-4-ylmethoxy)-piperidine-1-carbonyl]-phenyl}-2,3-dihydro-isoindol-1-one, 5-(7-Chloro-1-oxo-1,3-dihydro-isoindol-2-yl)-1H-pyrazole-3-carboxylic acid [2-(3,4,5,6-tetrahydro-2H-[1,4′]bipyridinyl-4-yl)-ethyl]-amide, 3-(4-Chloro-1H-benzoimidazol-2-yl)-piperidine-1-carboxylic acid [2-(3,4,5,6-tetrahydro-2H-[1,4′]bipyridinyl-4-yl)-ethyl]-amide, 4-Chloro-2-{3-[5-(4-pyridin-4-yl-piperazin-1-ylmethyl)-furan-2-yl]-phenyl}-1H-benzoimidazole, 4-Chloro-2-{3-[5-(4-pyridin-4-yl-piperazin-1-ylmethyl)-1H-imidazol-2-yl]-phenyl)-1H-benzoimidazole, 4-{5-[3-(4-Chloro-1H-benzoimidazol-2-yl)-phenyl]-4H-[1,2,4]triazol-3-ylmethyl}-3,4,5,6-tetrahydro-2H-[1,4′]bipyridinyl, 4-{5-[3-(4-Chloro-1H-benzoimidazol-2-yl)-phenyl]-tetrazol-2-ylmethyl-3,4,5,6-tetrahydro-2H-[1,4′]bipyridinyl, 3-(7-Chloro-1-oxo-1,3-dihydro-isoindol-2-yl)-N-[2-(3,4,5,6-tetrahydro-2H-[1,4′]bipyridinyl-4-yl)ethyl]-benzamide, 3-(4-Chloro-benzothiazol-2-yl)-N-[2-(3,4,5,6-tetrahydro-2H-[1,4′]bipyridinyl-4-yl)-ethyl]-benzamide, 7-Chloro-2-[3-(1′-isopropyl-[4,4′]bipiperidinyl-1-carbonyl)-phenyl]-2,3-dihydro-isoindol-1-one, 4-Chloro-2-{3-[5-(4-pyridin-4-yl-piperazin-1-ylmethyl)-pyridin-3-yl]-phenyl}-1H-benzoimidazole, 4-Chloro-2-{3-[4-(4-pyridin-4-yl-piperazin-1-ylmethyl)-pyridin-2-yl]-phenyl}-1H-benzoimidazole, 4-Chloro-2-{3-[6-(4-pyridin-4-yl-piperazin-1-ylmethyl)-pyridin-2-yl]-phenyl}-1H-benzoimidazole, 5-(7-Chloro-1-oxo-1,3-dihydro-isoindol-2-yl)-thiophene-2-carboxylic acid [2-(3,4,5,6-tetrahydro-2H-[1,4′]bipyridinyl-4-yl)-ethyl]-amide, 3-(7-Chloro-1-oxo-1,3-dihydro-isoindol-2-yl)-N-[2-(4-piperidin-1-ylmethyl-phenyl)-ethyl]-benzamide, 5-(7-Chloro-1-oxo-1,3-dihydro-isoindol-2-yl)-2-hydroxy-N-[2-(3,4,5,6-tetrahydro-2H-[1,4′]bipyridinyl-4-yl)-ethyl]-benzamide, 4-Chloro-3-(4-chloro-1H-benzoimidazol-2-yl)-N-[2-(3,4,5,6-tetrahydro-2H-[1,4′]bipyridinyl-4-yl)-ethyl]-benzamide, {2-[3-(4-Chloro-1H-benzoimidazol-2-yl)-phenyl]-pyridin-4-yl}-(4-pyridin-4-yl-piperazin-1-yl)-methanone, 4-Chloro-2-(3-{5-[1-(4-pyridin-4-yl-piperazin-1-yl)-ethyl]-pyridin-3-yl}-phenyl)-1H-benzoimidazole, 7-Chloro-2-{3-[5-(4-pyridin-4-yl-piperazin-1-ylmethyl)-pyridin-3-yl]-phenyl}-2,3-dihydro-isoindol-1-one, 7-Chloro-2-(3-[4-(1-isopropyl-piperidin-4-yloxy)-piperidine-1-carbonyl]-phenyl}-2,3-dihydro-isoindol-1-one, 4-Chloro-2-(3-[5-(4-pyridin-4-yl-piperazin-1-ylmethyl)-pyridin-3-yl]-phenyl}-benzothiazole, 3-(7-Chloro-1-oxo-1,3-dihydro-isoindol-2-yl)-N-[2-(2′-cyano-3,4,5,6-tetrahydro-2H-[1,4′]bipyridinyl-4-yl)-ethyl]-benzamide, 7-Chloro-2-[3-(9-pyridin-4-yl-3,9-diaza-spiro[5.5]undecane-3-carbonyl)-phenyl]-2,3-dihydro-isoindol-1-one, [3-(4-Chloro-benzothiazol-2-yl)-phenyl]-(9-pyridin-4-yl-3,9-diaza-spiro[5.5]undec-3-yl)-methanone, 7-Chloro-2-[5-(9-pyridin-4-yl-3,9-diaza-spiro[5.5]undecane-3-carbonyl)-thiophen-2-yl]-2,3-dihydro-isoindol-1-one, 7-Chloro-2-[3-(3′,4′,5′,6′,3″,4″,5″,6″-octahydro-2′H,2″H-[4,1′;4′,4″]terpyridine-1″-carbonyl)-phenyl]-2,3-dihydro-isoindol-1-one, 7-Chloro-2-{5-[4-(pyridin-4-yloxy)piperidine-1-carbonyl]thiophen-2-yl}-2,3-dihydro-isoindol-1-one, 7-Chloro-2-{3-[5-(2-piperidin-4-ylvinyl)pyridin-3-yl]phenyl}-2,3-dihydroisoindol-1-one, 7-Chloro-2-[5-(1′-isopropyl-[4,4′]bipiperidinyl-1-carbonyl)thiophen-2-yl]-2,3-dihydroisoindol-1-one, 7-Chloro-2-{3-[5-(2-piperidin-4-yl-ethyl)pyridin-3-yl]phenyl}-2,3-dihydroisoindol-1-one, 7-Chloro-2-{3-[4-(4-isopropylpiperazin-1-yl)piperidine-1-carbonyl]phenyl}-2,3-dihydroisoindol-1-one, 2-Chloro-N-[5-(9-pyridin-4-yl-3,9-diaza-spiro[5.5]undecane-3-carbonyl)thiazol-2-yl]benzamide, 2-(2-Chlorobenzoylamino)thiazole-5-carboxylic acid [2-(3,4,5,6-tetrahydro-2H-[1,4′]bipyridinyl-4-yl)ethyl]amide, 7-Chloro-2-{3-[4-(1,2,3,4-tetrahydroisoquinolin-5-yloxy)piperidine-1-carbonyl]phenyl}-2,3-dihydroisoindol-1-one, 7-Chloro-2-{3-[5-(piperidin-4-ylmethoxy)pyridin-3-yl]phenyl}-2,3-dihydroisoindol-1-one, 2-Chloro-N-[4-methyl-5-(9-pyridin-4-yl-3,9-diaza-spiro[5.5]undecane-3-carbonyl)thiazol-2-yl]benzamide, 7-Chloro-2-{3-[5-(piperidin-4-yloxy)pyridin-3-yl]phenyl}-2,3-dihydroisoindol-1-one, 7-Chloro-2-[3-(5-piperidin-4-ylidenemethylpyridin-3-yl)phenyl]-2,3-dihydroisoindol-1-one, 7-Chloro-2-[5-(9-pyridin-4-yl-3,9-diaza-spiro[5.5]undecane-3-carbonyl)furan-2-yl]-2,3-dihydroisoindol-1-one, 7-Chloro-2-{3-[5-(3,4,5,6-tetrahydro-2H-[1,4′]bipyridinyl-4-ylmethoxy)pyridin-3-yl]phenyl}-2,3-dihydroisoindol-1-one, 7-Chloro-2-{3-[5-(4-pyridin-4-yl-piperazin-1-ylmethyl)thiazol-2-yl]phenyl}-2,3-dihydroisoindol-1-one, 7-Chloro-2-{3-[2-(4-pyridin-4-yl-piperazin-1-ylmethyl)thiazol-4-yl]phenyl}-2,3-dihydroisoindol-1-one, 7-Chloro-2-{3-[5-(4-pyridin-4-yl-piperazin-1-ylmethyl)furan-2-yl]phenyl}-2,3-dihydroisoindol-1-one, 7-Chloro-2-{3-[5-(3,4,5,6-tetrahydro-2H-[1,4′]bipyridinyl-4-yloxy)pyridin-3-yl]phenyl}-2,3-dihydroisoindol-1-one, 7-Chloro-2-[3-(9-pyridin-4-yl-3,9-diaza-spiro[5.5]undec-3-ylmethyl)phenyl]-2,3-dihydroisoindol-1-one, 7-Chloro-2-{3-[4-(4-pyridin-4-yl-piperazin-1-ylmethyl)pyridin-2-yl]phenyl)-2,3-dihydroisoindol-1-one, 2-[3-(9-Pyridin-4-yl-3,9-diaza-spiro[5.5]undecane-3-carbonyl)phenyl]-2,3-dihydroisoindol-1-one, 7-Chloro-2-{3-[5-(2,3,5,6-tetrahydro-[1,4′]bipyridinyl-4-ylidenemethyl)pyridin-3-yl]phenyl}-2,3-dihydroisoindol-1-one, 7-Chloro-2-{3-[5-(2-piperidin-4-ylethoxy)pyridin-3-yl]phenyl}-2,3-dihydroisoindol-1-one, 7-Chloro-2-(3-{5-[2-(3,4,5,6-tetrahydro-2H-[1,4′]bipyridinyl-4-yl)ethoxy]pyridin-3-yl}phenyl)-2,3-dihydro-isoindol-1-one, 7-Chloro-2-{3-[6-(piperidin-4-ylamino)pyrimidin-4-yl]phenyl}-2,3-dihydroisoindol-1-one, 7-Chloro-2-{3-[5-[(2S,6R)-dimethyl-4-pyridin-4-ylpiperazin-1-ylmethyl]pyridin-3-yl]phenyl}-2,3-dihydro-isoindol-1-one, and 7-Chloro-2-{3-[5-(3,4,5,6-tetrahydro-2H-[1,4′]bipyridinyl-4-ylmethyl)pyridin-3-yl]phenyl}-2,3-dihydroisoindol-1-one.

In another embodiment, compounds of formula (I) include 3-(1H-Indol-2-yl)-N-[2-(3,4,5,6-tetrahydro-2H-[1,4′]bipyridinyl-4-yl)-ethyl]-benzamide, 3-Benzothiazol-2-yl-N-[2-(3,4,5,6-tetrahydro-2H-[1,4′]bipyridinyl-4-yl)-ethyl]-benzamide, 2-{3-[4-(4-Amino-phenyl)-piperidine-1-carbonyl]-phenyl}-7-chloro-2,3-dihydro-isoindol-1-one, 3-(7-Chloro-1-oxo-1,3-dihydro-isoindol-2-yl)-N-[2-(5,6,7,8-tetrahydro-[1,8]naphthyridin-2-yl)-ethyl]-benzamide, 7-Chloro-2-{3-[4-(2-imidazol-1-yl-ethyl)-piperazine-1-carbonyl]-phenyl}-2,3-dihydro-isoindol-1-one, 7-Chloro-2-{3-[4-(1-methyl-piperidin-4-yl)-piperazine-1-carbonyl]-phenyl}-2,3-dihydro-isoindol-1-one, 7-Chloro-2-[3-(9-isopropyl-3,9-diaza-spiro[5.5]undecane-3-carbonyl)-phenyl]-2,3-dihydro-isoindol-1-one, 7-Chloro-2-{3-[4-(3-pyrrolidin-1-yl-propyl)-piperazine-1-carbonyl]-phenyl}-2,3-dihydro-isoindol-1-one, 7-Chloro-2-{3-[4-(1-methyl-piperidin-4-ylmethyl)-piperazine-1-carbonyl]-phenyl}-2,3-dihydro-isoindol-1-one, 7-Chloro-2-{3-[4-(piperidin-4-yloxymethyl)-piperidine-1-carbonyl]-phenyl}-2,3-dihydro-isoindol-1-one, 7-Chloro-2-[3-(5-pyridin-4-yl-3,4-dihydro-1H-isoquinoline-2-carbonyl)-phenyl]-2,3-dihydro-isoindol-1-one, 4-Chloro-2-[3′-(4-pyridin-4-yl-piperazin-1-ylmethyl)-biphenyl-3-yl]-1H-benzoimidazole, 7-Chloro-2-[3-(4-dimethylaminomethyl-[1,4′]bipiperidinyl-1′-carbonyl)-phenyl]-2,3-dihydro-isoindol-1-one, 7-Chloro-2-[3-(3′,4′,5′,6′,3″,4″,5″,6″-octahydro-2′H,2″H-[2,1′;4′,4″]terpyridine-1″-carbonyl)-phenyl]-2,3-dihydro-isoindol-1-one, 9-[3-(7-Chloro-1-oxo-1,3-dihydro-isoindol-2-yl)-benzoyl]-3,9-diaza-spiro[5.5]undecane-3-carboxylic acid tert-butyl ester, 4-{5-[3-(7-Chloro-1-oxo-1,3-dihydroisoindol-2-yl)phenyl]pyridin-3-yloxy}piperidine-1-carboxylic acid tert-butyl ester, 5-{1-[3-(7-Chloro-1-oxo-1,3-dihydro-isoindol-2-yl)benzoyl]piperidin-4-yloxy}-3,4-dihydro-1H-isoquinoline-2-carboxylic acid tert-butyl ester, 7-Chloro-2-{3-[4-(3,4,5,6-tetrahydro-2H-[1,4′]bipyridinyl-4-ylmethoxy)pyridin-3-yl]phenyl}-2,3-dihydroisoindol-1-one, 7-Chloro-2-{3-[4-(3,4,5,6-tetrahydro-2H-[1,4′]bipyridinyl-4-yloxy)pyridin-3-yl]phenyl}-2,3-dihydroisoindol-1-one, and 7-Chloro-2-(3-{4-[2-(3,4,5,6-tetrahydro-2H-[1,4′]bipyridinyl-4-yl)ethoxy]pyridin-3-ylphenyl)-2,3-dihydroisoindol-1-one.

In another embodiment, compounds of formula (I) include 7-Chloro-2-(3-pyridin-3-yl-phenyl)-2,3-dihydro-isoindol-1-one, 7-Chloro-2-[3-(4-isopropyl-piperazine-1-carbonyl)-phenyl]-2,3-dihydro-isoindol-1-one, 4-Chloro-2-[3-(5-piperidin-4-ylidenemethyl-pyridin-3-yl)-phenyl]-1H-benzoimidazole, 7-Chloro-2-[3-(3,9-diaza-spiro[5.5]undecane-3-carbonyl)-phenyl]-2,3-dihydro-isoindol-1-one, 3-Benzofuran-2-yl-N-[2-(3,4,5,6-tetrahydro-2H-[1,4′]bipyridinyl-4-yl)-ethyl]-benzamide, 3-(1H-Benzoimidazol-2-yl)-N-[2-(3,4,5,6-tetrahydro-2H-[1,4′]bipyridinyl-4-yl)-ethyl]benzamide, 3-Benzooxazol-2-yl-N-[2-(3,4,5,6-tetrahydro-2H-[1,4′]bipyridinyl-4-yl)-ethyl]-benzamide, 7-Chloro-2-{3-[4-(2-dimethylamino-ethyl)-piperazine-1-carbonyl]-phenyl}-2,3-dihydro-isoindol-1-one, 7-Chloro-2-[3-(4-pyridin-4-yl-piperazine-1-carbonyl)-phenyl]-2,3-dihydro-isoindol-1-one, 7-Chloro-2-[3-(4-pyridin-2-yl-piperazine-1-carbonyl)-phenyl]-2,3-dihydro-isoindol-1-one, 7-Chloro-2-[3-(4-pyrimidin-2-yl-piperazine-1-carbonyl)-phenyl]-2,3-dihydro-isoindol-1-one, 7-Chloro-2-[3-(4-cyclohexyl-piperazine-1-carbonyl)-phenyl]-2,3-dihydro-isoindol-1-one, 2-[3-([1,4′]Bipiperidinyl-1′-carbonyl)-phenyl]-7-chloro-2,3-dihydro-isoindol-1-one, 3-(1-Methyl-1H-benzoimidazol-2-yl)-N-[2-(3,4,5,6-tetrahydro-2H-[1,4′]bipyridinyl-4-yl)-ethyl]benzamide, 3-Benzo[b]thiophen-2-yl-N-[2-(3,4,5,6-tetrahydro-2H-[1,4′]bipyridinyl-4-yl)-ethyl]-benzamide, 2-[3-(4-Benzyl-piperazine-1-carbonyl)-phenyl]-7-chloro-2,3-dihydro-isoindol-1-one, 7-Chloro-2-[3-(4-phenoxy-piperidine-1-carbonyl)-phenyl]-2,3-dihydro-isoindol-1-one, 7-Chloro-2-{3-[4-(2-pyrrol-1-yl-ethyl)-piperazine-1-carbonyl]-phenyl)-2,3-dihydro-isoindol-1-one, 7-Chloro-2-[3-(4-cyclohexylmethyl-piperazine-1-carbonyl)-phenyl]-2,3-dihydro-isoindol-1-one, 7-Chloro-2-{3-[4-(pyrrolidine-1-carbonyl)-piperazine-1-carbonyl]-phenyl}-2,3-dihydro-isoindol-1-one, 7-Chloro-2-{3-[4-(piperidin-4-yloxy)-piperidine-1-carbonyl]-phenyl}-2,3-dihydro-isoindol-1-one, 7-Chloro-2-[3-(4-phenethyl-piperazine-1-carbonyl)-phenyl]-2,3-dihydro-isoindol-1-one, 1-[3-(4-Chloro-1H-benzoimidazol-2-yl)-piperidin-1-yl]-4-(3,4,5,6-tetrahydro-2H-[1,4′]bipyridinyl-4-yl)-butan-1-one, 4-{3-[3-(4-Chloro-1H-benzoimidazol-2-yl)-phenyl]-[1,2,4]oxadiazol-5-ylmethyl}-3,4,5,6-tetrahydro-2H-[1,4′]bipyridinyl, 3-(7-Chloro-benzothiazol-2-yl)-N-[2-(3,4,5,6-tetrahydro-2H-[1,4′]bipyridinyl-4-yl)-ethyl]-benzamide, 3-Benzothiazol-2-yl-4-chloro-N-[2-(3,4,5,6-tetrahydro-2H-[1,4′]bipyridinyl-4-yl)-ethyl]-benzamide, {6-[3-(4-Chloro-1H-benzoimidazol-2-yl)-phenyl]-pyridin-2-yl}-(3,4,5,6-tetrahydro-2H-[1,4′]bipyridinyl-4-yl)-amine, 2-[3-(4-Chloro-1H-benzoimidazol-2-yl)-piperidin-1-yl]-N-[2-(3,4,5,6-tetrahydro-2H-[1,4′]bipyridinyl-4-yl)-ethyl]-acetamide, 4-Chloro-2-{3-[5-(4-pyridin-4-yl-piperazin-1-ylmethyl)-thiophen-2-yl]-phenyl}-1H-benzoimidazole, 2-{4-[3-(7-Chloro-1-oxo-1,3-dihydro-isoindol-2-yl)-benzoyl]-piperazin-1-yl}-N-pyridin-2-yl-acetamide, (5-[3-(4-Chloro-1H-benzoimidazol-2-yl)-phenyl]-pyridin-3-yl)-(4-piperidin-1-ylmethyl-phenyl)-amine, 5-(7-Chloro-1-oxo-1,3-dihydro-isoindol-2-yl)-1H-pyrazole-3-carboxylic acid (2-oxo-5-phenyl-2,3-dihydro-1H-benzo[e][1,4]diazepin-3-yl)-amide, [6-Chloro-3′-(4-chloro-1H-benzoimidazol-2-yl)-biphenyl-3-yl]-(4-pyridin-4-yl-piperazin-1-yl)-methanone, 4-Bromo-5-(7-chloro-1-oxo-1,3-dihydro-isoindol-2-yl)-1H-pyrazole-3-carboxylic acid (2-oxo-5-phenyl-2,3-dihydro-1H-benzo[e][1,4]diazepin-3-yl)-amide, 7-Chloro-2-[3-[4-(piperidin-4-ylmethoxy)pyridin-3-yl]phenyl}-2,3-dihydroisoindol-1-one, 7-Chloro-2-{3-[4-(piperidin-4-yloxy)pyridin-3-yl]phenyl}-2,3-dihydroisoindol-1-one, 7-Chloro-2-[3-(4-hydroxymethylpyridin-2-yl)phenyl]-2,3-dihydroisoindol-1-one, 4-(2-{5-[3-(7-Chloro-1-oxo-1,3-dihydroisoindol-2-yl)phenyl]pyridin-3-yloxy)ethyl)piperidine-1-carboxylic acid tert-butyl ester, 7-Chloro-2-{3-[4-(2-piperidin-4-ylethoxy)pyridin-3-yl]phenyl}-2,3-dihydroisoindol-1-one, 7-Chloro-2-[3-(2-pyridin-4-yl-ethoxy)phenyl]-2,3-dihydroisoindol-1-one, 7-Chloro-2-{3-[2-(4-methylpiperazin-1-yl)ethoxy]phenyl}-2,3-dihydroisoindol-1-one, and 4-{6-[3-(7-Chloro-1-oxo-1,3-dihydroisoindol-2-yl)phenyl]pyrimidin-4-ylamino}piperidine-1-carboxylic acid tert-butyl ester.

Another embodiment of the present invention is a compound of formula (II), 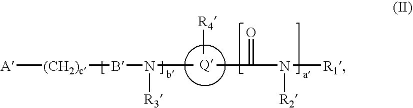
or a pharmaceutically acceptable salt thereof, wherein
a′ is 1;
b′ is 1;
c′ is 0, 1, or 2;
Q′ is selected from structure Q′(a), Q′(b), and Q′(c), 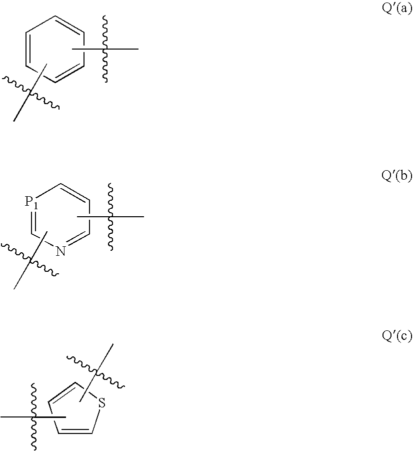
P₁ is selected from —CH— and —N—;
R₁′ is selected from R₁(a), R₁(b), R₁(c), R₁(d), R₁(e), R₁(f), R₁(g), and R₁(h), 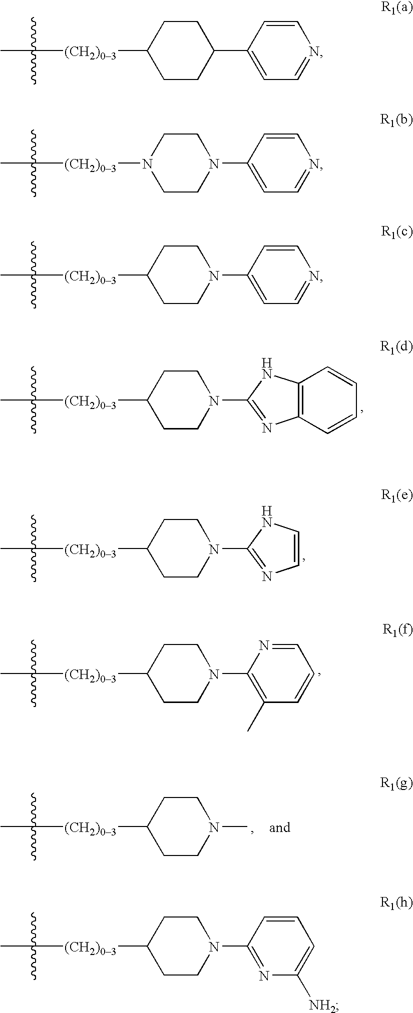
R₂′ is hydrogen;
or R₁′ and R₂′ together with the nitrogen to which they are attached form 9-pyridin-4-yl-3,9-diaza-spiro[5.5]undec-3-yl;
R₃′ is selected from hydrogen and alkyl;
R₄′ is selected from hydrogen and halogen;
B′ is selected from —C(O)— and —S(O)₂—; and
A′ is structure A(e), 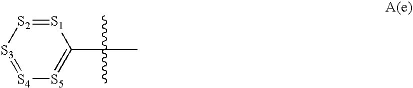
S₁ and S₄ are each independently selected from —CH—, —C(R₅₅′)—, and —N—;
S₂, S₃, and S₅ are each independently selected from —CH— and —C(R₅₅′)—; and
R₅₅′ at each occurrence is independently selected from halogen, alkyl, and —CF₃.

Among the compounds of formula (II), examples include 3-(2-chlorobenzoylamino)-2-chloro-N-[2-(3,4,5,6-tetrahydro-2H-[1,4′]bipyridinyl-4-yl)-ethyl]-benzamide, 3-(3-chlorobenzoylamino)-N-[2-(3,4,5,6-tetrahydro-2H-[1,4′]bipyridinyl-4-yl)-ethyl]-benzamide, 3-(3-chlorobenzoylamino)-2-chloro-N-[2-(3,4,5,6-tetrahydro-2H-[1,4′]bipyridinyl-4-yl)-ethyl]-benzamide, 3-[(4-chloro-2,5-dimethyl-benzenesulfonyl)-methyl-amino]-N-[2-(3,4,5,6-tetrahydro-2H-[1,4′]bipyridinyl-4-yl)-ethyl]-benzamide, 3-[(2-chlorobenzoyl)methylamino]-N-[2-(3,4,5,6-tetrahydro-2H-[1,4′]bipyridinyl-4-yl)-ethyl]-benzamide, 3-(2-chlorobenzoylamino)-N-[2-(3,4,5,6-tetrahydro-2H-[1,4′]bipyridinyl-4-yl)-ethyl]-benzamide, 3-(2,3-dichlorobenzoylamino)-N-[2-(3,4,5,6-tetrahydro-2H-[1,4′]bipyridinyl-4-yl)-ethyl]-benzamide, 3-(2,6-dichlorobenzoylamino)-N-[2-(3,4,5,6-tetrahydro-2H-[1,4′]bipyridinyl-4-yl)-ethyl]-benzamide, 3-(2-chlorobenzoylamino)-4-chloro-N-[2-(3,4,5,6-tetrahydro-2H-[1,4′]bipyridinyl-4-yl)-ethyl]-benzamide, 4-chloro-3-(4-chloro-2,5-dimethyl-benzenesulfonylamino)-N-[2-(3,4,5,6-tetrahydro-2H-[1,4′]bipyridinyl-4-yl)-ethyl]-benzamide, 3-(2-trifluoromethylbenzoylamino)-N-[2-(3,4,5,6-tetrahydro-2H-[1,4′]bipyridinyl-4-yl)-ethyl]-benzamide, 4-chloro-3-[(4-chloro-2,5-dimethyl-benzenesulfonyl)-methyl-amino]-N-[2-(3,4,5,6-tetrahydro-2H-[1,4′]bipyridinyl-4-yl)-ethyl]-benzamide, 3-(2-chlorobenzoylamino)-4-fluoro-N-[2-(3,4,5,6-tetrahydro-2H-[1,4′]bipyridinyl-4-yl)-ethyl]-benzamide, 4-chloro-3-[(4-chloro-2,5-dimethyl-benzenesulfonyl)-methyl-amino]-N-(3,4,5,6-tetrahydro-2H-[1,4′]bipyridinyl-4-ylmethyl)-benzamide, N-{2-[1-(1H-benzoimidazol-2-yl)-piperidin-4-yl]-ethyl}-4-chloro-3-[(4-chloro-2,5-dimethyl-benzenesulfonyl)-methyl-amino]-benzamide, 4-chloro-3-[(2,3-dichloro-benzenesulfonyl)-methyl-amino]-N-[2-(3,4,5,6-tetrahydro-2H-[1,4′]bipyridinyl-4-yl)-ethyl]-benzamide, 3-(2-chlorobenzoylamino)-4-bromo-N-[2-(3,4,5,6-tetrahydro-2H-[1,4′]bipyridinyl-4-yl)-ethyl]-benzamide, 4-chloro-3-[(4-chloro-2,5-dimethyl-benzenesulfonyl)-methyl-amino]-N-{2-[1-(1H-imidazol-2-yl)-piperidin-4-yl]-ethyl}-benzamide, 4-chloro-3-[(3,4-dichloro-benzenesulfonyl)-methyl-amino]-N-[2-(3,4,5,6-tetrahydro-2H-[1,4′]bipyridinyl-4-yl)-ethyl]-benzamide, 4-bromo-3-[(4-chloro-2,5-dimethyl-benzenesulfonyl)-methyl-amino]-N-[2-(3,4,5,6-tetrahydro-2H-[1,4′]bipyridinyl-4-yl)-ethyl]-benzamide, 4-chloro-3-[(2,6-dichloro-benzenesulfonyl)-methyl-amino]-N-[2-(3,4,5,6-tetrahydro-2H-[1,4′]bipyridinyl-4-yl)-ethyl]-benzamide, 6-(2-chloro-benzoylamino)-N-[2-(3,4,5,6-tetrahydro-2H-[1,4′]bipyridinyl-4-yl)-ethyl]-nicotinamide, 2,4-dichloro-5-(4-chloro-2,5-dimethyl-benzenesulfonylamino)-N-[2-(3,4,5,6-tetrahydro-2H-[1,4′]bipyridinyl-4-yl)-ethyl]-benzamide, 2,4-dichloro-5-[(4-chloro-2,5-dimethyl-benzenesulfonyl)-methyl-amino]-N-[2-(3,4,5,6-tetrahydro-2H-[1,4′]bipyridinyl-4-yl)-ethyl]-benzamide, 5-(2-chlorobenzoylamino)-2,4-dichloro-N-[2-(3,4,5,6-tetrahydro-2H-[1,4′]bipyridinyl-4-yl)-ethyl]-benzamide, 3-chloro-pyrazine-2-carboxylic acid {2-chloro-5-[2-(3,4,5,6-tetrahydro-2H-[1,4′]bipyridinyl-4-yl)-ethylcarbamoyl]-phenyl}-amide, 6-(2-chlorobenzoylamino)pyridine-2-carboxylic acid [2-(3,4,5,6-tetrahydro-2H-[1,4′]bipyridinyl-4-yl)-ethyl]-amide, 4-(2-chlorobenzoylamino)pyridine-2-carboxylic acid [2-(3,4,5,6-tetrahydro-2H-[1,4′]bipyridinyl-4-yl)-ethyl]-amide, 2-(2-chlorobenzoylamino)-N-[2-(3,4,5,6-tetrahydro-2H-[1,4′]bipyridinyl-4-yl)-ethyl]-isonicotinamide, 5-(2-chlorobenzoylamino)-N-[2-(3,4,5,6-tetrahydro-2H-[1,4′]bipyridinyl-4-yl)-ethyl]-nicotinamide, 3-(2-chlorobenzoylamino)-4-chloro-N-[2-(3′-methyl-3,4,5,6-tetrahydro-2H-[1,2′]bipyridinyl-4-yl)-ethyl]-benzamide, 3-(2-chlorobenzoylamino)-4-chloro-N-[2-(1-methyl-piperidin-4-yl)-ethyl]-benzamide, 3-(2-chlorobenzoylamino)-4-chloro-N-[2-(4-pyridin-4-yl-piperazin-1-yl)-ethyl]-benzamide, 2-chloro-N-[3-(2-3,4,5,6-tetrahydro-2H-[1,4′]bipyridinyl-4-yl-acetylamino)-phenyl]-benzamide, 2-chloro-N-[2-chloro-5-(9-pyridin-4-yl-3,9-diaza-spiro[5.5]undecane-3-carbonyl)-phenyl]-benzamide, 3-(2-chlorobenzoylamino)-N-[2-(6′-amino-3,4,5,6-tetrahydro-2H-[1,2′]bipyridinyl-4-yl)-ethyl]-4-chloro-benzamide, and 5-(2-chloro-benzoylamino)-thiophene-2-carboxylic acid [2-(3,4,5,6-tetrahydro-2H-[1,4′]bipyridinyl-4-yl)-ethyl]-amide, and the like, or pharmaceutically acceptable salts thereof.

Another embodiment of the present invention, is a method of preventing or treating at least one condition which benefits from inhibition of the bradykinin B1 receptor, comprising: administering to a host in need thereof a composition comprising a therapeutically effective amount of at least one compound of formula (II), or a pharmaceutically acceptable salt thereof.

Another embodiment of the present invention is a pharmaceutical composition comprising, a pharmaceutically acceptable carrier and a therapeutically effective amount of at least one compound of formula (I) or formula (II), or mixtures thereof, effective to treat or ameliorate adverse symptoms in mammals mediated by bradykinin B₁ receptor.

Another embodiment of the present invention is a method of preventing or treating conditions which benefit from inhibition of the bradykinin B₁ receptor, comprising:

administering to a host in need thereof a composition comprising a therapeutically effective amount of at least one compound of formula (I) or formula (II), or pharmaceutically acceptable salts thereof.

In another embodiment, the present invention provides an article of manufacture, comprising (a) at least one dosage form of at least one compound of formula (I) or formula (II), or pharmaceutically acceptable salt thereof, optionally in combination with one or more active and/or inactive pharmaceutical agents, (b) a package insert providing that a dosage form comprising at least one compound of formula (I) or formula (II) should be administered to a patient in need of therapy for disorders, conditions or diseases which benefit from inhibition of the bradykinin B₁ receptor, and (c) at least one container in which at least one dosage form of at least one compound of formula (I) or formula (II), optionally in combination with one or more active and/or inactive pharmaceutical agents, is stored.

In another embodiment, the present invention provides a packaged pharmaceutical composition for treating diseases, disorders, and conditions, which benefit from inhibition of the bradykinin B₁ receptor, (a) a container which holds an effective amount of at least one compound of formula (I) or formula (II), or a pharmaceutically acceptable salt thereof, and (b) instructions for using the pharmaceutical composition.

BK is a kinin that plays an important role in the patho-physiological processes accompanying acute and chronic pain and inflammation. BKs, like other related kinins, are autocoid peptides produced by the catalytic action of kallikrein enzymes on plasma and tissue precursors termed kininogens. Inhibition of bradykinin B1 receptors by compounds that are bradykinin B1 antagonists or inverse agonists would provide relief from maladies that mediate undesirable symptoms through a bradykinin B1 receptor pathway.

The compounds of this invention are bradykinin B₁ receptor antagonists and therefore are suitable for use in blocking or ameliorating pain as well as hyperalgesia in mammals. Such compounds would be effective in the treatment or prevention of pain including, for example, bone and joint pain (osteoarthritis), repetitive motion pain, dental pain, pain associated with cancer, myofascial pain (muscular injury, fibromyalgia), perioperative pain (general surgery, gynecological) and chronic pain. In particular, inflammatory pain such as, for example, inflammatory airways disease (chronic obstructive pulmonary disease) would be effectively treated by bradykinin B1 antagonist compounds.

The compounds of this invention are also useful in the treatment of disease conditions in a mammal that are mediated, at least in part, by a bradykinin B₁ receptor. Examples of such disease conditions include asthma, inflammatory bowel disease, rhinitis, pancreatitis, cystitis (interstitial cystitis), uveitis, inflammatory skin disorders, rheumatoid arthritis and edema resulting from trauma associated with burns, sprains or fracture. They may be used subsequent to surgical intervention (e.g., as post-operative analgesics) and to treat inflammatory pain of varied origins (e.g., osteoarthritis, rheumatoid arthritis, rheumatic disease, tenosynovitis and gout), as well as for the treatment of pain associated with angina, menstruation, or cancer. They may also be used to treat diabetic vasculopathy, post capillary resistance or diabetic symptoms associated with insulitis (e.g., hyperglycemia, diuresis, proteinuria and increased nitrite and kallikrein urinary excretion). They may be used as smooth muscle relaxants for the treatment of spasm of the gastrointestinal tract or uterus or in the therapy of Crohn's disease, ulcerative colitis or pancreatitis.

Such compounds may also be used therapeutically to treat hyperreactive airways and to treat inflammatory events associated with diseases or conditions affecting the airways (e.g., asthma), and to control, restrict or reverse airway hyperreactivity in asthma. They may be used to treat intrinsic and extrinsic asthma, including allergic asthma (atopic or non-atopic), as well as exercise-induced asthma, occupational asthma, asthma post-bacterial infection, other non-allergic asthmas and “wheezy-infant syndrome”. They may also be effective against pneumoconiosis, including aluminosis, anthracosis, asbestosis, chalicosis, ptilosis, siderosis, silicosis, tabacosis and byssinosis, as well as adult respiratory distress syndrome, chronic obstructive pulmonary or diseases or conditions affecting the airways, bronchitis, allergic rhinitis, and vasomotor rhinitis. Additionally, they may be effective against liver disease, multiple sclerosis, atherosclerosis, Alzheimer's disease, septic shock (e.g., as anti-hypovolemic and/or anti-hypotensive agents), cerebral edema, headache, migraine, closed head trauma, irritable bowel syndrome and nephritis. Finally, such compounds are also useful as research tools (in vivo and in vitro).

As noted above, the compounds of this invention are typically administered to the mammal in the form of a pharmaceutical composition. Pharmaceutical compositions of the invention are suitable for use in a variety of drug delivery systems. Suitable formulations for use in the present invention are found in Remington's Pharmaceutical Sciences, Mace Publishing Company, Philadelphia, Pa., 17th ed. (1985).

To enhance serum half-life, the compounds may be encapsulated, introduced into the lumen of liposomes, prepared as a colloid, or other conventional techniques may be employed which provide an extended serum half-life of the compounds. A variety of methods are available for preparing liposomes, as described in, e.g., Szoka, et al., U.S. Pat. Nos. 4,235,871, 4,501,728 and 4,837,028 (each of which is incorporated herein by reference in full).

The amount administered to the patient will vary depending upon what is being administered, the purpose of the administration, such as prophylaxis or therapy, the state of the patient, the manner of administration, and the like all of which are within the skill of the attending clinician. In therapeutic applications, compositions are administered to a patient already suffering from a disease in an amount sufficient to cure or at least partially arrest the symptoms of the disease and its complications. An amount adequate to accomplish this is defined as “therapeutically effective dose.” Amounts effective for this use will depend on the disease condition being treated as well as by the judgment of the attending clinician depending upon factors such as the severity of the inflammation, the age, weight and general condition of the patient, and the like.

The compositions administered to a patient are in the form of pharmaceutical compositions described above. These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of the compounds of the present invention will vary according to, for example, the particular use for which the treatment is made, the manner of administration of the compound, the health and condition of the patient, and the judgment of the prescribing physician. For example, for intravenous administration, the dose will typically be in the range of about 20 Fg to about 500 Fg per kilogram body weight, preferably about 100 Fg to about 300 Fg per kilogram body weight. Suitable dosage ranges for intranasal administration are generally about 0.1 pg to 1 mg per kilogram body weight. Effective doses can be extrapolated from dose-response curves derived from in vitro or animal model test systems.

In those cases where the compounds of formula (I) or formula (II) exist as tautomers, optical isomers or geometric isomers, the above formulas are intended to represent isomer mixtures as well as the individual isomeric bradykinin B₁ receptor antagonist or intermediate isomers, all of which are encompassed within the scope of this invention.

Further, references to the compounds of formula (I) or formula (II) with respect to pharmaceutical applications thereof are also intended to include pharmaceutically acceptable salts of the compounds of formula (I) or formula (II).

In an embodiment, the present invention provides compounds of formula (I) or formula (II) that are selective antagonists of bradykinin B₁ receptor over bradykinin B₂ receptor.

In another embodiment, the present invention provides a method for selectively inhibiting bradykinin B₁ receptor over bradykinin B₂ receptor by administering to a host in need thereof an effective amount of at least one compound of formula (I) or formula (II), or pharmaceutically acceptable salts thereof.

In another embodiment, the present invention provides a pharmaceutical composition comprising a pharmaceutically acceptable carrier and a therapeutically effective amount of a compound of formula (I) or formula (II), or mixtures thereof, effective to treat or ameliorate adverse symptoms in mammals mediated by bradykinin B₁ receptor.

In another embodiment, the present invention provides a method for treating or ameliorating adverse symptoms in mammals mediated at least in part by bradykinin B₁ receptor comprising, administering a therapeutically effective amount of a compound of formula (I) or formula (II), or mixtures thereof, or as is more generally the case a pharmaceutical composition.

In another embodiment, the present invention provides a pharmaceutical composition comprising a pharmaceutically acceptable carrier and a therapeutically effective amount of a compound of formula (I) or formula (II), or mixtures thereof, to treat or ameliorate adverse symptoms in mammals associated with up-regulating bradykinin B₁ receptor following tissue damage or inflammation.

In another embodiment, the present invention provides a method for treating or ameliorating adverse symptoms in mammals associated with up-regulating bradykinin B₁ receptor following tissue damage or inflammation comprising, administering a therapeutically effective amount of a compound of formula (I) or formula (II), or mixtures thereof, or as is more generally the case a pharmaceutical composition.

In another embodiment, the present invention provides a method for treating or ameliorating adverse symptoms associated with the presence or secretion of bradykinin B₁ receptor agonists in mammals comprising, administering a therapeutically effective amount of a compound of formula (I) or formula (II), or mixtures thereof, or as is more generally the case a pharmaceutical composition.

In another embodiment, the present invention provides a method for treating or ameliorating pain, inflammation, septic shock or the scarring process in mammals mediated at least in part by bradykinin B₁ receptor in such mammals comprising, administering a therapeutically effective amount of a compound of formula (I) or formula (II), or mixtures thereof, or as is more generally the case the pharmaceutical composition.

In another embodiment, the present invention provides a method for treating or ameliorating adverse symptoms associated with up-regulating bradykinin B₁ receptor relative to burns, perioperative pain, migraine, shock, central nervous system injury, asthma, rhinitis, premature labor, inflammatory arthritis, inflammatory bowel disease, neuropathic pain or multiple sclerosis, comprising, administering a therapeutically effective amount of a compound of formula (I) or formula (II) or mixtures thereof or as is more generally the case the pharmaceutical composition.

In another embodiment, the present invention provides a method for treating or ameliorating adverse symptoms associated with the presence or secretion of bradykinin B₁ receptor agonists in mammals comprising, administering a therapeutically effective amount of a compound of formula (I) or formula (II) or mixtures thereof or as is more generally the case the pharmaceutical composition.

In another embodiment, the present invention provides a method for determining bradykinin B₁ receptor agonist levels in a biological sample comprising, contacting said biological sample with a compound of formula (I) or formula (II), at a predetermined concentration.

### Definitions

Throughout the specification and claims, including the detailed description below, the following definitions apply.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

Where multiple substituents are indicated as being attached to a structure, it is to be understood that the substituents can be the same or different.

Unless otherwise expressly defined with respect to a specific occurrence of the term, the following terms as used herein shall have the following meanings regardless of whether capitalized or not:

The term “alkyl” or the prefix “alk” in the present invention refers to straight or branched chain alkyl groups having 1 to 20 carbon atoms. An alkyl group may optionally comprise at least one double bond and/or at least one triple bond. The alkyl groups herein are unsubstituted or substituted in one or more positions with various groups. For example, such alkyl groups may be optionally substituted with at least one group selected from alkyl, alkoxy, —C(O)H, carboxy, alkoxycarbonyl, cycloalkyl, heterocycloalkyl, aryl, heteroaryl, amido, alkanoylamino, amidino, alkoxycarbonylamino, N-alkyl amidino, N-alkyl amido, N,N′-dialkylamido, aralkoxycarbonylamino, halogen, alkyl thio, alkylsulfinyl, alkylsulfonyl, hydroxy, cyano, nitro, amino, monoalkylamino, dialkylamino, halo alkyl, halo alkoxy, aminoalkyl, monoalkylaminoalkyl, dialkylaminoalkyl, and the like. Additionally, at least one carbon within any such alkyl may be optionally replaced with —C(O)—.

Examples of alkyls include methyl, ethyl, ethenyl, ethynyl, propyl, 1-ethyl-propyl, propenyl, propynyl, isopropyl, n-butyl, isobutyl, sec-butyl, tert-butyl, 2-methylbutyl, 3-methyl-butyl, 1-but-3-enyl, butynyl, pentyl, 2-pentyl, isopentyl, neopentyl, 3-methylpentyl, 1-pent-3-enyl, 1-pent-4-enyl, pentyn-2-yl, hexyl, 2-hexyl, 3-hexyl, 1-hex-5-enyl, formyl, acetyl, acetylamino, trifluoromethyl, propionic acid ethyl ester, trifluoroacetyl, methylsulfonyl, ethylsulfonyl, 1-hydroxy-1-methylethyl, 2-hydroxy-1,1,-dimethyl-ethyl, 1,1-dimethyl-propyl, cyano-dimethyl-methyl, propylamino, and the like.

In an embodiment, alkyls may be selected from the group comprising sec-butyl, isobutyl, ethynyl, 1-ethyl-propyl, pentyl, 3-methyl-butyl, pent-4-enyl, isopropyl, tert-butyl, 2-methylbutane, and the like.

In another embodiment, alkyls may be selected from formyl, acetyl, acetylamino, trifluoromethyl, propionic acid ethyl ester, trifluoroacetyl, methylsulfonyl, ethylsulfonyl, 1-hydroxy-1-methylethyl, 2-hydroxy-1,1-dimethyl-ethyl, 1,1-dimethyl-propyl, cyano-dimethyl-methyl, propylamino, and the like.

In an embodiment, “alkyl” or “alk” may be selected from alkyl groups having from 1 to 6 carbon atoms.

In an embodiment, an alkyl may optionally be substituted with at least one group independently selected from alkoxy, acyl, acylamino, thiocarbonylamino, acyloxy, amino, alkyl amino, amidino, alkylamidino, thioamidino, aminoacyl, aminocarbonylamino, aminothiocarbonylamino, aminocarbonyloxy, aryl, aryloxy, substituted aryloxy, aryloxylaryl, cyano, halogen, hydroxyl, nitro, oxo, thioxo, carboxyl, carboxylalkyl, carboxyl-cycloalkyl, carboxylaryl, carboxylheteroaryl, carboxylheterocyclic, cycloalkyl, guanidino, guanidinosulfone, thiol, thioalkyl, thioalkoxy, thioaryl, thiocycloalkyl, thioheteroaryl, thioheterocyclic, heteroaryl, heterocyclic, cycloalkoxy, heteroaryloxy, heterocyclyloxy, oxycarbonylamino, oxythiocarbonylamino, —OS(O)₂-alkyl, —OS(O)₂-aryl, —OS(O)₂—OS(O)₂-heteroaryl, —OS(O)₂-heterocyclic, —OSO₂—NRR where R is hydrogen or alkyl, —NRS(O)₂-alkyl, —NRS(O)₂-aryl, —NRS(O)₂-heteroaryl, —NRS(O)₂-heterocyclic, —NRS(O)₂—NR-alkyl, —NRS(O)₂—NR-aryl, —NRS(O)₂—NR-heteroaryl, and —NRS(O)₂—NR-heterocyclic, where R is hydrogen or alkyl.

The term “alkoxy” in the present invention refers to straight or branched chain alkyl groups, wherein an alkyl group is as defined above, and having 1 to 20 carbon atoms, attached through at least one divalent oxygen atom, such as, for example, methoxy, ethoxy, propoxy, propenoxy, isopropoxy, n-butoxy, sec-butoxy, tert-butoxy, pentoxy, isopentoxy, neopentoxy, neopetynoxy, hexyloxy, heptyloxy, allyloxy, 2-(2-methoxy-ethoxy)-ethoxy, benzyloxy, 3-methylpentoxy, and the like.

In an embodiment, alkoxy groups may be selected from the group comprising allyloxy, hexyloxy, heptyloxy, 2-(2-methoxy-ethoxy)-ethoxy, and benzyloxy.

The term “—C(O)-alkyl” or “alkanoyl” refers to an acyl radical derived from an alkylcarboxylic acid, a cycloalkylcarboxylic acid, a heterocycloalkylcarboxylic acid, an arylcarboxylic acid, an arylalkylcarboxylic acid, a heteroarylcarboxylic acid, or a heteroarylalkylcarboxylic acid, examples of which include formyl, acetyl, 2,2,2-trifluoroacetyl, propionyl, butyryl, valeryl, 4-methylvaleryl, and the like.

The term “cycloalkyl” refers to an optionally substituted carbocyclic ring system of one or more 3, 4, 5, 6, or 7 membered rings. A cycloalkyl can further include 9, 10, 11, 12, 13, and 14 membered fused ring systems. A cycloalkyl can be saturated or partially unsaturated. A cycloalkyl may be monocyclic, bicyclic, tricyclic, and the like. Bicyclic and tricyclic as used herein are intended to include both fused ring systems, such as adamantyl, octahydroindenyl, decahydro-naphthyl, and the like, substituted ring systems, such as cyclopentylcyclohexyl and the like, and spirocycloalkyls such as spiro[2.5]octane, spiro[4.5]decane, 1,4-dioxa-spiro[4.5]decane, and the like. A cycloalkyl may optionally be a benzo fused ring system, which is optionally substituted as defined herein with respect to the definition of aryl. At least one —CH₂— group within any such cycloalkyl ring system may be optionally replaced with —C(O)—, —C(S)—, —C(═N—H)—, —C(═N—OH)—, —C(═N-alkyl)- (optionally substituted as defined herein with respect to the definition of alkyl), or —C(═N—O-alkyl)- (optionally substituted as defined herein with respect to the definition of alkyl).

Further examples of cycloalkyl radicals include cyclopropyl, cyclobutyl, cyclopentyl, cyclohexyl, octahydronaphthyl, 2,3-dihydro-1H-indenyl, and the like.

In an embodiment a cycloalkyl may be selected from the group comprising cyclopentyl, cyclohexyl, cycloheptyl, adamantenyl, bicyclo[2.2.1]heptyl, and the like.

The cycloalkyl groups herein are unsubstituted or substituted in at least one position with various groups. For example, such cycloalkyl groups may be optionally substituted with alkyl, alkoxy, —C(O)H, carboxy, alkoxycarbonyl, cycloalkyl, heterocycloalkyl, aryl, heteroaryl, amido, alkanoylamino, amidino, alkoxycarbonylamino, N-alkyl amidino, N-alkyl amido, N,N′-dialkylamido, aralkoxycarbonylamino, halogen, alkylthio, alkylsulfinyl, alkylsulfonyl, hydroxy, cyano, nitro, amino, monoalkylamino, dialkylamino, haloalkyl, haloalkoxy, aminoalkyl, monoalkylaminoalkyl, dialkylaminoalkyl, and the like.

The term “cycloalkylcarbonyl” refers to an acyl radical of the formula cycloalkyl-C(O)— in which the term “cycloalkyl” has the significance given above, such as cyclopropylcarbonyl, cyclohexylcarbonyl, adamantylcarbonyl, 1,2,3,4-tetrahydro-2-naphthoyl, 2-acetamido-1,2,3,4-tetrahydro-2-naphthoyl, 1-hydroxy-1,2,3,4-tetrahydro-6-naphthoyl, and the like.

The term “heterocycloalkyl,” “heterocycle,” or “heterocyclyl,” refers to a monocyclic, bicyclic, or tricyclic heterocycle radical, containing at least one nitrogen, oxygen, or sulfur atom ring member and having 3, 4, 5, 6, 7, or 8 ring members in each ring, wherein at least one ring in the heterocycloalkyl ring system may optionally contain at least one double bond. At least one —CH₂— group within any such heterocycloalkyl ring system may be optionally replaced with —C(O)—, —C(S)—, —C(═N—H)—, —C(═N—OH)—, —C(═N-alkyl)-, (optionally substituted as defined herein with respect to the definition of alkyl) or —C(═N—O-alkyl)- (optionally substituted as defined herein with respect to the definition of alkyl).

The term “bicyclic” and “tricyclic” as used herein are intended to include both fused ring systems, such as 2,3-dihydro-1H-indole, and the like, substituted ring systems, such as bicyclohexyl, and the like, and spiro-ring systems, such as 3,9-diaza-spiro[5.5]undec-3-yl, and the like. At least one —CH₂— group within any such heterocycloalkyl ring system may be optionally replaced with —C(O)—, —C(N)— or —C(S)—. Heterocycloalkyl is intended to include sulfones, sulfoxides, N-oxides of tertiary nitrogen ring members, carbocyclic fused and benzo fused ring systems wherein the benzo fused ring system is optionally substituted as defined herein with respect to the definition of aryl, and the like. Such heterocycloalkyl radicals may be optionally substituted on one or more carbon atoms by halogen, alkyl, alkoxy, cyano, nitro, amino, alkylamino, dialkylamino, monoalkylaminoalkyl, dialkylaminoalkyl, haloalkyl, haloalkoxy, aminohydroxy, oxo, aryl, aralkyl, heteroaryl, heteroaralkyl, amidino, N-alkylamidino, alkoxycarbonylamino, alkylsulfonylamino, and the like, and/or on a secondary nitrogen atom (i.e., —NH—) by hydroxy, alkyl, aralkoxycarbonyl, alkanoyl, heteroaralkyl, phenyl, phenylalkyl, and the like.

Examples of a heterocycloalkyl include morpholinyl, thiomorpholinyl, thiomorpholinyl S-oxide, thiomorpholinyl S,S-dioxide, piperazinyl, homopiperazinyl, pyrrolidinyl, pyrrolinyl, 2,5-dihydro-pyrrolyl, tetrahydropyranyl, pyranyl, thiopyranyl, piperidinyl, tetrahydrofuranyl, tetrahydrothienyl, imidazolidinyl, homopiperidinyl, 1,2-dihyrdo-pyridinyl, homomorpholinyl, homothiomorpholinyl, homothiomorpholinyl S,S-dioxide, oxazolidinonyl, dihydropyrazolyl, dihydropyrrolyl, 1,4-dioxa-spiro[4.5]decyl, dihydropyrazinyl, dihydropyridinyl, dihydropyrimidinyl, dihydrofuryl, dihydropyranyl, tetrahydrothienyl S-oxide, tetrahydrothienyl S,S-dioxide, homothiomorpholinyl S-oxide, 2-oxo-piperidinyl, 5-oxo-pyrrolidinyl, 2-oxo-1,2-dihydro-pyridinyl, 6-oxo-6H-pyranyl, 1,1-dioxo-hexahydro-thiopyranyl, 1-acetyl-piperidinyl, 1-methanesulfonylpiperidinyl, 1-ethanesulfonylpiperidinyl, 1-oxo-hexahydro-thiopyranyl, 1-(2,2,2-trifluoroacetyl)-piperidinyl, 1-formyl-piperidinyl, and the like.

In an embodiment, a heterocycloalkyl may be selected from pyrrolidinyl, 2,5-dihydro-pyrrolyl, piperidinyl, 1,2-dihyrdo-pyridinyl, pyranyl, piperazinyl, imidazolidinyl, thiopyranyl, tetrahydropyranyl, 1,4-dioxa-spiro[4.5]decyl, and the like.

In another embodiment, a heterocycloalkyl may be selected from 2-oxo-piperidinyl, 5-oxo-pyrrolidinyl, 2-oxo-1,2-dihydro-pyridinyl, 6-oxo-6H-pyranyl, 1,1-dioxo-hexahydro-thiopyranyl, 1-acetyl-piperidinyl, 1-methanesulfonyl piperidinyl, 1-ethanesulfonylpiperidinyl, 1-oxo-hexahydro-thiopyranyl, 1-(2,2,2-trifluoroacetyl)-piperidinyl, 1-formyl-piperidinyl, and the like.

The term “aryl” refers to an aromatic carbocyclic group having a single ring (e.g., phenyl) or multiple condensed rings in which at least one ring is aromatic. The aryl may be monocyclic, bicyclic, tricyclic, etc. Bicyclic and tricyclic as used herein are intended to include both fused ring systems, such as naphthyl and β-carbolinyl, and substituted ring systems, such as biphenyl, phenylpyridyl, diphenylpiperazinyl, tetrahydronaphthyl, and the like. Preferred aryl groups of the present invention include phenyl, 1-naphthyl, 2-naphthyl, indanyl, indenyl, dihydronaphthyl, fluorenyl, tetralinyl, 6,7,8,9-tetrahydro-5H-benzo[a]cycloheptenyl, and the like. The aryl groups herein are unsubstituted or substituted in one or more positions with various groups. For example, such aryl groups may be optionally substituted with alkyl, alkoxy, —C(O)H, carboxy, alkoxycarbonyl, aryl, heteroaryl, cycloalkyl, heterocycloalkyl, amido, alkanoylamino, amidino, alkoxycarbonylamino, N-alkyl amidino, N-alkyl amido, N,N′-dialkylamido, aralkoxycarbonylamino, halogen, alkyl thio, alkylsulfinyl, alkylsulfonyl, hydroxy, cyano, nitro, amino, monoalkylamino, dialkylamino, aralkoxycarbonylamino, halo alkyl, halo alkoxy, aminoalkyl, monoalkylaminoalkyl, dialkylaminoalkyl, and the like. The term “aryl” further include alkaryl groups, including benzyl, 2-phenylethyl, 3-phenyl-n-propyl, and the like.

Examples of aryl groups include phenyl, naphth-2-yl, naphth-1-yl; and the like. Some preferred substituted aryl groups include monosubstituted phenyls, disubstituted phenyls and trisubstituted phenyls such as 5-dimethylaminonaphth-1-yl, 2-chlorophenyl, 2-fluorophenyl, 2-bromophenyl, 2-hydroxyphenyl, 2-nitrophenyl, 2-methylphenyl, 2-methoxyphenyl, 2-phenoxyphenyl, 2-trifluoromethylphenyl, 4-fluorophenyl, 4-chlorophenyl, 4-bromophenyl, 4-nitrophenyl, 4-methylphenyl, 4-hydroxyphenyl, 4-methoxyphenyl, 4-ethoxyphenyl, 4-butoxyphenyl, 4-isopropylphenyl, 4-phenoxyphenyl, 4-trifluoromethylphenyl, 4-hydroxymethylphenyl, 3-methoxyphenyl, 3-hydroxyphenyl, 3-nitrophenyl, 3-fluorophenyl, 3-chlorophenyl, 3-bromophenyl, 3-phenoxyphenyl, 3-thiomethoxyphenyl, 3-methylphenyl, 3-trifluoromethylphenyl, 2,3-dichlorophenyl, 2,3-difluorophenyl, 2,4-dichlorophenyl, 2,5-dimethoxyphenyl, 3,4-dichlorophenyl, 3,4-difluorophenyl, 3,4-methylenedioxyphenyl, 3,4-dimethoxyphenyl, 3,5-difluorophenyl, 3,5-dichlorophenyl, 3,5-di-(trifluoromethyl)phenyl, 3,5-dimethoxyphenyl, 2,4-dichlorophenyl, 2,4-difluorophenyl, 2,6-difluorophenyl, 3,4,5-trifluorophenyl, 3,4,5-trimethoxyphenyl, 3,4,5-tri-(trifluoromethyl)phenyl, 2,4,6-trifluorophenyl, 2,4,6-trimethylphenyl, 2,4,6-tri-(trifluoromethyl)phenyl, 2,3,5-trifluorophenyl, 2,4,5-trifluorophenyl, 2,5-difluorophenyl, 2-fluoro-3-trifluoromethylphenyl, 4-fluoro-2-trifluoromethylphenyl, 2-fluoro-4-trifluoromethylphenyl, 4-benzyloxyphenyl, 2-chloro-6-fluorophenyl, 2,3,4,5,6-pentafluorophenyl, 2,5-dimethylphenyl, 4-phenylphenyl and 2-fluoro-3-trifluoromethylphenyl, 2-(quinolin-8-yl)sulfanylmethyl)phenyl, 2-((3-methylphen-1-ylsufanyl)methyl)phenyl, and the like.

In an embodiment, an aryl may optionally be substituted with at least one group independently selected from hydroxy, acyl, acylamino, thiocarbonylamino, acyloxy, alkyl, alkoxy, alkenyl, alkynyl, amidino, alkylamidino, thioamidino, amino, aminoacyl, aminocarbonyloxy, aminocarbonylamino, aminothiocarbonylamino, aryl, aryloxy, cycloalkoxy, heteroaryloxy, heterocyclyloxy, carboxyl, carboxylalkyl, carboxyl-cycloalkyl, carboxylaryl, carboxylheteroaryl, carboxylheterocyclic, cyano, thiol, thioalkyl, thioaryl, thioheteroaryl, thiocycloalkyl, thioheterocyclic, cycloalkyl, substituted cycloalkyl, guanidino, guanidinosulfone, halogen, nitro, heteroaryl, heterocyclic, oxycarbonylamino, oxythiocarbonylamino, —S(O)₂-alkyl, —S(O)₂-cycloalkyl, —S(O)₂— —S(O)₂-alkenyl, —S(O)₂-aryl, —S(O)₂-heteroaryl, —S(O)₂-heterocyclic, —OS(O)₂-alkyl, —OS(O)₂-aryl, —OS(O)₂-heteroaryl-OS(O)₂-heterocyclic, —OSO₂—NRR where R is hydrogen or alkyl, —NRS(O)₂-alkyl, —NRS(O)₂-aryl, —NRS(O)₂-heteroaryl, —NRS(O)₂-heterocyclic, —NRS(O)₂—NR-alkyl, —NRS(O)₂—NR-aryl, —NRS(O)₂—NR-heteroaryl, —NRS(O)₂—NR-heterocyclic, where R is hydrogen or alkyl, and wherein each of the terms is as defined herein.

Examples of aryl radicals include phenyl, p-tolyl, 4-methoxyphenyl, 4-(tert-butoxy)phenyl, 3-methyl-4-methoxyphenyl, 4-CF₃-phenyl, 4-fluorophenyl, 4-chlorophenyl, 3-nitrophenyl, 3-aminophenyl, 3-acetamidophenyl, 4-acetamidophenyl, 2-methyl-3-acetamidophenyl, 2-methyl-3-aminophenyl, 3-methyl-4-aminophenyl, 2-amino-3-methylphenyl, 2,4-dimethyl-3-aminophenyl, 4-hydroxyphenyl, 3-methyl-4-hydroxyphenyl, 1-naphthyl, 2-naphthyl, 3-amino-1-naphthyl, 2-methyl-3-amino-1-naphthyl, 6-amino-2-naphthyl, 4,6-dimethoxy-2-naphthyl, piperazinylphenyl, and the like.

Further examples of aryl radicals include 3-tert-butyl-1-fluoro-phenyl, 1,3-difluoro-phenyl, (1-hydroxy-1-methyl-ethyl)-phenyl, 1-fluoro-3-(2-hydroxy-1,1-dimethyl-ethyl)-phenyl, (1,1-dimethyl-propyl)-phenyl, cyclobutyl-phenyl, pyrrolidin-2-yl-phenyl, (5-oxo-pyrrolidin-2-yl)-phenyl, (2,5-dihydro-1H-pyrrol-2-yl)-phenyl, (1H-pyrrol-2-yl)-phenyl, (cyano-dimethyl-methyl)-phenyl, tert-butyl-phenyl, 1-fluoro-2-hydroxy-phenyl, 1,3-difluoro-4-propylamino-phenyl, 1,3-difluoro-4-hydroxy-phenyl, 1,3-difluoro-4-ethylamino-phenyl, 3-isopropyl-phenyl, (3H-[1,2,3]triazol-4-yl)-phenyl, [1,2,3]triazol-1-yl-phenyl, [1,2,4]thiadiazol-3-yl-phenyl, [1,2,4]thiadiazol-5-yl-phenyl, (4H-[1,2,4]triazol-3-yl)-phenyl, [1,2,4]oxadiazol-3-yl-phenyl, imidazol-1-yl-phenyl, (3H-imidazol-4-yl)-phenyl, [1,2,4]triazol-4-yl-phenyl, [1,2,4]oxadiazol-5-yl-phenyl, isoxazol-3-yl-phenyl, (1-methyl-cyclopropyl)-phenyl, isoxazol-4-yl-phenyl, isoxazol-5-yl-phenyl, 1-cyano-2-tert-butyl-phenyl, 1-trifluoromethyl-2-tert-butyl-phenyl, 1-chloro-2-tert-butyl-phenyl, 1-acetyl-2-tert-butyl-phenyl, 1-tert-butyl-2-methyl-phenyl, 1-tert-butyl-2-ethyl-phenyl, 1-cyano-3-tert-butyl-phenyl, 1-trifluoromethyl-3-tert-butyl-phenyl, 1-chloro-3-tert-butyl-phenyl, 1-acetyl-3-tert-butyl-phenyl, 1-tert-butyl-3-methyl-phenyl, 1-tert-butyl-3-ethyl-phenyl, 4-tert-butyl-1-imidazol-1-yl-phenyl, ethylphenyl, isobutylphenyl, isopropylphenyl, 3-allyloxy-1-fluoro-phenyl, (2,2-dimethyl-propyl)-phenyl, ethynylphenyl, 1-fluoro-3-heptyloxy-phenyl, 1-fluoro-3-[2-(2-methoxy-ethoxy)-ethoxy]-phenyl, 1-benzyloxy-3-fluoro-phenyl, 1-fluoro-3-hydroxy-phenyl, 1-fluoro-3-hexyloxy-phenyl, (4-methyl-thiophen-2-yl)-phenyl, (5-acetyl-thiophen-2-yl)-phenyl, furan-3-yl-phenyl, thiophen-3-yl-phenyl, (5-formyl-thiophen-2-yl)-phenyl, (3-formyl-furan-2-yl)-phenyl, acetylamino-phenyl, trifluoromethylphenyl, sec-butyl-phenyl, pentylphenyl, (3-methyl-butyl)-phenyl, (1-ethyl-propyl)-phenyl, cyclopentyl-phenyl, 3-pent-4-enyl-phenyl, phenyl propionic acid ethyl ester, pyridin-2-yl-phenyl, (3-methyl-pyridin-2-yl)-phenyl, thiazol-2-yl-phenyl, (3-methyl-thiophen-2-yl)-phenyl, fluoro-phenyl, adamantan-2-yl-phenyl, 1,3-difluoro-2-hydroxy-phenyl, cyclopropyl-phenyl, 1-bromo-3-tert-butyl-phenyl, (3-bromo-[1,2,4]thiadiazol-5-yl)-phenyl, (1-methyl-1H-imidazol-2-yl)-phenyl, (3,5-dimethyl-3H-pyrazol-4-yl)-phenyl, (3,6-dimethyl-pyrazin-2-yl)-phenyl, (3-cyano-pyrazin-2-yl)-phenyl, thiazol-4-yl-phenyl, (4-cyano-pyridin-2-yl)-phenyl, pyrazin-2-yl-phenyl, (6-methyl-pyridazin-3-yl)-phenyl, (2-cyano-thiophen-3-yl)-phenyl, (2-chloro-thiophen-3-yl)-phenyl, (5-acetyl-thiophen-3-yl)-phenyl, cyano-phenyl, and the like.

Alkyl, and cycloalkyl groups include, by way of example, isopropyl, n-propyl, n-butyl, isobutyl, sec-butyl, t-butyl, —CH₂CH═CH₂, —CH₂CH═CH(CH₂)₄CH₃, cyclopropyl, cyclobutyl, cyclohexyl, cyclopentyl, cyclohex-1-enyl, —CH₂-cyclopropyl, —CH₂-cyclobutyl, —CH₂-cyclohexyl, —CH₂-cyclopentyl, —CH₂CH₂-cyclopropyl, —CH₂CH₂-cyclobutyl, —CH₂CH₂-cyclohexyl, —CH₂CH₂-cyclopentyl, and the like.

The term “heteroaryl” refers to an aromatic heterocycloalkyl radical as defined above. The heteroaryl groups herein are unsubstituted or substituted in at least one position with various groups. For example, such heteroaryl groups may be optionally substituted with, for example, alkyl, alkoxy, halogen, hydroxy, cyano, nitro, amino, monoalkylamino, dialkylamino, haloalkyl, haloalkoxy, —C(O)H, carboxy, alkoxycarbonyl, cycloalkyl, heterocycloalkyl, aryl, heteroaryl, amido, alkanoylamino, amidino, alkoxycarbonylamino, N-alkyl amidino, N-alkyl amido, N,N′-dialkylamido, alkyl thio, alkylsulfinyl, alkylsulfonyl, aralkoxycarbonylamino, aminoalkyl, monoalkylaminoalkyl, dialkylaminoalkyl, and the like.

Examples of heteroaryl groups include pyridyl, pyrimidyl, furanyl, imidazolyl, thienyl, oxazolyl, thiazolyl, pyrazinyl, 3-methyl-thienyl, 4-methyl-thienyl, 3-propyl-thienyl, 2-chloro-thienyl, 2-chloro-4-ethyl-thienyl, 2-cyano-thienyl, 5-acetyl-thienyl, 5-formyl-thienyl, 3-formyl-furanyl, 3-methyl-pyridinyl, 3-bromo-[1,2,4]thiadiazolyl, 1-methyl-1H-imidazole, 3,5-dimethyl-3H-pyrazolyl, 3,6-dimethyl-pyrazinyl, 3-cyano-pyrazinyl, 4-tert-butyl-pyridinyl, 4-cyano-pyridinyl, 6-methyl-pyridazinyl, 2-tert-butyl-pyrimidinyl, 4-tert-butyl-pyrimidinyl, 6-tert-butyl-pyrimidinyl, 5-tert-butyl-pyridazinyl, 6-tert-butyl-pyridazinyl, quinolinyl, benzothienyl, indolyl, indolinyl, pyridazinyl, isoindolyl, isoquinolyl, quinazolinyl, quinoxalinyl, phthalazinyl, imidazolyl, isoxazolyl, pyrazolyl, indolizinyl, indazolyl, benzothiazolyl, benzimidazolyl, benzofuranyl, thienyl, pyrrolyl, oxadiazolyl, thiadiazolyl, triazolyl, tetrazolyl, oxazolopyridinyl, imidazopyridinyl, isothiazolyl, naphthyridinyl, cinnolinyl, carbazolyl, beta-carbolinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, phenoxazinyl, phenothiazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, coumarinyl, isocoumarinyl, chromonyl, chromanonyl, pyridinyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide, tetrahydrocarbazole, tetrahydrobetacarboline, and the like.

Further examples of heteroaryl include, by way of example, pyrid-2-yl, pyrid-3-yl, pyrid-4-yl, fluoropyridyls (including 5-fluoropyrid-3-yl), chloropyridyls (including 5-chloropyrid-3-yl), thiophen-2-yl, thiophen-3-yl, benzothiazol-4-yl, 2-phenylbenzoxazol-5-yl, furan-2-yl, benzofuran-2-yl, thionaphthen-2-yl, 2-chlorothiophen-5-yl, 3-methylisoxazol-5-yl, 2-(thiophenyl)thiophen-5-yl, 6-methoxythionaphthen-2-yl, 3-phenyl-1,2,4-thiooxadiazol-5-yl, 2-phenyloxazol-4-yl, 5-chloro-1,3-dimethylpyrazol-4-yl, 2-methoxycarbonyl-thiophen-3-yl, 2,3-dimethylimidazol-5-yl, 2-methylcarbonylamino-4-methyl-thiazol-5-yl, quinolin-8-yl, thiophen-2-yl, 1-methylimidiazol-4-yl, 3,5-dimethylisoxazol-4-yl, and the like.

In an embodiment, a heteroaryl group may be selected from pyridyl, pyrimidyl, furanyl, imidazolyl, thienyl, oxazolyl, thiazolyl, pyrazinyl, and the like.

In another embodiment, a heteroaryl group may be selected from 3-methyl-thienyl, 4-methyl-thienyl, 3-propyl-thienyl, 2-chloro-thienyl, 2-chloro-4-ethyl-thienyl, 2-cyano-thienyl, 5-acetyl-thienyl, 5-formyl-thienyl, 3-formyl-furanyl, 3-methyl-pyridinyl, 3-bromo-[1,2,4]thiadiazolyl, 1-methyl-1H-imidazole, 3,5-dimethyl-3H-pyrazolyl, 3,6-dimethyl-pyrazinyl, 3-cyano-pyrazinyl, 4-tert-butyl-pyridinyl, 4-cyano-pyridinyl, 6-methyl-pyridazinyl, 2-tert-butyl-pyrimidinyl, 4-tert-butyl-pyrimidinyl, 6-tert-butyl-pyrimidinyl, 5-tert-butyl-pyridazinyl, 6-tert-butyl-pyridazinyl, and the like.

Further examples of heterocycloalkyls and heteroaryls may be found in Katritzky, A. R. et al., Comprehensive Heterocyclic Chemistry: The Structure, Reactions, Synthesis and Use of Heterocyclic Compounds, Vol. 1-8, New York: Pergamon Press, 1984.

The term “aralkoxycarbonyl” refers to a radical of the formula aralkyl-O—C(O)— in which the term “aralkyl” is encompassed by the definitions above for aryl and alkyl. Examples of an aralkoxycarbonyl radical include benzyloxycarbonyl, 4-methoxyphenylmethoxycarbonyl, and the like.

The term “aryloxy” refers to a radical of the formula —O-aryl in which the term aryl is as defined above.

The term “aralkanoyl” refers to an acyl radical derived from an aryl-substituted alkanecarboxylic acid such as phenylacetyl, 3-phenylpropionyl(hydrocinnamoyl), 4-phenylbutyryl, (2-naphthyl)acetyl, 4-chlorohydrocinnamoyl, 4-aminohydrocinnamoyl, 4-methoxyhydrocinnamoyl, and the like.

The term “aroyl” refers to an acyl radical derived from an arylcarboxylic acid, “aryl” having the meaning given above. Examples of such aroyl radicals include substituted and unsubstituted benzoyl or naphthoyl such as benzoyl, 4-chlorobenzoyl, 4-carboxybenzoyl, 4-(benzyloxycarbonyl)benzoyl, 1-naphthoyl, 2-naphthoyl, 6-carboxy-2 naphthoyl, 6-(benzyloxycarbonyl)-2-naphthoyl, 3-benzyloxy-2-naphthoyl, 3-hydroxy-2-naphthoyl, 3-(benzyloxyformamido)-2-naphthoyl, and the like.

The term “haloalkyl” refers to an alkyl radical having the meaning as defined above wherein one or more hydrogens are replaced with a halogen. Examples of such haloalkyl radicals include chloromethyl, 1-bromoethyl, fluoromethyl, difluoromethyl, trifluoromethyl, 1,1,1-trifluoroethyl, and the like.

The term “epoxide” refers to chemical compounds or reagents comprising a bridging oxygen wherein the bridged atoms are also bonded to one another either directly or indirectly. Examples of epoxides include epoxyalkyl (e.g., ethylene oxide and 1,2-epoxybutane), epoxycycloalkyl (e.g., 1,2-epoxycyclohexane and 1,2-epoxy-1-methylcyclohexane), and the like.

The term “structural characteristics” refers to chemical moieties, chemical motifs, and portions of chemical compounds. These include R groups, such as those defined herein, ligands, appendages, and the like. For example, structural characteristics may be defined by their properties, such as, but not limited to, their ability to participate in intermolecular interactions including Van der Waal's interactions (e.g., electrostatic interactions, dipole-dipole interactions, dispersion forces, hydrogen bonding, and the like). Such characteristics may have an increased ability to cause the desired effect and thus prevent or treat the targeted diseases or conditions.

The term “halo” or “halogen” refers to fluoro, chloro, bromo or iodo.

The term “oxo” refers to an oxygen atom bound to an atom such as, but not limited to, carbon or nitrogen, through a double bond.

In an embodiment, examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydro-isoquinoline, 4,5,6,7-tetrahydrobenzo[b]thiophene, thiazole, thiazolidine, thiophene, benzo[b]thiophene, morpholino, thiomorpholino, piperidinyl, pyrrolidine, tetrahydrofuranyl, and the like.

Amino acid refers to any of the naturally occurring amino acids, as well as synthetic analogs (e.g., D-stereoisomers of the naturally occurring amino acids, such as D-threonine, and L-stereoisomers of amino acids in proteins) and derivatives thereof. α-Amino acids comprise a carbon atom to which is bonded an amino group, a carboxyl group, a hydrogen atom, and a distinctive group referred to as a “side chain”. The side chains of naturally occurring amino acids are well known in the art and include, for example, hydrogen (e.g., glycine), alkyl (e.g., alanine, valine, leucine, isoleucine, proline), substituted alkyl (e.g., threonine, serine, methionine, cysteine, aspartic acid, asparagine, glutamic acid, glutamine, arginine, and lysine), arylalkyl (e.g., phenylalanine and tryptophan), substituted arylalkyl (e.g., tyrosine), and heteroarylalkyl (e.g., histidine). Unnatural amino acids are also known in the art, as set forth in, for example, Williams (ed.), Synthesis of Optically Active α-Amino Acids, Pergamon Press (1989); Evans et al., J. Amer. Chem. Soc., 112:4011-4030 (1990); Pu et al., J. Org Chem., 56:1280-1283 (1991); Williams et al., J. Amer. Chem. Soc., 113:9276-9286 (1991); and all references cited therein. The present invention includes the side chains of unnatural amino acids as well.

“Pharmaceutically acceptable salt” refers to pharmaceutically acceptable salts of a compound of formula (I) or formula (II) which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like.

In the examples below, the following abbreviations have the following meanings. If an abbreviation is not defined, it has its generally accepted meaning.
aq or aq.=aqueous
AcOH=acetic acid
Bd=broad doublet
bm=broad multiplet
bs=broad singlet
Bn=benzyl
Boc=N-tertbutoxylcarbonyl
Boc₂O=di-tert-butyl dicarbonate
BOP=benzotriazol-1-yloxy-tris(dimethylamino)phosphonium hexafluorophosphate
Cbz=carbobenzyloxy
CHCl₃=chloroform
CH₂Cl₂=dichloromethane
(COCl)₂=oxalyl chloride
d=doublet
dd=doublet of doublets
dt=doublet of triplets
DBU=1,8-diazabicyclo[5.4.0]undec-7-ene
DCC=1,3-dicyclohexylcarbodiimide
DMAP=4-N,N-dimethylaminopyridine
DME=ethylene glycol dimethyl ether
DMF=N,N-dimethylformamide
DMSO=dimethylsulfoxide
EDC=1-(3-dimethylaminopropyl)-3-ethylcarbodiimide hydrochloride
Et₃N=triethylamine
Et₂O=diethyl ether
EtOAc=ethyl acetate
EtOH=ethanol
eq or eq.=equivalent
Fmoc=N-(9-fluorenylmethoxycarbonyl)
FmocONSu=N-(9-fluorenylmethoxycarbonyl)-succinimide
g=gram(s)
h=hour(s)
H₂O=water
HBr=hydrobromic acid
HCl=hydrochloric acid
HOBT=1-hydroxybenzotriazole hydrate
Hr=hour
K₂CO₃=potassium carbonate
L=liter
m=multiplet
MeOH=methanol
Mg=milligram
MgSO₄=magnesium sulfate
mL=milliliter
mm=millimeter
mM=millimolar
mmol=millimol
mp=melting point
N=normal
NaCl=sodium chloride
Na₂CO₃=sodium carbonate
NaHCO₃=sodium bicarbonate
NaOEt=sodium ethoxide
NaOH=sodium hydroxide
NH₄Cl=ammonium chloride
NMM=N-methylmorpholine
Phe=L-phenylalanine
Pro=L-proline
Psi=pounds per square inch
PtO₂=platinum oxide
q=quartet
quint=quintet
rt=room temperature
s=singlet
sat=saturated
t=triplet
t-BuOH=tert-butanol
TFA=trifluoroacetic acid
THF=tetrahydrofuran
TLC or tlc=thin layer chromatography
Ts=tosyl
TsCl=tosyl chloride
TsOH=toluene sulfonic acid
μL=microliter

### Compound Preparation

The compounds of the present invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

The compounds of this invention may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

In the following examples and procedures, the term “Aldrich” indicates that the compound or reagent used in the procedure is commercially available from Aldrich Chemical Company, Inc., Milwaukee, Wis. 53233 USA; the term “Sigma” indicates that the compound or reagent is commercially available from Sigma, St. Louis Mo. 63178 USA; the term “TCI” indicates that the compound or reagent is commercially available from TCI America, Portland Oreg. 97203; the term “Frontier” or “Frontier Scientific” indicates that the compound or reagent is commercially available from Frontier Scientific, Utah, USA; the term “Bachem” indicates that the compound or reagent is commercially available from Bachem, Torrance, Calif., USA. The term “Matrix” or “Matrix Scientific” indicates that the compound or reagent is commercially available from Matrix Scientific, Columbia, S.C., USA. The term “Ambinter” indicates that the compound or reagent is commercially available from Ambinter Paris, France. The term “Lancaster” indicates that the compound or reagent is commercially available from Lancaster Synthesis, Inc., Windham, N.H., USA. The term “Oakwood” indicates that the compound or reagent is commercially available from Oakwood Products, Inc., West Columbia, S.C., USA. The term “Syntech” indicates that the compound or reagent is commercially available from Syntech Development Company, Franklin Park, N.J., USA. The term “J & W PharmLab” indicates that the compound or reagent is commercially available from J & W PharmLab LLC, Morrisville, Pa., USA.

### EXAMPLE 1

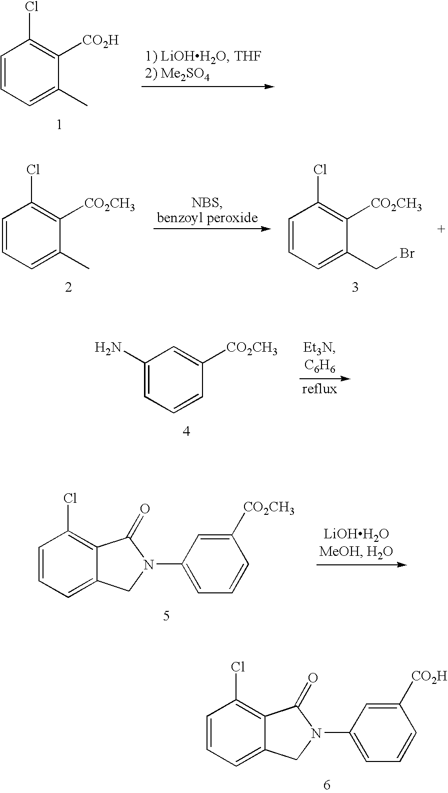 Preparation of 2-Chloro-6-methyl-benzoic acid methyl ester (2). A solution of 5.811 g (34.1 mmol) of 2-chloro-6-methy benzoic acid (Lancaster) and 1.430 g (34.1 mmol) of LiOH.H₂O in 50 mL of THF was stirred at room temperature. After 1 h, 3.55 mL (37.4 mmol) of dimethylsulfate (Aldrich) was added and the reaction mixture was refluxed. After 17 h, the mixture was allowed to cool to room temperature and quenched by the addition of 30 mL of NH₄OH and 30 mL of water. After stirring for 30 min the mixture was concentrated by rotary evaporation. The residue was diluted with water and extracted with EtOAc. The organic layer was dried over MgSO₄ and filtered. The filtrate was concentrated by rotary evaporation. The product was isolated as a yellow oil by flash chromatography on silica gel using 10:90 EtOAc:hexanes as eluant.
Preparation of 2-Bromomethyl-6-chloro-benzoic acid methyl ester (3). A suspension of 8.819 g (47.8 mmol) of 2-chloro-6-methyl-benzoic acid methyl ester (2), 10.193 g (57.3 mmol) of NBS (Aldrich) and 0.220 g (0.9 mmol) of benzoyl peroxide (Aldrich) in 75 mL of CCl₄ was stirred and refluxed for 16 h. The reaction mixture was cooled to room temperature and concentrated by rotary evaporation. The residue was diluted with CHCl₃ and filtered to remove an undissolved solid and re-concentrated by rotary evaporation. The product was isolated as a clear, colorless oil by flash chromatography on silica gel using 5:95 EtOAc:hexanes as eluant.
Preparation of 3-(7-Chloro-1-oxo-1,3-dihydro-isoindol-2-yl)benzoic acid methyl ester (5). A solution of 8.012 g (30.4 mmol) of 2-bromomethyl-6-chloro-benzoic acid methyl ester (3), 4.597 g (30.4 mmol) of 3-amino-benzoic acid methyl ester (4) (Fluka), and 4.40 mL (31.6 mmol) of Et₃N in 60 mL of benzene was refluxed for 22 h. The reaction mixture was cooled to room temperature and a solid formed. The mixture was diluted with water and CH₂Cl₂. The layers were separated and the aqueous layer was extracted with CH₂Cl₂. The organic layers were combined, washed with brine, dried over MgSO₄ and filtered. The solvent was removed by rotary evaporation to afford a brown solid. The solid was dissolved in a minimum of CHCl₃ and hexanes was added until a solid precipitated. The solid was recovered by filtration and washed with ether to afford the product as a white solid. See also: Egbertson, M. S., et al., Bioorg. Med. Chem. Lett., 1996, 6, 2519.
Preparation of 3-(7-Chloro-1-oxo-1,3-dihydro-isoindol-2-yl)benzoic acid (6). A suspension of 7.100 g (23.5 mmol) of 3-(7-chloro-1-oxo-1,3-dihydro-isoindol-2-yl)benzoic acid methyl ester (5) and 5.059 g (120.6 mmol) of LiOH.H₂O in 180 mL of MeOH and 60 mL of H₂O was stirred at room temperature for 6 days. The reaction was quenched by the addition of 150 mL of 1 M HCl. A white solid formed which was collected by filtration. The solid was stirred in 700 mL of acetone and 100 mL of MeOH at 50° C. for 1 h and filtered. The solid which collected on the filter was kept and the filtrate was concentrated by rotary evaporation and a solid precipitated which was collected by filtration. The solids were combined to afford the product as a white solid.

### EXAMPLE 2

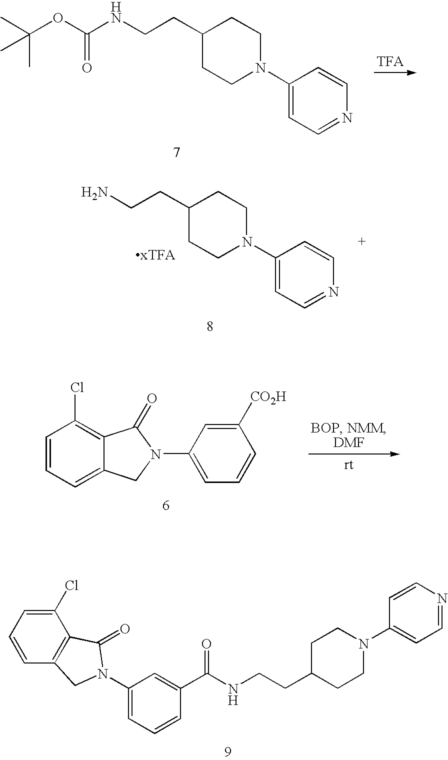 Preparation of 3-(7-Chloro-1-oxo-1,3-dihydro-isoindol-2-yl)-N-[2-(3,4,5,6-tetrahydro-2H-[1,4′]bipyridinyl-4-yl)ethyl]benzamide (9). [2-(3,4,5,6-Tetrahydro-2H-[1,4′]bipyridinyl-4-yl)-ethyl]carbamic acid tert-butyl ester (7, 0.319 g, 1.0 mmol) (see, e.g., WO 2004098589) was dissolved in neat TFA and stirred for 15 min until vigorous bubbling ceased. The TFA was then removed by rotary evaporation to afford 2-(3,4,5,6-tetrahydro-2H-[1,4′]bipyridinyl-4-yl)ethylamine trifluoroacetate (8). A solution of 8, 0.288 g (1.0 mmol) of 3-(7-chloro-1-oxo-1,3-dihydro-isoindol-2-yl)benzoic acid (6), 0.550 g (1.2 mmol) of BOP and 0.60 mL (5.5 mmol) of NMM in 6.0 mL of DMF was stirred at room temperature for 16 h. The reaction mixture was then added dropwise to a sat. aq. solution of NaHCO₃, sonicated and filtered to give a sticky brown solid. The brown solid was sonicated in CH₃CN and filtered to afford the product as a tan solid.

### EXAMPLE 3

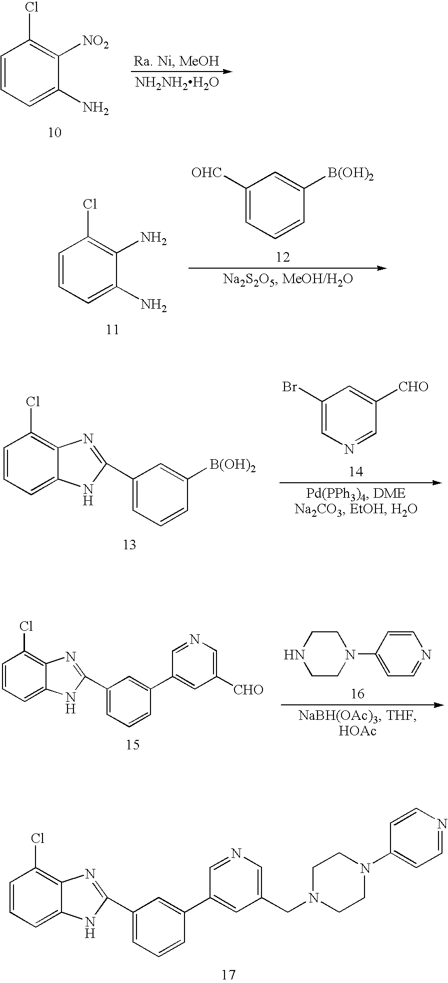 Preparation of 3-Chloro-benzene-1,2-diamine (11). To a solution of 3-chloro-2-nitro-phenylamine (10, 50.0 g, 289.7 mmol) (Oakwood) and hydrazine monohydrate (27.8 mL, 573.1 mmol) in methanol (250 mL) was added Raney Ni dropwise (slurry in water, 3 pipette) at room temperature under a nitrogen atmosphere. The mixture was stirred at rt overnight. The catalyst was filtered off through Celite® and the solvent was removed under reduced pressure to give the desired product as a black liquid, which was used for the subsequent synthesis without further purification.
Preparation of [3-(4-Chloro-1H-benzoimidazol-2-yl)-phenyl]boronic acid (13). To a solution of 3-chloro-benzene-1,2-diamine (11, 12.3 g, 85.9 mmol) and 3-formylphenylboronic acid (12, 14.2 g, 94.7 mmol) (Aldrich) in MeOH (250 mL) was added a solution of sodium metabisulfite (9.0 g, 47.3 mmol) in water (200 mL). The mixture was then heated at 60° C. overnight. The reaction mixture was concentrated to about half of the original volume and the pH adjusted to about 6.5 by glacial acetic acid. The resulting solid was collected by filtration and washed with a small amount of water to afford the desired product as an amber solid.
Preparation of 5-[3-(4-Chloro-1H-benzoimidazol-2-yl)-phenyl]pyridine-3-carbaldehyde (15). To a solution of tetrakis(triphenylphosphine)palladium (133 mg, 0.12 mmol) in DME (3 mL) was added a solution of 5-bromo-3-carbaldehydepyridine (14, 442.7 mg, 2.38 mmol) (Frontier Scientific) in DME (7 mL). The mixture was stirred at room temperature under a nitrogen atmosphere for 30 min. Then a solution of boronic acid 13 (778.0 mg, 2.86 mmol) in ethanol (3 mL) was added to the above mixture. After stirring at room temperature for an additional 20 min, the mixture was treated with sodium carbonate (2.0 M, 8 mL) and the resulting mixture was heated under reflux for overnight. Solvent was removed under reduced pressure and the residue was triturated with diethyl ether (5×20 mL). The organic solvents were combined and dried (MgSO₄). The solvent was removed under reduced pressure to give the desired product as a yellow solid, which was used directly for the subsequent reaction without further purification.
Preparation of 4-Chloro-2-{3-[5-(4-pyridin-4-yl-piperazin-1-ylmethyl)-pyridin-3-yl]-phenyl}-1H-benzoimidazole (17). Aldehyde 15 (324 mg, 0.97 mmol) was mixed with 1-pyridin-4-yl-piperazine (16,158 mg, 0.97 mmol) in THF (5 mL) and was treated with sodium triacetoxyborohydride (308.4 mg, 1.46 mmol) and glacial acetic acid (60 mg, 1.0 mmol). The mixture was stirred at room temperature for overnight. The reaction was quenched by addition of sat. NaHCO₃ (5 mL). EtOAC was added to this mixture and a precipitate formed. The solid was isolated through filtration to afford a nice product as a yellow solid, which was purified by prep HPLC to give pure product as a white solid (TFA salt).

### EXAMPLE 4

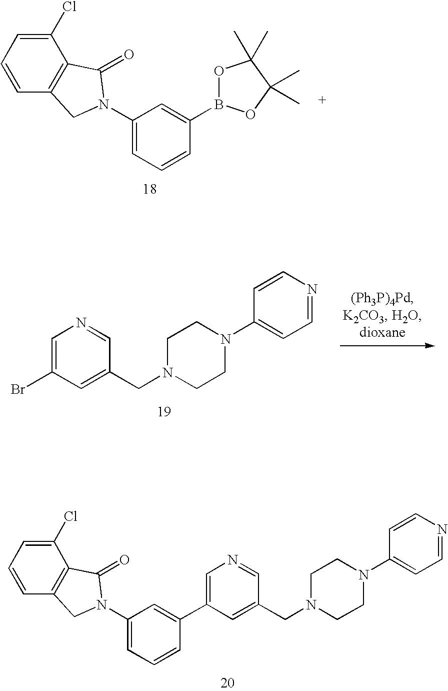 Preparation of 7-Chloro-2-{3-[5-(4-pyridin-4-yl-piperazin-1-yl methyl)pyridin-3-yl]-phenyl}-2,3-dihydro-isoindol-1-one (20). A solution of 0.848 g (2.3 mmol) of dioxaborolane 18, prepared as described for compound 5 in EXAMPLE 1 using ester 3 and 3-(4,4,5,5-tetramethyl-1,3,2-dioxaborolan-2-yl)aniline (Aldrich), 0.891 g (2.7 mmol) of bromide 19, prepared as described for compound 17 in EXAMPLE 3 using piperazine 16 and aldehyde 14, 0.055 g (0.5 mmol) of (Ph₃P)₄Pd and 0.351 g (2.5 mmol) of K₂CO₃ in 4.0 mL of water and 16.0 mL of 1,4-dioxane was refluxed for 16 h. The solid that formed was collected by filtration and washed with CH₃CN and water. The solid was then dissolved in 1 M HCl and the solution washed with CHCl₃. The pH of the aqueous solution was raised to 8 with solid NaHCO₃ and extracted with CHCl₃. The organic layer was dried over MgSO₄, filtered and the solvent removed by rotary evaporation to afford crude 20. The product was purified by preparative HPLC to give the product as a brown solid TFA salt.

### EXAMPLE 5

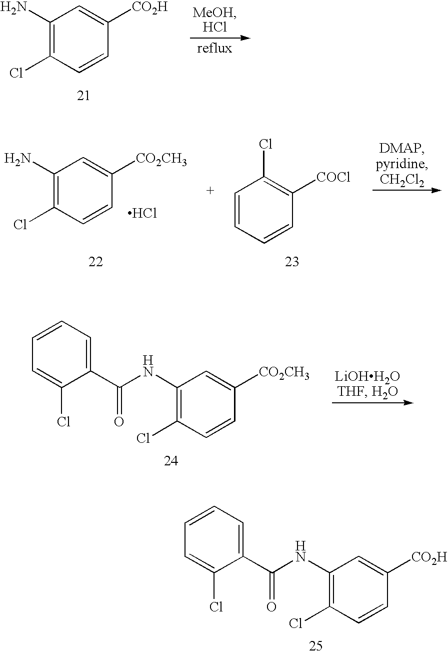 Preparation of 3-Amino-4-chloro-benzoic acid methyl ester hydrochloride (22). A solution of 15.556 g (90.7 mmol) of 3-amino-4-chlorobenzoic acid (Aldrich) in 500 mL of MeOH was stirred at rt as HCl (gas) was bubbled through the solution for 2 min. The solution was then refluxed for 16 h. The reaction mixture was cooled to rt and the solvent removed by rotary evaporation to afford 22 as a white solid.
Preparation of 4-Chloro-3-(2-chlorobenzoylamino)benzoic acid methyl ester (24). A solution of 20.30 g (91.4 mmol) of ester 22, 37.00 mL (475.5 mmol) of pyridine and 1.154 g (9.4 mmol) of DMAP in 500 mL of CH₂Cl₂ was stirred at 0° C. as 14.00 mL (110.2 mmol) of 2-chlorobenzoyl chloride (23) (Aldrich) was slowly added. The reaction mixture was warmed to room temperature and stirred for 64 h. The solvent was removed by rotary evaporation and the residue was dissolved in 300 mL of CH₂Cl₂ and washed with water followed by 1 M HCl and sat. aq. NaHCO₃. The organic layer was dried over MgSO₄, filtered and the solvent removed by rotary evaporation to afford the product as an off-white solid.
Preparation of 4-Chloro-3-(2-chlorobenzoylamino)benzoic acid (25). A mixture of 29.35 g (90.5 mmol) of ester 24 and 21.25 g (506.4 mmol) of LiOH.H₂O in 200 mL of THF and 100 mL of water was stirred at room temperature for 16 h. The THF was removed by rotary evaporation and the remaining solution was diluted to 300 mL with water. The pH of the solution was lowered to 7 with conc. HCl. A voluminous, white precipitate formed and additional water was added. The solid was collected by filtration and washed with 1 M HCl to afford the product as a white solid.

### EXAMPLE 6

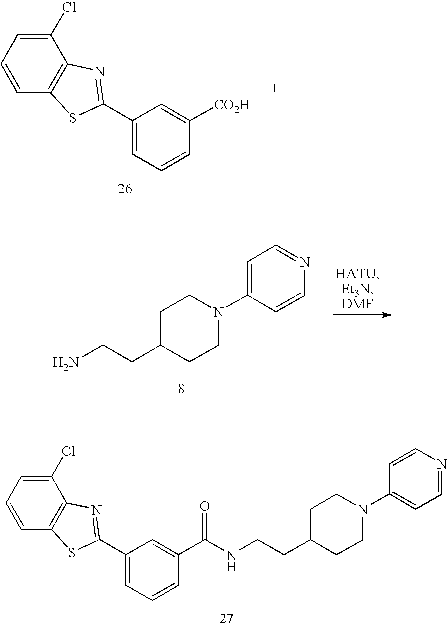 Preparation of 3-(4-Chloro-benzothiazol-2-yl)-N-[2-(3,4,5,6-tetrahydro-2H-[1,4′]bipyridinyl-4-yl)ethyl]benzamide (27). A solution of 0.070 g (0.25 mmol) of benzoic acid 26, 0.045 g (0.22 mmol) of 2-(3,4,5,6-tetrahydro-2H-[1,4′]bipyridinyl-4-yl)ethylamine (8), 0.095 g (0.25 mmol) of HATU and 0.017 mL (0.25 mmol) of Et₃N was dissolved in 2 mL of DMF and stirred at 25° C. for 1 hour. The DMF was then removed by rotary evaporation under high vacuum to obtain an oil. The product was isolated by reverse phase HPLC.

### EXAMPLE 7

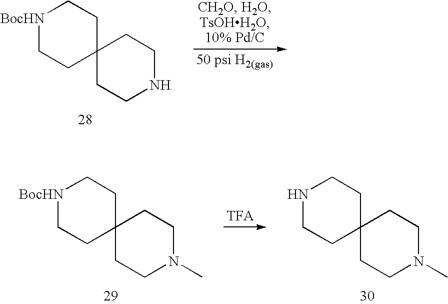 Preparation of 3-Methyl-3,9-diaza-spiro[5.5]undecane (30). A mixture of 0.625 g (2.5 mmol) of 3,9-diaza-spiro[5.5]undecane-3-carboxylic acid tert-butyl ester (28) (Syntech) and 0.470 g (2.5 mmol) of TsOH.H₂O in 25 mL of water was sonicated with heating to afford a solution. To this solution was added 2.0 mL (24.6 mmol) of formaldehyde followed by 0.629 g of 10% Pd/C. The mixture was agitated under a 50 psi atmosphere of hydrogen for 16 h. The reaction mixture was filtered through Celite and the solution adjusted to pH 11 with 1 M NaOH. The mixture was extracted with CHCl₃, dried over MgSO₄, filtered and the solvent removed by rotary evaporation to afford compound 29. Stirring compound 29 in neat TFA for 15 min followed by removal of the TFA by rotary evaporation afforded compound 30.

### EXAMPLE 8

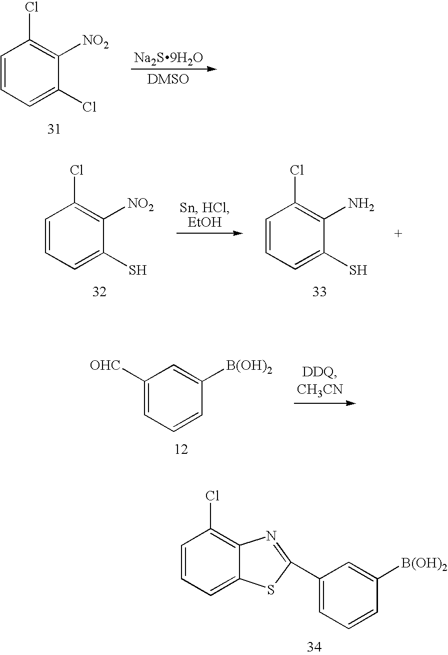 Preparation of 3-Chloro-2-nitro-benzenethiol (32). To a solution of 2,6-dichloronitrobenzene (31, 5.0 g, 26 mmol) in DMSO (30 mL) was added sodium sulfide nonahydrate (6.15 g, 26 mmol). The mixture was stirred at room temperature overnight. The small amount of precipitation was filtered off and water (210 mL) was added to the filtrate. The solution was then concentrated under reduced pressure to about 40 mL. Concentrated HCl was used to adjust the pH to 3-4 and the desired product was precipitated out as a yellow solid.
Preparation of 2-Amino-3-chlorobenzenethiol (33). To a suspension of 3-chloro-2-nitrobenzenethiol (32, 2.0 g, 85.9 mmol) in ethanol (10 mL) was added tin (5.57 g, 46.9 mmol) and conc. HCl (8.3 mL). The mixture was then heated at 70° C. overnight. The solid was filtered off and the filtrate was concentrated under reduced pressure to about 10 mL. This hot solution was treated with 10 mL of conc. HCl. Upon cooling, the yellow solid was separated by filtration and washed with a small amount of water. The solid was redissolved in EtOH (5 mL) and treated with conc. HCl (5 mL). The resulting yellow solid was separated again by filtration to give the desired product.
Preparation of [3-(4-Chloro-1H-benzothiazol-2-yl)phenyl]boronic acid (34). To a suspension of 2-amino-3-chlorobenzenethiol (33, 2.4 g, 15.1 mmol) in acetonitrile (10 mL) was added aldehyde 12 (2.48 g, 16.6 mmol) and DDQ (3.7 g, 16.5 mmol). The mixture was then stirred at room temperature for 3 h. The solid was isolated by filtration and washed with EtOAc to give the desired product as a blue solid.

### EXAMPLE 9

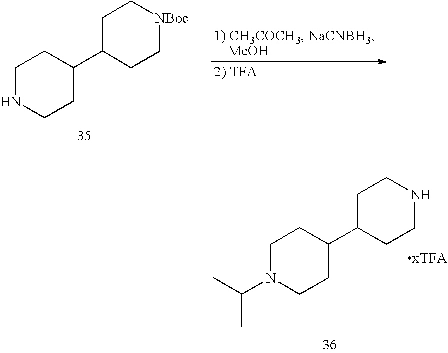 Preparation of 1-Isopropyl-[4,4′]bipiperidine triflouroacetate (36). A solution of 0.175 g (0.65 mmol) of piperidine 35 in 2 mL of dry MeOH was stirred under N₂(g). Dry acetone (0.5 mL, 6.5 mmol) was added via syringe and the solution was left to stir for 30 min. Sodium cyanoborohydride (0.041 g, 0.65 mmol) was then quickly added and the solution was left to stir at rt overnight. The MeOH was then removed by rotary evaporation. The reaction mixture was partitioned between EtOAc and H₂O and the layers separated. The organic layer was washed with brine, dried over MgSO₄ and filtered. The EtOAc was then removed by rotary evaporation. TFA was added to the material via pipette and the solution left to stir for 5 min. The TFA was then removed by rotary evaporation using an oil pump and drying under vacuum to yield the product as a yellow oil.

### EXAMPLE 10

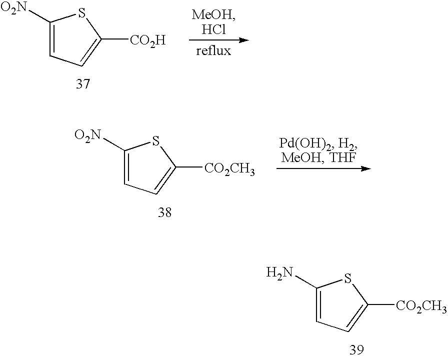 Preparation of 5-Nitrothiophene-2-carboxylic acid methyl ester (38). A solution of 20.0 g of 5-nitro-thiophene-2-carboxylic acid (37, 116 mmol) in 500 mL of absolute MeOH was stirred at room temperature as HCl gas was bubbled through the solution for 3 min. The reaction mixture was refluxed overnight and then cooled to room temperature. The solvent was removed by rotary evaporation. The product was isolated by flash chromatography on silica gel using 20:80 EtOAc:hexanes as eluant to afford the product as a yellow solid.
Preparation of 5-Aminothiophene-2-carboxylic acid methyl ester (39). To a solution of 5.0 grams of 5-nitro-thiophene-2-caroboxylic acid methyl ester (38, 26.7 mmol) in 50 mL of MeOH and 25 mL of THF was added 1.0 g of Pd(OH)₂ (20 wt % on carbon, wet). The mixture was placed on a Parr shaker at 55 psi H₂ and shaken overnight. The mixture was filtered through Celite and the solvent was removed by rotary evaporation. The residue was dissolved in EtOAc and washed with sat. aq. NaHCO₃. The organic layer was dried over Na₂SO₄, filtered, and the solvent was removed by rotary evaporation to afford the product as a brown solid.

### EXAMPLE 11

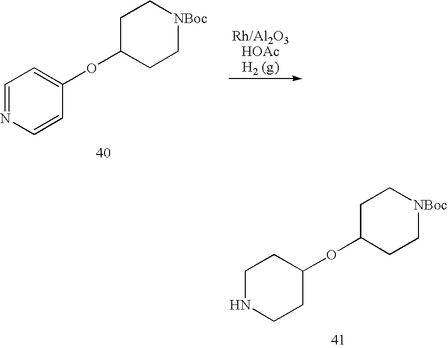

Preparation of 4-(Piperidin-4-yloxy)piperidine-1-carboxylic acid tert-butyl ester (41). To 1.0 g (3.6 mmol) of 4-(pyridin-4-yloxy)piperidine-1-carboxylic acid tert-butyl ester (40) dissolved in 4.0 mL of HOAc was added 0.2 g of 5% Rh/Al₂O₃. The reaction mixture was put under 50 psi of H₂ gas overnight, filtered through Celite® and the solvent was removed by rotary evaporation to yield 41 as a colorless oil.

### EXAMPLE 12

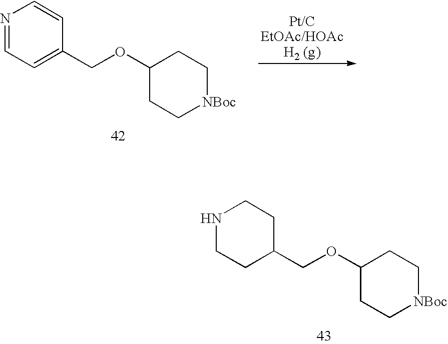

Preparation of 4-(Piperidin-4-ylmethoxy)piperidine-1-carboxylic acid tert-butyl ester (43). To 2.2 g (7.5 mmol) of 4-(pyridin-4-ylmethoxy)piperidine-1-carboxylic acid tert-butyl ester (42) dissolved in 15 mL of 1:1 EtOAc:HOAc was added 0.22 g of 5% Pt on activated carbon. The reaction mixture was stirred vigorously under 1 atm of H₂ gas for 4 hrs and then filtered through Celite®. The solvent was removed by rotary evaporation to yield 43 as a colorless oil.

### EXAMPLE 13

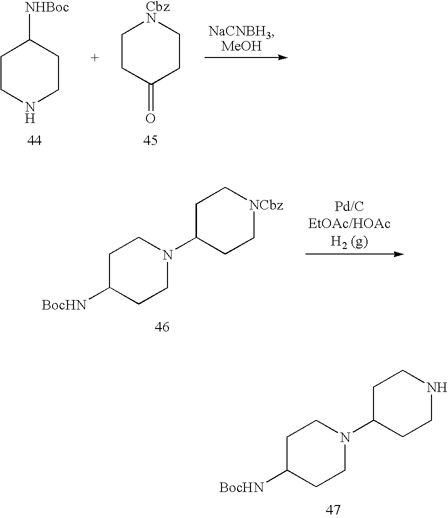

Preparation of [1,4′]Bipiperidinyl-4-yl-carbamic acid tert-butyl ester (47). An oven dried round bottom flask and stir bar were cooled under N₂ gas. To 1.0 g (5.0 mmol) of 4-N-Boc-amino-piperidine (44) (Astatech) dissolved in 2 mL dry MeOH was added 1.2 g (5.0 mmol) of benzyl 4-oxo-1-piperidinecarboxylate (45) (Aldrich). The mixture was left to stir for 30 min under N₂ gas. Then 0.31 g (5.0 mmol) of NaCNBH₃ was added and the reaction mixture was left to stir overnight. The MeOH was removed by rotary evaporation and the residue partitioned between EtOAc and H₂O, washed with brine, dried over MgSO₄, filtered and the solvent was removed by rotary evaporation to yield 4-tert-butoxycarbonylamino-[1,4′]bipiperidinyl-1′-carboxylic acid benzyl ester (46) as a white oil. To 1.2 g (3.0 mmol) of crude 46 dissolved in 3 mL 1:1 EtOAc:HOAc was added 0.24 g of 5% Pd on activated carbon. The mixture was left to stir vigorously under H₂ gas at 1 atm overnight. The mixture was filtered through Celite® and the solvent was removed by rotary evaporation to yield the product as a yellow oil.

### EXAMPLE 14

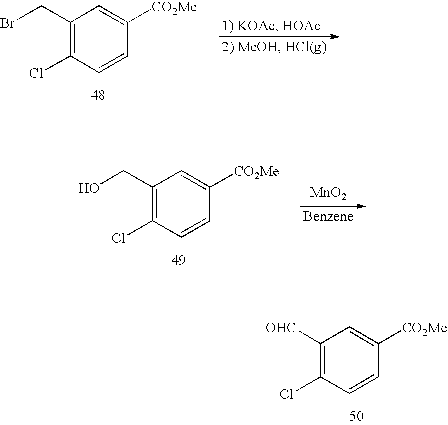 Preparation of 4-Chloro-3-hydroxymethylbenzoic acid methyl ester (49). A round bottom flask was charged with the bromide 48 (1.18 g, 4.48 mmol) (see, e.g., WO 2002057222), anhydrous potassium acetate (1.18 g) and glacial acetic acid (10 mL) and was refluxed for 4 hours and then stirred at room temperature overnight. The solvent was removed under reduced pressure and the residue was treated with EtOAc, washed with water, brine and dried (MgSO₄). The solvent was removed to give a green oil which was dissolved in MeOH. To this solution was bubbled in HCl gas for 20 min and the resulting solution was refluxed for 3 h. The solvent was removed under reduced pressure and the residue was dissolved in EtOAc, washed with water, sat. NaHCO₃, brine and dried (MgSO₄). After removal of the solvent, the residue was purified via flash column chromatography, eluted with EtOAc/hexane, to give the product as a white solid.
Preparation of 4-Chloro-3-formylbenzoic acid methyl ester (50). The alcohol 49 (280 mg, 1.4 mmol) from the previous step was dissolved in benzene (5 mL) and mixed with activated MnO₂ (365 mg, 3.0 eq). The mixture was refluxed overnight. The solid was removed by filtration. The solvent was evaporated via rotavapor to give the product as a white solid.

### EXAMPLE 15

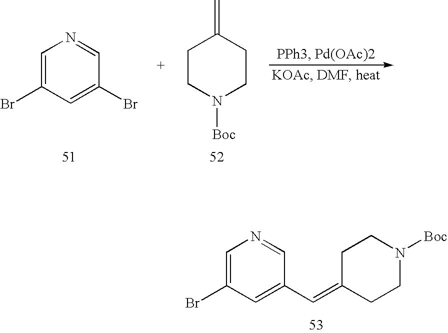 Preparation of 4-(5-Bromopyridin-3-ylmethylene)piperidine-1-carboxylic acid tert-butyl ester (53). To a solution of 3,5-dibromopyridine (1.07 g, 4.5 mmol) (Aldrich) in DMF (15 mL) was added triphenylphosphine (236 mg, 0.2 eq), palladium(II) acetate (101 mg, 0.1 eq), potassium acetate (1.1 g, 2.0 eq) and 1-N-Boc-4-methylenepiperidine (887.8 mg, 1.0 eq). The mixture was heated at 90° C. overnight and extracted with EtOAc. The organic layer was washed with water and dried (MgSO₄). After solvent evaporation, the residue was subjected flash column chromatography, eluting with EtOAc:hexane (1:2) to give the product as a white solid.

### EXAMPLE 16

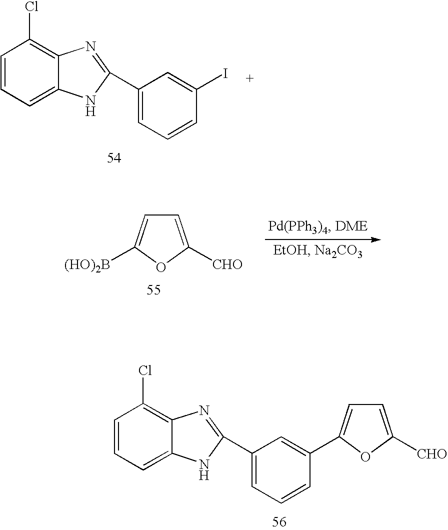 Preparation of 5-[3-(4-Chloro-1H-benzoimidazol-2-yl)phenyl]furan-2-carbaldehyde (56). To a solution of Pd(PPh₃)₄ (133 mg, 0.12 mmol) in DME (3 mL) was added a solution of iodide 54 (840 mg, 2.38 mmol) in DME (7 mL). After stirring at room temperature for 30 min under a nitrogen atmosphere, the mixture was treated with a suspension of boronic acid 55 (500 mg, 3.57 mmol) (Aldrich) in EtOH (3 mL). The resulting mixture was stirred for 20 min at room temperature, and treated with 2.0 M of Na₂CO₃ (8 mL) and then refluxed overnight. After cooling, the mixture was extracted with diethyl ether. The combined organic layers were washed with water and brine and dried (MgSO₄). The solvent was removed under reduced pressure to give the product as a reddish liquid.

### EXAMPLE 17

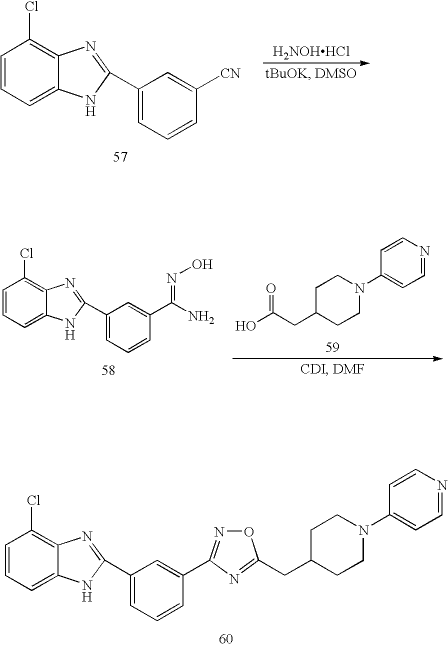 Preparation of 3-(4-Chloro-1H-benzoimidazol-2-yl)-N-hydroxybenzamidine (58). A mixture of hydroxylamine hydrochloride (1.04 g, 15 mmol) in anhydrous DMSO (8 mL) was cooled to 5° C. under nitrogen atmosphere and was then treated with potassium tert-butoxide (1.68 g, 15 mmol) in portions. After stirring 30 min at 5° C., nitrile 57 (380.5 mg, 1.5 mmol) was added and the resulting mixture was stirred at room temperature overnight. The reaction mixture was then poured into ice-water and the precipitate was isolated by filtration. The solid was washed with water and ethanol, and dried under vacuum yielding the product which was sufficiently pure for further manipulation.
Preparation of 4-{3-[3-(4-Chloro-1H-benzoimidazol-2-yl)phenyl]-[1,2,4]oxadiazol-5-ylmethyl}-3,4,5,6-tetrahydro-2H-[1,4′]bipyridinyl (60). To the acid 59 (187.1 mg, 0.85 mmol) in DMF (2 mL) was added CDI (151.6 mg, 0.94 mmol). After stirring for 30 min at room temperature the mixture was treated with a solution of the compound 58 (269.5 mg, 0.94 mmol) in DMF (2 mL) and the resulting solution was stirred at room temperature for 4 h. More CDI (151.6 mg, 0.94 mmol) in DMF (2 mL) was added and the reaction mixture was heated at 115° C. for 6 h to effect cyclodehydration. Solvent was removed and the residue was subjected flash chromatography, eluting with MeOH/DCM/Et₃N to give the final product as a pale yellow solid.

### EXAMPLE 18

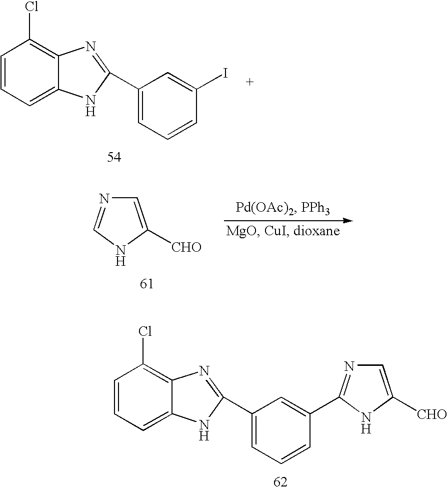 Preparation of 2-[3-(4-Chloro-1H-benzoimidazol-2-yl)phenyl]-3H-imidazole-4(5)-carbaldehyde (62). In a flask, 4(5)-imidazolecarboxaldehyde (115.3 mg, 1.2 mmol) (Aldrich) and MgO (58.1 mg, 1.44 mmol) were suspended in 2 mL of dry dioxane and stirred at rt for 30 min to get a homogeneous suspension. Pd(OAc)₂ (53.76 mg, 20% mmol) and PPh₃ (157.4 mg, 50% mmol) were added to this mixture under nitrogen atmosphere with vigorous stirring. Iodide 54 (354.6 mg, 1.0 mmol) dissolved in 2 mL of dry dioxane was added to the above mixture. The mixture was then heated at 150° C. in a sealed tube for 36 h. The solid was filtered and washed with MeOH. The filtrate was concentrated to give the product as a yellow oil which was used without further purification.

### EXAMPLE 19

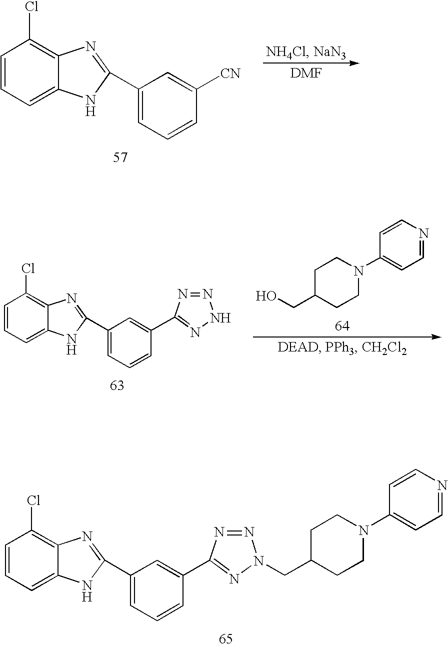 Preparation of 4-Chloro-2-[3-(2H-tetrazol-5-yl)phenyl]-1H-benzoimidazole (63). The nitrile 57 (852.4 mg, 3.36 mmol), NH₄Cl (900 mg, 16.8 mmol) and NaN₃ were mixed in dry DMF (15 mL) and heated at 120° C. for 6 h. After removal of the solvent, the residue was suspended in water (100 mL) and basified with 5% NaOH (5 mL). The clear solution was extracted with diethyl ether and separated. The aqueous layer was then treated with charcoal and filtered. The filtrate was acidified to pH 2 with 10% HCl. The solid was isolated via filtration to give the product as a white solid.
Preparation of 4-{5-[3-(4-Chloro-1H-benzoimidazol-2-yl)phenyl]tetrazol-2-ylmethyl}-3,4,5,6-tetrahydro-2H-[1,4′]bipyridinyl (65). To a stirred mixture of the tetrazole 63 (231.5 mg, 0.78 mmol) and alcohol 64 (150 mg, 0.78 mmol, J & W PharmLab) in DCM (10 mL) at 5° C. under nitrogen atmosphere was added Ph₃P (205 mg, 0.78 mmol) in one portion, followed by dropwise addition of DEAD (0.125 mL, 0.78 mmol) over a few min. The resulting mixture was stirred at 5° C., then room temperature overnight. The mixture was diluted with DCM (20 mL), washed with water, brine and dried (MgSO₄). After evaporation of the solvent, the residue was subjected to prep HPLC to give the product as a white solid.

### EXAMPLE 20

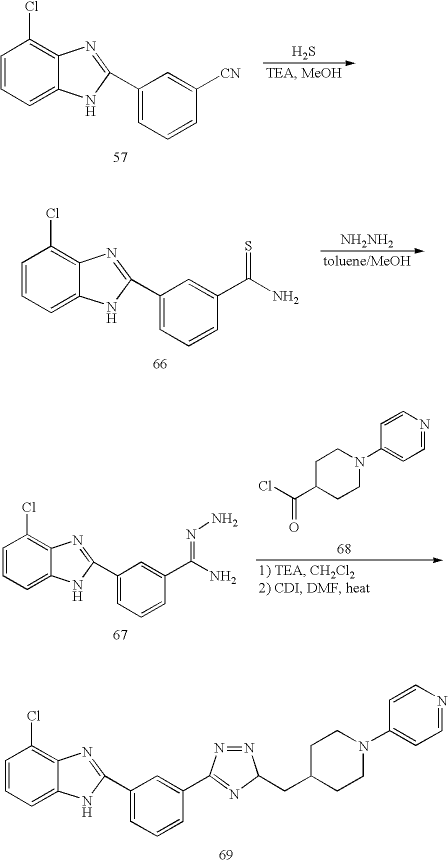 Preparation of 3-(4-Chloro-1H-benzoimidazol-2-yl)thiobenzamide (66). To a solution of nitrile 57 (2.0 g, 7.9 mmol) and TEA (5.8 mL) in MeOH (15 mL) was bubbled H₂S gas to the point of saturation. The mixture was then stirred at room temperature overnight. The solvent was removed under reduced pressure to give the desired product.
Preparation of 3-(4-Chloro-1H-benzoimidazol-2-yl)-N-amine-benzamidine (67). The thioamide 66 (2.2 g, 7.64 mmol) was dissolved in a mixture of toluene and methanol (4:1. 50 mL) and treated with anhydrous hydrazine (0.25 g, 7.64 mmol) at ° C. The mixture was stirred at this temperature for 8 h. The solvent was removed and triturated with MeOH. The product was isolated by filtration as a tan solid.
Preparation of 4-{5-[3-(4-Chloro-1H-benzoimidazol-2-yl)-phenyl]-3H-[1,2,4]triazol-3-ylmethyl}-3,4,5,6-tetrahydro-2H-[1,4′]bipyridinyl (69). To a solution of the aminoamidine 67 (229 mg, 0.8 mmol) and acid chloride 68 (221 mg. 0.8 mmol) in DCM (10 mL) at 0° C. was added TEA (0.17 mL). The mixture was stirred at this temperature for 1 h. The solvent was removed under reduced pressure. The residue was dissolved in DMF (5 mL) and treated with CDI (129 mg, 0.8 mmol). The mixture was heated at 115° C. for 6 h. The solvent was removed and the residue was subjected preparative HPLC to give the product as a white solid.

### EXAMPLE 21

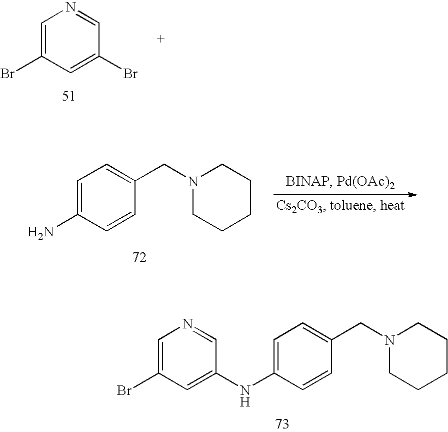 Preparation of (5-Bromopyridin-3-yl)-4-piperidin-1-ylmethylphenyl)amine (73). A vial was charged with palladium acetate (16.8 mg, 5 mol %), (+/−)BINAP (46.7 mg, 5 mol %) and toluene (5 mL). The mixture was stirred and flushed with nitrogen for 10 min. Another round bottom flask was charged with 3,5-dibromopyridine (355.4 mg, 1.5 mmol), amine 72 (342.5 mg, 1.8 mmol) and cesium carbonate (2.44 g, 7.5 mmol). Then the Pd(OAc)₂/BINAP solution was added to the flask and the vial was rinsed with additional toluene (5 mL). The resulting mixture was flushed with nitrogen for 5 min and subsequently heated in a preheated oil bath at 120° C. under vigorous stirring overnight. After cooling, the mixture was filtered and the solid was washed with CH₂Cl₂. The organic layers were combined and evaporated under reduced pressure to give a brown solid. The solid was purified via flash column chromatography, eluting with hexane/EtOAc to give the product as a yellow oil.

### EXAMPLE 22

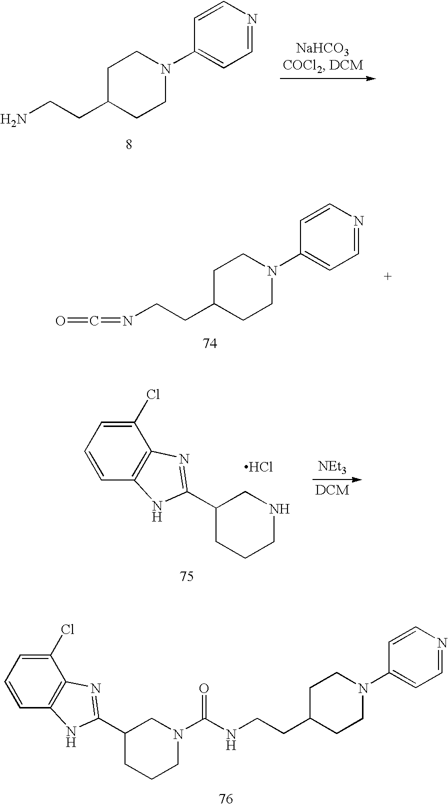 Preparation of 4-(2-Isocyanatoethyl)-3,4,5,6-tetrahydro-2H-[1,4′]bipyridine (74).

In an oven-dried round-bottomed flask was suspended 0.364 g (4.33 mmol) of NaHCO₃ in 21.6 mL dry DCM added via syringe under N₂ gas. The mixture was cooled to 0° C. and 0.910 mL (1.73 mmol) of COCl₂ in toluene was added via syringe. Then, 0.089 g (0.433 mmol) of amine 8 dissolved in 2.16 mL of DCM was added via syringe and left to stir for 30 min. The DCM was removed by rotary evaporation and the crude residue was dissolved in EtOAc, washed with 0.2 M citric acid, distilled H₂O and brine, dried over MgSO₄, filtered and the solvent removed by rotary evaporation to yield the product as a pale yellow oil.

Preparation of 3-(4-Chloro-1H-benzoimidazol-2-yl)-piperidine-1-carboxylic acid [2-(3,4,5,6-tetrahydro-2H-[1,4′]bipyridinyl-4-yl)ethyl]amide (76). In an oven-dried round-bottomed flask was dissolved 0.0570 g (0.210 mmol) of 4-chloro-2-piperidin-3-yl-1H-benzoimidazole hydrochloride (75, prepared as described for compound 13 in EXAMPLE 3 using diamine 11 and 1-N-Boc-3-formylpiperidine (Syntech)) in 2 mL dry DCM added via syringe under N₂ gas. 0.600 mL (4.33 mmol) of NEt₃ was added via syringe and the solution was cooled to 0° C. 0.100 g (0.433 mmol) of 4-(2-isocyanatoethyl)-3,4,5,6-tetrahydro-2H-[1,4′]bipyridinyl dissolved in 2 mL dry DCM was added via syringe and the solution left to stir overnight. The reaction mixture was washed with distilled H₂O, saturated NaHCO₃, distilled H₂O and brine, dried over MgSO₄, filtered and the solvent removed by rotary evaporation to yield a yellow oil. The crude product was purified by preparative HPLC.

### EXAMPLE 23

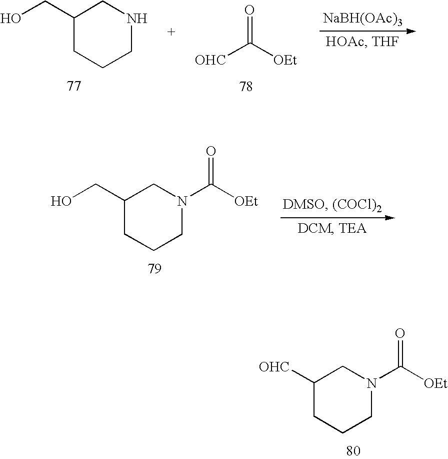 Preparation of 3-Hydroxymethyl-piperidine-1-carboxylic acid ethyl ester (79). To a solution of 3-piperidinemethanol (3.0 g, 26.04 mmol) in THF (25 mL) was added the aldehyde 78 (5.85 mL, 1.1 eq). After stirring at room temperature for 10 min, sodium triacetoxyborohydride (8.28 g, 1.5 eq) and glacial acetic acid (3.5 mL) were added to the above mixture. The mixture was stirred at room temperature overnight and quenched by addition of saturated NaCO₃. The mixture was extracted with EtOAc and the solvent was evaporated to give the product as a colorless oil.
Preparation of 3-Formyl-piperidine-1-carboxylic acid ethyl ester (80). To a solution of oxalyl chloride (0.52 mL, 5.92 mmol) in anhydrous DCM (25 mL) was added DMSO (0.82 mL, 11.52 mmol) at −78° C. After 5 min, a solution of alcohol 79 (1.0 g, 5.34 mmol) in DCM (20 mL) was added. The mixture was stirred for 2 h at −78° C. and then quenched with TEA (12.9 mL). The reaction mixture was warmed up to room temperature and diluted with DCM (50 mL). The organic layer was washed with saturated NH₄Cl, brine and dried (MgSO₄). The solvent was evaporated and the crude product was purified via flash column chromatography, eluting with EtOAc/hexane to give the product as a yellow oil.

### EXAMPLE 24

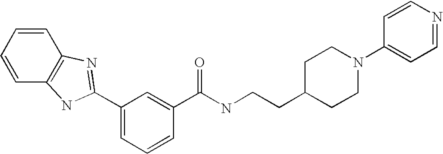 Preparation of 3-(1H-Benzoimidazol-2-yl)-N-[2-(3,4,5,6-tetrahydro-2H-[1,4′]bipyridinyl-4-yl)-ethyl]benzamide. Prepared as described for compound 27 in EXAMPLE 6 using amine 8 and 3-(1H-benzoimidazol-2-yl)benzoic acid, prepared as described for compound 13 in EXAMPLE 3 using 1,2-phenylenediamine (Aldrich) and methyl 3-formylbenzoate (Fluka) followed by ester hydrolysis. ¹H NMR (CD₃OD) 8.66 (s, 1H), 8.28 (d, J=7.5 Hz, 1H), 8.17 (d, J=7.8 Hz, 1H), 8.06 (d, J=7.5 Hz, 2H), 7.90-7.82 (m, 3H), 7.68-7.60 (m, 2H), 7.14 (d, J=7.8 Hz, 2H), 4.27 (d, J=14.4 Hz, 2H), 3.53 (t, J=7.5 Hz, 2H), 3.25-3.15 (m, 2H), 2.10-2.00 (m, 1H), 1.85-1.75 (m, 2H), 1.66 (q, J=6.6 Hz, 2H), 1.40-1.25 (m, 2H). MS (M+H)=426.2.

### EXAMPLE 25

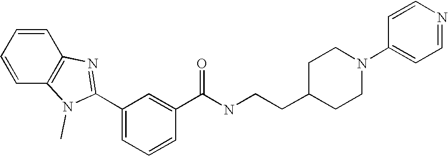 Preparation of 3-(1-Methyl-1H-benzoimidazol-2-yl)-N-[2-(3,4,5,6-tetrahydro-2H-[1,4′]bipyridinyl-4-yl)-ethyl]benzamide. Prepared as described for compound 27 in EXAMPLE 6 using amine 8 and 3-(1-Methyl-1H-benzoimidazol-2-yl)benzoic acid, prepared as described for compound 13 in EXAMPLE 3 using N-methyl-1,2-phenylenediamine (Aldrich) and methyl 3-formylbenzoate (Fluka) followed by ester hydrolysis. ¹H NMR (CD₃OD) 8.37 (t, J=2.1 Hz, 1H), 8.23 (dt, J=0.9, 7.5 Hz, 1H), 8.11-8.03 (m, 3H), 8.00-7.94 (m, 1H), 7.92-7.82 (m, 2H), 7.76-7.68 (m, 2H), 7.13 (d, J=7.5 Hz, 2H), 4.27 (d, J=13.8 Hz, 2H), 4.10 (s, 3H), 3.53 (t, J=6.6 Hz, 2H), 3.25-3.15 (m, 2H), 2.10-2.00 (m, 2H), 1.95-1.80 (m, 1H), 1.65 (q, J=6.6 Hz, 2H), 1.40-1.25 (m, 2H). MS (M+H)=440.2.

### EXAMPLE 26

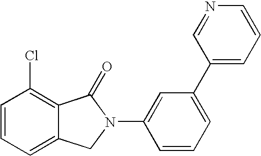

Preparation of 7-Chloro-2-(3-pyridin-3-yl-phenyl)-2,3-dihydro-isoindol-1-one. Prepared as described for compound 15 in EXAMPLE 3 using 3-pyridineboronic acid (Aldrich) and 7-chloro-2-(3-iodophenyl)-2,3-dihydro-isoindol-1-one, which was prepared as described for compound 5 in Scheme 1 using ester 3 and 3-iodoaniline (Aldrich). ¹H NMR (CDCl₃) δ 8.90-8.89 (m, 1H), 8.62 (dd, J=4.8, 1.2 Hz, 1H), 8.32 (bs, 1H), 7.96 (dt, J=6.6, 1.9 Hz, 1H), 7.75 (dt, J=8.1, 0.9 Hz, 1H), 7.57-7.36 (m, 6H), 4.90 (s, 2H). MS (M+H)=321.2.

### EXAMPLE 27

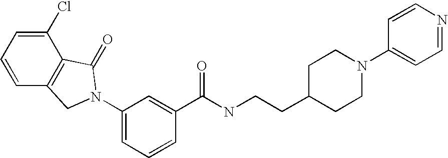 Preparation of 3-(7-Chloro-1-oxo-1,3-dihydro-isoindol-2-yl)-N-[2-(3,4,5,6-tetrahydro-2H-[1,4′]bipyridinyl-4-yl)ethyl]-benzamidev. Prepared as described in EXAMPLE 2. ¹H NMR (CD₃OD): δ 8.33 (s, 1H), 8.06 (d, J=7.4 Hz, 2H), 8.02 (m, 1H), 7.67-7.50 (m, 5H), 7.14 (d, J=7.4 Hz, 2H), 5.01 (s, 2H), 4.28 (bd, J=13.5 Hz, 2H), 3.50 (m, 2H), 3.23 (m, 2H), 2.02 (m, 2H), 1.87 (m, 1H), 1.64 (dt(app. q), J=7.0 Hz, 2H), 1.33 (m, 2H). MS (M+H)=475.1.

### EXAMPLE 28

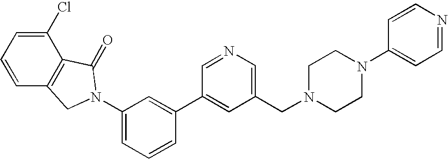 Preparation of 7-Chloro-2-{3-[5-(4-pyridin-4-yl-piperazin-1-ylmethyl)-pyridin-3-yl]-phenyl}-2,3-dihydro-isoindol-1-one. Prepared as described in EXAMPLE 4. ¹H NMR (CD₃OD): δ 8.56 (s, 1H), 8.45 (s, 1H), 8.22 (d, J=7.5 Hz, 2H), 7.87 (m, 2H), 7.65 (m, 5H), 7.53 (m, 1H), 7.26 (d, J=7.5 Hz, 2H), 5.08 (s, 2H), 4.27 (s, 2H), 3.96 (m, 4H), 3.18 (m, 4H). MS (M+H)=496.3.

### EXAMPLE 29

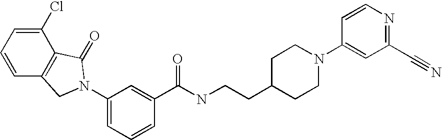 Preparation of 3-(7-Chloro-1-oxo-1,3-dihydro-isoindol-2-yl)-N-[2-(2′-cyano-3,4,5,6-tetrahydro-2H-[1,4′]bipyridinyl-4-yl)-ethyl]-benzamide. Prepared as described for compound 9 in EXAMPLE 2 using 4-(2-aminoethyl)-1-(2-cyanopyrid-4-yl)piperidine and carboxylic acid 6. ¹H NMR (CD₃OD): δ 8.32 (m, 1H), 8.15 (d, J=7.3 Hz, 1H), 8.01 (m, 1H), 7.67-7.49 (m, 6H), 7.15 (dd, J=7.4, 3.2 Hz, 1H), 5.00 (s, 2H), 1.83 (m, 1H), 1.64 (q, J=6.6 Hz, 2H), 1.32 (m, 2H).

### EXAMPLE 30

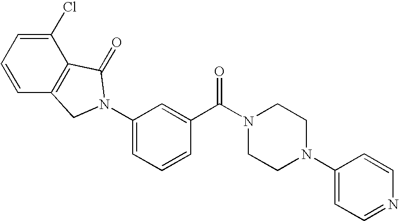 Preparation of 7-Chloro-2-[3-(4-pyridin-4-yl-piperazine-1-carbonyl)-phenyl]-2,3-dihydro-isoindol-1-one. Prepared as described for compound 9 in EXAMPLE 2 using 1-(4-pyridyl)piperazine (Fluka) and carboxylic acid 6. ¹H NMR(CDCl₃): δ 8.29 (m, 2H), 8.09 (s, 1H), 7.82 (d, J=8.0 Hz, 1H), 7.55-7.41 (m, 4H), 7.28 (s, 1H), 6.65 (d, J=6.1 Hz, 2H), 4.84 (s, 2H), 3.90 (bs, 2H), 3.69 (bs, 2H), 3.39 (bs, 4H). MS (M+H)=433.1.

### EXAMPLE 31

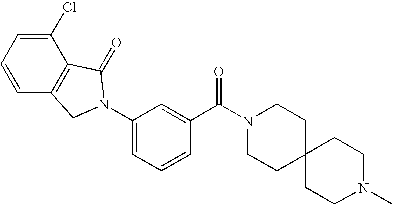 Preparation of 7-Chloro-2-[3-(9-methyl-3,9-diaza-spiro[5.5]undecane-3-carbonyl)-phenyl]-2,3-dihydro-isoindol-1-one. Prepared as described for compound 9 in EXAMPLE 2 using 3-methyl-3,9-diaza-spiro[5.5]undecane (30), which was prepared as described in EXAMPLE 7, and carboxylic acid 6. ¹H NMR (CD₃OD) 8.10 (m, 1H), 7.83 (dd, J=8.1, 1.8 Hz, 1H), 7.65-7.48 (m, 4H), 7.26 (d, J=7.6 Hz, 1H), 4.98 (s, 3H), 3.78 (bs, 2H), 3.50 (bs, 2H), 3.41-3.37 (m, 1H), 3.22-3.07 (m, 3H), 2.88 (m, 2H), 2.06 (d, J=14.2 Hz, 1H), 1.80 (m, 2H), 1.71-1.45 (m, 3H). MS (M+H)=438.2.

### EXAMPLE 32

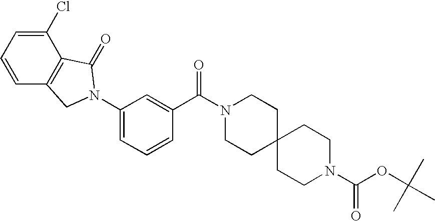 Preparation of 9-[3-(7-Chloro-1-oxo-1,3-dihydro-isoindol-2-yl)-benzoyl]-3,9-diaza-spiro[5.5]undecane-3-carboxylic acid tert-butyl ester. Prepared as described for compound 9 in EXAMPLE 2 using 3,9-diaza-spiro[5.5]undecane-3-carboxylic acid tert-butyl ester (Syntech) and carboxylic acid 6. (CDCl3) 7.95 (dd(app t), J=1.4 hz, 1H), 7.86 (ddd, J=8.2 Hz, 1H), 7.55-7.41 (m, 4H), 7.21 (ddd(app dt), J=7.6, 1.3 Hz, 1H), 4.84 (s, 2H), 3.75 (bs, 2H), 3.41 (m, 6H), 1.65-1.47 (m, 8H), 1.45 (s, 9H). MS (M+H)=524.

### EXAMPLE 33

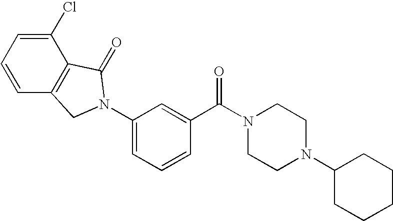 Preparation of 7-Chloro-2-[3-(4-cyclohexyl-piperazine-1-carbonyl)-phenyl]-2,3-dihydro-isoindol-1-one. Prepared as described for compound 9 in EXAMPLE 2 using 1-cyclohexylpiperazine (Fluka) and carboxylic acid 6. (CDCl3) 7.98 (m, 1H), 7.84 (s, 1H), 7.55-7.41 (m, 4H), 7.20 (d, J=7.4 Hz, 1H), 4.84 (s, 2H), 3.79 (bs, 2H), 3.48 (bs, 2H), 2.65 (bs, 2H), 2.52 (bs, 2H), 2.30 (m, 1H), 1.83 (m, 4H), 1.65 (m, 1H), 1.21 (m, 5H). MS (M+H)=438.

### EXAMPLE 34

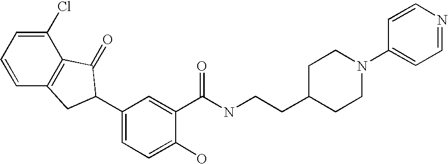 Preparation of 5-(7-Chloro-1-oxo-1,3-dihydro-isoindol-2-yl)-2-hydroxy-N-[2-(3,4,5,6-tetrahydro-2H-[1,4′]bipyridinyl-4-yl)-ethyl]-benzamide. Prepared as described for compound 9 in EXAMPLE 2 using compound 8 and 5-(7-chloro-1-oxo-1,3-dihydro-isoindol-2-yl)-2-hydroxybenzoic acid, which was prepared as described for compound 6 in EXAMPLE 1 using bromide 3 and methyl 5-aminosalicylate (Aldrich) followed by hydrolysis as described. ¹H NMR (CD₃OD) 8.10 (d, J=3.2 Hz, 1H), 8.05 (d, J=7.7 Hz, 2H), 7.79 (dd, J=8.9, 2.6 Hz, 1H), 7.64-7.55 (m, 2H), 7.49 (dd, J=7.3 1.2 Hz, 1H), 7.13 (d, J=7.8 Hz, 2H), 6.99 (d, J=9.2 Hz, 1H), 4.92 (s, 2H), 4.27 (d, J=14.2 Hz, 2H), 3.51 (t, J=7.2 Hz, 2H), 3.21 (m, 2H), 2.01 (bd, J=13.1 Hz, 2H), 1.85 (m, 1H), 1.64 (dt (app. q), J=7.0 Hz, 2H), 1.32 (m, 2H). MS (M+H)=491.1.

### EXAMPLE 35

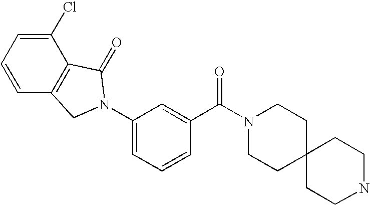 Preparation of 7-Chloro-2-[3-(3,9-diaza-spiro[5.5]undecane-3-carbonyl)-phenyl]-2,3-dihydro-isoindol-1-one. Prepared as described for compound 8 in EXAMPLE 2 using 9-[3-(7-chloro-1-oxo-1,3-dihydro-isoindol-2-yl)benzoyl]-3,9-diaza-spiro[5.5]undecane-3-carboxylic acid tert-butyl ester. ¹H NMR (CD₃OD) 8.11 (m, 1H), 7.84 (m, 1H), 7.66-7.49 (m, 5H), 7.27 (ddd(app. dt), J=7.5, 1.2 Hz, 1H), 5.00 (s, 2H), 3.79 (bs, 2H), 3.52 (bs, 2H), 3.21 (m, 4H), 1.82 (m, 4H), 1.71 (bs, 2H), 1.62 (bs, 2H).

### EXAMPLE 36

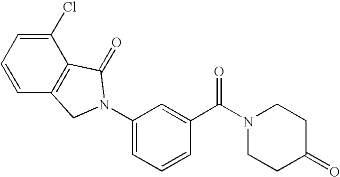 Preparation of 7-Chloro-2-[3-(4-oxo-piperidine-1-carbonyl)-phenyl]-2,3-dihydro-isoindol-1-one. Prepared as described for compound 9 in EXAMPLE 2 using carboxylic acid 6 and 4-piperidinone hydrochloride monohydrate (TimTec). (CDCl3), 8.19 (s, 1H), 7.79 (dd, J=8.3, 1.6 Hz, 2H), 7.55-7.48 (m, 2H), 7.45-7.41 (m, 2H), 7.29 (d, J=8.0 Hz, 1H), 3.91 (m, 4H), 2.54 (bs, 4H). MS (M+H)=369.3.

### EXAMPLE 37

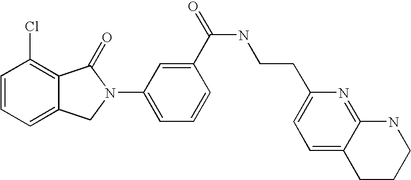 Preparation of 3-(7-Chloro-1-oxo-1,3-dihydro-isoindol-2-yl)-N-[2-(5,6,7,8-tetrahydro-[1,8]naphthyridin-2-yl)-ethyl]-benzamide. Prepared as described for compound 9 in EXAMPLE 2 using 5,6,7,8-tetrahydro-1,8-naphthyridin-2-ethylamine (AstaTech) and carboxylic acid 6. ¹H NMR (CD₃OD), 8.36 (s, 1H), 7.94 (d, J=9.0 Hz, 1H), 7.68-7.53 (m, 6H), 6.67 (d, J=7.5 Hz, 1H), 5.03 (s, 2H), 3.76 (t, J=6.6 Hz, 2H), 3.53 (t, J=11.1 Hz, 2H), 3.52 (t, J=6.3 Hz, 2H), 2.85 (t, J=6.0 Hz, 2H), 2.01-1.96(m, 2H). MS (M+H)=447.4.

### EXAMPLE 38

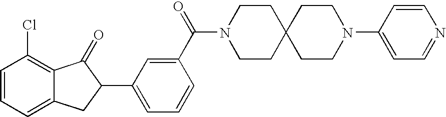 Preparation of 7-Chloro-2-[3-(9-pyridin-4-yl-3,9-diaza-spiro[5.5]undecane-3-carbonyl)-phenyl]-2,3-dihydro-isoindol-1-one. Prepared as described for compound 9 in EXAMPLE 2 using 3-pyridin-4-yl-3,9-diaza-spiro[5.5]undecane (see Smyth, M. S.; Rose, J.; Mehrotra, M. M.; Heath, J.; Ruhter, G.; Schotten, T.; Seroogy, J.; Volkots, D.; Pandey, A.; Scarborough, R. M. Bioorg. Med. Chem. Lett. 2001, 11, 1289) and carboxylic acid 6. ¹H NMR (CD₃OD) 8.15 (s, 1H), 8.10 (d, J=6.3 Hz, 2H), 7.87 (d, J=11.1 Hz, 1H), 7.70-7.51 (m, 4H), 7.30 (d, J=7.8 Hz, 1H), 7.16 (d, J=7.5 Hz, 2H), 5.02 (s, 2H), 3.83 (m, 2H), 3.75 (m, 4H), 3.56 (m, 2H), 1.80 (m, 4H), 1.66 (m, 2H). MS (M+H)=501.2.

### EXAMPLE 39

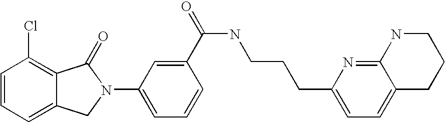 Preparation of 3-(7-Chloro-1-oxo-1,3-dihydro-isoindol-2-yl)-N-[3-(5,6,7,8-tetrahydro-[1,8]naphthyridin-2-yl)-propyl]-benzamide. Prepared as described for compound 9 in EXAMPLE 2 using 5,6,7,8-Tetrahydro-1,8-Naphthyridin-2-propylamine (AstaTech) and carboxylic acid 6. (CDCl3), 8.40(s, 1H), 8.01 (dd, J=1.2, 6.9 Hz, 1H), 7.69-7.52 (m, 6H), 6.70 (d, J=7.2 Hz, 1H), 5.03 (s, 2H), 3.50 (q, J=6.0 Hz, 4H), 2.80(q, J=5.7 Hz, 4H), 2.10-2.03 (m, 2H), 1.97-1.91(m, 2H). MS (M+H)=461.2.

### EXAMPLE 40

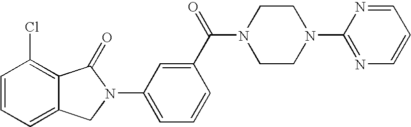 Preparation of 7-Chloro-2-[3-(4-pyrimidin-2-yl-piperazine-1-carbonyl)-phenyl]-2,3-dihydro-isoindol-1-one. Prepared as described for compound 9 in EXAMPLE 2 using 1-(2-pyrimidyl)piperazine (Aldrich) and carboxylic acid 6. (DMSO-d6) 8.40 (d, J=4.8 Hz, 2H), 8.00 (s, 1H), 7.94 (d, J=9.6 Hz, 1H), 7.65 (m, 2H), 7.57 (m, 2H), 7.39 (d, J=4.8 Hz, 1H), 6.68 (t, J=4.8 Hz, 1H), 5.06 (s, 2H), 3.95-3.68 (m, 6H), 3.48 (m, 2H). MS (M+H)=434.3.

### EXAMPLE 41

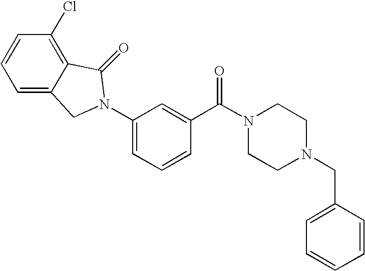 Preparation of 2-[3-(4-Benzyl-piperazine-1-carbonyl)-phenyl]-7-chloro-2,3-dihydro-isoindol-1-one. Prepared as described for compound 9 in EXAMPLE 2 using 1-benzylpiperazine (Aldrich) and carboxylic acid 6. ¹H NMR (CD₃OD) 8.05 (s, 1H), 7.93 (d, J=7.8 Hz, 1H), 7.65-7.51 (m, 4H), 7.38-7.26 (m, 6H), 5.01 (s, 2H), 3.82 (m, 2H), 3.70 (s, 2H), 3.53 (m, 2H), 2.59 (m, 2H), 2.30 (m, 2H). MS (M+H)=446.3.

### EXAMPLE 42

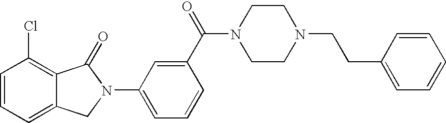 Preparation of 7-Chloro-2-[3-(4-phenethyl-piperazine-1-carbonyl)-phenyl]-2,3-dihydro-isoindol-1-one. Prepared as described for compound 9 in EXAMPLE 2 using 1-(2-phenylethyl)piperazine (Aldrich) and carboxylic acid 6. ¹H NMR (CD₃OD) 8.07 (s, 1H), 7.94 (d, J=8.1 Hz, 1H), 7.65-7.51 (m, 4H), 7.31-7.19 (m, 6H), 5.02 (s, 2H), 3.86 (m, 2H), 3.56 (m, 2H), 3.33 (m, 2H), 2.86 (m, 2H), 2.71-2.61 (m, 4H). MS (M+H)=460.3.

### EXAMPLE 43

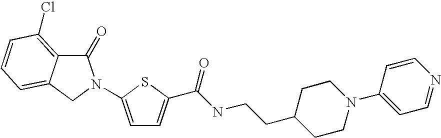 Preparation of 5-(7-Chloro-1-oxo-1,3-dihydro-isoindol-2-yl)-thiophene-2-carboxylic acid [2-(3,4,5,6-tetrahydro-2H-[1,4′]bipyridinyl-4-yl)-ethyl]-amide. Prepared as described for compound 9 in EXAMPLE 2 using 5-(7-chloro-1-oxo-1,3-dihydro-isoindol-2-yl)-thiophene-2-carboxylic acid, which was prepared as described for compound 6 in EXAMPLE 1 using bromide 3 and ester 39, and amine 8. ¹H NMR (CD₃OD) 8.07 (d, J=7.8 Hz, 2H), 7.70-7.52 (m, 4H), 7.15(d, J=7.50 Hz, 2H), 6.90 (d, J=3.90 Hz, 1H), 5.02(s, 2H), 4.28 (s, J=12.31 Hz, 2H), 3.46 (t, J=7.20 Hz, 2H), 3.23 (t, J=10.51 Hz, 2H), 2.03 (d, J=12.31 Hz, 2H), 1.86 (m, 1H), 1.66-1.59 (m, 2H), 1.39-1.31 (m, 2H). MS (M+H)=481.1.

### EXAMPLE 44

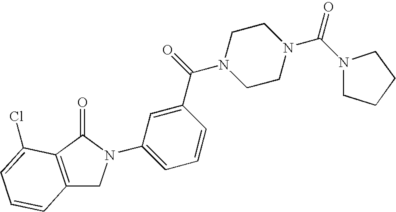

Preparation of 7-Chloro-2-(3-[4-(pyrrolidine-1-carbonyl)-piperazine-1-carbonyl]-phenyl}-2,3-dihydro-isoindol-1-one. Prepared as described for compound 9 in EXAMPLE 2 using piperazin-1-yl-pyrrolidin-1-yl-methanone (Oakwood) and carboxylic acid 6. (CDCl3), 7.96 (s, 1H), 7.72 (d, J=7.8 Hz, 1H), 7.55-7.41 (m, 3H), 7.23 (d, J=7.8 Hz, 2H), 4.84 (s, 2H), 3.79 (br, 2H), 3.52 (br, 2H), 3.28 (br, 6H), 1.86-1.84 (m, 4H), 1.64 (s, 2H). MS (M+H)=453.3.

### EXAMPLE 45

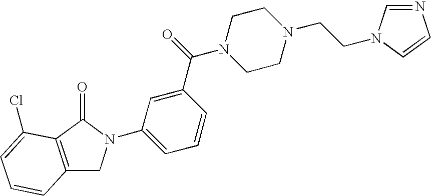 Preparation of 7-Chloro-2-{3-[4-(2-imidazol-1-yl-ethyl)-piperazine-1-carbonyl]-phenyl}-2,3-dihydro-isoindol-1-one. Prepared as described for compound 9 in EXAMPLE 2 using 1-(2-imidazol-1-yl-ethyl)-piperazine (Oakwood) and carboxylic acid 6. (CDCl3), 7.96 (s, 1H), 7.71 (d, J=8.4 Hz, 1H), 7.05 (s, 1H), 6.97 (s, 1H), 4.84 (s, 2H), 4.05 (t, J=6.0 Hz, 2H), 3.79 (br, 2H), 3.50 (br, 2H), 3.43 (br, 2H), 2.73 (t, J=6.6 Hz, 2H), 2.57 (br, 2H). MS (M+H)=450.3.

### EXAMPLE 46

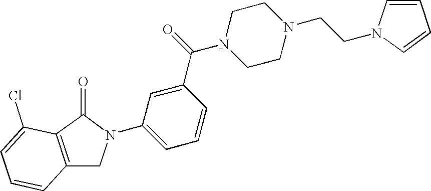 Preparation of 7-Chloro-2-{3-[4-(2-pyrrol-1-yl-ethyl)-piperazine-1-carbonyl]-phenyl}-2,3-dihydro-isoindol-1-one. Prepared as described for compound 9 in EXAMPLE 2 using 1-(2-pyrrol-1-yl-ethyl)piperazine (Oakwood) and carboxylic acid 6. (DMSO-d6), 7.93 (s, 1H), 7.89 (d, J=8.7 Hz, 1H), 7.68-7.63 (m, 2H), 7.17 (d, J=7.5 Hz, 1H), 6.75 (d, J=4.5 Hz, 2H), 5.93 (d, J=4.2 Hz, 2H), 5.02 (s, 2H), 3.97 (t, J=6.6 Hz, 2H), 3.61 (br, 2H), 3.40 (br, 2H), 2.63 (t, J=6.6 Hz, 2H), 2.50-2.38 (br, 4H). MS (M+H)=449.4.

### EXAMPLE 47

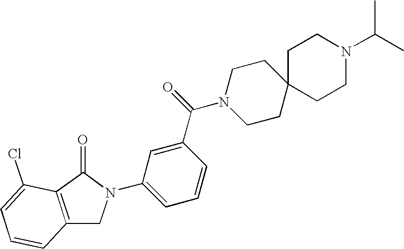 Preparation of 7-Chloro-2-[3-(9-isopropyl-3,9-diaza-spiro[5.5]undecane-3-carbonyl)-phenyl]-2,3-dihydro-isoindol-1-one. Prepared as described for compound 9 in EXAMPLE 2 using 3-isopropyl-3,9-diaza-spiro[5.5]undecane trifluoroacetate, prepared as described for compound 36 in EXAMPLE 9 using 3,9-diaza-spiro[5.5]undecane-3-carboxylic acid tert-butyl ester (Syntech), and carboxylic acid 6. (CDCl3), 8.07-8.03 (m, 1H), 7.77(d, J=8.4 Hz, 1H), 7.56-7.42 (m, 4H), 7.25-7.19 (m, 1H), 4.84 (s, 2H), 3.73 (br, 2H), 3.59-3.34 (m, 2H), 3.35 (d, J=11.4 Hz, 2H), 2.46-2.95 (br, 2H), 1.91 (br, 2H), 1.58-1.45 (br, 6H), 1.45 (s, 1H), 1.34 (s, 6H). MS (M+H)=466.4.

### EXAMPLE 48

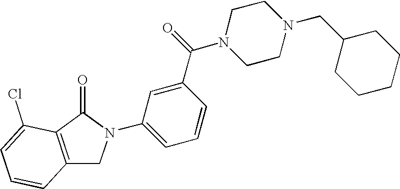 Preparation of 7-Chloro-2-[3-(4-cyclohexylmethyl-piperazine-1-carbonyl)-phenyl]-2,3-dihydro-isoindol-1-one. Prepared as described for compound 9 in EXAMPLE 2 using 1-(cyclohexylmethyl)piperazine (Aldrich) and carboxylic acid 6. (CDCl3), 7.97 (d, J=8.4 Hz, 1H), 7.86 (s, 1H), 7.52-7.41 (m, 4H), 7.21 (d, J=6.8 Hz, 1H), 4.84 (s, 2H), 3.78 (br, 2H), 2.47 (br, 2H), 2.34 (br, 2H), 2.15 (d, J=6.9 Hz, 2H), 1.78-1.63 (m, 4H), 1.47 (m, 1H), 1.24-1.17 (m, 4H), 0.91-0.84 (m, 2H). MS (M+H)=452.4.

### EXAMPLE 49

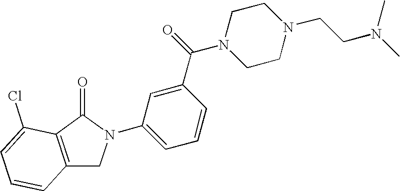 Preparation of 7-Chloro-2-{3-[4-(2-dimethylamino-ethyl)-piperazine-1-carbonyl]-phenyl}-2,3-dihydro-isoindol-1-one. Prepared as described for compound 9 in EXAMPLE 2 using 1-[2-(dimethylamino)ethyl]piperazine (Fluka) and carboxylic acid 6. (DMSO-d6), 7.95 (s, 1H), 7.90(d, J=8.40 Hz, 1H), 7.70-7.62 (m, 2H), 7.57-7.49(m, 2H), 7.19 (d, J=7.5 Hz, 1H), 5.05 (s, 2H), 3.62 (br, 2H), 3.41-3.29 (br, 1H), 2.50-2.27 (m, 8H), 2.13 (s, 6H). MS (M+H)=427.4.

### EXAMPLE 50

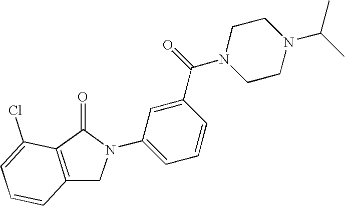 Preparation of 7-Chloro-2-[3-(4-isopropyl-piperazine-1-carbonyl)-phenyl]-2,3-dihydro-isoindol-1-one. Prepared as described for compound 9 in EXAMPLE 2 using 1-isopropylpiperazine (Aldrich) and carboxylic acid 6. (CDCl3) 9.00 (d, J=5.1 Hz, 1H), 7.85 (s, 1H), 7.53-7.43 (m, 4H), 7.22 (d, J=7.8 Hz, 1H), 4.84 (s, 2H), 3.80 (br, 2H), 3.48 (br, 2H), 2.74 (m, 1H), 2.60 (br, 2H), 2.43 (br, 2H), 1.05 (d, J=6.6 Hz, 6H). MS (M+H)=398.3.

### EXAMPLE 51

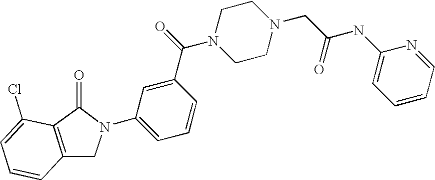 Preparation of 2-{4-[3-(7-Chloro-1-oxo-1,3-dihydro-isoindol-2-yl)-benzoyl]-piperazin-1-yl}-N-pyridin-2-yl-acetamide. Prepared as described for compound 9 in EXAMPLE 2 using 2-(piperazin-1-yl)acetic acid N-(2-pyridyl)amide trihydrochloride dihydrate (Oakwood) and carboxylic acid 6. (CDCl3) 8.63 (d, J=8.7 Hz, 1H), 8.27 (d, J=4.8 Hz, 1H), 8.17 (t, J=7.20 Hz, 1H), 8.01 (s, 1H), 7.90 (d, J=10.5 Hz, 1H), 7.56-7.37 (m, 4H), 7.25 (d, J=6.0 Hz, 2H), 4.85 (s, 2H), 4.06 (br, 2H), 3.83 (s, 2H), 3.13 (br, 4H). MS (M+H)=490.3.

### EXAMPLE 52

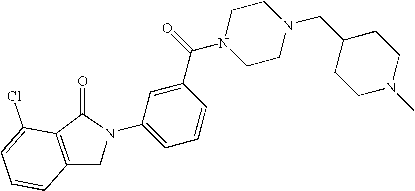 Preparation of 7-Chloro-2-{3-[4-(1-methyl-piperidin-4-ylmethyl)-piperazine-1-carbonyl]-phenyl}-2,3-dihydro-isoindol-1-one. Prepared as described for compound 9 in EXAMPLE 2 using 1-(N-nethylpiperidin-4-yl-methyl)piperazine (Oakwood) and carboxylic acid 6. (CDCl3) 8.26 (s, 1H), 7.66 (d, J=6.6 Hz, 1H), 7.57-7.43 (m, 3H), 7.24(d, J=12.0 Hz, 2H), 4.85 (s, 2H), 3.92(br, 4H), 3.57 (d, J=11.70 Hz, 2H), 3.28 (m, 4H), 2.97 (d, J=5.4 Hz, 2H), 2.77-2.66 (m, 3H), 2.71 (m, 2H), 2.34 (br, 1H), 2.09 (d, J=12.6 Hz, 2H), 1.94 (t, J=12.0 Hz, 2H). MS (M+H)=467.2.

### EXAMPLE 53

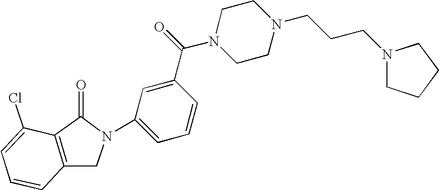 Preparation of 7-Chloro-2-(3-[4-(3-pyrrolidin-1-yl-propyl)-piperazine-1-carbonyl]-phenyl}-2,3-dihydro-isoindol-1-one. Prepared as described for compound 9 in EXAMPLE 2 using 1-(3-pyrrolidinopropyl)piperazine (Oakwood) and carboxylic acid 6. (CDCl3) 8.07(s, 1H), 7.82 (d, J=8.4 Hz, 1H), 7.55-7.42 (m, 4H), 7.23 (d, J=9.9 Hz, 1H), 4.83 (s, 2H), 4.20-3.50 (br, 8H), 3.23-3.12 (m, 8H), 2.33 (m, 2H), 2.09 (m, 4H). MS (M+H)=467.4.

### EXAMPLE 54

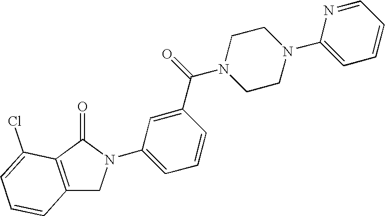 Preparation of 7-Chloro-2-[3-(4-pyridin-2-yl-piperazine-1-carbonyl)-phenyl]-2,3-dihydro-isoindol-1-one. Prepared as described for compound 9 in EXAMPLE 2 using 1-(2-pyridyl)piperazine (Aldrich) and carboxylic acid 6. (CDCl3) 8.21 (d, J=5.10 Hz, 1H), 7.94 (d, J=10.5 Hz, 1H), 7.55-7.47 (m, 5H), 7.25 (d, J=5.1 Hz, 2H), 6.69-6.65 (m, 2H), 4.84 (s, 2H), 3.90 (br, 2H), 3.63 (br, 6H). MS (M+H)=433.3.

### EXAMPLE 55

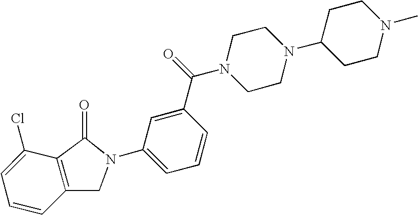 Preparation of 7-Chloro-2-{3-[4-(1-methyl-piperidin-4-yl)-piperazine-1-carbonyl]-phenyl}-2,3-dihydro-isoindol-1-one. Prepared as described for compound 9 in EXAMPLE 2 using 1-(1-methyl-4-piperidinyl)piperazine (Fluka) and carboxylic acid 6. (DMSO-d6) 8.41 (d, J=5.8 Hz, 1H), 7.87 (s, 1H), 7.65-7.60 (m, 2H), 7.54-7.47 (m, 2H), 7.17 (d, J=7.8 Hz, 1H), 5.02 (s, 2H), 3.60(br, 2H), 3.31 (br, 2H), 2.75 (bd, J=10.8 Hz, 2H), 2.52-2.38 (m, 4H), 2.14 (m, 1H), 2.09(s, 3H), 1.79(t, J=10.8 Hz, 2H), 1.67 (bd, J=9.3 Hz, 2H), 1.42-1.36 (m, 2H). MS (M+H)=453.2.

### EXAMPLE 56

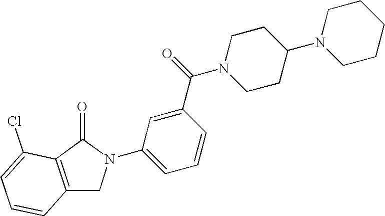 Preparation of 2-[3-([1,4′]Bipiperidinyl-1′-carbonyl)-phenyl]-7-chloro-2,3-dihydro-isoindol-1-one. Prepared as described for compound 9 in EXAMPLE 2 using 4-piperidinopiperidine (Aldrich) and carboxylic acid 6. (DMSO-d6) 7.93 (s, 1H), 7.86 (d, J=9.3 Hz, 1H), 7.68-7.60 (m, 2H), 7.54-7.46 (m, 2H), 7.72 (d, J=7.20 Hz, 1H), 5.02 (s, 2H), 4.49 (br, 2H), 3.65 (br, 2H), 3.02 (br, 2H), 2.71 (br, 2H), 1.80 (br, 2H), 1.66 (br, 2H), 1.47-1.37 (m, 7H). MS (M+H)=438.4.

### EXAMPLE 57

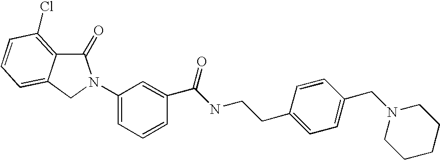 Preparation of 3-(7-Chloro-1-oxo-1,3-dihydro-isoindol-2-yl)-N-[2-(4-piperidin-1-ylmethyl-phenyl)-ethyl]-benzamide. Prepared as described for compound 9 in EXAMPLE 2 using carboxylic acid 6 and 2-(4-piperidin-1-ylmethylphenyl)ethylamine (WO 2004039764). ¹H NMR (CD₃OD) 8.27 (s, 1H), 7.97 (d, J=10.2 Hz, 1H), 7.66-7.52 (m, 5H), 7.45 (s, 4H), 5.02 (s, 2H), 4.26 (s, 2H), 3.68 (t, J=6.90 Hz, 2H), 3.42 (d, J=12.31 Hz, 2H), 3.04-2.89 (m, 4H), 1.94-1.49 (m, 6H). MS (M+H)=488.2.

### EXAMPLE 58

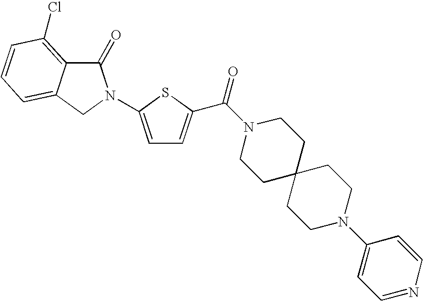 Preparation of 7-Chloro-2-[5-(9-pyridin-4-yl-3,9-diaza-spiro[5.5]undecane-3-carbonyl)-thiophen-2-yl]-2,3-dihydro-isoindol-1-one. Prepared as described for compound 9 in EXAMPLE 2 using 3-pyridin-4-yl-3,9-diaza-spiro[5.5]undecane (see Smyth, M. S.; Rose, J.; Mehrotra, M. M.; Heath, J.; Ruhter, G.; Schotten, T.; Seroogy, J.; Volkots, D.; Pandey, A.; Scarborough, R. M. Bioorg. Med. Chem. Lett. 2001, 11, 1289) and 5-(7-chloro-1-oxo-1,3-dihydro-isoindol-2-yl)-thiophene-2-carboxylic acid, which was prepared as described for compound 6 in EXAMPLE 1 using bromide 3 and ester 39. ¹H NMR (CD₃OD) 8.11 (d, J=7.20 Hz, 2H), 7.70-7.63 (m, 2H), 7.55 (d, J=7.50 Hz, 1H), 7.36 (s, 1H), 7.17 (d, J=7.5 Hz, 2H), 6.90 (s, 1H), 5.03 (s, 2H), 3.84 (m, 4H), 3.76 (m, 4H), 1.80 (m, 4H), 1.72 (m, 4H). MS (M+H)=507.2.

### EXAMPLE 59

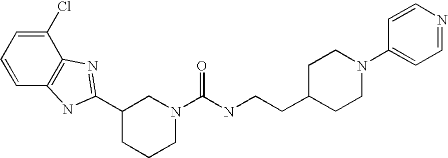 Preparation of 3-(4-Chloro-1H-benzoimidazol-2-yl)-piperidine-1-carboxylic acid [2-(3,4,5,6-tetrahydro-2H-[1,4′]bipyridinyl-4-yl)-ethyl]-amide. Prepared as described in EXAMPLE 22. ¹H NMR (CD₃OD) 8.06 (d, J=7.8 Hz, 2H), 7.66 (dd, J=1.5, 8.1 Hz, 1H), 7.53-7.43 (m, 2H), 7.12 (d, J=8.1 Hz, 2H), 4.24 (d, J=13.2 Hz, 4H), 3.92-3.82 (m, 1H), 3.45-3.30 (m, 1H), 3.25-3.10 (m, 5H), 2.35-2.25 (m, 1H), 2.15-2.00 (m, 1H), 1.94 (d, J=13.2 Hz, 2H), 1.85-1.75 (m, 2H), 1.70-1.60 (m, 1H), 1.51 (q, J=7.2 Hz, 2H), 1.27 (dq, J=3.9, 12.0 Hz, 2H).

### EXAMPLE 60

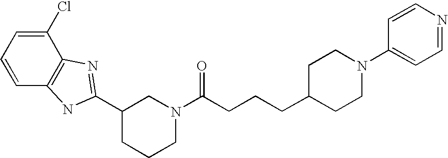 Preparation of 1-[3-(4-Chloro-1H-benzoimidazol-2-yl)-piperidin-1-yl]-4-(3,4,5,6-tetrahydro-2H-[1,4′]bipyridinyl-4-yl)-butan-1-one. Prepared as described for compound 27 in EXAMPLE 6 using 4-(3,4,5,6-Tetrahydro-2H-[1,4′]bipyridinyl-4-yl)-butyric acid, prepared as described for compound 13 in EXAMPLE 3 using diamine 11 and 1-N-Boc-3-formylpiperidine (Syntech) followed by deprotection with TFA and 4-(3,4,5,6-tetrahydro-2H-[1,4′]bipyridinyl-4-yl)butyric acid. MS (M+H)=466.2.

### EXAMPLE 61

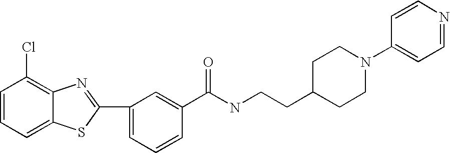 Preparation of 3-(4-Chloro-benzothiazol-2-yl)-N-[2-(3,4,5,6-tetrahydro-2H-[1,4′]bipyridinyl-4-yl)-ethyl]-benzamide. Prepared as described in EXAMPLE 6. (DMSO-d6) 8.77 (t, J=6.9 Hz, 1H), 8.52 (t, J=1.5 Hz, 1H), 8.24 (d, J=8.4 Hz, 1H), 8.17 (d, J=6.6 Hz, 3H), 8.04 (d, J=8.4 Hz, 1H), 7.70 (d, J=8.4 Hz, 2H), 7.67 (dd, J=1.2, 7.8 Hz, 1H), 7.75 (t, J=8.0 Hz, 1H), 4.23 (d, J=12.9 Hz, 2H), 3.30-3.20 (m, 2H), 3.13 (dt, J=2.4, 11.7 Hz, 2H), 1.89 (d, J=11.4 Hz, 2H), 1.80-1.65 (m, 1H), 1.53 (q, J=6.6 Hz, 2H), 1.25-1.10 (m, 2H).

### EXAMPLE 62

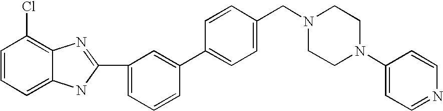 Preparation of 4-Chloro-2-[4′-(4-pyridin-4-yl-piperazin-1-ylmethyl)-biphenyl-3-yl]-1H-benzoimidazole. Prepared as described for compound 17 in EXAMPLE 3 using amine 16 and 3′-(4-chloro-1H-benzoimidazol-2-yl)biphenyl-4-carbaldehyde, prepared as described for compound 15 using 4-formylphenylboronic acid (Aldrich) and 4-chloro-2-(3-iodophenyl)-1H-benzoimidazole, prepared as described for compound 13 using 3-iodobenzaldehyde (Aldrich) in place of comound 12. (DMSO) 8.56 (d, J=1.2 Hz, 1H), 8.37 (d, J=6.6 Hz, 2H), 8.25 (dd, J=0.9, 7.6 Hz, 1H), 7.95-7.85 (m, 3H), 7.71-7.64 (m, 3H), 7.62-7.54 (m, 1H), 7.32-7.20 (m, 5H), 4.44 (s, 2H), 4.20-3.60 (br, 4H), 3.50-3.20 (br, 4H). MS (M+H)=480.4.

### EXAMPLE 63

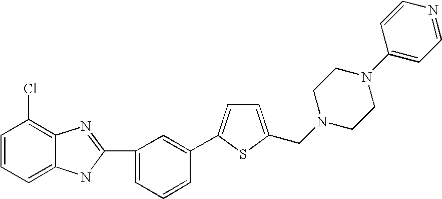

Preparation of 4-Chloro-2-{3-[5-(4-pyridin-4-yl-piperazin-1-ylmethyl]thiophen-2-yl]-phenyl}-1H-benzoimidazole. Prepared as described for compound 17 in EXAMPLE 3 using piperazine 16 and 5-[3-(4-chloro-1H-benzoimidazol-2-yl)phenyl]thiophene-2-carbaldehyde, prepared as described for compound 15 using 5-formyl-2-thiopheneboronic acid (Aldrich) and 4-chloro-2-(3-iodo-phenyl)-1H-benzoimidazole, prepared as described for compound 13 using diamine 11 and 3-iodobenzaldehyde (Aldrich). ¹H NMR (CD₃OD) 8.55 (t, J=1.8 Hz, 1H), 8.24 (d, J=7.5 Hz, 2H), 8.10 (d, J=7.5 Hz, 1H), 7.94-7.87 (m, 1H), 7.70-7.60 (m, 3H), 7.44-7.34 (m, 3H), 7.32-7.26 (m, 2H), 4.55 (s, 2H), 4.05-3.95 (m, 4H), 3.43-3.35 (m, 4H). MS (M+H)=486.3.

### EXAMPLE 64

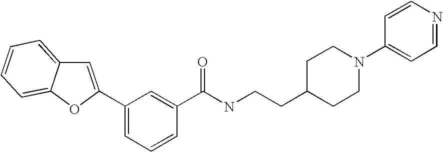 Preparation of 3-Benzofuran-2-yl-N-[2-(3,4,5,6-tetrahydro-2H-[1,4′]bipyridinyl-4-yl)-ethyl]-benzamide. Prepared as described for compound 27 in EXAMPLE 6 using amine 8 and 3-(2-benzofuranyl)benzoic acid (WO 2005009993). ¹H NMR (CD₃OD) 8.70-8.63 (m, 1H), 8.37 (t, J=1.8 Hz, 1H), 8.10-8.02 (m, 2H), 7.82 (dt, J=1.2, 7.8 Hz, 1H), 7.65-7.52 (m, 3H), 7.35-7.22 (m, 3H), 7.14 (d, J=8.4 Hz, 2H), 4.30-4.20 (m, 2H), 3.56-3.48 (m, 2H), 3.25-3.15 (m, 2H), 2.03 (dd, J=1.2, 13.5 Hz, 2H), 1.95-1.78 (m, 1H), 1.65 (q, J=6.9 Hz, 2H), 1.33 (dq, J=1.2, 12.6 Hz, 2H). MS (M+H)=426.2.

### EXAMPLE 65

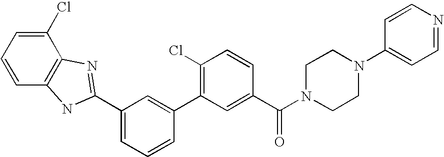 Preparation of [6-Chloro-3′-(4-chloro-1H-benzoimidazol-2-yl)-biphenyl-3-yl]-(4-pyridin-4-yl-piperazin-1-yl)-methanone. Prepared as described for compound 15 in EXAMPLE 3 using boronic acid 13 and (4-chloro-3-iodophenyl)-(4-pyridin-4-yl-piperazin-1-yl)methanone, prepared as described for compound 27 in EXAMPLE 6 using 4-chloro-3-iodobenzoic acid (Oakwood) and 1-(4-pyridyl)piperazine (Fluka). ¹H NMR (CD₃OD) 8.33-8.29 (m, 1H), 8.26-8.16 (m, 3H), 7.80-7.70 (m, 3H), 7.68-7.62 (m, 2H), 7.61-7.58 (m, 1H), 7.48-7.35 (m, 2H), 7.22-7.18 (m, 2H), 4.00-3.70 (br, 8H). MS (M+H)=528.3.

### EXAMPLE 66

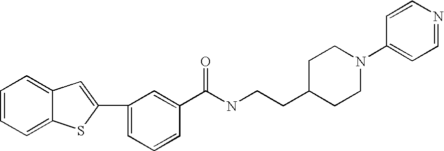 Preparation of 3-Benzo[b]thiophen-2-yl-N-[2-(3,4,5,6-tetrahydro-2H-[1,4′]bipyridinyl-4-yl)-ethyl]-benzamide. Prepared as described for compound 27 in EXAMPLE 6 using amine 8 and 3-benzo[b]thiophen-2-ylbenzoic acid, prepared as described for compound 15 in EXAMPLE 3 using thianaphthene-2-boronic acid (Aldrich) in place of compound 13 and methyl 3-iodobenzoate (Ambinter). ¹H NMR (CD₃OD) 8.70-8.62 (m, 1H), 8.22 (t, J=1.8 Hz, 1H), 8.06 (d, J=7.8 Hz, 2H), 7.96-7.91 (m, 1H), 7.89-7.84 (m, 1H), 7.84-7.80 (m, 1H), 7.95-7.76 (m, 1H), 7.55 (t, J=7.5 Hz, 1H), 7.38-7.33 (m, 2H), 7.94 (d, J=7.2 Hz, 2H), 4.30-4.22 (m, 2H), 3.52 (dq, J=1.2, 7.2 Hz, 2H), 3.23 (dt, J=2.4, 13.8 Hz, 2H), 2.08-1.95 (m, 2H), 1.86-1.78 (m, 1H), 1.65 (q, J=7.2 Hz, 2H), 1.35 (dq, J=1.6, 12.7 Hz, 2H). MS (M+H)=442.6.

### EXAMPLE 67

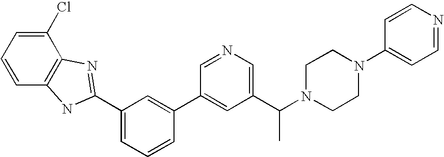 Preparation of 4-Chloro-2-(3-{5-[1-(4-pyridin-4-yl-piperazin-1-yl]ethyl]-pyridin-3-yl}-phenyl)-1H-benzoimidazole. Prepared as described for compound 17 in EXAMPLE 3 using piperazine 16 and 1-{5-[3-(4-chloro-1H-benzoimidazol-2-yl)phenyl]pyridin-3-yl}ethanone, prepared as described for compound 15 using 3-acetyl-5-bromopyridine (Lancaster) and compound 13. ¹H NMR (CD₃OD) 9.04 (s, 1H), 8.72 (s, 1H), 8.60 (s, 1H), 8.53 (s, 1H), 8.19 (d, J=7.97 Hz, 1H), 8.12 (d, J=7.64 Hz, 2H), 7.96 (d, J=8.35 Hz, 1H), 7.74 (t, J=7.88 Hz, 1H), 7.57 (d, J=6.26 Hz, 1H), 7.35-7.29 (m, 2H), 7.15 (d, J=7.70 Hz, 2H), 4.24 (q, J=6.95 Hz, 1H), 3.86 (m, 4H), 3.19-3.12 (m, 2H), 3.01-2.93 (m, 2H), 1.69 (d, J=6.8 Hz, 3H). MS (M+H)=495.4.

### EXAMPLE 68

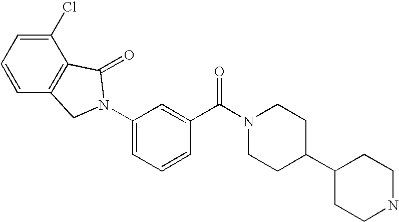 Preparation of 2-[3-([4,4′]Bipiperidinyl-1-carbonyl)-phenyl]-7-chloro-2,3-dihydro-isoindol-1-one. Prepared as described for compound 9 in EXAMPLE 2 using 1-(tert-butoxycarbonyl)-4,4′-bipiperidine (AB Chem) and carboxylic acid 6 followed by treatement with trifluoroacetic acid. ¹H NMR (CD₃OD) 8.11 (t, J=1.5 Hz, 1H), 7.82 (ddd, J=1.2, 2.4, 9.6 Hz, 1H), 7.65-7.48 (m, 4H), 7.24 (dd, J=1.2, 7.8 Hz, 1H), 4.99 (s, 2H), 4.76-4.66 (m, 1H), 3.95-3.80 (m, 1H), 3.45-3.35 (m, 2H), 3.13 (dt, J=1.2, 13.2 Hz, 1H), 2.96 (dt, J=1.2, 12.6 Hz, 2H), 2.95-2.78 (m, 1H), 2.05-1.95 (m, 2H), 1.95-1.85 (m, 1H), 1.80-1.70 (m, 1H), 1.55-1.40 (m, 4H), 1.40-1.20 (m, 2H). MS (M+H)=438.0.

### EXAMPLE 69

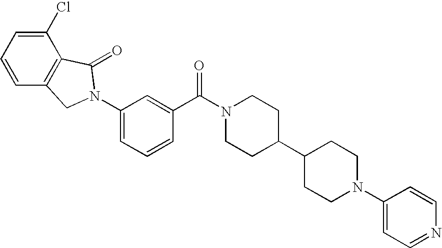 Preparation of 7-Chloro-2-[3-(3′,4′,5′,6′,3″,4″,5″,6″-octahydro-2′H,2″H-[4,1′;4′,4″]terpyridine-1″-carbonyl)-phenyl]-2,3-dihydro-isoindol-1-one. Prepared as described for compound 27 in EXAMPLE 6 using 1-(pyrid-4-yl)-4,4′-bipiperidine and carboxylic acid 6. MS (M+H)=515.2.

### EXAMPLE 70

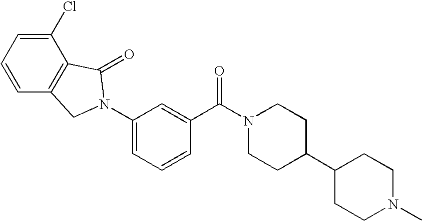 Preparation of 7-Chloro-2-[3-(1′-methyl-[4,4′]bipiperidinyl-1-carbonyl)-phenyl]-2,3-dihydro-isoindol-1-one. Prepared as described for compound 27 in EXAMPLE 6 using carboxylic acid 6 and 1-methyl-[4,4′]bipiperidine, prepared as described for compound 36 in EXAMPLE 9 using piperidine 35 and paraformaldehyde. MS (M+H)=452.2.

### EXAMPLE 71

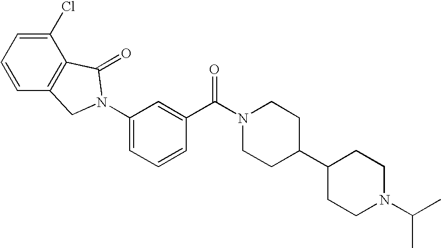 Preparation of 7-Chloro-2-[3-1′-isopropyl-[4,4′]bipiperidinyl-1-carbonyl)-phenyl]-2,3-dihydro-isoindol-1-one. Prepared as described for compound 27 in EXAMPLE 6 using carboxylic acid 6 and amine 36. ¹H NMR (CD₃OD) 8.11 (t, J=1.5 Hz, 1H), 7.83 (ddd, J=0.9, 2.1, 8.1 Hz, 1H), 7.65-7.48 (m, 4H), 7.24 (dd, J=0.9, 7.5 Hz, 1H), 4.99 (s, 2H), 4.75-4.65 (m, 1H), 3.90-3.80 (m, 1H), 3.50-3.40 (m, 3H), 3.18-3.05 (m, 1H), 3.05-2.95 (m, 2H), 2.90-2.75 (m, 1H), 2.15-2.04 (m, 2H), 1.98-1.85 (m, 1H), 1.80-1.72 (m, 1H), 1.58-1.42 (m, 4H), 1.35-1.22 (m, 8H). MS (M+H)=480.2.

### EXAMPLE 72

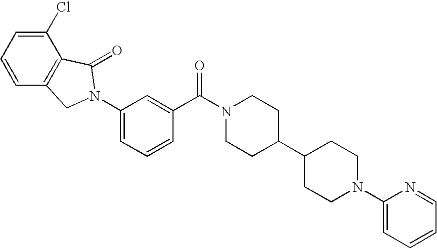 Preparation of 7-Chloro-2-[3-(3′,4′,5′,6′,3″,4″,5″,6″-octahydro-2′H,2″H-[2,1′;4′,4″]terpyridine-1″-carbonyl)-phenyl]-2,3-dihydro-isoindol-1-one. Prepared as described for compound 9 in EXAMPLE 2 using 2-[4,4′-bipiperidin]-1-yl-pyridine (Yoo, K. H.; Choi, H. S.; Kim, D. C.; Shin, K. J.; Kim, D. J.; Song, Y. S.; Jin, C. Archiv der Pharmazie (Weinheim, Germany) 2003, 336, 208. ¹H NMR (CD₃OD) 8.11 (t, J=1.5 Hz, 1H), 8.02-7.95 (m, 1H), 7.89-7.81 (m, 2H), 7.65-7.49 (m, 4H), 7.39 (d, J=9.3 Hz, 1H), 7.25 (dt, J=1.2, 7.2 Hz, 1H), 6.93 (t, J=6.6 Hz, 1H), 4.99 (s, 2H), 4.78-4.65 (m, 1H), 4.25-4.15 (m, 2H), 3.92-3.83 (m, 1H), 3.28-3.05 (m, 3H), 2.90-2.75 (m, 1H), 2.05-1.90 (m, 3H), 1.85-1.75 (m, 1H), 1.68-1.45 (m, 2H), 1.45-1.28 (m, 4H). MS (M+H)=515.4.

### EXAMPLE 73

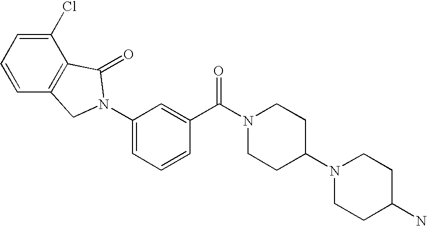 Preparation of 2-[3-(4-Amino-[1,4′]bipiperidinyl-1′-carbonyl)-phenyl]-7-chloro-2,3-dihydro-isoindol-1-one. Prepared as described for compound 27 in EXAMPLE 6 using carboxylic acid 6 and [1,4′]bipiperidinyl-4-yl-carbamic acid tert-butyl ester (47), prepared as described in EXAMPLE 13, followed by deprotection with TFA. ¹H NMR (CD₃OD) 8.19 (t, J=1.5 Hz, 1H), 7.84-7.78 (m, 1H), 7.66-7.49 (m, 4H), 7.29 (dd, J=1.2, 7.8 Hz, 1H), 5.00 (s, 2H), 4.10-3.95 (m, 1H), 3.75-3.65 (m, 2H), 3.65-3.40 (m, 4H), 3.30-3.15 (m, 3H), 3.05-2.95 (m, 1H), 2.35-2.30 (m, 3H), 2.30-2.20 (m, 1H), 2.20-2.10 (m, 1H), 2.10-1.92 (m, 2H), 1.92-1.78 (m, 2H). MS (M+H)=453.4.

### EXAMPLE 74

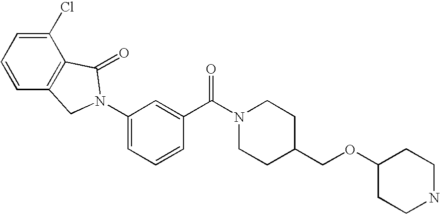 Preparation of 7-Chloro-2-{3-[4-(piperidin-4-yloxymethyl)-piperidine-1-carbonyl]-phenyl}-2,3-dihydro-isoindol-1-one. Prepared as described for compound 27 in EXAMPLE 6 using carboxylic acid 6 and 4-(piperidin-4-ylmethoxy)piperidine-1-carboxylic acid tert-butyl ester (43), prepared as described in EXAMPLE 12, followed by deprotection with TFA. ¹H NMR (CD₃OD) 8.13 (t, J=1.5 Hz, 1H), 7.84-7.79 (m, 1H), 7.65-7.48 (m, 4H), 7.24 (dd, J=1.2, 7.8 Hz, 1H), 4.99 (s, 2H), 4.72-4.60 (m, 1H), 3.88-3.75 (m, 1H), 3.68-3.60 (m, 1H), 3.41 (d, J=6.0 Hz, 2H), 3.28-3.25 (m, 2H), 3.18-3.05 (m, 3H), 2.95-2.80 (m, 1H), 2.05-1.85 (m, 6H), 1.80-1.70 (m, 1H), 1.45-1.25 (m, 2H). MS (M+H)=468.2.

### EXAMPLE 75

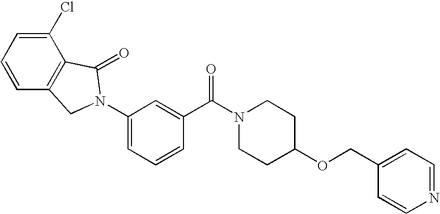 Preparation of 7-Chloro-2-(3-[4-(pyridin-4-ylmethoxy)-piperidine-1-carbonyl]-phenyl)-2,3-dihydro-isoindol-1-one. Prepared as described for compound 9 in EXAMPLE 2 using 4-(4-piperidinyloxy)pyridine (Microchemistry) and carboxylic acid 6. ¹H NMR (CD₃OD) 8.74 (d, J=6.6 Hz, 2H), 8.12 (t, J=1.5 Hz, 1H), 7.97 (d, J=6.6 Hz, 2H), 7.86 (ddd, J=1.2 Hz, 2.1, 8.1 Hz, 1H), 7.66-7.49 (m, 4H), 7.28 (dd, J=0.9, 7.8 Hz, 1H), 5.00 (s, 2H), 4.90-4.80 (m, 2H), 4.15-4.05 (m, 1H), 3.95-3.85 (m, 1H), 3.80-3.70 (m, 1H), 3.68-3.55 (m, 1H), 2.15-1.95 (m, 2H), 1.90-1.73 (m, 2H). MS (M+H)=468.2.

### EXAMPLE 76

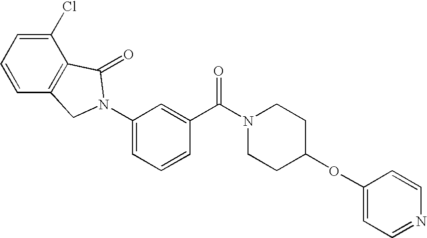 Preparation of 7-Chloro-2-{3-[4-(pyridin-4-yloxy)-piperidine-1-carbonyl]-phenyl}-2,3-dihydro-isoindol-1-one. Prepared as described for compound 27 in EXAMPLE 6 using carboxylic acid 6 and 4-(4-piperidinyloxy)pyridine (Microchemistry). ¹H NMR (CD₃OD) 8.67-8.62 (m, 2H), 8.16 (t, J=2.4 Hz, 1H), 7.85 (ddd, J=0.9, 2.1, 8.1 Hz, 1H), 7.65-7.56 (m, 5H), 7.54-7.46 (m, 1H), 7.31 (dd, J=1.2, 7.8 Hz, 1H), 5.20-5.10 (m, 1H), 5.00 (s, 2H), 4.08-4.00 (m, 1H), 3.85-3.74 (m, 2H), 3.63-3.50 (m, 1H), 2.30-2.10 (m, 2H), 2.05-1.85 (m, 2H). MS (M+H)=448.1.

### EXAMPLE 77

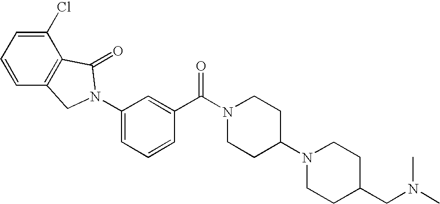 Preparation of 7-Chloro-2-[3-(4-dimethylaminomethyl-[1,4′]bipiperidinyl-1′-carbonyl)-phenyl]-2,3-dihydro-isoindol-1-one. Prepared as described for compound 27 in EXAMPLE 6 using carboxylic acid 6 and [1,4′]bipiperidinyl-4-ylmethyldimethylamine, prepared as described for compound 36 in EXAMPLE 9 using N,N-dimethyl-4-piperidinemethanamine (Microchemistry) and 1-Boc-4-piperidone (Aldrich). ¹H NMR (CD₃OD) 8.17 (t, J=1.8 Hz, 1H), 7.85-7.81 (m, 1H), 7.66-7.49 (m, 4H), 7.29 (dd, J=1.2, 6.9 Hz, 1H), 5.00 (s, 2H), 4.05-3.90 (m, 1H), 3.70-3.60 (m, 2H), 3.60-3.52 (m, 1H), 3.20-3.05 (m, 5H), 2.92 (s, 6H), 2.30-2.15 (m, 2H), 2.15-2.05 (m, 3H), 1.90-1.75 (m, 2H), 1.75-1.55 (m, 2H). MS (M+H)=495.4.

### EXAMPLE 78

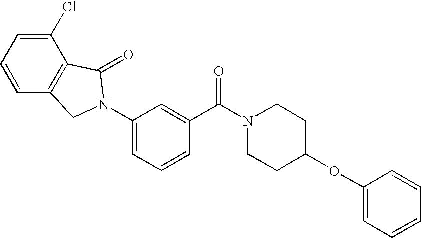 Preparation of 7-Chloro-2-[3-(4-phenoxy-piperidine-1-carbonyl)-phenyl]-2,3-dihydro-isoindol-1-one. Prepared as described for compound 27 in EXAMPLE 6 using carboxylic acid 6 and 4-phenoxypiperidine (Microchemistry). ¹H NMR (CD₃OD) 8.07 (t, J=1.5 Hz, 1H), 7.94-7.88 (m, 1H), 7.64-7.47 (m, 4H), 7.32-7.22 (m, 3H), 7.05-6.88 (m, 3H), 5.00 (s, 2H), 4.75-4.65 (m, 1H), 4.05-3.93 (m, 1H), 3.85-3.65 (m, 2H), 3.55-3.43 (m, 1H), 2.15-1.95 (m, 2H), 1.95-1.75 (m, 2H). MS (M+H)=447.1.

### EXAMPLE 79

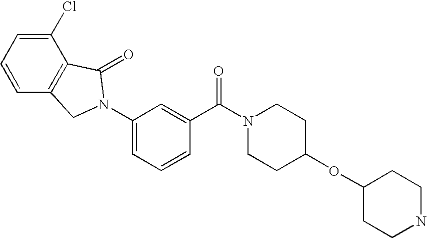 Preparation of 7-Chloro-2-{3-[4-(piperidin-4-yloxy)-piperidine-1-carbonyl]-phenyl}-2,3-dihydro-isoindol-1-one. Prepared as described for compound 27 in EXAMPLE 6 using carboxylic acid 6 and 4-(piperidin-4-yloxy)piperidine-1-carboxylic acid tert-butyl ester (41), prepared as described in EXAMPLE 11, followed by deprotection with TFA. ¹H NMR (CD₃OD) 8.11 (t, J=2.1 Hz, 1H), 7.84 (ddd, J=0.9, 2.1, 8.4 Hz, 1H), 7.65-7.40 (m, 4H), 7.28-7.24 (m, 1H), 4.99 (s, 2H), 4.10-3.95 (m, 1H), 3.90-3.75 (m, 2H), 3.75-3.65 (m, 1H), 3.65-3.55 (m, 1H), 3.40-3.30 (m, 2H), 3.15-3.05 (m, 3H), 2.05-1.90 (m, 3H), 1.90-1.75 (m, 3H), 1.75-1.55 (m, 2H). MS (M+H)=455.1.

### EXAMPLE 80

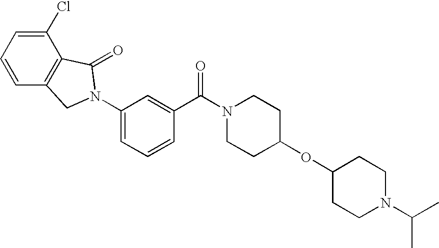 Preparation of 7-Chloro-2-{3-[4-(1-isopropyl-piperidin-4-yloxy)-piperidine-1-carbonyl]-phenyl}-2,3-dihydro-isoindol-1-one. Prepared as described for compound 27 in EXAMPLE 6 using carboxylic acid 6 and 1-(1-methylethyl)-4-(4-piperidinyloxy)piperidine (WO 2005014571). ¹H NMR (CD₃OD) 8.08-8.05 (m, 1H), 7.88 (dt, J=1.2, 8.4 Hz, 1H), 7.64-7.47 (m, 4H), 7.25 (dd, J=1.2, 7.8 Hz, 1H), 4.98 (s, 2H), 4.10-3.98 (m, 1H), 3.98-3.90 (m, 1H), 3.90-3.65 (m, 4H), 3.60-3.40 (m, 3H), 3.40-3.32 (m, 1H), 3.15-3.05 (m, 1H), 2.33-2.22 (m, 1H), 2.20-2.05 (m, 1H), 2.05-1.80 (m, 3H), 1.75-1.55 (m, 3H), 1.34 (t, J=6.6 Hz, 6H). MS (M+H)=496.2.

### EXAMPLE 81

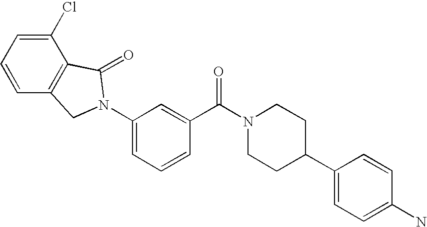 Preparation of 2-{3-[4-(4-Amino-phenyl)-piperidine-1-carbonyl]-phenyl}-7-chloro-2,3-dihydro-isoindol-1-one. Prepared as described for compound 27 in EXAMPLE 6 using carboxylic acid 6 and (4-piperidin-4-yl-phenyl)carbamic acid tert-butyl ester, prepared as described for compound 43 in EXAMPLE 12 using (4-pyridin-4-yl-phenyl)carbamic acid tert-butyl ester (Lamothe, M.; Pauwels, P. J.; Belliard, K.; Schambel, P.; Halazy, S. J. Med. Chem. 1997, 40, 3542. ¹H NMR (CD₃OD) 8.15-8.05 (m, 1H), 7.95-7.85 (m, 1H), 7.60-7.35 (m, 4H), 7.32-7.25 (m, 3H), 7.21-7.10 (m, 2H), 5.03 (s, 2H), 4.85-4.75 (m, 1H), 4.00-3.85 (m, 1H), 3.30-3.20 (m, 1H), 3.05-2.85 (m, 2H), 2.05-1.90 (m, 1H), 1.90-1.70 (m, 3H). MS (M+H)=446.2.

### EXAMPLE 82

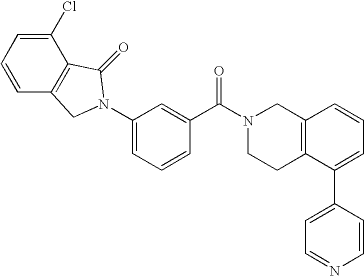 Preparation of 7-Chloro-2-[3-(5-pyridin-4-yl-3,4-dihydro-1H-isoquinoline-2-carbonyl)-phenyl]-2,3-dihydro-isoindol-1-one. Prepared as described for compound 27 in EXAMPLE 6 using carboxylic acid 6 and 5-pyridin-4-yl-1,2,3,4-tetrahydro-isoquinoline, prepared as described for compound 15 in EXAMPLE 3 using 5-bromo-3,4-dihydro-1H-isoquinoline-2-carboxylic acid tert-butyl ester (WO 2002053558) and 4-pyridineboronic acid (Aldrich) followed by deprotection with TFA. ¹H NMR (CD₃OD) 8.70-8.55 (m, 2H), 8.10-8.00 (m, 1H), 8.00-7.90 (m, 1H), 7.70-7.60 (m, 2H), 7.60-7.48 (m, 2H), 7.45-7.30 (m, 3H), 7.30-7.22 (m, 1H), 7.20-7.09 (m, 2H), 5.06 (s, 2H), 4.88-4.80 (m, 1H), 4.75-4.65 (m, 1H), 3.85-3.75 (m, 1H), 3.65-3.50 (m, 1H), 2.90-2.75 (m, 2H). MS (M+H)=480.1.

### EXAMPLE 83

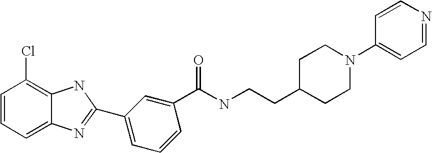 Preparation of 3-(7-Chloro-1H-benzoimidazol-2-yl)-N-[2-(3,4,5,6-tetrahydro-2H-[1,4′]bipyridinyl-4-yl)-ethyl]-benzamide. Prepared as described for compound 27 in EXAMPLE 6 using amine 8 and 3-(7-chloro-1H-benzoimidazol-2-yl)benzoic acid, prepared as described for compound 13 in EXAMPLE 3 using methyl 3-formylbenzoate (Fluka) in place of compound 12 followed by ester hydrolysis. ¹H NMR (DMSO) 8.76-8.72 (m, 2H), 8.37 (d, J=8.1 Hz, 1H), 8.23 (d, J=7.8 Hz, 2H), 8.01 (d, J=8.4 Hz, 1H0, 7.71 (t, J=7.8 Hz, 1H), 7.60 (d, J=7.8 Hz, 1H), 7.35 (d, J=7.4 Hz, 1H), 7.29 (d, J=7.4 Hz, 1H), 7.23 (d, J=7.8 Hz, 2H), 4.28 (d, J=12.9 Hz, 2H), 3.45-3.38 (m, 2H), 3.18 (t, J=12.9 Hz, 2H), 1.98-1.91 (m, 2H), 1.81 (bs, 1H), 1.62-1.52 (m, 2H), 1.27-1.15 (m, 2H). MS (M+H)=460.2.

### EXAMPLE 84

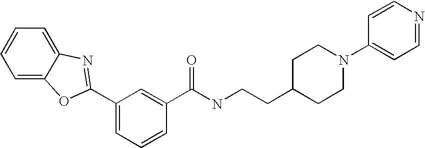 Preparation of 3-Benzooxazol-2-yl-N-[2-(3,4,5,6-tetrahydro-2H-[1,4′]bipyridinyl-4-yl)-ethyl]-benzamide. Prepared as described for compound 27 in EXAMPLE 6 using amine 8 and 3-benzooxazol-2-yl-benzoic acid, prepared as described for compound 34 in EXAMPLE 8 using methyl 3-formylbenzoate (Fluka) and 2-aminophenol (Aldrich) followed by ester hydrolysis. ¹H NMR (DMSO) 8.79 (t, J=5.7 Hz, 1H), 8.68 (s, 1H), 8.33 (d, J=7.5 Hz, 1H), 8.17 (d, J=7.8 Hz, 2H), 8.08 (d, J=7.5 Hz, 1H), 7.85-7.81 (m, 2H), 7.71 (t, J=7.5 Hz, 1H), 7.48-7.41 (m, 2H), 7.18 (d, J=7.8 Hz, 2H), 4.23 (d, 2H, J=13.5 Hz, 2H), 3.39-3.33 (m, 2H), 3.13 (t, J=13.5 Hz, 2H), 1.91-1.87 (m, 2H), 1.75 (bs, 1H), 1.56-1.49)m, 2H), 1.21-1.10 (m, 2H). MS (M+H)=427.1.

### EXAMPLE 85

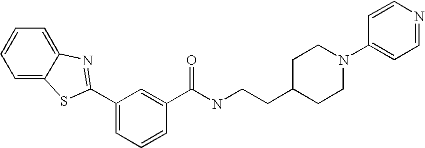 Preparation of 3-Benzothiazol-2-yl-N-[2-(3,4,5,6-tetrahydro-2H-[1,4′]bipyridinyl-4-yl)-ethyl]-benzamide. Prepared as described for compound 27 in EXAMPLE 6 using amine 8 and 3-benzothiazol-2-yl-benzoic acid, prepared as described for compound 34 in EXAMPLE 8 using 2-aminothiophenol in place of compound 33 and methyl 3-formylbenzoate (Fluka) in place of compound 12 followed by ester hydrolysis. ¹H NMR (DMSO) 8.82 (t, J=5.7 Hz, 1H), 8.59 (s, 1H), 8.29-8.22 (m, 4H), 8.14 (d, J=7.2 Hz, 2H), 8.08 (dd, J=1.2, 8.4 Hz, 1H), 7.72 (t, J=7.5 Hz, 1H), 7.63 (t, J=7.2 Hz, 1H), 7.54 (t, J=7.2 Hz, 1H), 7.23 (d, J=7.2 Hz, 2H), 4.28 (d, J=13.2 Hz, 2H), 3.45-3.39 (m, 2H), 3.18 (t, J=12.6 Hz, 2H), 1.94 (d, J=12.6 Hz, 2H), 1.80 (bs, 1H), 1.578 (q, J=7.2 Hz, 2H), 1.27-1.15 (m, 2H). MS (M+H)=443.1.

### EXAMPLE 86

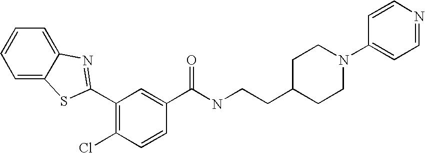 Preparation of 3-Benzothiazol-2-yl-4-chloro-N-[2-(3,4,5,6-tetrahydro-2H-[1,4′]bipyridinyl-4-yl)-ethyl]-benzamide. Prepared as described for compound 27 in EXAMPLE 6 using amine 8 and 3-benzothiazol-2-yl-4-chlorobenzoic acid, prepared as described for compound 34 in EXAMPLE 8 using aldehyde 50 (EXAMPLE 14) in place of boronic acid 12 followed by ester hydrolysis. ¹H NMR (DMSO) 8.84 (t, J=5.1 Hz, 1H), 8.72 (s, 1H), 8.30-8.21 (m, 4H), 8.09-8.04 (m, 1H), 7.88 (dd, J=1.8, 8.4 Hz, 1H), 7.68-7.57 (m, 2H), 7.22 (d, J=7.2 Hz, 2H), 4.27 (d, J=13.8 Hz, 2H), 3.40 (q, J=6.0 Hz, 2H), 3.18 (t, J=12.6 Hz, 2H), 1.92 (d, J=12.3 Hz, 2H), 1.79 (bs, 1H), 1.55 (q, J=6.6 Hz, 2H), 1.28-1.12 (m, 2H). MS (M+H)=477.1.

### EXAMPLE 87

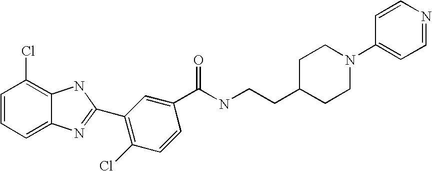 Preparation of 4-Chloro-3-(4-chloro-1H-benzoimidazol-2-yl)-N-[2-(3,4,5,6-tetrahydro-2H-[1,4′]bipyridinyl-4-yl)-ethyl]-benzamide. Prepared as described for compound 27 in EXAMPLE 6 using amine 8 and 4-chloro-3-(4-chloro-1H-benzoimidazol-2-yl)benzoic acid, prepared as described for compound 13 in EXAMPLE 3 using diamine 11 and aldehyde 50 (EXAMPLE 14) followed by ester hydrolysis. ¹H NMR (DMSO) 13.2 (s, 1H), 8.73 (t, J=6.0 Hz, 1H0, 8.38 (d, J=3.0 Hz, 1H), 8.20 (d, J=6.0 Hz, 2H), 8.04 (dd, J=3.0, 9.0 Hz, 1H0, 7.81 (d, J=9.0 Hz, 1H), 7.62 (dd, J=3.0, 9.0 Hz, 1H), 7.38-7.29 (m, 3H), 1.20 (d, J=9.0 Hz, 2H), 4.24 (d, J=12.0 Hz, 2H), 4.38 (q, J=8.0 Hz, 2H), 3.19-3.12 (m, 2H), 1.91 (d, J=15.0 Hz, 2H), 1.77 (bs, 1H), 1.54 (q, J=8.0 Hz, 2H), 1.25-1.17 (m, 2H). MS (M+H)=494.1.

### EXAMPLE 88

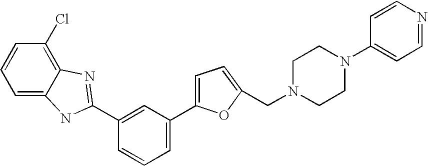 Preparation of 4-Chloro-2-{3-[5-(4-pyridin-4-yl-piperazin-1-ylmethyl)-furan-2-yl]-phenyl}-1H-benzoimidazole. Prepared as described for compound 17 in EXAMPLE 3 using piperazine 16 and 5-[3-(4-chloro-1H-benzoimidazol-2-yl)phenyl]furan-2-carbaldehyde, prepared as described in EXAMPLE 16. MS (M+H)=470.1.

### EXAMPLE 89

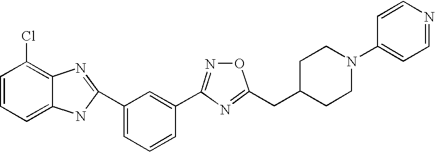 Preparation of 4-{3-[3-(4-Chloro-1H-benzoimidazol-2-yl)-phenyl]-[1,2,4]oxadiazol-5-ylmethyl}-3,4,5,6-tetrahydro-2H-[1,4′]bipyridinyl. Prepared as described in EXAMPLE 17. ¹H NMR (CDCl₃) 8.81 (s, 1H), 8.40 (d, J=7.8 Hz, 1H), 8.31 (d, J=6.0 Hz, 2H), 8.10 (d, J=7.5 Hz, 1H), 7.57-47 (m, 2H), 7.25 (d, J=6.6 Hz, 1H), 7.15 (t, J=7.8 Hz, 1H), 6.67 (d, J=6.0 Hz, 2H), 3.87 (d, J=13.2 Hz, 2H), 2.89-2.81 (m, 2H), 1.85-1.81 (m, 2H), 1.45-0.131 (m, 2H). MS (M+H)=471.1.

### EXAMPLE 90

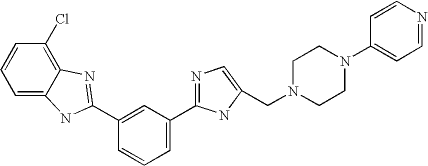 Preparation of 4-Chloro-2-(3-[5-(4-pyridin-4-yl-piperazin-1-ylmethyl)-1H-imidazol-2-yl]-phenyl}-1H-benzoimidazole. Prepared as described for compound 17 in EXAMPLE 3 using piperidine 16 and aldehyde 62 (EXAMPLE 18). ¹H NMR (CD₃OD) 8.90 (bs, 1H), 8.25-8.21 (m, 3H), 8.09 (bs, 1H), 7.81 (bs, 1H), 7.63 (bs, 2H), 7.37-7.18 (m, 5H), 4.05 (bs, 2H), 3.95 (bs, 4H), 3.15 (bs, 4H). MS (M+H)=470.1.

### EXAMPLE 91

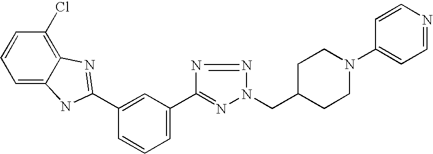 Preparation of 4-{5-[3-(4-Chloro-1H-benzoimidazol-2-yl)-phenyl]-tetrazol-2-ylmethyl}-3,4,5,6-tetrahydro-2H-[1,4′]bipyridinyl. Prepared as described in EXAMPLE 19. ¹H NMR (CDCl₃) 8.81 (s, 1H), 8.40 (d, J=7.8 Hz, 1H), 8.31 (d, J=6.0 Hz, 2H), 8.10 (d, J=7.5 Hz, 1H), 7.57-47 (m, 2H), 7.25 (d, J=6.6 Hz, 1H), 7.15 (t, J=7.8 Hz, 1H), 6.67 (d, J=6.0 Hz, 2H), 3.87 (d, J=13.2 Hz, 2H), 2.89-2.81 (m, 2H), 1.85-1.81 (m, 2H), 1.45-0.131 (m, 2H). MS (M+H)=471.1.

### EXAMPLE 92

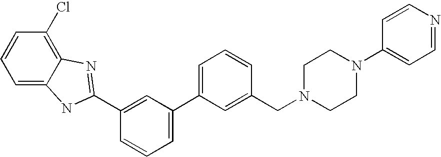 Preparation of 4-Chloro-2-[3′-(4-pyridin-4-yl-piperazin-1-ylmethyl)-biphenyl-3-yl]-1H-benzoimidazole. Prepared as described for compound 17 in EXAMPLE 3 using amine 16 and 3′-(4-chloro-1H-benzoimidazol-2-yl)biphenyl-3-carbaldehyde, prepared as described for compound 15 using 3-formylphenylboronic acid (Aldrich) and 4-chloro-2-(3-iodophenyl)-1H-benzoimidazole, prepared as described for compound 13 using 3-iodobenzaldehyde (Aldrich) in place of compound 12. ¹H NMR (CD₃OD) 8.62-8.61 (m, 1H), 8.30 (d, J=7.8 Hz, 2H), 8.22-8.18 (m, 1H), 8.03-7.98 (m, 3H), 7.81-7.62 (m, 4H), 7.50-7.41 (m, 2H), 7.32 (d, J=7.8 Hz, 2H), 4.56 (bs, 2H), 4.09 (bs, 4H), 3.57-3.52 (m, 4H). MS (M+H)=480.1.

### EXAMPLE 93

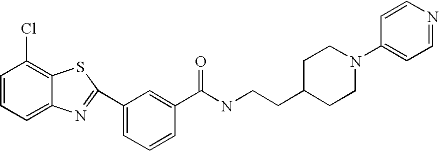 Preparation of 3-(7-Chloro-benzothiazol-2-yl)-N-[2-(3,4,5,6-tetrahydro-2H-[1,4′]bipyridinyl-4-yl)-ethyl]-benzamide. Prepared as described for compound 27 in EXAMPLE 6 using amine 8 and 3-(7-chloro-benzothiazol-2-yl)benzoic acid, prepared as described for compound 34 in EXAMPLE 8 using 2-chloro-6-nitro-benzothiol (Ikeguchi, M.; Sawaki, M.; Maeda, K.; Kikugawa, H. Nippon Noyaku Gakkaishi 2001, 26, 21) in place of compound 32 and methyl 3-formylbenzoate (Fluka) in place of compound 12 followed by ester hydrolysis. ¹H NMR (CD₃OD) 8.80 (t, J=5.1 Hz, 1H), 8.63 (t, J=2.2 Hz, 1H), 8.33 (dd, J=1.8, 8.4 Hz, 1H), 8.11-8.03 (m, 4H), 7.71 (t, J=7.8 Hz, 1H), 7.63-7.53 (m, 2H), 7.18 (d, J=8.4 Hz, 2H), 4.35-4.27 (m, 2H), 3.6-3.54 (m, 2H), 3.27-3.20 (m, 2H), 2.09-2.04 (m, 2H), 1.95-1.84 (m, 1H), 1.70 (q, J=7.2 Hz, 2H), 1.37 (dq, J=3.9, 13.2 Hz, 2H). MS (M+H)=477.1.

### EXAMPLE 94

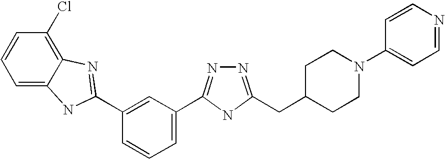 Preparation of 4-{5-[3-(4-Chloro-1H-benzoimidazol-2-yl)-phenyl]-4H-[1,2,4]triazol-3-ylmethyl}-3,4,5,6-tetrahydro-2H-[1,4′]bipyridinyl. ¹H NMR (CD₃OD) 8.85 (t, J=1.8 Hz, 1H), 8.32 (d, J=6.6 Hz, 1H), 8.23 (d, J=7.8 Hz, 1H), 8.10 (d, J=7.8 Hz, 2H), 7.81-7.71 (m, 2H), 7.53-7.49 (m, 2H), 7.18 (d, J=7.8 Hz, 2H), 4.33 (d, J=13.8 Hz, 2H), 3.24 (d, J=13.2 Hz, 2H), 2.90 (dd, J=2.7, 7.2 Hz, 2H), 2.39-2.31 (m, 1H), 1.98 (d, J=13.5 Hz, 2H), 1.52-1.40 (m, 2H). MS (M+H)=470.2.

### EXAMPLE 95

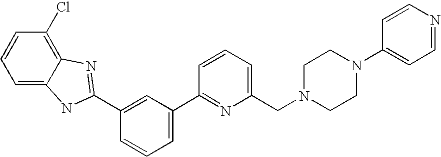 Preparation of 4-Chloro-2-{3-[6-(4-pyridin-4-yl-piperazin-1-ylmethyl)-pyridin-2-yl]-phenyl}-1H-benzoimidazole. Prepared as described for compound 17 in EXAMPLE 3 using 6-bromo-2-pyridine carboxaldehyde (Aldrich) in place of compound 14. ¹H NMR 9.25 (t, J=1.5 Hz, 1H), 8.42 (d, J=6.6 Hz, 1H), 8.31 (d, J=7.8 Hz, 2H), 8.26-8.19 (m, 2H), 8.11 (t, J=7.8 Hz, 1H0, 7.81 (t, J=7.8 Hz, 1H), 7.67 (dd, J=2.4, 6.6 Hz, 1H), 7.57 (d, J=7.2 Hz, 1H), 7.44-7.39 (m, 2H), 7.34 (d, J=7.5 Hz, 2H), 4.79 (s, 2H), 4.27-4.23 (m, 4H), 3.79-3.75 (m, 4H). MS (M+H)=481.1.

### EXAMPLE 96

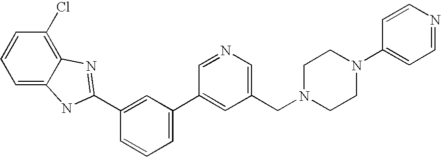 Preparation of 4-Chloro-2-{3-[5-(4-pyridin-4-yl-piperazin-1-ylmethyl)-pyridin-3-yl]-phenyl}-1H-benzoimidazole. Prepared as described in EXAMPLE 3. ¹H NMR (CD₃OD) 8.86 (d, J=2.4 Hz, 1H), 8.52 (dd, J=1.8, 7.8 Hz, 2H), 8.20-8.15 (m, 2H), 8.08 (d, J=6.6 Hz, 2H), 7.83 (d, J=7.8 Hz, 1H), 7.66 (t, J=7.8 Hz, 1H), 7.52 (dd, J=1.2, 7.8 Hz, 1H), 7.28-7.19 (m, 2H), 6.79 (d, J=6.6 Hz, 2H), 3.68 (s, 2H), 3.42-3.37 (m, 4H), 2.63-2.59 (m, 4H). MS (M+H)=481.2.

### EXAMPLE 97

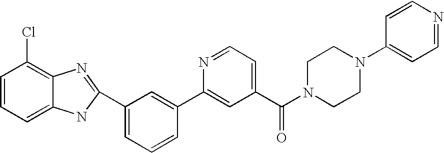 Preparation of {2-[3-(4-Chloro-1H-benzoimidazol-2-yl]phenyl}-pyridin-4-yl)-(4-pyridin-4-yl-piperazin-1-yl)-methanone. Prepared as described for compound 27 in EXAMPLE 6 using piperazine 16 and 2-[3-(4-chloro-1H-benzoimidazol-2-yl)phenyl]isonicotinic acid, prepared as described for compound 15 in EXAMPLE 3 using methyl 2-chloroisonicotinate (Oakwood) in place of compound 12 followed by ester hydrolysis. ¹H NMR (CD₃OD) 8.91 (bs, 1H), 8.84 (d, J=4.8 Hz, 1H), 8.27 (t, J=7.5 Hz, 2H), 8.19-8.15 (m, 4H), 7.73 (t, J=7.2 Hz, 1H), 7.57 (d, J=7.2 Hz, 1H), 7.49 (dd, J=1.2, 5.1 Hz, 1H), 7.33-7.24 (m, 2H), 6.90 (d, 6.9 Hz, 2H), 3.98 (bs, 2H) 3.65 (bs, 4H), 3.51 (bs, 2H). MS (M+H)=495.1.

### EXAMPLE 98

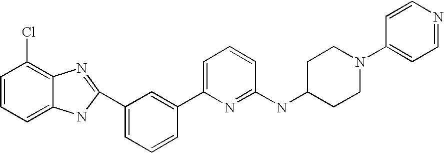 Preparation of {6-[3-(4-Chloro-1H-benzoimidazol-2-yl)-phenyl]-pyridin-2-yl}-(3,4,5,6-tetrahydro-2H-[1,4′]bipyridinyl-4-yl)-amine. Prepared as described for compound 15 in EXAMPLE 3 using boronic acid 13 and (5-bromopyridin-3-yl)-(3,4,5,6-tetrahydro-2H-[1,4′]bipyridinyl-4-yl)amine, prepared as described for compound 73 in EXAMPLE 21 using dibromide 51 and 1-(4-pyridinyl)-4-piperidinamine (Microchemistry). ¹H NMR (CD₃OD) 8.87 (s, 1H), 8.24 (d, J=7.8 Hz, 1H), 8.16-8.12 (m, 3H), 7.66 (t, J=7.8 Hz, 1H), 7.59-7.53 (m, 2H), 7.34-7.25 (m, 3H), 6.91 (d, J=5.1 Hz, 2H), 6.55 (d, J=8.1 Hz, 1H), 4.30 (bs, 1H), 4.07 (d, J=13.2 Hz, 2H), 3.22-3.11 (m, 2H), 2.27 (d, J=13.5 Hz, 2H), 1.67-1.62 (m, 2H). MS (M+H)=481.4.

### EXAMPLE 99

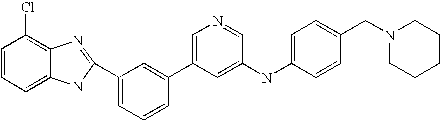 Preparation of {5-[3-(4-Chloro-1H-benzoimidazol-2-yl)-phenyl]-pyridin-3-yl}-(4-piperidin-1-ylmethyl-phenyl)-amine. Prepared as described for compound 15 in EXAMPLE 3 using boronic acid 13 and bromide 73 (EXAMPLE 21). ¹H NMR (DMSO) 8.40 (s, 1H), 8.30 (d, J=1.8 Hz, 1H), 8.26 (d, J=2.4 Hz, 1H), 8.13 (d, J=7.8 Hz, 1H), 7.79-7.7.72 (m, 2H), 7.60 (t, J=7.8 Hz, 1H), 7.49 (d, J=7.5 Hz, 1H), 7.22-7.04 (m, 6H), 3.26 (s, 2H), 2.40 (bs, 4H), 1.57-1.53 (m, 4H), 1.41 (bs, 2H). MS (M+H)=494.4.

### EXAMPLE 100

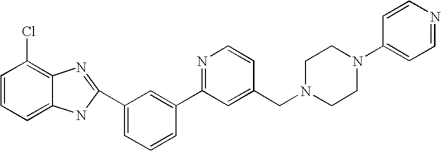

Preparation of 4-Chloro-2-{3-[4-(4-pyridin-4-yl-piperazin-1-ylmethyl)pyridin-2-yl]-phenyl}-1H-benzoimidazole. Prepared as described for compound 15 in EXAMPLE 3 using 2-bromopyridine-4-carboxaldehyde (Lancaster) and compound 13 followed by reductive amination with piperazine 16. ¹H NMR (CD₃OD) 8.84 (t, J=1.8 Hz, 1H), 8.68 (d, J=4.8 Hz, 1H), 8.27-8.20 (m, 2H), 8.15-8.10 (m, 2H), 7.74 (t, J=7.5 Hz, 1H), 7.61-7.51 (m, 2H), 7.35-7.26 (m, 2H), 6.89 (d, J=6.6 Hz, 2H), 3.78 (s, 2H), 3.53-3.50 (m, 4H), 2.72-2.69 (m, 4H). MS (M+H)=481.3.

### EXAMPLE 101

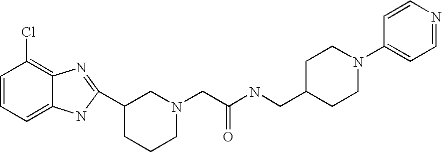 Preparation of 2-[3-(4-Chloro-1H-benzoimidazol-2-yl)-piperidin-1-yl]-N-(3,4,5,6-tetrahydro-2H-[1,4′]bipyridinyl-4-ylmethyl)-acetamide. Prepared as described for compound 27 in EXAMPLE 6 using 1-(4-pyridinyl)-4-piperidinemethanamine (see, e.g., WO 2004098589) and 3-(4-chloro-1H-benzoimidazol-2-yl)piperidine-1-carboxylic acid, prepared as described for compound 13 in Scheme 3 using diamine 11 and aldehyde 80 (EXAMPLE 23) followed by ester hydrolysis. ¹H NMR (CDCl₃) 8.19 (d, J=6.6 Hz, 2H), 7.48 (d, J=7.8 Hz, 1H), 7.21 (d, J=7.8 Hz, 1H), 7.12 (t, J=7.8 Hz, 1H), 6.63 (d, J=6.6 Hz, 2H), 3.86 (d, J=13.2 Hz, 2H), 3.28-3.16 (m, 4H), 3.08-2.98 (m, 2H), 2.88-2.80 (m, 2H), 2.74-2.71 (m, 2H), 2.60-2.52 (m, 4H), 2.0-1.60 (m, 6H), 1.32-1.20 (m, 4H). MS (M+H)=467.4.

### EXAMPLE 102

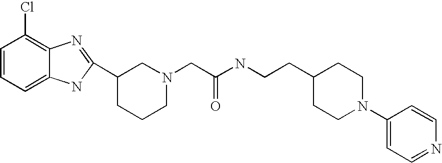 Preparation of 2-[3-(4-Chloro-1H-benzoimidazol-2-yl)-piperidin-1-yl]-N-[2-(3,4,5,6-tetrahydro-2H-[1,4′]bipyridinyl-4-yl)-ethyl]-acetamide. Prepared as described for compound 27 in EXAMPLE 6 using amine 8 and 3-(4-chloro-1H-benzoimidazol-2-yl)piperidine-1-carboxylic acid, prepared as described for compound 13 in EXAMPLE 3 using diamine 11 and aldehyde 80 (EXAMPLE 23) followed by ester hydrolysis. ¹H NMR (DMSO) 10.07 (bs, 1H), 8.68 (bs, 1H), 8.23 (d, J=6.0 Hz, 2H), 7.51 (d, J=7.8 Hz, 1H), 7.28-7.18 (m, 4H), 4.24 (d, J=13.2 Hz, 2H), 4.05 (s, 2H), 3.81 (bs, 1H), 3.60-3.52 (m, 3H), 3.28-3.21 (m, 2H), 3.15-3.05 (m, 4H), 2.28-2.23 (m, 1H), 2.00 (bs 2H), 1.85 (d, J=12.3 Hz, 2H), 1.71 (bs, 2H), 1.48-1.41 (m, 1H). MS (M+H)=481.2.

### EXAMPLE 103

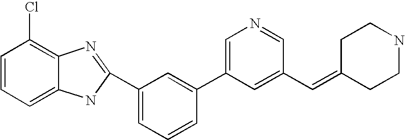 Preparation of 4-Chloro-2-[3-(5-piperidin-4-ylidenemethyl-pyridin-3-yl)-phenyl]-1H-benzoimidazole. Prepared as described for compound 15 in EXAMPLE 3 using boronic acid 13 and bromide 53 (EXAMPLE 15) followed by deprotection with TFA. MS (M+H)=401.3.

### EXAMPLE 104

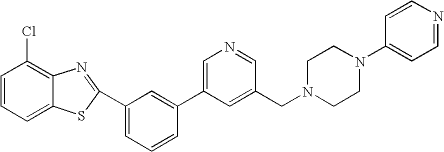 Preparation of 4-Chloro-2-{3-[5-(4-pyridin-4-yl-piperazin-1-ylmethyl)-pyridin-3-yl]-phenyl}-benzothiazole. Prepared as described for compound 17 in EXAMPLE 3 using boronic acid 34 and amine 16. ¹H NMR (CD₃OD) 9.12 (d, J=2.4 Hz, 1H), 8.80 (d, J=1.5 Hz, 1H), 8.60-8.58 (m, 2H), 8.27-8.22 (m, 3H), 8.05-8.22 (m, 2H), 7.79 (t, J=7.8 Hz, 1H), 7.63 (dd, J=1.2, 7.8 Hz, 1H), 7.47 (t, J=7.8 Hz, 1H), 7.27 (d, J=7.8 Hz, 2H), 4.25 (s, 2H), 3.96 (t, J=5.2 Hz, 4H), 3.15 (t, J=5.2 Hz, 4H). MS (M+H)=498.4.

### EXAMPLE 105

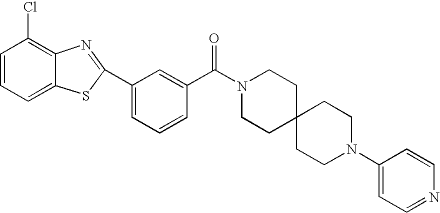 Preparation of [3-(4-Chloro-benzothiazol-2-yl)-phenyl]-(9-pyridin-4-yl-3,9-diaza-spiro[5.5]undec-3-yl)-methanone. Prepared as described for compound 27 in EXAMPLE 6 using 3-pyridin-4-yl-3,9-diaza-spiro[5.5]undecane (see Smyth, M. S.; Rose, J.; Mehrotra, M. M.; Heath, J.; Ruhter, G.; Schotten, T.; Seroogy, J.; Volkots, D.; Pandey, A.; Scarborough, R. M. Bioorg. Med. Chem. Lett. 2001, 11, 1289) and carboxylic acid 26. ¹H NMR (CD₃OD) 8.27-8.23 (m, 2H), 8.10 (d, J=7.8 Hz, 2H), 8.01 (dd, J=1.2, 7.8 Hz, 1H), 7.73-7.60 (m, 3H), 7.45 (t, J=7.8 Hz, 1H), 7.15 (d, J=7.8 Hz, 2H), 3.87 (bs, 2H), 3.77-3.73 (m, 4H), 3.56 (bs, 2H), 1.80 (bs, 6H), 1.65 (bs, 2H). MS (M+H)=503.3.

### EXAMPLE 106

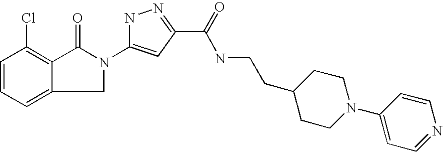 Preparation of 5-(7-Chloro-1-oxo-1,3-dihydro-isoindol-2-yl)-1H-pyrazole-3-carboxylic acid [2-(3,4,5,6-tetrahydro-2H-[1,4′]bipyridinyl-4-yl)-ethyl]-amide. Prepared as described for compound 9 in EXAMPLE 2 using amine 8 and 5-(7-chloro-1-oxo-1,3-dihydro-isoindol-2-yl)-1H-pyrazole-3-carboxylic acid, prepared as described for compound 6 in EXAMPLE 1 using 5-amino-1H-pyrazole-3-carboxylic acid methyl ester (see, e.g., WO 2003101993) in place of benzoate 4. ¹H NMR (DMSO-d6) 8.62 (m, 1H), 8.15 (d, J=5.4 Hz, 2H), 7.65 (m, 2H), 7.51 (m, 1H), 7.00 (d, J=5.4 Hz, 2H), 4.93 (s, 2H), 3.34 (m, 2H), 2.98 (m, 2H), 1.84 (m, 2H), 1.68 (m, 1H), 1.50 (m, 2H), 1.15 (m, 2H). MS (M+H)=465.1.

### EXAMPLE 107

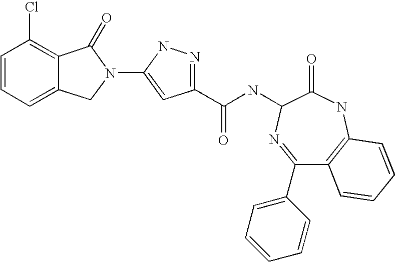 Preparation of 5-(7-Chloro-1-oxo-1,3-dihydro-isoindol-2-yl)-1H-pyrazole-3-carboxylic acid (2-oxo-5-phenyl-2,3-dihydro-1H-benzo[e][1,4]diazepin-3-yl)-amide. Prepared as described for compound 9 in EXAMPLE 2 using 3-amino-5-phenyl-1,3-dihydro-2H-1,4-benzodiazepin-2-one (Tyger) and 5-(7-chloro-1-oxo-1,3-dihydro-isoindol-2-yl)-1H-pyrazole-3-carboxylic acid, prepared as described for compound 6 in EXAMPLE 1 using 5-amino-1H-pyrazole-3-carboxylic acid methyl ester (see, e.g., WO 2003101993) in place of benzoate 4. ¹H NMR (DMSO-d6) 7.81 (m, 1H), 7.66 (m, 3H), 7.49 (m, 5H), 7.34 (m, 3H), 5.49 (s, 1H), 4.97 (s, 2H). MS (M+H)=511.3.

### EXAMPLE 108

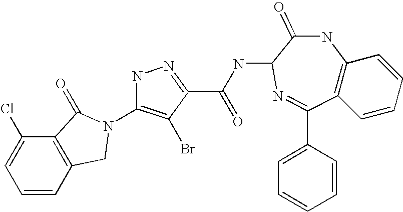 Preparation of 5-(7-Chloro-1-oxo-1,3-dihydro-isoindol-2-yl)-1H-pyrazole-3-carboxylic acid (2-oxo-5-phenyl-2,3-dihydro-1H-benzo[e][1,4]diazepin-3-yl)-amide. Prepared as described for compound 9 in EXAMPLE 2 using 3-amino-5-phenyl-1,3-dihydro-2H-1,4-benzodiazepin-2-one (Tyger) and 4-bromo-5-(7-chloro-1-oxo-1,3-dihydro-isoindol-2-yl)-1H-pyrazole-3-carboxylic acid, prepared as described for compound 6 in EXAMPLE 1 using 5-amino-4-bromo-1H-pyrazole-3-carboxylic acid methyl ester in place of benzoate 4. ¹H NMR (CD₃OD) 7.65 (m, 3H), 7.53 (m, 3H), 7.38 (m, 6H), 5.57 (s, 1H), 4.97 (s, 2H). MS (M+H)=589.2.

### Pharmaceutical Formulations

When employed as pharmaceuticals, the compounds of formula (I) or formula (II) are usually administered in the form of pharmaceutical compositions. These compounds can be administered by a variety of routes including oral, rectal, transdermal, subcutaneous, intravenous, intramuscular, and intranasal. These compounds are effective as both injectable and oral compositions. Such compositions are prepared in a manner well known in the pharmaceutical art and comprise at least one active compound.

This invention also includes pharmaceutical compositions that contain, as the active ingredient, one or more of the compounds of formula (I) or formula (II) above associated with pharmaceutically acceptable carriers. In making the compositions of this invention, the active ingredient is usually mixed with an excipient, diluted by an excipient or enclosed within such a carrier which can be in the form of a capsule, sachet, paper or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, it may be necessary to mill the active compound to provide the appropriate particle size prior to combining with the other ingredients. If the active compound is substantially insoluble, it ordinarily is milled to a particle size of less than 200 mesh. If the active compound is substantially water soluble, the particle size is normally adjusted by milling to provide a substantially uniform distribution in the formulation, e.g., about 40 mesh.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained and/or delayed release of the active ingredient after administration to the patient by employing procedures known in the art.

The compositions are preferably formulated in a unit dosage form, each dosage containing 5 mg to about 100 mg, more usually about 10 mg to about 30 mg, of the active ingredient. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient.

The active compound is effective over a wide dosage range and is generally administered in a pharmaceutically effective amount. It will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing from, for example, 0.1 mg to about 500 mg of the active ingredient of the present invention.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the novel compositions of the present invention may be incorporated for administration orally or by injection include aqueous solutions suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, preferably orally or nasally, from devices that deliver the formulation in an appropriate manner.

The following formulation examples illustrate the pharmaceutical compositions of the present invention.

### FORMULATION EXAMPLE 1

Hard gelatin capsules containing the following ingredients are prepared:

| | Quantity |
|---|---|
| Ingredient | (mg/capsule) |
| Active Ingredient | 30.0 |
| Starch | 305.0 |
| Magnesium stearate | 5.0 |

The above ingredients are mixed and filled into hard gelatin capsules in 340 mg quantities.

### FORMULATION EXAMPLE 2

A tablet formula is prepared using the ingredients below:

| | Quantity |
|---|---|
| Ingredient | (mg/tablet) |
| Active Ingredient | 25.0 |
| Cellulose, microcrystalline | 200.0 |
| Colloidal silicon dioxide | 10.0 |
| Stearic acid | 5.0 |

The components are blended and compressed to form tablets, each weighing 240 mg.

### FORMULATION EXAMPLE 3

A dry powder inhaler formulation is prepared containing the following components:

| Ingredient | Weight % |
|---|---|
| Active Ingredient | 5 |
| Lactose | 95 |

The active mixture is mixed with the lactose and the mixture is added to a dry powder inhaling appliance.

### FORMULATION EXAMPLE 4

Tablets, each containing 30 mg of active ingredient, are prepared as follows:

| | Quantity | |
|---|---|---|
| Ingredient | (mg/tablet) | |
| Active Ingredient | 30.0 | mg |
| Starch | 45.0 | mg |
| Microcrystalline cellulose | 35.0 | mg |
| Polyvinylpyrrolidone | 4.0 | mg |
| (as 10% solution in water) | | |
| Sodium carboxymethyl starch | 4.5 | mg |
| Magnesium stearate | 0.5 | mg |
| Talc | 1.0 | mg |
| Total | 120 | mg |

The active ingredient, starch and cellulose are passed through a No. 20 mesh U.S. sieve and mixed thoroughly. The solution of polyvinyl-pyrrolidone is mixed with the resultant powders, which are then passed through a 16 mesh U.S. sieve. The granules so produced are dried at 50E to 60EC and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 150 mg.

### FORMULATION EXAMPLE 5

Capsules, each containing 40 mg of medicament are made as follows:

| | Quantity | |
|---|---|---|
| Ingredient | (mg/capsule) | |
| Active Ingredient | 40.0 | mg |
| Starch | 109.0 | mg |
| Magnesium stearate | 1.0 | mg |
| Total | 150.0 | mg |

The active ingredient, cellulose, starch, an magnesium stearate are blended, passed through a No. 20 mesh U.S. sieve, and filled into hard gelatin capsules in 150 mg quantities.

### FORMULATION EXAMPLE 6

Suppositories, each containing 25 mg of active ingredient are made as follows:

| Ingredient | Amount | |
|---|---|---|
| Active Ingredient | 25 | mg |
| Saturated fatty acid glycerides | to 2,000 | mg |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

### FORMULATION EXAMPLE 7

Suspensions, each containing 50 mg of medicament per 5.0 mL dose are made as follows:

| Ingredient | Amount | |
|---|---|---|
| Active Ingredient | 50.0 | mg |
| Xanthan gum | 4.0 | mg |
| Sodium carboxymethyl cellulose (11%) | 50.0 | mg |
| Microcrystalline cellulose (89%) | | |
| Sucrose | 1.75 | g |
| Sodium benzoate | 10.0 | mg |
| Flavor and Color | q.v. | |
| Purified water | to 5.0 | mL |

The medicament, sucrose and xanthan gum are blended, passed through a No. 10 mesh U.S. sieve, and then mixed with a previously made solution of the microcrystalline cellulose and sodium carboxymethyl cellulose in water. The sodium benzoate, flavor, and color are diluted with some of the water and added with stirring. Sufficient water is then added to produce the required volume.

### FORMULATION EXAMPLE 8

| | Quantity | |
|---|---|---|
| Ingredient | (mg/capsule) | |
| Active Ingredient | 15.0 | mg |
| Starch | 407.0 | mg |
| Magnesium stearate | 3.0 | mg |
| Total | 425.0 | mg |

The active ingredient, cellulose, starch, and magnesium stearate are blended, passed through a No. 20 mesh U.S. sieve, and filled into hard gelatin capsules in 560 mg quantities.

### FORMULATION EXAMPLE 9

An intravenous formulation may be prepared as follows:

| Ingredient | Quantity | |
|---|---|---|
| Active Ingredient | 250.0 | mg |
| Isotonic saline | 1000 | mL |

### FORMULATION EXAMPLE 10

A topical formulation may be prepared as follows:

| Ingredient | Quantity | |
|---|---|---|
| Active Ingredient | 1-10 | g |
| Emulsifying Wax | 30 | g |
| Liquid Paraffin | 20 | g |
| White Soft Paraffin | to 100 | g |

The white soft paraffin is heated until molten. The liquid paraffin and emulsifying wax are incorporated and stirred until dissolved. The active ingredient is added and stirring is continued until dispersed. The mixture is then cooled until solid.

Another preferred formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. No. 5,023,252, issued Jun. 11, 1991, which is incorporated herein by reference in its entirety. Such patches may be constructed for continuous, pulsatile, or on demand delivery of pharmaceutical agents.

When it is desirable or necessary to introduce the pharmaceutical composition to the brain, either direct or indirect techniques may be employed. Direct techniques usually involve placement of a drug delivery catheter into the host's ventricular system to bypass the blood-brain barrier. One such implantable delivery system used for the transport of biological factors to specific anatomical regions of the body is described in U.S. Pat. No. 5,011,472 which is incorporated herein by reference in its entirety.

Indirect techniques, which are generally preferred, usually involve formulating the compositions to provide for drug latentiation by the conversion of hydrophilic drugs into lipid-soluble drugs. Latentiation is generally achieved through blocking of the hydroxy, carbonyl, sulfate, and primary amine groups present on the drug to render the drug more lipid soluble and amenable to transportation across the blood-brain barrier. Alternatively, the delivery of hydrophilic drugs may be enhanced by intra-arterial infusion of hypertonic solutions which can transiently open the blood-brain barrier.

The following synthetic and biological examples are offered to illustrate this invention and are not to be construed in any way as limiting the scope of this invention. Unless otherwise stated, all temperatures are in degrees Celsius.

### BIOLOGICAL EXAMPLES

The potency and efficacy to inhibit the bradykinin B₁ receptor was determined for the compounds of this invention in a cell-based fluorescent calcium-mobilization assay. The assay measures the ability of test compounds to inhibit bradykinin B₁ receptor agonist-induced increase of intracellular free Ca⁺² in a native human bradykinin B₁ receptor-expressing cell line.

In this example, the following additional abbreviations have the meanings set forth below. Abbreviations heretofore defined are as defined previously. Undefined abbreviations have the art-recognized meanings.
BSA=bovine serum albumin
DMSO=dimethylsulfoxide
FBS=fetal bovine serum
MEM=minimum essential medium
mM=millimolar
ng=nanogram
μg=micrograms
μM=micromolar

Specifically, calcium indicator-loaded cells are pre-incubated in the absence or presence of different concentrations of test compounds followed by stimulation with selective bradykinin B₁ receptor agonist peptide while Ca-dependent fluorescence is monitored.

IMR-90 human lung fibroblast cells (CCL 186, American Type Tissue Collection) are grown in MEM supplemented with 10% FBS as recommended by ATCC. Confluent cells are harvested by trypsinization and seeded into black walvclear bottom 96-well plates (Costar #3904) at approximately 13,000 cells/well. The following day, cells are treated with 0.35 ng/mL interleukin-1β in 10% FBS/MEM for 2 hours to up-regulate bradykinin B₁ receptors. Induced cells are loaded with fluorescent calcium indicator by incubation with 2.3 μM Fluo-4/AM (Molecular Probes) at 37° C. for 1.5 hrs in the presence of an anion transport inhibitor (2.5 mM probenecid in 1% FBS/MEM). Extracellular dye is removed by washing with assay buffer (2.5 mM probenecid, 0.1% BSA, 20 mM HEPES in Hank's Balanced Salt Solution without bicarbonate or phenol red, pH 7.5) and cell plates are kept in dark until used. Test compounds are assayed at 7 concentrations in triplicate wells. Serial dilutions are made in half log-steps at 100-times final concentration in DMSO and then diluted in assay buffer. Compound addition plates contain 2.5-times final concentrations of test compounds or controls in 2.5% DMSO/assay buffer. Agonist plates contain 5-times the final concentration of 2.5 nM (3×EC50) bradykinin B₁ receptor agonist peptide des-Arg¹⁰-kallidin (DAKD, Bachem) in assay buffer. Addition of test compounds to cell plate, incubation for 5 min at 35° C., followed by the addition of bradykinin B₁ receptor agonist DAKD is carried out in the Fluorometric Imaging Plate Reader (FLIPR, Molecular Devices) while continuously monitoring Ca-dependent fluorescence. Peak height of DAKD-induced fluorescence is plotted as function of concentration of test compounds. IC₅₀ values are calculated by fitting a 4-parameter logistic function to the concentration-response data using non-linear regression (Xlfit, IDBS (ID Business Solutions Ltd.)).

In an embodiment of the present invention, an active compound is selected from compounds that exhibit a human FLIPR IC₅₀ value less than or equal to 30 μM.

Typical potencies observed for bradykinin B₁ receptor agonist peptides are EC₅₀ approximately 0.8 nM and approximately 100 nM for des-Arg¹⁰-kallidin and des-Arg⁹-bradykinin, respectively, while for bradykinin B₁ receptor antagonist peptide des-Arg¹⁰, Leu⁹-kallidin IC₅₀ is approximately 1 nM.

1. A method of preventing or treating at least one condition which benefits from inhibition of the bradykinin B1 receptor, comprising:
administering to a host in need thereof a composition comprising a therapeutically effective amount of at least one compound of formula (I), 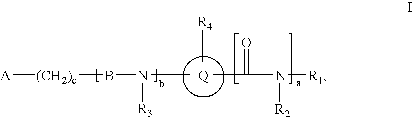
or a pharmaceutically acceptable salt thereof, wherein
A, B, R₁, R₂, R₃, R₄, Q, a, b, and c are as defined in claim 36.

2. A compound according to claim 1 wherein R₁ is selected from (1-(benzyloxyacetyl)-azepan-3-yl)amino; (1,5-dimethyl-2,3,4,5-tetrahydro-1H-benzo[b][1,4]diazepin-3-yl)amino; (1,5-dimethyl-2-oxo-2,3,4,5-tetrahydro-1H-benzo[b][1,4]diazepin-3-yl)amino; (1-cyclopropylmethyl-2-oxo-azepan-3-yl)amino; (1-cyclopropylmethyl-5-methyl-2-oxo-2,3,4,5-tetrahydro-1H-benzo[b][1,4]diazepin-3-yl)amino; (1-cyclopropylmethyl-azepan-3-yl)amino; (1-ethyl-2-oxo-5-phenyl-2,3-dihydro-1H-benzo[e][1,4]diazepin-3-yl)amino; (1′-methyl-[1,4′]bipiperidinyl-4-yl)methylamino; (1 -methyl-piperidin-4-ylmethyl)amino; (1 -pyridin-4-ylmethyl-piperidin-4-yl)amino; (1-pyridin-4-ylmethyl-piperidin-4-ylmethyl)amino; (2-oxo-1-propyl-azepan-3-yl)amino; (2-oxo-5-phenethyl-1 -propyl-2,3-dihydro-1H-benzo[e][1,4]diazepin-3-yl)amino; (3,4,5,6-tetrahydro-2H-[1,4′]bipyridinyl-4-yl)methylamino; (5-methyl-2-oxo-2,3,4,5-tetrahydro-1H-benzo[b][1,4]diazepin-3-yl)amino; (5-methyl-6-oxo-6,7,8,9-tetrahydro-5H-pyrido[3,2-b]azepin-7-yl)amino; (indan-2-yl)amino; (N-(benzyloxyacetyl)piperidin-4-yl)amino; (N-(pyridin-4-ylcarbonyl)piperidin-4-yl)methylamino; [1-(2-dimethylamino-ethyl)-2-oxo-azepan-3-yl]amino; [1-(2-pyridin-4-yl-ethyl)-piperidin-4-yl]amino; [1-(2-pyridin-4-yl-ethyl)-piperidin-4-ylmethyl]amino; [1-(pyridin-4-ylcarbonyl)-piperidin-4-yl]amino; [2-(1′-methyl-[1,4′]bipiperidinyl-4-yl)-ethyl]amino; [2-(1-pyridin-4-ylmethyl-piperidin-4-yl)-ethyl]amino; [2-(4-pyridin-4-yl-piperazin-1-yl)ethyl]amino; [2-(pyridine-4-yl)ethyl]amino; [5-(3-aza-bicyclo[3.2.2]non-3-yl)-1 -methyl-2-oxo-2,3-dihydro-1H-benzo [e][1,4]diazepin-3-yl]amino; [5-(benzyloxycarbonyl)-2-oxo-2,3,4,5-tetrahydro-1H-benzo[b][1,4]diazepin-3-yl]amino; { 2-[1-(2-pyridin-4-yl-ethyl)-piperidin-4-yl]-ethyl}amino; { 2-[1 -(N,N-dimethylaminocarbonyl)-piperidin-4-yl]ethyl}amino; { 2-[1 -(pyridin-4-ylcarbonyl)-piperidin-4-yl]ethyl}amino; 2-(3-methoxy-4-hydroxy-phenyl)ethylamino; 2-(N-(4-1H-benzimidazol-2-yl)piperin-4-yl)ethylamino; 2-(N-(4-benzimidazol-2-yl)piperin-4-yl)ethylamino; 2-(N-methyl-N-pyridin-4-yl)ethylamino; 2-[1,4′]bipiperidinyl-2-cyano-ethylamino; 2-[1,4′]bipiperidinylethylamino; 2-[2-phenyl-1H-benzo[d]imidazole]-ethylamino; 2-[4-(pyridin-4-yl)piperidin-1-yl]ethylamino; 2-[N-((pyridin-4-yl)acetyl)piperidin-4-yl]ethylamino; 2-[N-(2,2,2-trichloroethoxyacetyl) piperidin-4-yl]ethylamino; 5-(t-butoxycarbonyl)aminopentylamino; 5-aminopentylamino; N-((pyridin-4-yl)acetyl)piperidin-4-ylamino; and piperidin-4-ylamino.

3. A compound according to claim 1 wherein R₁ is selected from 1-(2-Aminoethyl)piperidine; 1-(2-Pyridinyl)-4-piperidinamine; 1-(2-Pyridinyl)-4-piperidinethanamine; 1-(4-Chlorophenyl)ethylamine; 1-(4-Fluorophenyl)ethylamine; 1-(4-Methoxyphenyl)ethylamine; 1-(4-Methyl)-4-piperidinepropan-2-amine; 1-(4-Pyridinyl)-4-piperidinamine; 1-(4-pyridyl)-4-piperidineethanamine; 1,5-Dimethyl-1H-pyrazole-3-methanamine; 1-Amino-2-indanol; 1-Aminopiperidine; 1-Benzyl-3-aminopyrrolidine; 1-Dimethylamino-2-propylamine; 1-Methyl-1H-pyrrole-2-methanamine; 1-Methyl-3-piperidinamine; 1-Methyl-4-piperidineethanamine; 1-Methylpiperazine; 1-phenyl-4-(2-aminoethyl)piperidine; 1 -Phenylpiperazine; alpha-methyl-1-Piperidineethanamine ; 2-(2-aminoethyl)-1-methylpyrrolidine; 2-(4-Benzylpiperazin-1 -yl)ethylamine; 2-(4-Methylpiperazin-1-yl)ethylamine; 2-(Aminomethyl)-1-ethylpyrrolidine; 2-(Aminomethyl)-5-methylpyrazine; 2-Amino-4-phenyl-1-piperidin-1-ylbutane; 2-Benzyloxycyclopentylamine; 2-Methylcyclohexylamine; 2-phenylglycinol; 2-Picolylamine; 3-(1H-Pyrrol-1-yl)-benzenemethanamine; 3-amino-1,3,4,5-tetrahydro-2H-1-benzazepin-2-one; 3-Amino-1,3-dihydro-1-methyl-5-phenyl-2H-1,4-benzodiazepin-2-one; 3-Amino-1,3-dihydro-5-phenyl-2H-1,4-benzodiazepin-2-one; 3-Amino-1,3-dihydro-5-cyclohexyl-2H-1,4-benzodiazepin-2-one; 3-Amino-1-ethylhexahydro-2H-azepin-2-one; 3-Amino-1-methyl-2-piperidinone; 3-Amino-2-oxo-1,2,3,4-tetrahydroquinoline; 3-Amino-3-methyl-2-piperidone; 3-Amino-7-chloro-1,3-dihydro-5-phenyl-2H-1,4-benzodiazepin-2-one; 3-Amino-7-chloro-5-(2-chlorophenyl)- i ,3-dihydro-2H-1,4-benzodiazepin-2-one; 3-Aminohexahydro-1-(phenylmethyl)-2H-azepin-2-one; 3-Aminomethylbenzothiophene; 3-aminoquinuclidine; 3-Dimethylamino-1-propylamine; 3-Morpholinopropylamine; 3-Picolylamine; 4-(1-Aminoethyl)phenol; 4-(2-Aminoethyl)morpholine; 4-(2-Aminoethyl)pyridine; 4-Amino-1-benzylpiperidine; 4-Amino-2-butanol; 4-Picolylamine; 1-methyl-4-Piperidinamine; 5-Methyl-3-Isoxazolemethanamine; Alaninol; alpha-N,N-Dimethylbenzylamine; alpha-Amine-epsilon-N-methyl-caprolactam; alpha-Aminodiphenylmethane; alpha-Amino-epsilon-caprolactam; alpha-methyl-4-Morpholineethanamine; alpha-Methylbenzylamine; Azepan-3-ylamine; benzylamine; beta-methyl-1-pyrrolidineethanamine; Cumylamine; cyclohexylamine; endo-8-Methyl-8-azabicyclo[3.2.1 ]octan-3 -amine; Ethanolamine; Hexahydro-1-methyl-i H-azepin-3-amine; histamine; Isopropylamine; methylamine; morpholine; N-(2-aminoethyl)-2-Benzyl-N-methylaniline; N-(2-Aminoethyl)acetamide; N-(2-Aminoethyl)pyrrolidine; N,N,N′-Trimethylethylenediamine; N,N-Dimethylethylenediamine; N,O-Dimethylhydroxylamine; N-alpha-dimethylbenzylamine; phenethylamine; trans-2-Aminocyclohexanol; trans-4-Aminocyclohexanol; Tryptamine; Tyramine; Valinol; N,N-diethyl-1,2-propanediamine; N-ethyl-N-methyl-1,2-propanediamine; 1 -phenylsulfonyl-4-piperidineamine; alpha-phenyl-1-piperidineethanamine; N,N-dimethyl-1,2-butanediamine; 3,4-dihydro-1 -(2H)-quinolineethanamine; 1-Amino-2-propanol;beta-alaninamide; beta-alanine t-butyl ester; alpha-methyl-4-(methylsulfonyl)-benzenemethanamine; 1-[2-pyrrolidinylmethyl]-pyrrolidine; alpha-methylbenzylamine; alpha methyl-1-pyrrolidineethanamine; N,N-dimethyl-4-phenyl-1,2-butanediamine; N-acetyl-N-methyl-1,2-propanediamine; N-methyl-N-phenyl-1,2-ethanediamine; N-cyclopropyl-N-methyl-1,2-propanediamine; (4-Phenyl-morpholin-2-yl)-methylamine; 1-(1-Naphthyl)ethylamine; 1,2,3,4-Tetrahydro-1-naphthylamine; 1-Aminoethylphosphonic acid; 1-Cyclohexylethylamine; 1-Ethynylcyclohexylamine; 1-Methoxy-3-phenyl-2-propylamine; 2-(Aminomethyl)benzimidazole; 2-(Diisobutylamino)ethylamine; 2-(Diisopropylamino)ethylamine; 2,2,2-Trifluoroethylamine;2,2-Diphenylethylamine; 2,6-Bis(dimethylamino)benzylamine; 2-[2-(Aminomethyl)phenylthio]benzyl alcohol; 2-amino-1,2-diphenylethanol; 2-Amino-4′-bromoacetophenone; 2-Aminoacetophenone; 2-(Aminoethyl)-2-thiopseudourea; 2-Aziridinoethylamine; 2-Methoxyisopropylamine; 2-Methylallylamine; 3,3-Diphenylpropylamine; 3,4-Methylenedioxyamphetamine; 3-Aminocyclohexanecarboxylic acid; 3-Aminopyrrolidine; 3-Nitrophenacylamine; 4-(2-aminoethyl)-1-methylpiperidine; 4-(2-Aminoethyl)benzenesulfonamide; 4-Amino-1-diethylaminopentane; 7-Amino-5-methyl-5H,7H-dibenzo[b,d]azepin-6-one; Agmatine; alpha-1-Amino-2-propanol; alpha-Ethylbenzylamine; Aminoacetamidine; Aminoacetonitrile; beta-Methylphenethylamine; Cathinone; Cyclobutylamine; Cyclohexanemethylamine; Cyclopropylamine; Cycloserine; Homocysteine thiolactone; Menthylamine; Methioninol; Muscimol; N-(3′-Aminopropyl)-2-pyrrolidinone; N-(3-Aminopropyl)diethanolamine; N,N-Dimethyl-1,4-diaminobutane; N-Benzylethylenediamine; N-Ethyl-N-Butylethylenediamine; Norephedrine; O-Benzylhydroxylamine; Phenylisopropylamine; p-Methoxyamphetamine; and Tetrahydrofurfurylamine.

4. The method according to claim 1 wherein the at least one condition which benefits from inhibition of the bradykinin B1 receptor is selected from asthma, inflammatory bowel disease, rhinitis, pancreatitis, cystitis, uveitis, inflammatory skin disorders, rheumatoid arthritis and edema resulting from trauma associated with burns, sprains or fracture, osteoarthritis, rheumatoid arthritis, rheumatic disease, tenosynovitis, gout, pain associated with angina, menstruation, or cancer, diabetic vasculopathy, post capillary resistance or diabetic symptoms associated with insulitis, spasm of the gastrointestinal tract or uterus, Crohn's disease, ulcerative colitis or pancreatitis, liver disease, multiple sclerosis, atherosclerosis, Alzheimer's disease, septic shock, cerebral edema, headache, migraine, closed head trauma, irritable bowel syndrome and nephritis.

5. The compound according to claim 36 of formula (I), 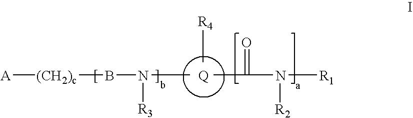
or a pharmaceutically acceptable salt thereof, wherein
a is 1;
b is 1;
c is 0, 1 or 2;
Q is selected from structures Q(a), Q(b), and Q(c), 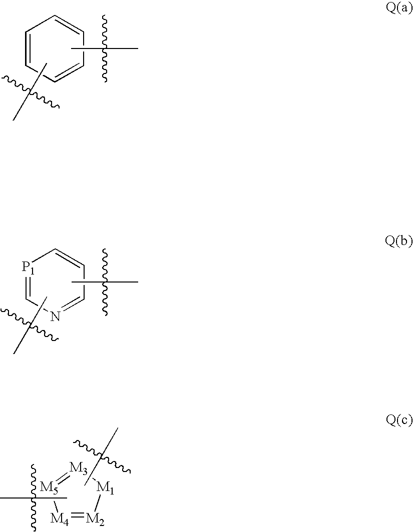
wherein the two 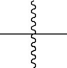
in structures Q(a), Q(b), and Q(c) are not attached to adjacent atoms; wherein structures Q(a), Q(b), and Q(c) are optionally substituted with at least one R₄ group
independently selected from alkyl, halogen, —CF₃, and —OH;
wherein Q(c) is not pyrazol;
M₁ is selected from —NH—, —O—, and —S—;
M₂, M₃, M₄, and M₅ are each independently selected from —C—, —CH—, and —N—;
P₁ is selected from —CH— and —N—;
R₁ is selected from
-alkyl,
-cycloalkyl,
-heterocycloalkyl,
-alkoxy,
-aryl, and
-heteroaryl;
wherein the alkyl, cycloalkyl, heterocycloalkyl, alkoxy, aryl, and heteroaryl within R₁ are each optionally substituted with at least one group independently selected from R₂₀₀;
Ra and Rb are independently selected from
-hydrogen (wherein Ra and Rb are not simultaneously hydrogen),
-alkyl,
-alkoxy,
-cycloalkyl, and
-heterocycloalkyl;
wherein the alkyl, alkoxy, cycloalkyl, and heterocycloalkyl within R^{a} and R^{b} are each optionally substituted with at least one group independently selected from R₂₀₀;
or R^{a} and R^{b} together with the nitrogen atom to which they are attached form a heteroaryl (optionally substituted with at least one group independently selected from R₂₀₀) or heterocycloalkyl (optionally substituted with at least one group independently selected from R₂₀₀);
R₂ is selected from
—H,
-alkyl,
-cycloalkyl,
-heterocycloalkyl,
-aryl, and
-heteroaryl;
wherein the alkyl, cycloalkyl, heterocycloalkyl, aryl, and heteroaryl within R₂ are each optionally substituted with at least one group independently selected from R₂₀₀;
or R₁ and R₂ together with the nitrogen to which they are attached form a heterocycloalkyl (optionally substituted with R₂₀₀) or a heteroaryl (optionally substituted with R₂₀₀);
R₃ is selected from hydrogen and alkyl;
B is selected from —C(O)— and —S(O)₂—; and
A is selected from structure A(a), 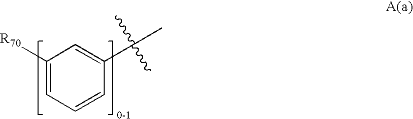
R₇₀ is 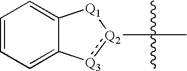
Q₁ is selected from —C(R₆₀)₂—, —O—, —S—, —N(R₆₀)—, and —C(O)—;
Q₂ is selected from —C—, —CH— and —N—; and
Q₃ is selected from —C(R₆₀)₁₋₂—, and —N(R₆₀)₀₋₁—;
wherein structure A(a) is optionally substituted with at least one group independently selected from halogen and alkyl;
wherein the dashed line in R₇₀ is optionally a double bond;
R₆₀ at each occurrence is independently selected from hydrogen, halogen, hydroxy, C₁-C₅ alkyl, cycloalkyl, heterocycloalkyl, heteroaryl, cycloalkoxy, and haloalkyl, or two R₆₀ groups together with the atom to which they are attached form a cycloalkyl or heterocycloalkyl ring;
R₂₀₀ at each occurrence is independently selected from
-alkyl optionally substituted with at least one group independently selected from R₂₀₅,
—OH,
—NH₂,
-halogen,
—CN,
-(C₁-C₄ alkyl)₀₋₁-C(O)—NR₂₁₀R₂₁₅,
-(C₁-C₄ alkyl)₀₋₁-(C(O))₀₋₁-R₂₀₅,
-(C₁-C₄ alkyl)₀₋₁-(C(O))₀₋₁-cycloalkyl,
-(C₁-C₄ alkyl)₀₋₁-(C(O))₀₋₁-heterocycloalkyl,
-(C₁-C₄ alkyl)₀₋₁-(C(O))₀₋₁-heterocycloalkyl-heteroaryl,
-(C₁-C₄ alkyl)₀₋₁-(C(O))₀₋₁-aryl,
-(C₁-C₄ alkyl)₀₋₁-(C(O))₀₋₁-heteroaryl,
-(C₁-C₄ alkyl)₀₋₁-N(H or R₂₀₅)—C(O)—R₂₁₀,
-(C₁-C₄ alkyl)₀₋₁-NR₂₁₀R₂₁₅,
-(C₁-C₄ alkyl)₀₋₁-O—(R₂₀₅),
-(C₁-C₄ alkyl)₀₋₁-S—(R₂₀₅), and
-(C₁-C₄ alkyl)₀₋₁-O-(alkyl optionally substituted with at least one halogen);
wherein each aryl or heteroaryl group included within R₂₀₀ is optionally substituted with at least one group independently selected from R₂₀₅ and alkyl (optionally substituted with at least one group independently selected from R₂₀₅);
wherein each cycloalkyl or heterocycloalkyl group included within R₂₀₀ is optionally substituted with at least one group independently selected from R₂₀₅ and alkyl (optionally substituted with at least one group independently selected from R₂₀₅);
R₂₀₅ at each occurrence is independently selected from
-alkyl,
-heteroaryl,
-heterocycloalkyl,
-aryl,
—(CH₂)₀₋₃-cycloalkyl,
-halogen,
—(C₁-C₆ alkyl)₀₋₁-CN,
—OH,
—O-alkyl, and
—NR₂₁₀R₂₁₅,
R₂₁₀ and R₂₁₅ at each occurrence are independently selected from
—H,
-alkyl,
-aminoalkyl,
—(C₁-C₄ alkyl)₀₋₁-C(O)—NH₂,
—(C₁-C₄ alkyl)₀₋₁-C(O)—NH(alkyl) (wherein alkyl is optionally substituted with at least one group independently selected from R₂₀₅),
-(C₁-C₄ alkyl)₀₋₁-C(O)—N(alkyl)(alkyl),
—(CH₂)₀₋₂-cycloalkyl,
-alkyl-O-alkyl,
—O-alkyl,
-aryl,
-heteroaryl, and
-heterocycloalkyl; or
R₂₁₀ and R₂₁₅ and the nitrogen to which they are attached form a heterocycloalkyl optionally substituted with at least one R₂₀₅ group;
wherein the aryl, heteroaryl and heterocycloalkyl groups included within R₂₁₀ and R₂₁₅ are each optionally substituted with at least one group independently selected from R₂₀₅.

6. The compound according to claim 5 wherein c is 0.

7. The compound according to claim 5 wherein R₇₀ is selected from structures R₇₀(a), R₇₀(b), R₇₀(c), and R₇₀(d), 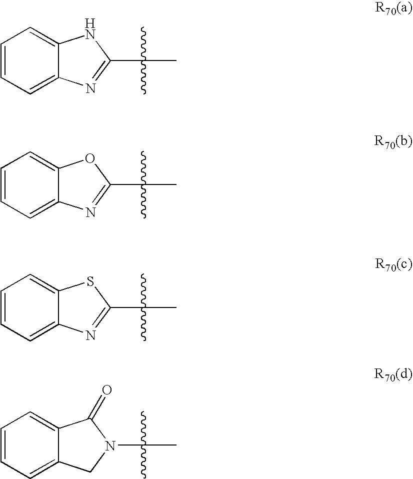
optionally substituted with halogen.

8. The compound according to claim 5 wherein R₁ is selected from 4-Pyridin-4-yl-piperazin-1-ylmethyl, 2-(3,4,5,6-Tetrahydro-2H-[1,4′]bipyridinyl-4-yl)-ethyl, 1-(4-Pyridin-4-yl-piperazin-1-yl)-ethyl, 3,4,5,6-Tetrahydro-2H-[1,4′]bipyridinyl-4-ylmethyl, 3,4,5,6-Tetrahydro-2H-[1,4′]bipyridinyl-4-ylamino, 2-Piperidin-4-ylidenemethyl, 4-Pyridin-4-yl-piperazin-1-yl, 3,4,5,6-Tetrahydro-2H-[1,4′]bipyridinyl-4-yl, 4-(4-Pyridin-4-yl-piperazin-1-yl)-phenyl, 2-[1-(1H-Imidazol-2-yl)-piperidin-4-yl]-ethyl, 2-(4-Pyridin-4-yl-piperazin-1-yl)-ethyl, 2-[1-(1H-Benzoimidazol-2-yl)-piperidin-4-yl]-ethyl, 3-(3,4,5,6-Tetrahydro-2H-[1,4′]bipyridinyl-4-yl)-propyl, 2-(3′-Methyl-3,4,5,6-tetrahydro-2H-[1,2′]bipyridinyl-4-yl)-ethyl, 2-[4-(4-Methylpiperazin-1-yl)-phenyl]-ethyl, 2-(4-Pyridin-4-yl-phenyl)-ethyl, 4-(3-Amino-propyl)-phenyl, 2-(1-Methyl-piperidin-4-yl)-ethyl, 2-(4-Acetylamino-phenyl)-ethyl, Azepan-3-yl, 2-(4-Aminophenyl)-ethyl, 2-(2′-Cyano-3,4,5,6-tetrahydro-2H-[1,4′]bipyridinyl-4-yl)-ethyl, 3-(5,6,7,8-Tetrahydro-[1,8]naphthyridin-2-yl)-propyl, and 2-Oxo-5-phenyl-2,3-dihydro-1H-benzo[e]+81,4]diazepin-3-yl.

9. The compound according to claim 5 wherein R₁ and R₂ together with the nitrogen to which they are attached form a ring structure selected from 9-Pyridin-4-yl-3,9-diaza-spiro[5.5]undec-3-yl, 9-Methyl-3 ,9-diaza-spiro[5 .5]undec-3-yl, 9-Isopropyl-3,9-diaza-spiro[5.5]undec-3-yl, 9-tert-Butoxycarbonyl-3,9-diaza-spiro[5.5]undec-3-yl, 4-Pyridin-4-yl-piperazin-1-yl, (3′,4′,5′,6′,3″,4″,5″,6″-Octahydro-2′H,2″H-[4,1′;4′,4″]terpyridinyl), (3′,4′,5′,6′,3″,4″,5″,6″-Octahydro-2′H,2″H-[2,1′;4′,4″9 terpyridinyl), 1 ′-Isopropyl-[4,4′]bipiperidinyl, 1′-Methyl-[4,4′]bipiperidinyl, [4,4′]Bipiperidinyl, 4-Amino-[1,4′]bipiperidinyl, 4-(2-Imidazol-1-yl-ethyl)-piperaz-1-yl, 4-(1-Methyl-piperidin-4-ylmethyl)-piperaz-1-yl, 4-(3-Pyrrolidin-1-yl-propyl)-piperaz-1-yl, 4-phenethyl-piperaz-1-yl, 4-Cyclohexylmethyl-piperaz-1--yl, 4-Cyclohexyl-piperaz-1-yl, 4-(2-Dimethylamino-ethyl)-piperaz-1-yl, 4-(pyridin-2-ylcarbamoylmethyl)-piperaz-1-yl, 4-Benzyl-piperaz-1-yl, 4-(pyrrolidine-1-carbonyl)-piperaz-1-yl, 4-pyridin-2-yl-piperaz-1-yl, 4-Isopropyl-piperaz-1-yl, 4-phenyl-piperaz-1-yl, 4-pyrimidin-2-yl-piperaz-1-yl, and 4-(2-pyrrol-1-yl-ethyl)-piperaz-1-yl.

10. The compound according to claim 36 of formula (I), 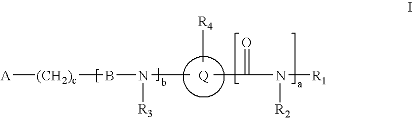
or a pharmaceutically acceptable salt thereof, wherein
a is 1;
b is 0;
c is 0, 1 or 2;
Q is selected from structures Q(a), Q(b), and Q(c), 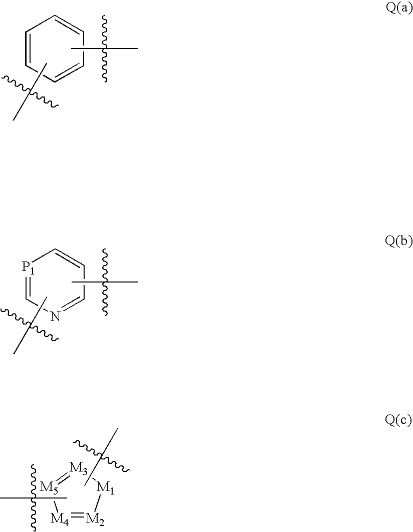
wherein the two 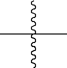
in structures Q(a), Q(b), and Q(c) are not attached to adjacent atoms;
wherein structures Q(a), Q(b), and Q(c) are optionally substituted with at least one R₄ group independently selected from alkyl, halogen, CF₃, and —OH; with the proviso that R₄ is not OH when R₁ is phenylC₀₋₃alkyl
M₁ is selected from —NH—, —O—, and —S—; and
M₂, M₃, M₄, and M₅ are each independently selected from —C—, —CH—, and —N—;
P₁ is selected from —CH— and —N—;
R₁ is selected from
-alkyl,
-cycloalkyl,
-heterocycloalkyl,
-alkoxy,
-aryl, and
-heteroaryl;
wherein the alkyl, cycloalkyl, heterocycloalkyl, alkoxy, aryl, and heteroaryl within R₁ are each optionally substituted with at least one group independently selected from R₂₀₀;
R^{a} and R^{b} are independently selected from
-hydrogen (wherein R^{a} and R^{b} are not simultaneously hydrogen),
-alkyl,
-alkoxy,
-cycloalkyl, and
-heterocycloalkyl,
wherein the alkyl, alkoxy, cycloalkyl, and heterocycloalkyl within R^{a} and R^{b} are each optionally substituted with at least one group independently selected from R₂₀₀;
or R^{a} and R^{b} together with the nitrogen atom to which they are attached form a heteroaryl (optionally substituted with at least one group independently selected from R₂₀₀) or heterocycloalkyl (optionally substituted with at least one group independently selected from R₂₀₀);
R₂ is selected from
—H,
-alkyl,
-cycloalkyl,
-heterocycloalkyl,
-aryl, and
-heteroaryl;
wherein the alkyl, cycloalkyl, heterocycloalkyl, aryl, and heteroaryl within R₂ are each optionally substituted with at least one group independently selected from R₂₀₀;
or R₁ and R₂ together with the nitrogen to which they are attached form a heterocycloalkyl optionally substituted with at least one group independently selected from R₂₀₀ or a heteroaryl optionally substituted with at least one group independently selected from R₂₀₀;
R₃ is absent
B is selected from —C(O)— and —S(O)₂—; and
A is selected from structure A(a), 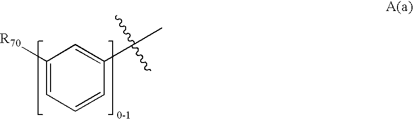
R₇₀ is 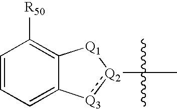
wherein structure A(a) is substituted with at least one R₅₀ group;
wherein the dashed line in R₇₀ is optionally a double bond;
R₅₀ is selected from hydrogen, halogen, and alkyl; and
Q₁ is selected from —CH₂—, —O—, —S—, and —NH—;
Q₂ is selected from —C—, —CH— and —N—; and
Q₃ is selected from —CH—, —CH₂—, —N—, and —NH—; or
Q₁ is selected from —C(O)—;
Q₂ is selected from —C—, —CH— and —N—; and
Q₃ is selected from —C(R₆₀)₁₋₂— and —N(R₆₀)₀₋₁—;
R₆₀ at each occurrence is independently selected from hydrogen, halogen, hydroxy, C₁-C₅ alkyl, cycloalkyl, heterocycloalkyl, heteroaryl, cycloalkoxy, and haloalkyl, or two R₆₀ groups together with the atom to which they are attached forin a cycloalkyl or heterocycloalkyl ring;
wherein when A(a) is 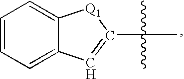
Q₁ is not —NH- or —N(R₅₀)—;
wherein when Q is Q(a), A is A(a), Q₁ is —C(O)—, Q₂ is -C(H)—, and Q₃ is —CH₂—, then R₅₀ is selected from halogen and alkyl, or Q₃ is substituted with alkyl, cycloalkyl, heterocycloalkyl, or heteroaryl, each optionally substituted with at least one group independently selected from R₂₀₀;
R₂₀₀ at each occurrence is independently selected from
-alkyl optionally substituted with at least one group independently selected from R₂₀₅,
—OH,
—NH₂,
-halogen,
—CN,
-(C₁-C₄ alkyl)₀₋₁-C(O)—NR₂₁₀R₂₁₅,
-(C₁-C₄ alkyl)₀₋₁-(C(O))₀₋₁—R₂₀₅,
-(C₁-C₄ alkyl)₀₋₁-(C(O))₀₋₁-cycloalkyl,
-(C₁-C₄ alkyl)₀₋₁-(C(O))₀₋₁-heterocycloalkyl,
-(C₁-C₄ alkyl)₀₋₁-(C(O))₀₋₁-heterocycloalkyl-heteroaryl,
-(C₁-C₄ alkyl)₀₋₁-(C(O))₀₋₁-aryl,
-(C₁-C₄ alkyl)₀₋₁-(C(O))₀₋₁-heteroaryl,
-(C₁-C₄ alkyl)₀₋₁-N(H or R₂₀₅)—C(O)—R₂₁₀,
-(C₁-C₄ alkyl)₀₋₁-NR₂₁₀R₂₁₅,
-(C₁-C₄ alkyl)₀₋₁-O—(R₂O₅),
-(C₁-C₄ alkyl)₀₋₁-S—(R₂₀₅), and
-(C₁-C₄ alkyl)₀₋₁-O-(alkyl optionally substituted with at least one halogen);
wherein each aryl or heteroaryl group included within R₂₀₀ is optionally substituted with at least one group independently selected from R₂₀₅ and alkyl (optionally substituted with at least one group independently selected from R₂₀₅);
wherein each cycloalkyl or heterocycloalkyl group included within R₂₀₀ is optionally substituted with at least one group independently selected from R₂₀₅ and alkyl (optionally substituted with at least one group independently selected from R₂₀₅);
R₂₀₅ at each occurrence is independently selected from
-alkyl,
-heteroaryl,
-heterocycloalkyl,
-aryl,
—(CH₂)₀₋₃-cycloalkyl,
-halogen,
-(C₁-C₆ alkyl)₀₋₁-CN,
—OH,
—O-alkyl, and
—NR₂₁₀R₂₁₅,
R₂₁₀ and R₂₁₅ at each occurrence are independently selected from
—H,
-alkyl,
-aminoalkyl,
-(C₁-C₄ alkyl)₀₋₁-C(O)—NH₂,
-(C₁-C₄ alkyl)₀₋₁-C(O)—NH(alkyl) (wherein alkyl is optionally substituted with at least one group independently selected from R₂₀₅),
-(C₁-C₄ alkyl)₀₋₁-C(O)—N(alkyl)(alkyl),
—(CH₂)₀₋₂-cycloalkyl,
-alkyl-O-alkyl,
—O-alkyl,
-aryl,
-heteroaryl, and
-heterocycloalkyl; or
R₂₁₀ and R₂₁₅ and the nitrogen to which they are attached form a heterocycloalkyl optionally substituted with at least one R₂₀₅ group;
wherein the aryl, heteroaryl and heterocycloalkyl groups included within R₂₁₀ and R₂₁₅ are each optionally substituted with at least one group independently selected from R₂₀₅.

11. The compound according to claim 10 wherein c is 0.

12. The compound according to claim 10 wherein R₇₀ is selected from structures R₇₀(a), R₇₀(b), R₇₀(c), and R₇₀(d), 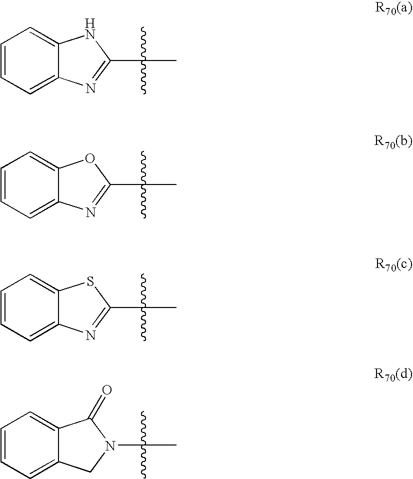
all optionally substituted with halogen.

13. The compound according to claim 10 wherein R₁ is selected from 4-Pyridin-4-yl-piperazin-1-ylmethyl, 2-(3,4,5,6-Tetrahydro-2H-[ 1,4′]bipyridinyl-4-yl)-ethyl, 1-(4-Pyridin-4-yl-piperazin-1-yl)-ethyl, 3,4,5,6-Tetrahydro-2H-[1,4′]bipyridinyl-4-ylmethyl, Piperidin-4-ylidenemethyl, 4-Pyridin-4-yl-piperazin-1-yl, 3,4,5,6-Tetrahydro-2H-[1,4′]bipyridinyl-4-yl, 4-(4-Pyridin-4-yl-piperazin-1-yl)-phenyl, 2-[1-(1H-Imidazol-2-yl)-piperidin-4-yl]-ethyl, 2-(4-Pyridin-4-yl-piperazin-1-yl)-ethyl, 2-[1-(1H-Benzoimidazol-2-yl)-piperidin-4-yl]-ethyl, 3-(3,4,5,6-Tetrahydro-2H-[1,4′]bipyridinyl-4-yl)propyl, 2-(3′-Methyl-3,4,5,6-tetrahydro-2H-[1,2′]bipyridinyl-4-yl)-ethyl, 2-[4-(4-Methylpiperazin-1-yl)-phenyl]-ethyl, 2-(4-Pyridin-4-yl-phenyl)-ethyl, 4-(3-Amino-propyl)-phenyl, 2-(1-Methyl-piperidin-4-yl)-ethyl, 2-(4-Acetylamino-phenyl)-ethyl, Azepan-3-yl, 2-(4-Aminophenyl)-ethyl, 2-(2′-Cyano-3,4,5,6-tetrahydro-2H-[1,4′]bipyridinyl-4-yl)-ethyl, 3-(5,6,7,8-Tetrahydro-[1,8]naphthyridin-2-yl)-propyl, and 2-Oxo-5-phenyl-2,3-dihydro-1H-benzo[e]+81,4]diazepin-3-yl.

14. The compound according to claim 10 wherein R₁ and R₂ together with the nitrogen to which they are attached form a ring structure selected from 9-Pyridin-4-yl-3,9-diaza-spiro[5.5]undec-3-yl, 9-Methyl-3 ,9-diaza-spiro[5 .5]undec-3-yl, 9-Isopropyl-3 ,9-diaza-spiro[5.5]undec-3-yl, 9-tert-Butoxycarbonyl-3,9-diaza-spiro[5.5]undec-3-yl, 4-Pyridin-4-yl-piperazin-1-yl, (3′,4′,5′,6′,3″,4″,5″,6″-Octahydro-2′H,2″H-[4,1′;4′,4″terpyridinyl), (3′,4′,5′,6′,3″,4″,5″,6″-Octahydro-2′H,2″H-[2,1′;4′,4″]terpyridinyl), 1′-Isopropyl-[4,4′]bipiperidinyl, 1′-Methyl-[4,4′]bipiperidinyl, [4,4′]Bipiperidinyl, 4-Amino-[1,4′]bipiperidinyl, 4-(2-Imidazol-1-yl-ethyl)-piperaz-1-yl, 4-(1-Methyl-piperidin-4-ylmethyl)-piperaz-1-yl, 4-(3-Pyrrolidin-1-yl-propyl)-piperaz-1-yl, 4-phenethyl-piperaz-1-yl, 4-Cyclohexylmethyl-piperaz-1-yl, 4-Cyclohexyl-piperaz-1-yl, 4-(2-Dimethylamino-ethyl)-piperaz-1-yl, 4-(pyridin-2-ylcarbamoylmethyl)-piperaz-1-yl, 4-Benzyl-piperaz-1-yl, 4-(pyrrolidine-1-carbonyl)-piperaz-1-yl, 4-pyridin-2-yl-piperaz-1-yl, 4-Isopropyl-piperaz-1-yl, 4-phenyl-piperaz-1-yl, 4-pyrimidin-2-yl-piperaz-1-yl, 4-(2-pyrrol-1-yl-ethyl)-piperaz-1-yl, 4-(pyridin-4-yloxy)-piperidyl, 4-(4-isopropylpiperazin-1-yl)piperidyl, and 4-(1,2,3,4-tetrahydroisoquinolin-5-yloxy)piperidyl..

15. The compound according to claim 36 of formula (I), 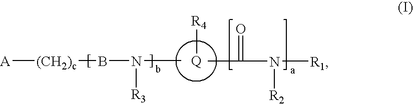
or a pharmaceutically acceptable salt thereof, wherein
a is 0;
b is 0;
c is 0, 1 or 2;
Q is selected from structures Q(b) and Q(c), 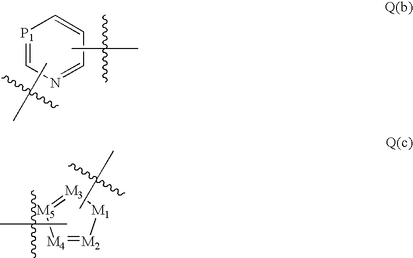
wherein structures Q(b) and Q(c) are optionally substituted with at least one R₄ group independently selected from alkyl, halogen, —CF₃, and —OH;
M₁ is selected from —NH—, —O—, and —S—; and
M₂, M₃, M₄, and M₅ are each independently selected from —C—, —CH—, and —N—;
P₁ is selected from —CH— and —N—;
R₁ is selected from
—NR^{a}R^{b},
-alkyl,
-cycloalkyl,
-heterocycloalkyl,
-alkoxy,
-aryl, and
-heteroaryl;
wherein the alkyl, cycloalkyl, heterocycloalkyl, alkoxy, aryl, and heteroaryl within R₁ are each optionally substituted with at least one group independently selected from R₂₀₀;
R^{a} and R^{b} are independently selected from
-hydrogen (wherein R^{a} and R^{b} are not simultaneously hydrogen),
-alkyl,
-alkoxy,
-cycloalkyl, and
-heterocycloalkyl;
wherein the alkyl, alkoxy, cycloalkyl, and heterocycloalkyl within R^{a} and R^{b} are each optionally substituted with at least one group independently selected from R₂₀₀;
or R^{a} and R^{b} together with the nitrogen atom to which they are attached form a heteroaryl (optionally substituted with at least one group independently selected from R₂₀₀) or heterocycloalkyl (optionally substituted with at least one group independently selected from R₂₀₀);
R₂ is absent;
R₃ is absent;
B is selected from —C(O)— and —S(0)₂—; and
A is selected from structure A(b), 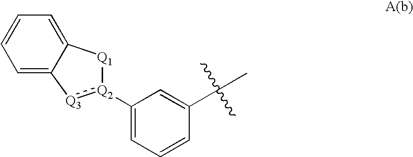
Q₁ is selected from —CH₂—, —O—, —S—, and —NH—;
Q₂ is selected from —C—, —CH— and —N—; and
Q₃ is selected from —CH—, —CH₂—, —N—, and —NH—; or
Q₁ is selected from —C(O)—;
Q₂ is selected from —C—, —CH— and —N—; and
Q₃ is selected from —C(R₆₀)₁₋₂— and —N(R₆₀)₀₋₁;
R₆₀ at each occurrence is independently selected from hydrogen, halogen, hydroxy, C₁-C₅ alkyl, cycloalkyl, heterocycloalkyl, heteroaryl, cycloalkoxy, and haloalkyl, or two R₆₀ groups together with the atom to which they are attached form a cycloalkyl or heterocycloalkyl ring;
structure A(b) is optionally substituted with at least one group independently selected from halogen and alkyl;
R₂₀₀ at each occurrence is independently selected from
-alkyl optionally substituted with at least one group independently selected from R₂₀₅,
—OH,
—NH₂,
-halogen,
—CN,
-(C₁-C₄ alkyl)₀₋₁—C(O)—NR₂₁₀R₂₁₅,
-(C₁-C₄ alkyl)₀₋₁-(C(O))₀₋₁-R₂₀₅,
-(C₁-C₄ alkyl)₀₋₁-(C(O))₀₋₁-cycloalkyl,
-(C₁-C₄ alkyl)₀₋₁-(C(O))₀₋₁-heterocycloalkyl,
-(C₁-C₄ alkyl)₀₋₁-(C(O))₀₋₁-heterocycloalkyl-heteroaryl,
-(C₁-C₄ alkyl)₀₋₁-(C(O))₀₋₁-aryl,
-(C₁-C₄ alkyl)₀₋₁-(C(O))₀₋₁-heteroaryl,
-(C₁-C₄ alkyl)₀₋₁-N(H or R₂₀₅)—C(O)—R₂₁₀,
-(C₁-C₄ alkyl)₀₋₁-NR₂₁₀R₂₁₅,
-(C₁-C₄ alkyl)₀₋₁-O—(R₂₀₅),
-(C₁-C₄ alkyl)₀₋₁-S—(R₂₀₅), and
-(C₁-C₄ alkyl)₀₋₁-O-(alkyl optionally substituted with at least one halogen);
wherein each aryl or heteroaryl group included within R₂₀₀ is optionally substituted with at least one group independently selected from R₂₀₅ and alkyl (optionally substituted with at least one group independently selected from R₂₀₅);
wherein each cycloalkyl or heterocycloalkyl group included within R₂₀₀ is optionally substituted with at least one group independently selected from R₂₀₅ and alkyl (optionally substituted with at least one group independently selected from R₂₀₅);
R₂₀₅ at each occurrence is independently selected from
-alkyl,
-heteroaryl,
-heterocycloalkyl,
-aryl,
—(CH₂)₀₋₃-cycloalkyl,
-halogen,
-(C₁-C₆ alkyl)₀₋₁-CN,
—OH,
-13 O-alkyl, and
—NR₂₁₀R₂₁₅,
R₂₁₀ and R₂₁₅ at each occurrence are independently selected from
—H,
-alkyl,
-aminoalkyl,
-(C₁-C₄ alkyl)₀₋₁-C(O)—NH₂,
-(C₁-C₄ alkyl)₀₋₁-C(O)—NH(alkyl) (wherein alkyl is optionally substituted with at least one group independently selected from R₂₀₅),
-(C₁-C₄ alkyl)₀₋₁-C(O)—N(alkyl)(alkyl),
—(CH₂)0-2-cycloalkyl,
-alkyl-O-alkyl,
-13 O-alkyl,
-aryl,
-heteroaryl, and
-heterocycloalkyl; or
R₂₁₀ and R₂₁₅ and the nitrogen to which they are attached form a heterocycloalkyl optionally substituted with at least one R₂₀₅ group;
wherein the aryl, heteroaryl and heterocycloalkyl groups included within R₂₁₀ and R₂₁₅ are each optionally substituted with at least one group independently selected from R₂₀₅.

16. The compound according to claim 15 wherein c is 0.

17. The compound according to claim 15 wherein R₇₀ is selected from structures R₇₀(a), R₇₀(b), R₇₀(c), and R₇₀(d), 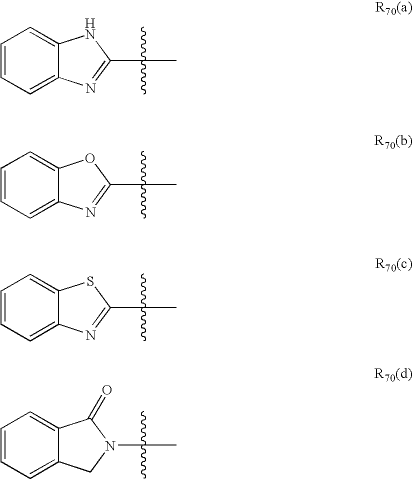
optionally substituted with halogen.

18. The compound according to claim 15 wherein R₁ is selected from 4-Pyridin-4-yl-piperazin-1-ylmethyl, 2-(3,4,5,6-Tetrahydro-2H-[ 1,4′]bipyridinyl-4-yl)-ethyl, 1-(4-Pyridin-4-yl-piperazin-1-yl)-ethyl, 3,4,5,6-Tetrahydro-2H-[1,4′]bipyridinyl-4-ylmethyl, 3,4,5,6-Tetrahydro-2H-[1,4′]bipyridinyl-4-ylamino, Piperidin-4-ylidenemethyl, 4-Pyridin-4-yl-piperazin-1-yl, 3,4,5,6-Tetrahydro-2H-[1,4′]bipyridinyl-4-yl, 2-(4-Pyridin-4-yl-piperazin-1-yl)-ethyl, 4-(4-Pyridin-4-yl-piperazin-1-yl)-phenyl, 2-piperidin-4-ylvinyl, 2-piperidin-4-yl-ethyl, piperidin-4-ylmethoxy, 1-(tert-butoxycarbonyl)piperidin-4-yloxy, piperidin-4-yloxy, 3,4,5,6-tetrahydro-2H-[1,4′]bipyridinyl-4-ylmethoxy, 3,4,5,6-tetrahydro-2H-[1,4′]bipyridinyl-4-yloxy, 2,3,5,6-tetrahydro-[1,4′]bipyridinyl-4-ylidenemethyl, 2-piperidin-4-ylethoxy, 2-(3,4,5,6-tetrahydro-2H-[1,4′]bipyridinyl-4-yl)ethoxy, and (2S,6R)-dimethyl-4-pyridin-4-ylpiperazin-i-ylmethyl.

19. The compound according to claim 36 of formula (I), 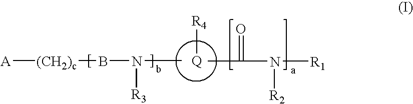
or a pharmaceutically acceptable salt thereof, wherein
a is 0;
b is 1;
c is 0, 1 or 2;
Q is selected from structures Q(b) and Q(c), 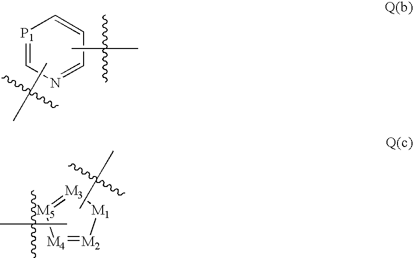
wherein the two 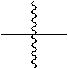
in structures Q(b) and Q(c) are not attached to adjacent atoms;
wherein structures Q(b) and Q(c) are optionally substituted with at least one R₄ group independently selected from alkyl, halogen, —CF₃, and —OH;
M₁ is selected from —NH—, —O—, and —S—;
M₂, M₃, M₄, and M₅ are each independently selected from —C—, —CH—, and —N—;
P₁ is selected from —CH— and —N—;
R₁ is selected from
—NR^{a}R^{b},
-alkyl,
-cycloalkyl,
-heterocycloalkyl,
-alkoxy,
-aryl, and
-heteroaryl;
wherein the alkyl, cycloalkyl, heterocycloalkyl, alkoxy, aryl, and heteroaryl within R₁ are each optionally substituted with at least one group independently selected from R₂₀₀;
R^{a} and R^{b} are independently selected from
-hydrogen (wherein R^{a} and R^{b} are not simultaneously hydrogen),
-alkyl,
-alkoxy,
-cycloalkyl, and
-heterocycloalkyl;
wherein the alkyl, alkoxy, cycloalkyl, and heterocycloalkyl within R^{a} and R^{b} are each optionally substituted with at least one group independently selected from R₂₀₀;
or R^{a} and R^{b} together with the nitrogen atom to which they are attached form a heteroaryl (optionally substituted with at least one group independently selected from R₂₀₀) or heterocycloalkyl (optionally substituted with at least one group independently selected from R₂₀₀);
R₂ is absent
R₃ is selected from hydrogen and alkyl;
B is selected from —C(O)— and —S(0)₂—; and
A is selected from structure A(b), 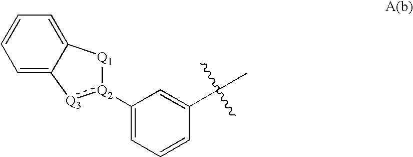
wherein structure A(b) is optionally substituted with at least one group independently selected from halogen and alkyl,
Q₁ is selected from —C(R₆₀)₂—, —O—, —S—, —N(R₆₀)—, and —C(O)—;
Q₂ is selected from —C—, —CH—, and —N—; and
Q₃ is selected from —C(R₆₀)₁₋₂— and —N(R₆₀)₀₋₁₋₂—;
R₆₀ at each occurrence is independently selected from hydrogen, halogen, hydroxy, CI-C₅ alkyl, cycloalkyl, heterocycloalkyl, heteroaryl, cycloalkoxy, and haloalkyl, or two R₆₀ groups together with the atom to which they are attached form a cycloalkyl or heterocycloalkyl ring;
R₂₀₀ at each occurrence is independently selected from
-alkyl optionally substituted with at least one group independently selected from R₂₀₅,
—OH,
—NH₂,
-halogen,
—CN,
-(C₁-C₄ alkyl)₀₋₁-C(O)—NR₂₁₀R₂₁₅,
-(C₁-C₄ alkyl)₀₋₁-(C(O))₀₋₁-R₂₀₅,
-(C₁-C₄ alkyl)₀₋₁-(C(O))₀₋₁-cycloalkyl,
-(C₁-C₄ alkyl)₀₋₁-(C(O))₀₋₁-heterocycloalkyl,
-(C₁-C₄ alkyl)₀₋₁-(C(O))₀₋₁-heterocycloalkyl-heteroaryl,
-(C₁-C₄ alkyl)₀₋₁-(C(O))₀₋₁-aryl,
-(C₁-C₄ alkyl)₀₋₁-(C(O))₀₋₁-heteroaryl,
-(C₁-C₄ alkyl)₀₋₁-N(H or R₂₀₅)—C(O)—R₂₁₀,
-(C₁-C₄ alkyl)₀₋₁-NR₂₁₀R₂₁₅,
-(C₁-C₄ alkyl)₀₋₁-O—(R₂₀₅),
-(C₁-C₄ alkyl)₀₋₁-S—(R₂₀₅), and
-(C₁-C₄ alkyl)₀₋₁-O-(alkyl optionally substituted with at least one halogen);
wherein each aryl or heteroaryl group included within R₂₀₀ is optionally substituted with at least one group independently selected from R₂₀₅ and alkyl (optionally substituted with at least one group independently selected from R₂₀₅);
wherein each cycloalkyl or heterocycloalkyl group included within R₂₀₀ is optionally substituted with at least one group independently selected from R₂₀₅ and alkyl (optionally substituted with at least one group independently selected from R₂₀₅);
R₂₀₅ at each occurrence is independently selected from
-alkyl,
-heteroaryl,
-heterocycloalkyl,
-aryl,
—(CH₂)₀₋₃-cycloalkyl,
-halogen,
-(C₁-C₆ alkyl)₀₋₁-CN,
—OH,
—O-alkyl, and
—NR₂₁₀R₂₁₅,
R₂₁₀ and R₂₁₅ at each occurrence are independently selected from
—H,
-alkyl,
-aminoalkyl,
-(C₁-C₄ alkyl)₀₋₁-C(O)—NH₂,
-(C₁-C₄ alkyl)₀₋₁-C(O)—NH(alkyl) (wherein alkyl is optionally substituted with at least one group independently selected from R₂₀₅),
-(C₁-C₄ alkyl)₀₋₁-C(O)—N(alkyl)(alkyl),
—(CH₂)₀₋₂-cycloalkyl,
-alkyl-O-alkyl,
—O-alkyl,
-aryl,
-heteroaryl, and
-heterocycloalkyl; or
R₂₁₀ and R₂₁₅ and the nitrogen to which they are attached form a heterocycloalkyl optionally substituted with at least one R₂₀₅ group;
wherein the aryl, heteroaryl and heterocycloalkyl groups included within R₂₁₀ and R₂₁₅ are each optionally substituted with at least one group independently selected from R₂₀₅.

20. The compound according to claim 19 wherein c is 0.

21. The compound according to claim 19 wherein R₇₀ selected from structures R₇₀(a), R₇₀(b), R₇₀(c), and R₇₀(d), 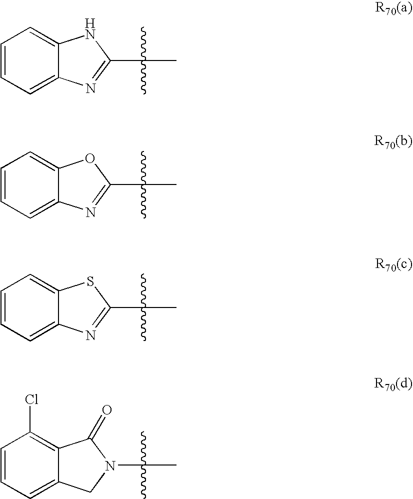
optionally substituted with halogen.

22. The compound according to claim 19 wherein R₁ is selected from 4-Pyridin-4-yl-piperazin-1-ylmethyl, 2-(3,4,5,6-Tetrahydro-2H-[1,4′]bipyridinyl-4-yl)-ethyl, 1-(4-Pyridin-4-yl-piperazin-1-yl)-ethyl, 3,4,5,6-Tetrahydro-2H-[1,4′]bipyridinyl-4-ylmethyl, 3,4,5,6-Tetrahydro-2H-[1,4′]bipyridinyl-4-ylamino, Piperidin-4-ylidenemethyl, 4-Pyridin-4-yl-piperazin-1-yl, 3,4,5,6-Tetrahydro-2H-[ 1,4′]bipyridinyl-4-yl, 2-(4-Pyridin-4-yl-piperazin-1-yl)-ethyl, 4-(4-Pyridin-4-yl-piperazin-1-yl)-phenyl, 2-piperidin-4-ylvinyl, 2-piperidin-4-yl-ethyl, piperidin-4-ylmethoxy, 1-(tert-butoxycarbonyl)piperidin-4-yloxy, piperidin-4-yloxy, 3,4,5,6-tetrahydro-2H-[1,4′]bipyridinyl-4-ylmethoxy, 3,4,5,6-tetrahydro-2H-[1,4′]bipyridinyl-4-yloxy, 2,3,5,6-tetrahydro-[1,4′]bipyridinyl-4-ylidenemethyl, 2-piperidin-4-ylethoxy, 2-(3,4,5,6-tetrahydro-2H-[1,4′]bipyridinyl-4-yl)ethoxy, and (2S,6R)-dimethyl-4-pyridin-4-ylpiperazin-1-ylmethyl.

23. The compound according to claim 36 of formula (I), 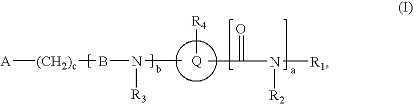
or a pharmaceutically acceptable salt thereof, wherein
a is 0;
b is 0;
c is 0, 1, or 2;
Q is structure Q(a), 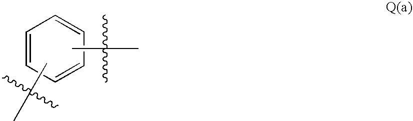
wherein the two 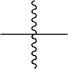
in structure Q(a) are not attached to adjacent atoms;
R₁ is selected from
—NR^{a}R^{b},
-alkyl,
-cycloalkyl,
-heterocycloalkyl,
-alkoxy,
-aryl, and
-heteroaryl;
wherein the alkyl, cycloalkyl, heterocycloalkyl, alkoxy, aryl, and heteroaryl within R₁ are each optionally substituted with at least one group independently selected from R₂₀₀;
R^{a} and R^{b} are independently selected from
-hydrogen (wherein R^{a} and R^{b} are not simultaneously hydrogen),
-alkyl,
-alkoxy,
-cycloalkyl, and
-heterocycloalkyl;
wherein the alkyl, alkoxy, cycloalkyl, and heterocycloalkyl within R^{a} and R^{b} are each optionally substituted with at least one group independently selected from R₂₀₀;
or R^{a} and R^{b} together with the nitrogen atom to which they are attached form a heteroaryl (optionally substituted with at least one group independently selected from R₂₀₀) or heterocycloalkyl (optionally substituted with at least one group independently selected from R₂₀₀);
R₂ is absent:
R₃ is absent;
wherein when A is A(b), R₄ is selected from hydrogen, OH, alkyl, aryl, halogen, alkoxy, nitro, CN, cycloalkyl, amino, monoalkylamino, dialkylamino, amino carbonyl, monoalkylamino carbonyl, and dialkylaminocarbonyl;
wherein when A is A(d), R₄ is selected from hydrogen, OH, alkyl, aryl, alkoxy, nitro, CN, cycloalkyl, amino carbonyl, monoalkylamino carbonyl, and dialkylaminocarbonyl;
B is selected from —C(O)— and —S(O)₂—; and
A is selected from structures A(b) and A(d), 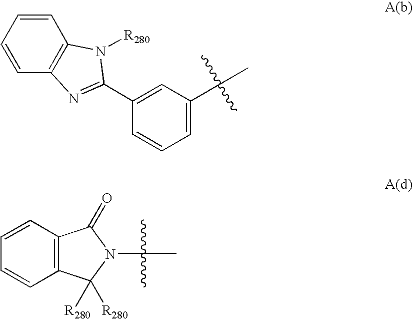
wherein structures A(b) and A(d) are optionally substituted with at least one group independently selected from halogen and alkyl;
R₂₈₀ at each occurrence is independently selected from hydrogen, halogen, hydroxy, C₁-C₅ alkyl, cycloalkyl, heterocycloalkyl, heteroaryl, cycloalkoxy, and haloalkyl, or two R₂₈₀ groups together with the atom to which they are attached form a cycloalkyl or heterocycloalkyl ring;
R₂₀₀ at each occurrence is independently selected from
-alkyl optionally substituted with at least one group independently selected from R₂₀₅,
—OH,
—NH₂,
-halogen,
—CN,
-(C₁-C₄ alkyl)₀₋₁-C(O)—NR₂₁oR₂₁₅,
-(C₁-C₄ alkyl)₀₋₁-(C(O))₀₋₁-R₂₁₅,
-(C₁-C₄ alkyl)₀₋₁-(C(O))₀₋₁-cycloalkyl,
-(C₁-C₄ alkyl)₀₋₁-(C(O))₀₋₁-heterocycloalkyl,
-(C₁-C₄ alkyl)₀₋₁-(C(O))₀₋₁-heterocycloalkyl-heteroaryl,
-(C₁-C₄ alkyl)-(C(O))₀₋₁-aryl,
-(C₁-C₄ alkyl)₀₋₁-(C(O))₀₋₁-heteroaryl,
-(C₁-C₄ alkyl)₀₋₁-N(H or R₂₀₅)—C(O)—R₂₁₀,
-(C₁-C₄ alkyl)₀₋₁-NR₂₁₀R₂₁₅,
-(C₁-C₄ alkyl)₀₋₁-O—(R₂₀₅),
-(C₁-C₄ alkyl)₀₋₁-S—(R₂₀₅), and
-(C₁-C₄ alkyl)₀₋₁-O-(alkyl optionally substituted with at least one halogen);
wherein each aryl or heteroaryl group included within R₂₀₀ is optionally substituted with at least one group independently selected from R₂₀₅ and alkyl (optionally substituted with at least one group independently selected from R₂₀₅);
wherein each cycloalkyl or heterocycloalkyl group included within R₂₀₀ is optionally substituted with at least one group independently selected from R₂₀₅ and alkyl (optionally substituted with at least one group independently selected from R₂₀₅);
R₂₀₅ at each occurrence is independently selected from
-alkyl,
-heteroaryl,
-heterocycloalkyl,
-aryl,
—(CH₂)₀₋₃-cycloalkyl,
-halogen,
-(C₁-C₆ alkyl)₀₋₁-CN,
—OH,
—O-alkyl, and
—NR₂₁₀R₂₁₅,
R₂₁₀ and R₂₁₅ at each occurrence are independently selected from
—H,
-alkyl,
-aminoalkyl,
-(C₁-C₄ alkyl)₀₋₁-C(O)—NH₂,
-(C₁-C₄ alkyl)₀₋₁-C(O)—NH(alkyl) (wherein alkyl is optionally substituted with at least one group independently selected from R₂₀₅),
-(C₁-C₄ alkyl)₀₋₁-C(O)—N(alkyl)(alkyl),
-(CH₂)₀₋₂-cycloalkyl,
-alkyl-O-alkyl,
—O-alkyl,
-aryl,
-heteroaryl, and
-heterocycloalkyl; or
R₂₁₀ and R₂₁₅ and the nitrogen to which they are attached form a heterocycloalkyl optionally substituted with at least one R₂₀₅ group;
wherein the aryl, heteroaryl and heterocycloalkyl groups included within R₂₁₀ and R₂,₅ are each optionally substituted with at least one group independently selected from R₂₀₅.

24. The compound according to claim 23 wherein c is 0.

25. The compound according to claim 36 of formula (I), 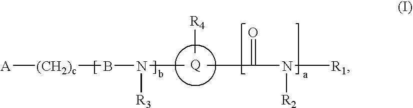
or a pharmaceutically acceptable salt thereof, wherein
a is 0;
b is 1;
c is 0, 1, or 2;
Q is structure Q(a), 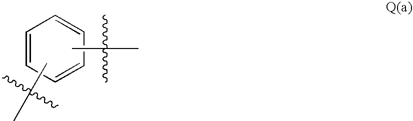
wherein the two 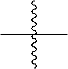
in structure Q(a) are not attached to adjacent atoms;
R₁ is selected from
—NR^{a}R^{b},
-alkyl,
-cycloalkyl,
-heterocycloalkyl,
-alkoxy,
-aryl, and
-heteroaryl;
wherein the alkyl, cycloalkyl, heterocycloalkyl, alkoxy, aryl, and heteroaryl within R₁ are each optionally substituted with at least one group independently selected from R₂₀₀;
R^{a} and R^{b} are independently selected from
-hydrogen (wherein R^{a} and R^{b} are not simultaneously hydrogen),
-alkyl,
-alkoxy,
-cycloalkyl, and
-heterocycloalkyl;
wherein the alkyl, alkoxy, cycloalkyl, and heterocycloalkyl within R^{a} and R^{b} are each optionally substituted with at least one group independently selected from R₂₀₀;
or R^{a} and R^{b} together with the nitrogen atom to which they are attached form a heteroaryl (optionally substituted with at least one group independently selected from R₂₀₀) or heterocycloalkyl (optionally substituted with at least one group independently selected from R₂₀₀);
R₂ is absent;
R₃ is selected from hydrogen and alkyl;
wherein when A is A(b), R₄ is selected from hydrogen, OH, alkyl, aryl, halogen, alkoxy, nitro, CN, cycloalkyl, amino, monoalkylamino, dialkylamino, amino carbonyl, monoalkylamino carbonyl, and dialkylaminocarbonyl;
wherein when A is A(d), R₄ is selected from hydrogen, OH, alkyl, aryl, alkoxy, nitro, CN, cycloalkyl, amino carbonyl, monoalkylamino carbonyl, and dialkylaminocarbonyl;
B is selected from —C(O)— and —S(O)₂—; and
A is selected from structures A(b) and A(d), 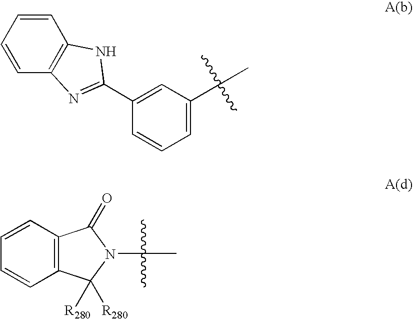
wherein structures A(b) and A(d) are optionally substituted with at least one group independently selected from halogen and alkyl;
R₂₈₀ at each occurrence is independently selected from hydrogen, halogen, hydroxy, C₁-C₅ alkyl, cycloalkyl, heterocycloalkyl, heteroaryl, cycloalkoxy, and haloalkyl, or two R₂₈₀ groups together with the atom to which they are attached form a cycloalkyl or heterocycloalkyl ring;
R₂₀₀ at each occurrence is independently selected from
-alkyl optionally substituted with at least one group independently selected from R₂₀₅,
—OH,
—NH₂,
-halogen,
—CN,
-(C₁-C₄ alkyl)₀₋₁-C(O)—NR₂₁OR₂₁₅,
-(C₁-C₄ alkyl)₀₋₁-(C(O))₀₋₁-R₂₀₅,
-(C₁-C₄ alkyl)₀₋₁-(C(O))₀₋₁-cycloalkyl,
-(C₁-C₄ alkyl)₀₋₁-(C(O))₀₋₁-heterocycloalkyl,
-(C₁-C₄ alkyl)₀₋₁-(C(O))₀₋₁-heterocycloalkyl-heteroaryl,
-(C₁-C₄ alkyl)₀₋₁-(C(O))₀₋₁-aryl,
-(C₁-C₄ alkyl)₀₋₁-(C(O))₀₋₁-heteroaryl,
-(C₁-C₄ alkyl)₀₋₁-N(H or R₂₀₅)—C(O)—R₂₁₀,
-(C₁-C₄ alkyl)₀₋₁-NR₂₁₀R₂₁₅,
-(C₁-C₄ alkyl)₀₋₁-O—(R₂₀₅),
-(C₁-C₄ alkyl)₀₋₁-S—(R₂₀₅), and
-(C₁-C₄ alkyl)₀₋₁-O-(alkyl optionally substituted with at least one halogen);
wherein each aryl or heteroaryl group included within R₂₀₀ is optionally substituted with at least one group independently selected from R₂₀₅ and alkyl (optionally substituted with at least one group independently selected from R₂₀₅);
wherein each cycloalkyl or heterocycloalkyl group included within R₂₀₀ is optionally substituted with at least one group independently selected from R₂₀₅ and alkyl (optionally substituted with at least one group independently selected from R₂₀₅);
R₂₀₅ at each occurrence is independently selected from
-alkyl,
-heteroaryl,
-heterocycloalkyl,
-aryl,
—(CH₂)₀₋₃-cycloalkyl,
-halogen,
-(C₁-C₆ alkyl)₀₋₁-CN,
—OH,
—O-alkyl, and
—NR₂₁₀R₂₁₅,
R₂₁₀ and R₂₁₅ at each occurrence are independently selected from
—H,
-alkyl,
-aminoalkyl,
-(C₁-C₄ alkyl)₀₋₁-C(O)—NH₂,
-(C₁-C₄ alkyl)₀₋₁-C(O)—NH(alkyl) (wherein alkyl is optionally substituted with at least one group independently selected from R₂₀₅),
-(C₁-C₄ alkyl)₀₋₁-C(O)—N(alkyl)(alkyl),
—(CH₂)₀₋₂-cycloalkyl,
-alkyl-O-alkyl,
—O-alkyl,
-aryl,
-heteroaryl, and
-heterocycloalkyl; or
R₂₁₀ and R₂₁₅ and the nitrogen to which they are attached form a heterocycloalkyl optionally substituted with at least one R₂₀₅ group;
wherein the aryl, heteroaryl and heterocycloalkyl groups included within R₂₁₀ and R₂,₁₅ are each optionally substituted with at least one group independently selected from R₂₀₅.

26. The compound according to claim 25 wherein c is 0.

27. The compound according to claim 25 wherein R₁ is selected from 4-Pyridin-4-yl-piperazin-1-ylmethyl, 2-(3,4,5,6-Tetrahydro-2H-[ 1,4′]bipyridinyl-4-yl)-ethyl, 1-(4-Pyridin-4-yl-piperazin-1-yl)-ethyl, 3,4,5,6-Tetrahydro-2H-[1,4′]bipyridinyl-4-ylmethyl, 3,4,5,6-Tetrahydro-2H-[1,4′]bipyridinyl-4-ylamino, Piperidin-4-ylidenemethyl, 4-Pyridin-4-yl-piperazin-1-yl, 3,4,5,6-Tetrahydro-2H-[1,4′]bipyridinyl-4-yl, 2-(4-Pyridin-4-yl-piperazin-1-yl)-ethyl 4-(4-Pyridin-4-yl-piperazin-1-yl)-phenyl, 2-piperidin-4-ylvinyl, 2-piperidin-4-yl-ethyl, piperidin-4-ylmethoxy, 1-(tert-butoxycarbonyl)piperidin-4-yloxy, piperidin-4-yloxy, 3,4,5,6-tetrahydro-2H-[1,4′]bipyridinyl-4-ylmethoxy, 3,4,5,6-tetrahydro-2H-[1,4′]bipyridinyl-4-yloxy, 2,3,5,6-tetrahydro-[1,4′]bipyridinyl-4-ylidenemethyl, 2-piperidin-4-ylethoxy, 2-(3,4,5,6-tetrahydro-2H-[1,4′]bipyridinyl-4-yl)ethoxy, and (2S,6R)-dimethyl-4-pyridin-4-ylpiperazin-1-ylmethyl.

28. A compound of formula (II), 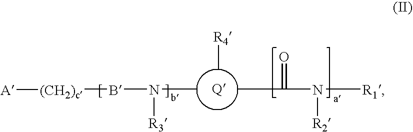
or a pharmaceutically acceptable salt thereof, wherein
a′ is 1;
b′ is 1;
c′ is 0, 1, or 2;
Q′ is selected from structure Q′(a), Q′(b), and Q′(c), 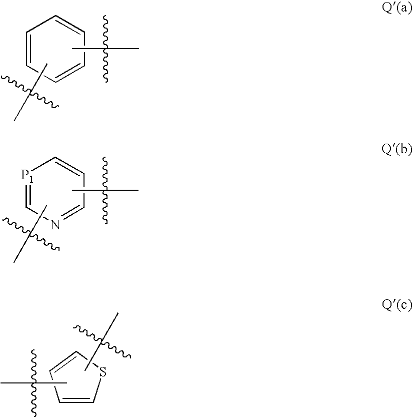
P₁ is selected from —CH— and —N—;
R₁′ is selected from R₁(a), R₁(b), R₁(c), R₁(d), R₁(e), R₁(f), R₁(g), and R₁(h), 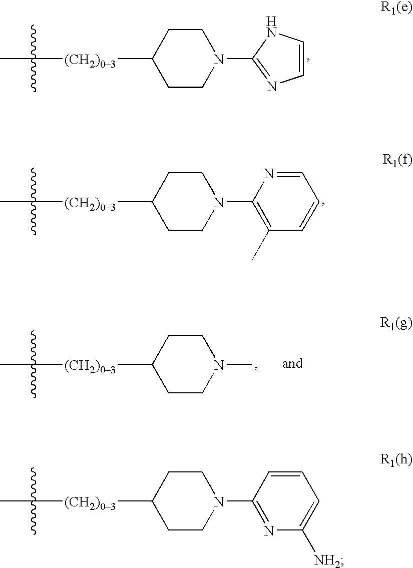
R₂′ is hydrogen;
or R₁′ and R₂′ together with the nitrogen to which they are attached form 9-pyridin-4-yl-3,9-diaza-spiro[5.5]undec-3-yl;
R₃′ is selected from hydrogen and alkyl;
R₄′ is selected from hydrogen and halogen;
B′ is selected from —C(O)— and —S(O)₂—; and
A′ is structure A(e), 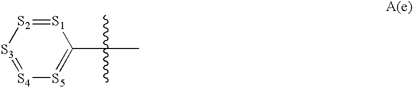
S₁ and S₄ are each independently selected from —CH—, —C(R₅₅′)—, and —N—;
S₂, S₃, and S₅ are each independently selected from —CH— and —C(R₅₅′)—;
R₅₅′ at each occurrence is independently selected from halogen ,alkyl, and —CF₃.

29. The compound according to claim 28, wherein the formula (II) compound is selected from 3-(2-chlorobenzoylamino)-2-chloro-N-[2-(3,4,5,6-tetrahydro-2H-[1,4′]bipyridinyl-4-yl)-ethyl]-benzamide, 3-(3-chlorobenzoylamino)—N-[2-(3,4,5,6-tetrahydro-2H-[1,4′]bipyridinyl-4-yl)-ethyl]-benzamide, 3-(3-chlorobenzoylamino)-2-chloro-N-[2-(3,4,5,6-tetrahydro-2H-[1,4′]bipyridinyl-4-yl)-ethyl]-benzamide, 3-[(4-chloro-2,5-dimethyl-benzenesulfonyl)-methylamino]—N-[2-(3,4,5,6-tetrahydro-2H-[1,4′]bipyridinyl-4-yl)-ethyl]-benzamide, 3-[(2-chlorobenzoyl)methylamino]—N-[2-(3,4,5,6-tetrahydro-2H-[1,4′]bipyridinyl-4-yl)-ethyl]-benzamide, 3-(2-chlorobenzoylamino)—N-[2-(3,4,5,6-tetrahydro-2H-[1,4′-bipyridinyl-4-yl)-ethyl1-benzamide, 3-(2,3-dichlorobenzoylamino)—N-[2-(3,4,5,6-tetrahydro-2H-[1,4′]bipyridinyl-4-yl)-ethyl]-benzamide, 3-(2,6-dichlorobenzoylamino)—N-[2-(3,4,5,6-tetrahydro-2H-[1,4′]bipyridinyl-4-yl)-ethyl]-benzamide, 3-(2-chlorobenzoylamino)-4-chloro-N-[2-(3,4,5,6-tetrahydro-2H-[1,4′]bipyridinyl-4-yl)-ethyl]-benzamide, 4-chloro-3-(4-chloro-2,5-dimethyl-benzenesulfonylamino)-N-[2-(3,4,5,6-tetrahydro-2H-[1,4′]bipyridinyl-4-yl)-ethyl]-benzamide, 3-(2-trifluoromethylbenzoylamino)—N-[2-(3,4,5,6-tetrahydro-2H-[1,4′]bipyridinyl-4-yl)-ethyl]-benzamide, 4-chloro-3-[(4-chloro-2,5-dimethyl-benzenesulfonyl)-methyl-amino]—N-[2-(3,4,5,6-tetrahydro-2H-[1,4′]bipyridinyl-4-yl)-ethyl]-benzamide, 3-(2-chlorobenzoylamino)-4-fluoro-N-[2-(3,4,5,6-tetrahydro-2H-[ 1,4′]bipyridinyl-4-yl)-ethyl]-benzamide, 4-chloro-3-[(4-chloro-2,5-dimethyl-benzenesulfonyl)-methyl-amino]-N-(3,4,5,6-tetrahydro-2H-[1,4′]bipyridinyl-4-ylmethyl)-benzamide, N-{2-[1-(1H-benzoimidazol-2-yl)-piperidin-4-yl]-ethyl}-4-chloro-3-[(4-chloro-2,5-dimethyl-benzenesulfonyl)-methyl-amino]-benzamide, 4-chloro-3-[(2,3-dichlorobenzenesulfonyl)-methyl-amino]-N-[2-(3,4,5,6-tetrahydro-2H-[1,4′]bipyridinyl-4-yl)-ethyl]-benzamide, 3-(2-chlorobenzoylamino)-4-bromo—N-[2-(3,4,5,6-tetrahydro-2H-[1,4′]bipyridinyl-4-yl)-ethyl]-benzamide, 4-chloro-3-[(4-chloro-2,5-dimethyl-benzenesulfonyl)-methyl-amino]-N-{2-[1-(1H-imidazol-2-yl)-piperidin-4-yl]-ethyl}-benzamide, 4-chloro-3-[(3,4-dichlorobenzenesulfonyl)-methyl-amino]-N-[2-(3,4,5,6-tetrahydro-2H-[1,4′]bipyridinyl-4-yl)-ethyl]-benzamide, 4-bromo-3-[(4-chloro-2,5-dimethyl-benzenesulfonyl)-methyl-amino]—N-[2-(3,4,5,6-tetrahydro-2H-[1,4′]bipyridinyl-4-yl)-ethyl]-benzarnide, 4-chloro-3-[(2,6-dichlorobenzenesulfonyl)-methyl-amino]-N-[2-(3,4,5,6-tetrahydro-2H-[1 ′]bipyridinyl-4-yl)-ethyl]-benzamide, 6-(2-chloro-benzoylamino)—N-[2-(3,4,5,6-tetrahydro-2H-[1,4′]bipyridinyl-4-yl)ethyl]-nicotinamide, 2,4-dichloro-5-(4-chloro-2,5-dimethyl-benzenesulfonylamino)—N-[2-(3,4,5,6-tetrahydro-2H-[1,4′]bipyridinyl-4-yl)-ethyl]-benzamide, 2,4-dichloro-5-[(4-chloro-2,5-dimethyl-benzenesulfonyl)-methyl-amino]-N-[2-(3,4,5,6-tetrahydro-2H-[1,4′]bipyridinyl-4-yl)ethyl]-benzamide, 5-(2-chlorobenzoylarnino)-2,4-dichloro-N-[2-(3,4,5,6-tetrahydro-2H-[1,4′]bipyridinyl-4-yl)-ethyl]-benzamide, 3-chloro-pyrazine-2-carboxylic acid {2-chloro-5-[2-(3,4,5,6-tetrahydro-2H-[1,4′]bipyridinyl-4-yl)-ethylcarbamoyl]-phenyl}-amide, 6-(2-chlorobenzoylamino)pyridine-2-carboxylic acid [2-(3,4,5,6-tetrahydro-2H-[1,4′]bipyridinyl-4-yl)ethyl]-arnide, 4-(2-chlorobenzoylamino)pyridine-2-carboxylic acid [2-(3,4,5,6-tetrahydro-2H-[1,4′]bipyridinyl-4-yl)-ethyl]-amide, 2-(2-chlorobenzoylamino)—N-[2-(3,4,5,6-tetrahydro-2H-[1,4′]bipyridinyl-4-yl)-ethyl]-isonicotinamide, 5-(2-chlorobenzoylamino)—N-[2-(3,4,5,6-tetrahydro-2H-[1,4′]bipyridinyl-4-yl)-ethyl]-nicotinamide, 3-(2-chlorobenzoylamino)-4-chloro-N-[2-(3′-methyl-3,4,5,6-tetrahydro-2H-[1,2′]bipyridinyl-4-yl)-ethyl]-benzamide, 3-(2-chlorobenzoylamino)-4-chloro-N-[2-(1-methyl-piperidin-4-yl)-ethyl]-benzamide, 3-(2-chlorobenzoylamino)-4-chloro-N-[2-(4-pyridin-4-yl-piperazin-1-yl)-ethyl]-benzamide, 2-chloro-N-[3-(2-3,4,5,6-tetrahydro-2H-[1,4′]bipyridinyl-4-yl-acetylamino)-phenyl]-benzamide, 2-chloro-N-[2-chloro-5-(9-pyridin-4-yl-3,9-diaza-spiro[5.5]undecane-3-carbonyl)-phenyl]-benzarnide, 3-(2-chlorobenzoylamino)—N-[2-(6′-amino-3,4,5,6-tetrahydro-2H-[1,2′]bipyridinyl-4-yl)-ethyl]-4-chloro-benzamide, and 5-(2-chloro-benzoylamino)-thiophene-2-carboxylic acid [2-(3,4,5,6-tetrahydro-2H-[ 1,4′]bipyridinyl-4-yl)-ethyl]-amide.

30. The compound according to claim 28 wherein c′ is 0.

31. A method of preventing or treating at least one condition which benefits from inhibition of the bradykinin B1 receptor, comprising:
administering to a host in need thereof a composition comprising a therapeutically effective amount of at least one compound according to claim 28 of formula (II), or a pharmaceutically acceptable salt thereof, as defined in claim 28.

32. A method for selectively inhibiting bradykinin B ₁ receptor over bradykinin B₂ receptor by administering to a host in need thereof an effective amount of at least one compound according to claim 36 of formula (I), 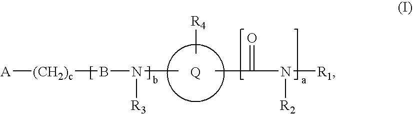
or a pharmaceutically acceptable salt thereof, wherein R₁, R₂, R₃, R₄, A, B, a, b, c and Q are defined as in claim 36.

33. A method for treating or ameliorating adverse symptoms associated with up-regulating bradykinin B₁ receptor relative to burns, perioperative pain, migraine, shock, central nervous system injury, asthma, rhinitis, premature labor, inflammatory arthritis, inflammatory bowel disease, neuropathic pain, or multiple sclerosis comprising, administering a therapeutically effective amount of at least one compound according to claim 36 of formula (I) 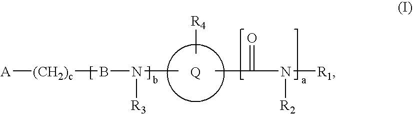
or a pharmaceutically acceptable salt thereof, wherein R₁, R₂, R₃, R_{4,} A, B, a, b, c and Q are defined as in claim 36.

34. A pharmaceutical composition comprising, a pharmaceutically acceptable carrier and a therapeutically effective amount of at least one compound according to claim 36 of formula (I), 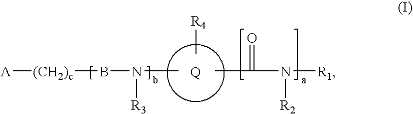
or mixtures thereof, effective to treat or ameliorate adverse symptoms in mammals mediated by bradykinin B₁ receptor, wherein R₁, R₂, R₃, R₄, A, B, a, b, c and Q are defined as in claim 36.

35. amended) An article of manufacture comprising:
(a) at least one dosage form of at least one compound according to claim 36 of formula (I), 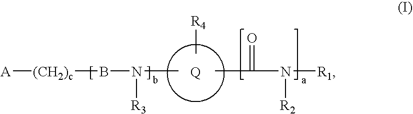
or pharmaceutically acceptable salt thereof, optionally in combination with one or more active and/or inactive pharmaceutical agents, wherein R₁, R₂, R₃, R₄, A, B, a, b, c and Q are defined as in claim 36;
(b) a package insert providing that a dosage form comprising at least one compound of formula (1) should be administered to a patient in need of therapy for disorders, conditions or diseases which benefit from inhibition of the bradykinin B₁ receptor; and
(c) at least one container in which at least one dosage form of at least one compound of formula (I), optionally in combination with one or more active and/or inactive pharmaceutical agents, is stored.

36. A compound of formula I 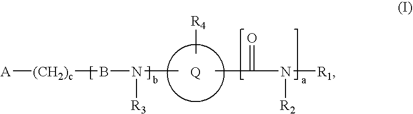
or a pharmaceutically acceptable salt thereof, wherein
a is 0 or 1;
b is 0 or 1;
c is 0, 1 or 2;
Q is an aryl, heteroaryl, cycloalkyl, or heterocycloalkyl ring;
R₁ is selected from
—NR^{a}R^{b},
-alkyl,
-cycloalkyl,
-heterocycloalkyl,
-alkoxy,
-aryl, and
-heteroaryl;
wherein the alkyl, cycloalkyl, heterocycloalkyl, alkoxy, aryl, and heteroaryl within R₁ are each optionally substituted with at least one group independently selected from R₂₀₀;
wherein when R₁ is an N-linked group then a is 0;
R^{a} and R^{b} are independently selected from
-hydrogen, wherein R^{a} and R^{b} are not simultaneously hydrogen,
-alkyl,
-alkoxy,
-cycloalkyl,
-aryl,
-heteroaryl, and
-heterocycloalkyl;
wherein the alkyl, alkoxy, cycloalkyl, aryl, heteroaryl and heterocycloalkyl within R^{a} and R are each optionally substituted with at least one group independently selected from R₂₀₀;
or R^{a} and R^{b} together with the nitrogen atom to which they are attached form a heteroaryl (optionally substituted with at least one group independently selected from R₂₀₀) or heterocycloalkyl (optionally substituted with at least one group independently selected from R₂₀₀);
R₂ is selected from hydrogen, alkyl, cycloalkyl, heterocycloalkyl, aryl, and heteroaryl;
wherein the alkyl, cycloalkyl, heterocycloalkyl, aryl, and heteroaryl within R₂ are each optionally substituted with at least one group independently selected from R₂₀₀;
or R₁ and R₂ together with the nitrogen to which they are attached form a heterocycloalkyl (optionally substituted with at least one group independently selected from R₂₀₀) or a heteroaryl (optionally substituted with at least one group independently selected from R₂₀₀);
R₃ is selected from hydrogen and alkyl;
R₄ is selected from hydrogen, OH, alkyl, aryl, halogen, alkoxy, nitro, CN, cycloalkyl, amino, monoalkylamino, dialkylamino, amino carbonyl, monoalkylamino carbonyl, and dialkylaminocarbonyl, with the proviso that R₄ is not OH when a=1, b=0 and R₁ is phenylC₀₋₃alkyl;
B is selected from —C(O)— and —S(O)₂—;
A is selected from
aryl substituted with formula A(a), wherein the aryl is optionally substituted with at least one group selected from R₅₀, and
formula A(a), 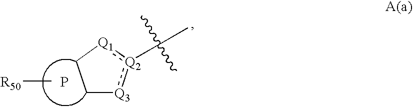
Q₁ and Q₃ are each independently selected from —C(R₆₀)₁₋₂—, —C(O)—, —O—, —N(R₆₀)₀₋₁—, and —S—;
Q₂ is selected from —CH—, —C— and —N—;
P is an aromatic or heteroaromatic ring;
wherein a dashed line in A(a) is optionally a double bond;
R₅₀ is selected from hydrogen, halogen, cyano, alkyl, alkylcycloalkyl, cycloalkyl, cycloalkoxy, alkoxy, alkylthio, hydroxy, amino, monoalkylamino, dialkylamino, heterocycloalkyl, nitro, haloalkyl, —CF₃, haloalkoxy, aryl, —COOR₅₁, and —C(O)R₅₂;
R₅₁ is selected from hydrogen and alkyl;
R₅₂ is selected from alkyl, amino, monoalkylamino, dialkylamino, and heterocycloalkyl; and
R₆₀ at each occurrence is independently selected from hydrogen, halogen, hydroxy, C₁-C₅ alkyl, cycloalkyl, heterocycloalkyl, heteroaryl, cycloalkoxy, and haloalkyl, or two R₆₀ groups together with the atom to which they are attached form a cycloalkyl or heterocycloalkyl ring;
R₂₀₀ at each occurrence is independently selected from
-alkyl optionally substituted with at least one group independently selected from R₂₀₅,
—OH,
—NH₂,
-halogen, —CN,
-(C₁-C₄ alkyl)₀₋₁-C(O)—NR₂₁₀R₂₁₅,
-(C₁-C₄ alkyl)₀₋₁-(C(O))₀₋₁-R₂₀₅,
-(C₁-C₄ alkyl)₀₋₁-(C(O))₀₋₁-cycloalkyl,
-(C₁-C₄ alkyl)₀₋₁-(C(O))₀₋₁-heterocycloalkyl,
-(C₁-C₄ alkyl)₀₋₁-(C(O))₀₋₁-heterocycloalkyl-heteroaryl,
-(C₁-C₄ alkyl)₀₋₁-(C(O))₀₋₁-aryl,
-(C₁-C₄ alkyl)₀₋₁-(C(O))₀₋₁-heteroaryl,
-(C₁-C₄ alkyl)₀₋₁-N(H or R₂₀₅)—C(O)—R₂₁₀,
-(C₁-C₄ alkyl)₀₋₁-NR₂₁₀R₂₁₅,
-(C₁-C₄ alkyl)₀₋₁-O—(R₂₀₅),
-(C₁-C₄ alkyl)₀₋₁-S—(R₂₀₅), and
-(C₁-C₄ alkyl)₀₋₁-O-(alkyl optionally substituted with at least one halogen);
wherein each aryl or heteroaryl group included within R₂₀₀ is optionally substituted with at least one group independently selected from R₂₀₅ and alkyl (optionally substituted with at least one group independently selected from R₂₀₅);
wherein each cycloalkyl or heterocycloalkyl group included within R₂₀₀ is optionally substituted with at least one group independently selected from R₂₀₅ and alkyl (optionally substituted with at least one group independently selected from R₂₀₅);
R₂₀₅ at each occurrence is independently selected from
-alkyl,
-heteroaryl,
-heterocycloalkyl,
-aryl,
-(CH₂)₀₋₃-cycloalkyl,
-halogen,
-(C₁-C₆ alkyl)₀₋₁-CN,
—OH,
—O-alkyl, and
—NR₂₁₀R₂₁₅,
R₂₁₀ and R₂₁₅ at each occurrence are independently selected from
—H,
-alkyl,
-aminoalkyl,
-(C₁-C₄ alkyl)₀₋₁-C(O)—NH₂,
-(C₁-C₄ alkyl)₀₋₁-C(O)—NH(alkyl) (wherein alkyl is optionally substituted with at least one group independently selected from R₂₀₅),
-(C₁-C₄ alkyl)₀₋₁-C(O)—N(alkyl)(alkyl),
—(CH₂)₀₋₂-cycloalkyl,
-alkyl-O-alkyl,
—O-alkyl,
-aryl,
-heteroaryl, and
-heterocycloalkyl; or
R₂₁₀ and R₂₁₅ and the nitrogen to which they are attached form a heterocycloalkyl optionally substituted with at least one R₂₀₅ group;
wherein the aryl, heteroaryl and heterocycloalkyl groups included within R₂₁₀ and R₂₁₅ are each optionally substituted with at least one group independently selected from R₂₀₅;
provided that
when a=1 and b=1, then Q is not pyrazol; and
when a=1 and b=1, then R₁ is not alkyl substituted with biphenyl; or
alkyl substituted with 5-phenylpyridin-2-yl; and
when a=1 and b=0, then A or A(a) is not indol.

37. A compound according to claim 36 wherein Q is selected from the structures: 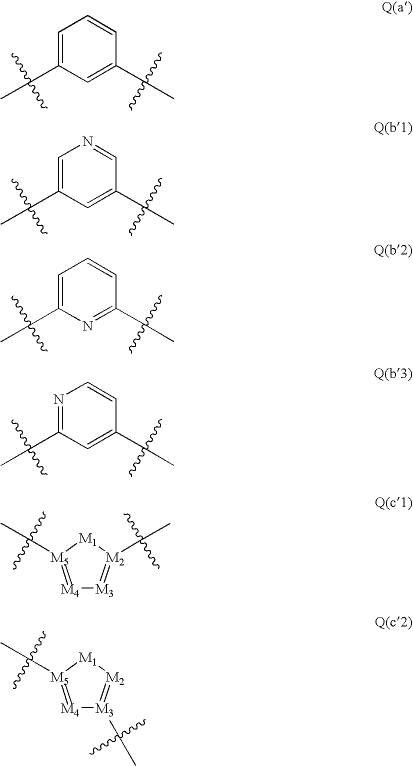
wherein structures Q(a′), Q(b′1), Q(b′2), Q(b′3), Q(c′1) and Q(c′2) are optionally substituted with at least one R₄ group independently selected from alkyl, halogen, —CF₃ and OH;
wherein R₄ is not OH when Q(a′) is present, a=1, b=0 and R₁ is phenylC₀₋₃alkyl;
M₁ is selected from —NH—, —O—, and —S—; and
M₂, M₃, M₄, and M₅ are each independently selected from —C—, —CH—, and —N—.

38. A compound according to claim 10 wherein Q is selected from the structures 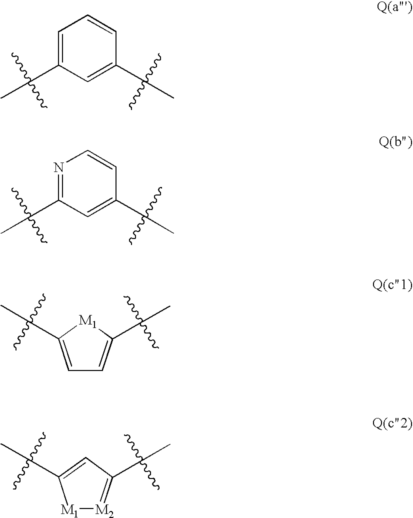
wherein
structures Q(a″), Q(b″), Q(c″1) and Q(c″2) are optionally substituted with at least one R₄ group independently selected from alkyl, halogen, —CF₃ and OH; wherein R₄ is not OH when Q(a″) is present and R₁ is phenylC₀₋₃alkyl;
M₁ is selected from —NH—, —O—, and —S—; and
M₂ is —N—.

39. The compound according to claim 10, wherein R₁ and R₂ together with the nitrogen to which they are attached form a spiro ring structure selected from:
9-methyl-3,9-diaza-spiro[5.5]undecan-3-yl;
9-pyridin-4-yl-3,9-diaza-spiro[5.5]undecane;
9-tert-butoxycarbonyl-3,9-diaza-spiro[5.5]undec-3-yl;
9-isopropyl-3,9-diaza-spiro[5.5]undec-3-yl;
3,9-diaza-spiro[5.5]undec-3-yl; and
9-( 1H-benzirnidazol-2-yl)-3,9-diaza-spiro[5.5]undec-3-yl.

40. The compound according to claim 10, wherein R₁ and R₂ together with the nitrogen to which they are attached form a ring structure selected from:
(3′,4′,5′,6′,3″,4″,5″,6″-octahydro-2′H,2″H-[4,1′;4′,4″]terpyridinyl);
(3′,4′,5′,6′,3″,4″,5″,6″-octahydro-2′H,2″H-[2,1′;4′,4″]terpyridinyl);
4-amino-[1,4′]bipiperidinyl;
[1,4′]bipiperidinyl;
1′-isopropyl-[4,4′]bipiperidinyl;
4-dimethylaminomethyl-[1,4′]bibpiperidnyl; and
1′-methyl-[4,4′]bipiperidinyl.

41. The compound according to claim 10, wherein R₁ and R₂ together with the nitrogen to which they are attached form a piperazine ring structure selected from:
4-pyridin-4-yl-piperazin-1-yl;
4-cyclohexyl-piperazin-1-yl;
4-pyrimidin-2-yl-piperazin-1-yl;
4-phentyl-piperazin-1-yl;
4-(pyrrolidine-1-carbonyl)-piperazin-1-yl;
4-(2-imidazol-1yl-ethyl)-piperazin-1-yl;
4-cyclohexylmethyl-piperazin-1-yl;
4-(2-dimethylamino-ethyl)-piperazin-1-yl;
4-(pyridin-2-yl-carbamoylmethyl)-piperazin-1-yl;
4-isopropyl-piperazin-1-yl;
4-pyridin-2-yl-piperazin-1-yl;
4-pyridin-4-yl-piperazin-1-yl; and
4-(1-methyl-piperidin-4-yl)-piperazin-1-yl.

42. The compound according to claim 10, wherein R₁ and R₂ together with the nitrogen to which they are attached form a piperidine ring structure selected from
4-piperidin-4-yloxymethyl-piperidin-1-yl
4-pyridin-4-ylmethoxy)-piperidin-1-yl
4-pyridin-4-yloxy)-piperidin-1-yl;
4-phenoxy-piperidin-1-yl; and
(1-isopropyl-piperidin-4-yloxy)-piperidin-1-yl.

43. The compound according to claim 10, wherein R₂ is hydrogen and R₁ is selected from:
2-(3,4,5,6-tetrahydro-2H-[1,4′]bipyridinyl-4-yl)-ethyl;
2-(2′ -cyano-3,4,5,6-Tetrahydro-2H-[1,4′]bipyridinyl-4-yl)-ethyl;
3,4,5,6-tetrahydro-2H-[1,4′]bipyridinyl-4-ylmethyl;
4-pyridin-4-yl-piperazin-1-ylmethyl; and
3,4,5,6-tetrahydro-2H-[1,4′]bipyridinyl-4-yl.

44. The compound according to claim 10 selected from:
3-(7-Chloro-1-oxo-1,3-dihydro-isoindol-2-yl)—N-[2-(3,4,5,6-tetrahydro-2H-[1,4′]bipyridinyl-4-yl)ethyl]benzamide;
3-(4-Chloro-benzothiazol-2-yl)-N-[2-(3,4,5,6-tetrahydro-2H-[1,4′]bipyridinyl-4-yl)ethyl]benzamide;
3-(1H-Benzoimidazol-2-yl)—N-[2-(3,4,5,6-tetrahydro-2H-[1,4′]bipyridinyl-4-yl)ethyl]benzamide;
3-(1-Methyl-1H-benzoimidazol-2-yl)—N-[2-(3,4,5,6-tetrahydro-2H-[1,4′]bipyridinyl-4-yl)ethyl]benzamide;
3-(7-Chloro-1-oxo-1,3-dihydro-isoindol-2-yl)—N-[2-(3,4,5,6-tetrahydro-2H-[1,4′]bipyridinyl-4-yl)ethyl]-benzamide;
3-(7-Chloro-1-oxo-1,3-dihydro-isoindol-2-yl)—N-[2-(2′-cyano-3,4,5,6-tetrahydro-2H-[1,4′]bipyridinyl-4-yl)-ethyl]-benzamide;
7-Chloro-2-[3-(4-pyridin-4-yl-piperazine-1-carbonyl)-phenyl]-2,3-dihydro-isoindol-1-one;
7-Chloro-2-[3-(9-methyl-3,9-diaza-spiro[5.5]undecane-3-carbonyl)-phenyl]-2,3-dihydroisoindol-1-one;
9-[3-(7-Chloro-1-oxo-1,3-dihydro-isoindol-2-yl)-benzoyl₁ -3,9-diaza-spiro[5 .5]undecane-3-carboxylic acid tert-butyl ester;
7-Chloro-2-[3-(4-cyclohexyl-piperazine-1-carbonyl)-phenyl]-2,3-dihydro-isoindol-1-one;
5-(7-Chloro-1-oxo-1,3-dihydro-isoindol-2-yl)-2-hydroxy—N-[2-(3,4,5,6-tetrahydro-2H-[1,4′]bipyridinyl-4-yl)-ethyl]-benzamide;
7-Chloro-2-[3-(3,9-diaza-spiro[5 .5]undecane-3-carbonyl)-phenyl]-2,3-dihydro-isoindol-1-one;
7-Chloro-2-[3-(4-oxo-piperidine-1-carbonyl)-phenyl]-2,3-dihydro-isoindol-1-one;
3-(7-Chloro-1-oxo-1,3-dihydro-isoindol-2-yl)—N-[2-(5,6,7,8-tetrahydro-[1,8]naphthyridin-2-yl)-ethyl]-benzamide;
7-Chloro-2-[3-(9-pyridin-4-yl-3,9-diaza-spiro[5.5]undecane-3-carbonyl)-phenyl]-2,3-dihydro-isoindol-1-one;
3-(7-Chloro-1-oxo-1,3-dihydro-isoindol-2-yl)—N-[3-(5,6,7,8-tetrahydro-[1,8]naphthyridin-2-yl)-propyl]-benzamide;
7-Chloro-2-[3-(4-pyrimidin-2-yl-piperazine-1-carbonyl)-phenyl]-2,3-dihydro-isoindol-1-one;
2-[3-(4-Benzyl-piperazine-1-carbonyl)-phenyl]-7-chloro-2,3-dihydro-isoindol-1-one; 7-Chloro-2-[3-(4-phenethyl-piperazine-1-carbonyl)-phenyl]-2,3-dihydro-isoindol-1-one;
5-(7-Chloro-1-oxo-1,3-dihydro-isoindol-2-yl)-thiophene-2-carboxylic acid [2-(3,4,5,6-tetrahydro-2H-[1,4′]bipyridinyl-4-yl)-ethyl]-amide;
7-Chloro-2-{3-[4-(pyrrolidine-1-carbonyl)-piperazine-1-carbonyl]-phenyl}-2,3-dihydro-isoindol-1-one;
7-Chloro-2-{3-[4-(2-imidazol-1-yl-ethyl)-piperazine-1-carbonyl]-phenyl}-2,3-dihydro-isoindol-1-one;
7-Chloro-2-{3-[4-(2-pyrrol-1-yl-ethyl)-piperazine-1-carbonyl]-phenyl}-2,3-dihydro-isoindol-1-one;
7-Chloro-2-[3-(9-isopropyl-3,9-diaza-spiro[5.5]undecane-3-carbonyl)-phenyl]-2,3-dihydro-isoindol-1-one;
7-Chloro-2-[3-(4-cyclohexylmethyl-piperazine-1-carbonyl)-phenyl]-2,3-dihydro-isoindol-1-one;
7-Chloro-2-{3-[4-(2-dimethylamino-ethyl)-piperazine-1-carbonyl]-phenyl}-2,3-dihydro-isoindol-1-one;
7-Chloro-2-[3-(4-isopropyl-piperazine-1-carbonyl)-phenyl]-2,3-dihydro-isoindol-1-one; 2-{4-[3-(7-Chloro-1-oxo-1,3-dihydro-isoindol-2-yl)-benzoyl]-piperazin-1-yl}-N-pyridin-2-yl-acetamide;
7-Chloro-2-{3-[4-(1-methyl-piperidin-4-ylmethyl)-piperazine-1-carbonyl]-phenyl}-2,3-dihydro-isoindol-1-one;
7-Chloro-2-{3-[4-(3-pyrrolidin-1-yl-propyl)-piperazine-1-carbonyl]-phenyl}-2,3-dihydro-isoindol-1-one;
7-Chloro-2-[3-(4-pyridin-2-yl-piperazine-1-carbonyl)-phenyl]-2,3-dihydro-isoindol-1-one;
7-Chloro-2-{3-[4-(1-methyl-piperidin-4-yl)-piperazine-1-carbonyl]-phenyl}-2,3-dihydro-isoindol-1-one;
2-[3-([1,4′]Bipiperidinyl-1 ′-carbonyl)-phenyl]-7-chloro-2,3-dihydro-isoindol-1-one;
3-(7-Chloro-1-oxo-1,3 -dihydro-isoindol-2-yl)—N-[2-(4-piperidin-1-ylmethyl-phenyl)-ethyl]-benzamide;
7-Chloro-2-[5-(9-pyridin-4-yl-3 ,9-diaza-spiro[5 .5]undecane-3-carbonyl)-thiophen-2-yl]-2,3-dihydro-isoindol-1-one;
3-(4-Chloro-benzothiazol-2-yl)—N-[2-(3,4,5,6-tetrahydro-2H-[1,4′]bipyridinyl-4-yl)-ethyl]-benzamide;
3-Benzofuran-2-yl—N-[2-(3,4,5,6-tetrahydro-2H-[1,4′]bipyridinyl-4-yl)-ethyl]-benzamide;
[6-Chloro-3′-(4-chloro-1H-benzoimidazol-2-yl)-biphenyl-3-yl]-(4-pyridin-4-yl-piperazin-1-yl)-methanone;
3-Benzo[b]thiophen-2-yl—N-[2-(3,4,5,6-tetrahydro-2H-[1,4′]bipyridinyl-4-yl)-ethyl-1-benzamide;
2-[3-([4,4′]Bipiperidinyl-1-carbonyl)-phenyl]-7-chloro-2,3-dihydro-isoindol-1-one;
7-Chloro-2-[3-(3′,4′,5′,6′,3″,4″,5″,6″-octahydro-2′H,2″H-[4,1′;4′,4″]terpyridine-1″-carbonyl)-phenyl]-2,3-dihydro-isoindol-1-one;
7-Chloro-2-[3-( 1 ′-methyl-[4,4′]bipiperidinyl-1-carbonyl)-phenyl]-2,3-dihydro-isoindol-1-one;
7-Chloro-2-[3-( 1 ′-isopropyl-[4,4′]bipiperidinyl-1-carbonyl)-phenyl]-2,3-dihydro-isoindol-1-one;
7-Chloro-2-[3-(3′,4′,5′,6′,3″,4″,5″,6″-octahydro-2′H,2″H-[2,1′;4′,4″]terpyridine-1″-carbonyl)-phenyl]-2,3-dihydro-isoindol-1-one;
2-[3-(4-Amino-[1,4′]bipiperidinyl-1 ′-carbonyl)-phenyl]-7-chloro-2,3-dihydro-isoindol-1-one;
7-Chloro-2-{3-[4-(piperidin-4-yloxymethyl)-piperidine-1-carbonyl]-phenyl}-2,3-dihydro-isoindol-1-one;
7-Chloro-2-{3-[4-(pyridin-4-ylmethoxy)-piperidine-1-carbonyl]-phenyl}-2,3-dihydro-isoindol-1-one;
7-Chloro-2-{3-[4-(pyridin-4-yloxy)-piperidine-1-carbonyl]-phenyl}-2,3-dihydro-isoindol-1-one;
7-Chloro-2-[3-(4-dimethylaminomethyl-[1,4′]bipiperidinyl-1 ′-carbonyl)-phenyl]-2,3-dihydro-isoindol-1-one;
7-Chloro-2-[3-(4-phenoxy-piperidine-1-carbonyl)-phenyl]-2,3-dihydro-isoindol-1-one;
7-Chloro-2-{3-[4-(piperidin-4-yloxy)-piperidine-1-carbonyl]-phenyl}-2,3-dihydro-isoindol-1-one;
7-Chloro-2-{3-[4-(1-isopropyl-piperidin-4-yloxy)-piperidine-1-carbonyl]-phenyl}-2,3-dihydro-isoindol-1-one;
2-{3-[4-(4-Amino-phenyl)-piperidine-1-carbonyl]-phenyl}-7-chloro-2 ,3-dihydro-isoindol-1-one;
7-Chloro-2-[3-(5-pyridin-4-yl-3,4-dihydro-1H-isoquinoline-2-carbonyl)-phenyl]-2,3-dihydro-isoindol-1-one;
3-(7-Chloro- 1 H-benzoimidazol-2-yl)—N-[2-(3,4,5,6-tetrahydro-2H-[1,4′]bipyridinyl-4-yl)-ethyl]-benzamide;
3-Benzooxazol-2-yl—N-[2-(3,4,5,6-tetrahydro-2H-[1,4′]bipyridinyl-4-yl)-ethyl]-benzamide;
3-Benzothiazol-2-yl—N-[2-(3,4,5,6-tetrahydro-2H-[1,4′]bipyridinyl-4-yl)-ethyl]-benzamide;
3-Benzothiazol-2-yl-4-chloro-N-[2-(3,4,5,6-tetrahydro-2H-[1,4′]bipyridinyl-4-yl)-ethyl]-benzamide;
4-Chloro-3 -(4-chloro-1H-benzoimidazol-2-yl)—N-[2-(3,4,5,6-tetrahydro-2H-[1,4′]bipyridinyl-4-yl)-ethyl]-benzamide;
3-(7-Chloro-benzothiazol-2-yl)—N-[2-(3,4,5,6-tetrahydro-2H-[1,4′]bipyridinyl-4-yl)-ethyl]-benzamide;
{2-[3-(4-Chloro-1H-benzoimidazol-2-yl)-phenyl]-pyridin-4-yl}-(4-pyridin-4-yl-piperazin-1-yl)-methanone;
[3-(4-Chloro-benzothiazol-2-yl)-phenyl]-(9-pyridin-4-yl-3 ,9-diaza-spiro[5 .5]undec-3-yl)-methanone;
5-(7-Chloro-1-oxo-1,3-dihydro-isoindol-2-yl)-1H-pyrazole-3 -carboxylic acid [2-(3,4,5,6-tetrahydro-2H-[1,4′]bipyridinyl-4-yl)-ethyl]-amide; and
5-(7-Chloro-1-oxo-1,3-dihydro-isoindol-2-yl)-1H-pyrazole-3-carboxylic acid (2-oxo-5-phenyl-2,3-dihydro- 1H-benzo[e]+81,4]diazepin-3-yl)-amide.

45. The compound according to claim 15 selected from:
4-Chloro-2-{3-[5-(4-pyridin-4-yl-piperazin-1-ylmethyl)-pyridin-3-yl]-phenyl}-1H-benzoimidazole;
7-Chloro-2-{3-[5-(4-pyridin-4-yl-piperazin-1-ylmethyl)pyridin-3-yl]-phenyl}-2,3-dihydro-isoindol-1-one;
4-{3-[3-(4-Chloro-1H-benzoimidazol-2-yl)phenyl]-[1,2,4]oxadiazol-5-ylmethyl}-3,4,5,6-tetrahydro-2H-[1,4′]bipyridinyl;
4-{5-[3-(4-Chloro- 1 H-benzoimidazol-2-yl)phenyl]tetrazol-2-ylmethyl}-3,4,5,6-tetrahydro-2H-[1,4′]bipyridinyl;
4-{5-[3-(4-Chloro- 1 H-benzoimidazol-2-yl)-phenyl]-3H-[1,2,4]triazol-3-ylmethyl}-3,4,5,6-tetrahydro-2H-[1,4′]bipyridinyl;
7-Chloro-2-{3-[5-(4-pyridin-4-yl-piperazin-1-ylmethyl)-pyridin-3-yl]-phenyl}-2,3-dihydro-isoindol-1-one;
4-Chloro-2-{3-[5-(4-pyridin-4-yl-piperazin-1-ylmethyl)-thiophen-2-yl]-phenyl}-1H-benzoimidazole;
4-Chloro-2-(3-{5-[1-(4-pyridin-4-yl-piperazin-1-yl)-ethyl]-pyridin-3-yl}-phenyl)-1H-benzoimidazole;
4-Chloro-2-{3-[5-(4-pyridin-4-yl-piperazin-1-ylmethyl)-furan-2-yl]-phenyl}-1H-benzoimidazole;
4-{3-[3-(4-Chloro-1H-benzoimidazol-2-yl)-phenyl]-[1,2,4]oxadiazol-5-ylmethyl}-3,4,5,6-tetrahydro-2H-[1,4′]bipyridinyl;
4-Chloro-2-{3-[5-(4-pyridin-4-yl-piperazin-1-ylmethyl)-1H-imidazol-2-yl]-phenyl}-1H-benzoimidazole;
4-{5-[3-(4-Chloro-1H-benzoimidazol-2-yl)-phenyl]-tetrazol-2-ylmethyl}-3,4,5,6-tetrahydro-2H-[1,4′]bipyridinyl;
4-Chloro-2-[3′-(4-pyridin-4-yl-piperazin-1-ylmethyl)-biphenyl-3-yl]-1H-benzoimidazole;
4-{5-[3-(4-Chloro-1H-benzoimidazol-2-yl)-phenyl]-4H-[1,2,4]triazol-3-ylmethyl}-3,4,5,6-tetrahydro-2H-[1,4′]bipyridinyl;
4-Chloro-2-{3-[6-(4-pyridin-4-yl-piperazin-1-ylmethyl)-pyridin-2-yl]-phenyl}-1H-benzoimidazole;
4-Chloro-2-{3-[5-(4-pyridin-4-yl-piperazin-1-ylmethyl)-pyridin-3-yl]-phenyl}-1H-benzoimidazole;
{6-[3-(4-Chloro-1H-benzoimidazol-2-yl)-phenyl]-pyridin-2-yl}-(3,4,5,6-tetrahydro-2H-[1,4′]bipyridinyl-4-yl)-aniine;
{5-[3-(4-Chloro-1H-benzoimidazol-2-yl)-phenyl]-pyridin-3-yl}-(4-piperidin-1-ylmethyl-phenyl)-amine;
4-Chloro-2-{3-[4-(4-pyridin-4-yl-piperazin-1-ylmethyl)-pyridin-2-yl]-phenyl}-1H-benzoimidazole;
4-Chloro-2-[3-(5-piperidin-4-ylidenemethyl-pyridin-3-yl)-phenyl]-1H-benzoimidazole; and
4-Chloro-2-{3-[5-(4-pyridin-4-yl-piperazin-1-ylmethyl)-pyridin-3-yl]-phenyl}-benzothiazole.

6. The compound according to claim 23 selected from:
4-Chloro-2-[4′-(4-pyridin-4-yl-piperazin-1-ylmethyl)-biphenyl-3-yl]-1H-benzoimidazole; and
4-Chloro-2-[3′-(4-pyridin-4-yl-piperazin-1-ylmethyl)-biphenyl-3-yl]-1H-benzoimidazole.

47. The compound according to claim 36 selected from:
3-(4-Chloro-1H-benzoimidazol-2-yl)-piperidine-1-carboxylic acid [2-(3,4,5,6-tetrahydro-2H-[1,4′]bipyridinyl-4-yl)ethyl]amide;
3-(4-Chloro-1H-benzoimidazol-2-yl)-piperidine-1-carboxylic acid [2-(3,4,5,6-tetrahydro-2H-[1,4′]bipyridinyl-4-yl)-ethyl]-amide;
1-[3-(4-Chloro-1H-benzoimidazol-2-yl)-piperidin-1-yl]-4-(3,4,5,6-tetrahydro-2H-[1,4′]bipyridinyl-4-yl)-butan-1-one;
2-[3-(4-Chloro-1H-benzoimidazol-2-yl)-piperidin-1-yl]—N-(3,4,5,6-tetrahydro-2H-[1,4′]bipyridinyl-4-ylmethyl)-acetamide; and
2-[3-(4-Chloro-1H-benzoimidazol-2-yl)-piperidin-1-yl]—N-[2-(3,4,5,6-tetrahydro-2H-[1,4]bipyridinyl-4-yl)-ethyl]-acetamide.
